SEQUENCE LISTING (728 sequences)

SEQ ID NO: 1 - DNA, 3768 bp, Homo sapiens
  CDS (77)..(3766)
gccttggattttcaggttttcatcctgatacttgtttacttttctggggcagaaaagcttgcactaattgctctccatggtggctaattttttcaagagcttgattttaccttacattcataagctttgcaaaggaatgtttacaaagaaattgggaaatacaaacaaaaacaaagagtatcgtcagcagaaaaaggatcaagacttccccactgctggccagaccaaatcccccaaattttcttacacttttaaaagcactgtaaagaagattgcaaagtgttcatccactcacaacttatccactgaggaagacgaggccagtaaagagttttccctctcaccaacattcagttaccgagtagctattgccaatggcctacaaaagaatgctaaagtaaccaccagtgataatgaggatctgcttcaagagctctcttcaatcgagagttcctactcagaatcattaaatgaactaaggagtagcacagaaaaccaggcacaatcaacacacacaatgccagttagacgcaacagaaagagttcaagcagccttgcaccctctgagggcagctctgacggggagcgtactctacatggcttaaaactgggagctttacgaaaactgagaaaatggaaaaagagtcaagaatgtgtctcctcagactcagagttaagcaccatgaaaaaatcctggggaataagaagtaagtctttggacagaactgtccgaaacccaaagacaaatgccctggagccagggttcagttcctctggctgcattagccaaacacatgatgtcatggaaatgatctttaaggaacttcagggaataagtcagattgaaacagaactttctgaactacgagggcacgtcaatgctctcaagcactccatcgatgagatctccagcagtgtggaggttgtacaaagtgaaattgagcagttgcgcacagggtttgtccagtctcggagggaaactagagacatccatgattatattaagcacttaggtcatatgggtagcaaggcaagcctgagatttttaaatgtgactgaagaaagatttgaatatgttgaaagcgtggtgtaccaaattctaatagataaaatgggtttttcagatgcaccaaatgctattaaaattgaatttgctcagaggataggacaccagagagactgcccaaatgcaaagcctcgacccatacttgtgtactttgaaacccctcaacaaagggattctgtcttaaaaaagtcatataaactcaaaggaacaggcattggaatctcaacagatattctaactcatgacatcagagaaagaaaagagaaagggataccatcctcccagacatatgagagcatggctataaagttgtctactccagagccaaaaatcaagaagaacaattggcagtcacctgatgacagtgatgaagatcttgaatctgacctcaatagaaacagttacgctgtgctttccaagtcagagcttctaacaaagggaagtacttccaagccaagctcaaaatcacacagtgctagatccaagaataaaactgctaatagcagcagaatttcaaataaatcagattatgataaaatctcctcacagttgccagaatcagatatcttggaaaagcaaaccacaacccattatgcagatgcaacacctctctggcactcacagagtgattttttcactgctaaacttagtcgttctgaatcagatttttccaaattgtgtcagtcttactcagaagatttttcagaaaatcagtttttcactagaactaatggaagctctctcctgtcatcttcggaccgggagctatggcagaggaaacaggaaggaacagcgaccctgtatgacagtcccaaggaccagcatttgaatggaagtgttcagggtatccaagggcagactgaaactgaaaacacagaaactgtggatagtggaatgagtaatggcatggtgtgtgcatctggagaccggagtcattacagtgattctcagctctctttacatgaggatctttctccatggaaggaatggaatcaaggagctgatttaggcttggattcatccacccaggaaggttttgattatgaaacaaacagtctttttgaccaacagcttgatgtttacaataaagacctagaatacttgggaaagtgccacagtgatcttcaagatgactcagagagctacgacttaactcaagatgacaattcttctccatgccctggcttggataatgaaccacaaggccagtgggttggccaatatgattcttatcagggagctaattctaatgagctataccaaaatcaaaaccagttgtccatgatgtatcgaagtcaaagtgaattgcaaagtgatgattcagaggatgccccacccaaatcatggcatagtcgattaagcattgacctttctgataagactttcagcttcccaaaatttggatctacactgcagagggctaaatcagccttggaagtagtatggaacaaaagcacacagagtctgagtgggtatgaggacagtggctcttcattaatggggagatttcggacattatctcaatcaactgcaaatgagtcaagtaccacacttgactctgatgtctacacggagccctattactataaagcagaggatgaggaagattatactgaaccagtggctgacaatgaaacagattatgttgaagtcatggaacaagtccttgctaaactagaaaacaggactagtattactgaaacagatgaacaaatgcaagcatatgatcacctttcatatgaaacaccttatgaaaccccacaagatgagggttatgatggtccagcagatgatatggttagtgaagaggggttagaacccttaaatgaaacatcagctgagatggaaataagagaagatgaaaaccaaaacattcctgaacagccagtggagatcacaaagccaaagagaattcgtccttctttcaaagaagcagctttaagggcctataaaaagcaaatggcagagttggaagagaagatcttggctggagatagcagttctgtggatgaaaaggctcgaatagtaagtggcaatgatttggatgcttccaaattttctgcactccaggtgtgtggtggggctggaggtggactttatggtattgacagcatgccggatcttcgcagaaaaaaaactttgcctattgtccgagatgtggccatgaccctggctgcccggaaatctggactctccctggctatggtgattaggacatccctaaataatgaggaactgaaaatgcacgtcttcaagaagaccttgcaggcactgatctaccctatgtcttctaccatcccacacaattttgaggtctggacggctaccacacccacctactgttatgagtgtgaagggctcctgtggggcattgcaaggcaaggcatgaagtgtctggagtgtggagtgaaatgccacgaaaagtgtcaggacctgctaaacgctgactgcttgcagagagcagcagaaaagagttctaaacatggtgccgaagacaagactcagaccattattacagcaatgaaagaaagaatgaagatcagggagaaaaaccggccagaagtatttgaagtaatccaggaaatgtttcagatttctaaagaagattttgtgcagtttacaaaggcggccaaacagagtgtactggatgggacatctaagtggtctgcaaaaataaccatcacagtggtttctgcacaagg

SEQ ID NO: 2 - protein, 1230 aa, Homo sapiens
MVANFFKSLILPYIHKLCKGMFTKKLGNTNKNKEYRQQKKDQDFPTAGQTKSPKFSYTFKSTVKKIAKCSSTHNLSTEEDEASKEFSLSPTFSYRVAIANGLQKNAKVTTSDNEDLLQELSSIESSYSESLNELRSSTENQAQSTHTMPVRRNRKSSSSLAPSEGSSDGERTLHGLKLGALRKLRKWKKSQECVSSDSELSTMKKSWGIRSKSLDRTVRNPKTNALEPGFSSSGCISQTHDVMEMIFKELQGISQIETELSELRGHVNALKHSIDEISSSVEVVQSEIEQLRTGFVQSRRETRDIHDYIKHLGHMGSKASLRFLNVTEERFEYVESVVYQILIDKMGFSDAPNAIKIEFAQRIGHQRDCPNAKPRPILVYFETPQQRDSVLKKSYKLKGTGIGISTDILTHDIRERKEKGIPSSQTYESMAIKLSTPEPKIKKNNWQSPDDSDEDLESDLNRNSYAVLSKSELLTKGSTSKPSSKSHSARSKNKTANSSRISNKSDYDKISSQLPESDILEKQTTTHYADATPLWHSQSDFFTAKLSRSESDFSKLCQSYSEDFSENQFFTRTNGSSLLSSSDRELWQRKQEGTATLYDSPKDQHLNGSVQGIQGQTETENTETVDSGMSNGMVCASGDRSHYSDSQLSLHEDLSPWKEWNQGADLGLDSSTQEGFDYETNSLFDQQLDVYNKDLEYLGKCHSDLQDDSESYDLTQDDNSSPCPGLDNEPQGQWVGQYDSYQGANSNELYQNQNQLSMMYRSQSELQSDDSEDAPPKSWHSRLSIDLSDKTFSFPKFGSTLQRAKSALEVVWNKSTQSLSGYEDSGSSLMGRFRTLSQSTANESSTTLDSDVYTEPYYYKAEDEEDYTEPVADNETDYVEVMEQVLAKLENRTSITETDEQMQAYDHLSYETPYETPQDEGYDGPADDMVSEEGLEPLNETSAEMEIREDENQNIPEQPVEITKPKRIRPSFKEAALRAYKKQMAELEEKILAGDSSSVDEKARIVSGNDLDASKFSALQVCGGAGGGLYGIDSMPDLRRKKTLPIVRDVAMTLAARKSGLSLAMVIRTSLNNEELKMHVFKKTLQALIYPMSSTIPHNFEVWTATTPTYCYECEGLLWGIARQGMKCLECGVKCHEKCQDLLNADCLQRAAEKSSKHGAEDKTQTIITAMKERMKIREKNRPEVFEVIQEMFQISKEDFVQFTKAAKQSVLDGTSKWSAKITITVVSAQ

SEQ ID NO: 3 - DNA, 10625 bp, Homo sapiens
  CDS (544)..(6960)
cttattttgaaaacatttacatagtgattagttaacccaacagaccaatcctgggaagacagccagagcctgcagcaccttagtaacagaaaaactgataattaggagaagagacctgtccaagaccaggaacctggaccaaaattgtgccatgttgctttactttaatgagtggccccagtaaaaactgagctgtatggcagagctgttcacatttatcttctgtgtccacccagttctgctgaaacccctggcaagatcgtggccctgttgtagcttgtcatgttttgaacagctgtctatggaaagaaagcaaacacaacctagagcaacattgatttgttttagaaagctcttttattttcagttctggctgtgttcaacatcttagcttacgtttttcatgttgtaatgatctgccgtatggacgatcacctctaagttagagagttctgtaatttggcttggattaaagatgcttggttagtgaaagctgctgctttttttatagtcaaaggactggttctgagagccttgttgcagatggctgaggtcaccgtcccaagggtgtatgtcgtgtttggcatccattgcatcatggcgaaggcatcttcagatgtgcaggtttcaggctttcatcggaaaatccagcacgttaaaaatgaactttgccacatgttgagcttggaggaggtggccccagtgctgcagcagacattacttcaggacaacctcttgggcagggtacattttgaccaatttaaagaagcattaatactcatcttgtccagaactctgtcaaatgaagaacactttcaagaaccagactgctcactagaagctcagcccaaatatgttagaggtgggaagcgttacggacgaaggtccttgcccgagttccaagagtccgtggaggagtttcctgaagtgacggtgattgagccactggatgaagaagcgcggccttcacacatcccagccggtgactgcagtgagcactggaagacgcaacgcagtgaggagtatgaagcggaaggccagttaaggttttggaacccagatgacttgaatgcttcacagagtggatcttcccctccccaagactggatagaagagaaactgcaagaagtttgtgaagatttggggatcacccgtgatggtcacctgaaccggaagaagctggtctccatctgtgagcagtatggtttacagaatgtggatggagagatgctcgaggaagtattccataatcttgatcctgacggtacaatgagtgtagaagattttttctatggtttgtttaaaaatggaaaatctcttacaccatcagcatctactccatatagacaactaaaaaggcacctttccatgcagtctttcgatgagagtggacgacgtaccacaacctcatcagcaatgacaagtaccattggctttcgggtcttctcctgcctggatgatgggatgggccatgcatctgtggagagaatactggacacctggcaggaagagggcattgagaacagccaggagatcctgaaggccttggatttcagcctcgatggaaacatcaatttgacagaattaacactggcccttgaaaatgaacttttggttaccaagaacagcattcaccaggcggctctggccagctttaaggctgaaatccggcatttgttggaacgagttgatcaggtggtcagagaaaaagagaagctacggtcagatctggacaaggccgagaagctcaagtctttaatggcctcggaggtggatgatcaccatgcggccatagagcggcggaatgagtacaacctcaggaaactggatggagagtacaaggagcgaatagcagccttaaaaaatgaactccgaaaagagagagagcagatcctgcagcaggcaggcaagcagcgtttagaacttgaacaggaaattgaaaaggcaaaaacagaagagaactatatccgggaccgccttgccctctctttaaaggaaaacagtcgtctggaaaatgagcttctagaaaatgcagagaagttggcagaatatgagaatctgacaaacaaacttcagagaaatttggaaaatgtgttagcagaaaagtttggtgacctcgatcctagcagtgctgagttcttcctgcaagaagagagactgacacagatgagaaatgaatatgagcggcagtgcagggtactacaagaccaagtagatgaactccagtctgagctggaagaatatcgtgcacaaggcagagtgctcaggcttccgttgaagaactcaccgtcagaagaagttgaggctaacagcggtggcattgagcccgaacacgggctcggttctgaagaatgcaatccattgaatatgagcattgaggcagagctggtcattgaacagatgaaagaacaacatcacagggacatatgttgcctcagactggagctcgaagataaagtgcgccattatgaaaagcagctggacgaaaccgtggtcagctgcaagaaggcacaggagaacatgaagcaaaggcatgagaacgaaacgcgcaccttagaaaaacaaataagtgaccttaaaaatgaaattgctgaacttcaggggcaagcagcagtgctcaaggaggcacatcatgaggccacttgcaggcatgaggaggagaaaaaacaactgcaagtgaagcttgaggaggaaaagactcacctgcaggagaagctgaggctgcaacatgagatggagctcaaggctagactgacacaggctcaagcaagctttgagcgggagagggaaggccttcagagtagcgcctggacagaagagaaggtgagaggcttgactcaggaactagagcagtttcaccaggagcagctgacaagcctggtggagaaacacactcttgagaaagaggagttaagaaaagagctcttggaaaagcaccaaagggagcttcaggagggaagggaaaaaatggaaacagagtgtaatagaagaacctctcaaatagaagcccagtttcagtctgattgtcagaaagtcactgagaggtgtgaaagcgctctgcaaagcctggaggggcgctaccgccaagagctgaaggacctccaggaacagcagcgtgaggagaaatcccagtgggaatttgagaaggacgagctcacccaggagtgtgcggaagcccaggagctgctgaaagagactcttaagagagagaaaacaacttctctggtcctgacccaggagagagagatgctggagaaaacatacaaagaacatttgaacagcatggtcgtcgagagacagcagctactccaagacctggaagacctaagaaatgtatctgaaacccagcaaagcctgctgtctgaccagatacttgagctgaagagcagtcacaaaagggaactgagggagcgtgaggaggtcctgtgccaggcaggggcttcggagcagctggccagccagcggctggaaagactagaaatggaacatgaccaggaaaggcaggaaatgatgtccaagcttctagccatggagaacattcacaaagcgacctgtgagacagcagatcgagaaagagccgagatgagcacagaaatctccagacttcagagtaaaataaaggaaatgcagcaggcaacatctcctctctcaatgcttcagagtggttgccaggtgataggagaggaggaggtggaaggagatggagccctgtccctgcttcagcaaggggagcagctgttggaagaaaatggggacgtcctcttaagcctgcagagagctcatgaacaggcagtgaaggaaaatgtgaaaatggctactgaaatttctagattgcaacagaggctacaaaagttagagccagggttagtaatgtcttcttgtttggatgagccagctactgagttttttggaaatactgcggaacaaacagagcagtttttacagcaaaaccgaacgaagcaagtagaaggtgtgaccaggcggcatgtcctaagtgacctggaagatgatgaggtccgggacctgggaagtacagggacgagctctgttcagagacaggaagtcaaaatagaggagtctgaagcttcagtagagggtttttctgagcttgaaaacagtgaagagaccaggactgaatcctgggagctgaagaatcagattagtcagcttcaggaacagctaatgatgttatgtgcggactgtgatcgagcttctgaaaagaaacaggacctactttttgatgtttctgtgctaaaaaagaaactgaagatgcttgagagaatccctgaggcttctcccaaatataagctgttgtatgaagatgtgagccgagaaaatgactgccttcaggaagagctgagaatgatggagacacgctacgatgaggcactagaaaataacaaagaactcactgcagaggttttcaggttgcaggatgagctgaagaaaatggaggaagtcactgaaacattcctcagcctggaaaagagttacgatgaggtcaaaatagaaaatgaggggctgaatgttctggttttgagacttcaaggcaagattgagaagcttcaggaaagcgtggtccagcggtgtgactgctgcttatgggaagccagtttagagaacctggaaatcgaacctgatggaaatatactccagctcaatcagacactggaagagtgtgtgcccagggttaggagtgtacatcatgtcatagaggaatgtaagcaagaaaaccagtaccttgaggggaacacacagctcttggaaaaagtaaaagcacatgaaattgcctggttacatggaacaattcagacacatcaagaaaggccaagagtacagaatcaagttatactggaggaaaacactactctcctaggctttcaagacaaacattttcagcatcaggccaccatagcagagttagaactggagaaaacaaagttacaggagctgactaggaagttgaaggagagagtcactattttagttaagcaaaaagatgtactttctcacggagaaaaggaggaagagctgaaggcaatgatgcatgacttgcagatcacgtgcagtgagatgcagcaaaaagttgaacttctgagatatgaatctgaaaagcttcaacaggaaaattctattttgagaaatgaaattactactttaaatgaagaagatagcatttctaacctgaaattagggacattaaatggatctcaggaagaaatgtggcaaaaaacggaaactgtaaaacaagaaaatgctgcagttcagaagatggttgaaaatttaaagaaacagatttcagaattaaaaatcaaaaaccaacaattggatttggaaaatacagaacttagccaaaagaactctcaaaaccaggaaaaactgcaagaacttaatcaacgtctaacagaaatgctatgccagaaggaaaaagagccaggaaacagtgcattggaggaacgggaacaagagaagtttaatctgaaagaagaactggaacgttgtaaagtgcagtcctccactttagtgtcttctctggaggcggagctctctgaagttaaaatacagacccatattgtgcaacaggaaaaccaccttctcaaagatgaactggagaaaatgaaacagctgcacagatgtcccgatctctctgacttccagcaaaaaatctctagtgttctaagctacaacgaaaaactgctgaaagaaaaggaagctctgagtgaggaattaaatagctgtgtcgataagttggcaaaatcaagtcttttagagcatagaattgcgacgatgaagcaggaacagaaatcctgggaacatcagagtgcgagcttaaagtcacagctggtggcttctcaggaaaaggttcagaatttagaagacaccgtgcagaatgtaaacctgcaaatgtcccggatgaaatctgacctacgagtgactcagcaggaaaaggaggctttaaaacaagaagtgatgtctttacataagcaacttcagaatgctggtggcaagagctgggccccagagatagctactcatccatcagggctccataaccagcagaaaaggctgtcctgggacaagttggatcatctgatgaatgaggaacagcagctgctttggcaagagaatgagaggctccagaccatggtacagaacaccaaagccgaactcacgcactcccgggagaaggtccgtcaattggaatccaatcttcttcccaagcaccaaaaacatctaaacccatcaggtaccatgaatcccacagagcaagaaaaattgagcttaaagagagagtgtgatcagtttcagaaagaacaatctcctgctaacaggaaggtcagtcagatgaattcccttgaacaagaattagaaacaattcatttggaaaatgaaggcctgaaaaagaaacaagtaaaactggatgagcagctcatggagatgcagcacctgaggtccactgcgacgcctagcccgtcccctcatgcttgggatttgcagctgctccagcagcaagcctgtccgatggtgcccagggagcagtttctgcagcttcaacgccagctgctgcaggcagaaaggataaaccagcacctgcaggaggaacttgaaaacaggacctccgaaaccaacacaccacagggaaaccaggaacaactggtaactgtcatggaggaacgaatgatagaagttgaacagaaactgaaactagtgaaaaggcttcttcaagagaaagtgaatcagctcaaagaacaactctgcaagaacactaaggcagacgcaatggtgaaggacttgtatgttgaaaatgcccagttgttgaaagctctggaagtgactgaacagcgacagaaaacagcagagaagaaaaattacctcctggaggagaagattgccagcctcagtaatatagttaggaatctgacaccagcgccattgacttctacacctcctttgaggtcatagccaaaccaaagggtacactcatatttgtgcactttactgaaatagatgaacatttcagtaggttctcaacttaaaattaagcctaacctaaaactgccagcaacacaactggagtttccatttatcataattagtttttctaaatagacccttatgggagtttgaaaataaatactcacatatttcactacttaaattattcccaagatttgaatttattttaaaattttaatagccaccaagaatgtggacatatgaaaattcaagaacctaaaaaataccagttttgaatgagtttttgtggttttggttttttaattattacaaatctatgtgtaaaatctagatatttgaagtttgagatctgatgagaatggttgttataaactttattttaaaaccaaatttaggtgttcttacatatttaaatactggaaagtcattataatagttttggttctttgaattggtagacaattagtagagtataattggttaggaggcagggcttattaagtggttattaaccgctgacatcagacaaacccaaatctgtagaattctaacctcctaacacctgtgacagtattaccactcttcttgtattatagatttagaactgatttactcaattgcactcttaactaatgttaaaagcttacttgctttaaacagccttttcttctttctcttaaaagtttcatttggggagctggtcttctaagaaacggataaagccacataattaaagcagttgaactagagggaaagcactgaacaaaccactttggagtaaatagctactcttagaaaagagggataagcagaccatgtaggttttctgtctctcaaatcttagagttcataaatttacttgaggttgcctcaagaactcagggaacaatactgtaaactgtcttcctgaactactgtagggcctctctaagaatttgaaatgtataaaccatgtgacctcatttatttgtcttatatatttacagccatactagaatttttatttctacgtttttagtaaatttaatattctgggggaaaaaaggccttgattttagggttaaaaacctgacttatagaagagtttatttaatataggtcaaaattttctgtgtttcttattccttctatacctcaaatctgattctaagaatttcttactgtgataatcattggcatgccacctgaggtcaaggagtgccaaataggactttccactcatgctcaagatcaaaactttatagaacagtcaacattttagattcggtaaccttttttttcttccaattataatctctgcttctagccacttccgccagcagttggtggaagacttactaggtgcagggcactttccaagttcatcacaacaacctgcttgttttcatgagacaataatccgaaaagttcgctttgatatattcctggagggccaagcccatctatttacaaaaggtgaacagcaaaatcaagcactgctttatgggcaggaacacaagagaaagcaaactgcccaagaagtcatcatgtcagaaactcaatctcaacaaaataatttccatcagggaacttcagggtttcttgggggcttatgagtctcaccggtcaacccaggaggcctcactacaagagccttgacaaggcactgttttttgtgggactgggagttcacactgatgaagcaaacctttgaatttttgcacagctcttgtcagaaagccctgagttccccctggataaagagttaattttaatccttccctataattatacttcaaaatatttgacatctgctattatgccttctttagatctttcttctgcggtgcagacatttctagtaagtgtttgactacttgtatggcattagctttcacagaaaattgtttcacttaaaactgtggattggcctaggctaaggacaaaaataaactaagtacctgtagtgtatttatgtgatatgtgtcaagttactcaaagttattgctgttggaactgaacaataatatttcccagatagctggccttagcatgtgatcacggttgttgtatttttaatttttgtcttttacagtatgagaggtgtaggttaatttgtttatttcctataaatttgtatttatgtgtatataaaatgtacaatgaatgtaaatatgactttctggaaagtttagactacatttagaatctctattcaaaatcaaaatgctgctcaaatgaatttaaccaacatctaggtgcttaatttctcattttatcccacttatgagattgggaaaaagatcaatatgagaaataccatacagataccttaaatgtatgcatttgtgcaacaatttttgagaaggtgagtggcaatttataatttagttggcaatttataatagaacttatagcttttaaaagactttttaaagacattaaatgtaaacttaaaaatgtttagatcttgtttcaaactttacaatagcattcttcaaaatattaagttatatattttataggcatttagttgcttattaaaagcactgattttcaaactttttgatttaagaacaattatttaagatcgtctcagaagatgggatcttcgtttcaagaaaagggaatcaagtttgcctttgagataatacgttacactaagaaaaggaaaatgtggatagtaaaacccacctctctcatcctattgtactctcttctgctttttagaagcctgcacttaagcttagatttgtgaagggagagtagaaggggagaagtagaaccacagtgttttatttatttttctaaaactcttactaaatccagattttttaaactgttttaaatgtgaattcttcccagaaatttcaatgcattgcatatttagccttcggcatatttttcatgaatagatcatgaagtcataggcttccaaggcataggaagagatcttgcaggtctagtattttaataatgcactattacccagggcagatattatgagaaactgtttcttctctaagggtttatggcagactttgcttttttaacatgtgagaaatgaattttttattttgtgatttatgtgatttcttttgctgagtgaaggaaaggagaaattgttgctattgtcagcatcttaaaggtatttccagtcaaggcaaggctaagtgctttgtgatagtattaagcaagtcatgttttgaatggattacctgtagtgactcattggaatgatataattatacaagtaatgccaaaaaccaagtcaaagcctaattaaccaaagcactcatttaaaaatcatcatgtttggacctatctggacctctcagcactgtaaaatagttttggttttgtggcatatgaatagctgtttaacaaatcaaagttagctttttgcttctcagcttttttgggcaatacaagttaagttcttaatggggagacattatcatggcatgacttaagggaacattggtttgtgaaggaaaaacagattatctaaagccatctctatgtttctgttcagataaagattaatgagttctgtgtttatatcagctttgtatatttcatcttagccattctatcctagaaagattttaatgtgagcttaagatgtaaataaataattttgcaaacatgaaaaaaaaaaaaaaaaa

SEQ ID NO: 4 - protein, 2139 aa, Homo sapiens
MAEVTVPRVYVVFGIHCIMAKASSDVQVSGFHRKIQHVKNELCHMLSLEEVAPVLQQTLLQDNLLGRVHFDQFKEALILILSRTLSNEEHFQEPDCSLEAQPKYVRGGKRYGRRSLPEFQESVEEFPEVTVIEPLDEEARPSHIPAGDCSEHWKTQRSEEYEAEGQLRFWNPDDLNASQSGSSPPQDWIEEKLQEVCEDLGITRDGHLNRKKLVSICEQYGLQNVDGEMLEEVFHNLDPDGTMSVEDFFYGLFKNGKSLTPSASTPYRQLKRHLSMQSFDESGRRTTTSSAMTSTIGFRVFSCLDDGMGHASVERILDTWQEEGIENSQEILKALDFSLDGNINLTELTLALENELLVTKNSIHQAALASFKAEIRHLLERVDQVVREKEKLRSDLDKAEKLKSLMASEVDDHHAAIERRNEYNLRKLDGEYKERIAALKNELRKEREQILQQAGKQRLELEQEIEKAKTEENYIRDRLALSLKENSRLENELLENAEKLAEYENLTNKLQRNLENVLAEKFGDLDPSSAEFFLQEERLTQMRNEYERQCRVLQDQVDELQSELEEYRAQGRVLRLPLKNSPSEEVEANSGGIEPEHGLGSEECNPLNMSIEAELVIEQMKEQHHRDICCLRLELEDKVRHYEKQLDETVVSCKKAQENMKQRHENETRTLEKQISDLKNEIAELQGQAAVLKEAHHEATCRHEEEKKQLQVKLEEEKTHLQEKLRLQHEMELKARLTQAQASFEREREGLQSSAWTEEKVRGLTQELEQFHQEQLTSLVEKHTLEKEELRKELLEKHQRELQEGREKMETECNRRTSQIEAQFQSDCQKVTERCESALQSLEGRYRQELKDLQEQQREEKSQWEFEKDELTQECAEAQELLKETLKREKTTSLVLTQEREMLEKTYKEHLNSMVVERQQLLQDLEDLRNVSETQQSLLSDQILELKSSHKRELREREEVLCQAGASEQLASQRLERLEMEHDQERQEMMSKLLAMENIHKATCETADRERAEMSTEISRLQSKIKEMQQATSPLSMLQSGCQVIGEEEVEGDGALSLLQQGEQLLEENGDVLLSLQRAHEQAVKENVKMATEISRLQQRLQKLEPGLVMSSCLDEPATEFFGNTAEQTEQFLQQNRTKQVEGVTRRHVLSDLEDDEVRDLGSTGTSSVQRQEVKIEESEASVEGFSELENSEETRTESWELKNQISQLQEQLMMLCADCDRASEKKQDLLFDVSVLKKKLKMLERIPEASPKYKLLYEDVSRENDCLQEELRMMETRYDEALENNKELTAEVFRLQDELKKMEEVTETFLSLEKSYDEVKIENEGLNVLVLRLQGKIEKLQESVVQRCDCCLWEASLENLEIEPDGNILQLNQTLEECVPRVRSVHHVIEECKQENQYLEGNTQLLEKVKAHEIAWLHGTIQTHQERPRVQNQVILEENTTLLGFQDKHFQHQATIAELELEKTKLQELTRKLKERVTILVKQKDVLSHGEKEEELKAMMHDLQITCSEMQQKVELLRYESEKLQQENSILRNEITTLNEEDSISNLKLGTLNGSQEEMWQKTETVKQENAAVQKMVENLKKQISELKIKNQQLDLENTELSQKNSQNQEKLQELNQRLTEMLCQKEKEPGNSALEEREQEKFNLKEELERCKVQSSTLVSSLEAELSEVKIQTHIVQQENHLLKDELEKMKQLHRCPDLSDFQQKISSVLSYNEKLLKEKEALSEELNSCVDKLAKSSLLEHRIATMKQEQKSWEHQSASLKSQLVASQEKVQNLEDTVQNVNLQMSRMKSDLRVTQQEKEALKQEVMSLHKQLQNAGGKSWAPEIATHPSGLHNQQKRLSWDKLDHLMNEEQQLLWQENERLQTMVQNTKAELTHSREKVRQLESNLLPKHQKHLNPSGTMNPTEQEKLSLKRECDQFQKEQSPANRKVSQMNSLEQELETIHLENEGLKKKQVKLDEQLMEMQHLRSTATPSPSPHAWDLQLLQQQACPMVPREQFLQLQRQLLQAERINQHLQEELENRTSETNTPQGNQEQLVTVMEERMIEVEQKLKLVKRLLQEKVNQLKEQLCKNTKADAMVKDLYVENAQLLKALEVTEQRQKTAEKKNYLLEEKIASLSNIVRNLTPAPLTSTPPLRS

SEQ ID NO: 5 - DNA, 9274 bp, Homo sapiens
  CDS (1)..(8505)
tggcttgtggaaaaagaacttatggtcagtgttcttgggcccttgtcaattgacccaaatatgctaaacacacaaaggcagcaggtgcagattttgctgcaagaattcgccactcggaaacctcaatatgaacagctgacagcagctggtcagggcattctgagcaggcctggagaagacccttctttacgtgggattgtgaaagagcaactggcagctgtgacccaaaaatgggatagcctaacagggcaattgagtgacagatgtgactggattgaccaagccattgttaaaagcacacagtatcaaagcctgctgagaagcctttctgataaactgagtgacttggataataaactcagcagcagtctggctgtgagcacgcaccctgatgctatgaaccaacagttggaaacagcccaaaaaatgaagcaggagatacagcaggaaaagaagcagataaaagtggcccaggcactctgtgaggatttgtcagcactggttaaagaagagtacttgaaagcagaacttagtaggcaactagaaggcatcttaaaatcatttaaggatgttgaacagaaagcagagaatcatgtccagcaccttcagtcggcctgtgcaagctctcatcaatttcagcaaatgtctagagattttcaggcttggctggatacaaagaaagaagagcaaaacaaatctcatccaatatctgccaaactcgatgtcttggagtcattaattaaagatcataaagactttagtaaaactttgaccgctcagtctcatatgtatgaaaaaaccattgcagaaggtgaaaatctgttattaaaaacacaagggtctgagaaggcagccttacagttacagcttaatacaattaaaaccaattgggatacatttaataagcaggtgaaagaaagagaaaacaagttaaaagagtcattggaaaaagcccttaagtataaagagcaagtagagactctctggccatggatagacaaatgccaaaacaacctggaggaaataaaattttgcttggatcctgctgaaggagagaattctattgccaagttaaagtctctgcagaaggaaatggaccaacactttggtatggtagaattactgaacaacacagccaatagcttgctcagtgtctgtgagatagataaagaagttgttacagatgagaataagtcactgatccagaaggtggacatggtcactgaacaacttcacagtaagaaattctgtctggagaacatgactcagaagtttaaagaatttcaagaagtttccaaagaatctaaaaggcagcttcagtgtgcaaaggagcagctagatatccatgattcgctgggatcccaggcttacagtaacaaatacctgaccatgttgcaaactcagcagaaatcacttcaggccttgaagcatcaggtagatttggctaaaagacttgcacaggaccttgtggtagaggcctcagactcaaagggaacctctgatgttttattacaagtggaaaccatagctcaagagcatagtacactaagtcagcaggttgatgaaaagtgttctttcttagaaaccaagcttcagggcattgggcatttccagaataccattcgagaaatgttttctcagttcgcagagtttgatgatgaactggatagcatggctccagtggggagagatgcagaaacattgcaaaagcaaaaggaaactataaaagcctttctaaagaaactagaagccctcatggcaagcaatgacaatgccaataaaacctgcaagatgatgttagccacagaagaaacctctcctgaccttgttggaatcaaaagggacttggaggccttaagcaaacaatgcaacaagttactggaccgagcccaagccagagaagagcaggttgaagggacaattaagcgccttgaagaattttacagcaaattgaaagaattttctattctgctccagaaagccgaagaacatgaagagtcacaaggtcctgttggtatggaaacggagacaattaatcagcagcttaacatgttcaaggtattccagaaagaagagattgaacccttgcaaggtaaacagcaagatgtaaactggttaggtcaaggccttattcagagtgctgccaaaagcactagcactcagggcttggagcatgacctggatgatgtcaatgcacggtggaagactctcaataagaaggtggctcagcgagcagcccagctgcaggaggccttgctgcactgtgggaggttccaggatgccctggagtccctgctcagctggatggtggacactgaggagcttgtggccaatcagaagcccccgtcggctgagttcaaagtggtaaaggcccagatacaagaacaaaagcttctccagagattgttggatgaccgaaaatctacggtggaggtaatcaaacgagaaggagaaaaaattgctacaacagcagagcccgcagataaagtgaagattttgaaacagctcagtctcttggatagcagatgggaggcattgcttaataaagctgaaacaaggaatcgtcagttggaaggtatctcggtggtagcacagcaatttcatgaaaccttagaaccactgaacgagtggcttacaaccatagaaaagaggctggtgaattgtgaacccataggaacccaagcatctaaacttgaggaacaaattgcacagcacaaagttctgcaagaggacatcttactcaggaaacaaaatgtagatcaggctttactaaatggtttagaactacttaaacaaaccacaggtgatgaagttttaataattcaagataaattggaagccattaaagcaaggtacaaagacattactaaactgagcactgatgtggccaagactctggaacaggcgctgcagcttgcaaggcggctgcactccacacacgaagagctgtgtacctggctggacaaagtggaggtggaattactttcatatgaaactcaggttctgaaaggagaagaagcaagtcaagcacaaatgagaccaaaggaactgaaaaaggaagctaagaacaacaaagccttactggactcccttaatgaagtgagcagtgctttgctggaactggtaccatggagggcaagagaaggacttgagaaaatggtagctgaggacaatgagcgctaccgattagtgagcgacaccatcactcagaaggtggaggagatcgatgcagccattctgcgatcacagcagtttgaccaagcagctgatgctgagttatcctggattactgaaacagaaaaaaaattgatgtctctgggtgacatcaggcttgagcaagaccagacttctgctcagcttcaagttcaaaagacattcaccatggagattttgagacacaaggatattattgatgaccttgttaaatctgggcataaaatcatgaccgcatgcagtgaagaggaaaagcaatcaatgaagaaaaaactggacaaggtactgaagaactatgataccatctgccagattaattcagaaaggtatctgcagctggaacgggcacagtccctggttaaccaattctgggaaacatatgaagaactttggccatggctgacagaaacacaatcaatcatctctcagcttcccgccccagcccttgaatatgaaactctaaggcagcagcaggaagaacatcggcaactgcgtgagttgatagctgaacacaagcctcatatagataagatgaacaaaactgggccacagttactggaattgagccctggggaaggcttttctatccaagagaagtatgtggcagccgacaccctttacagtcaaattaaagaagatgtcaaaaagcgtgctgtggcactggatgaagccatttctcaatcaactcagttccatgacaagatagatcagatccttgagagcctggaacgcatcgtggaacgtctgaggcagccaccctctatctctgcagaggttgagaagatcaaggaacagatcagtgaaaataagaatgtgtcagtagacatggaaaagctacagccgttgtatgaaactcttaaacagaggggagaggaaatgattgctagatctggggggactgataaagacatatctgccaaagctgttcaggataagcttgaccaaatggttttcatttgggagaacatacacacactggtggaagagagggaagccaaactactggatgtgatggagctagcagaaaagttctggtgtgatcacatgtcattgatagttaccattaaagatactcaagatttcatccgggacctggaagatcctggaattgatccttcagtagtaaaacaacagcaagaagcagcagagaccataagggaagaaatagatggactacaggaggagctggatatagttattaacctaggttctgaactcattgcggcatgtggggagcctgataaacccattgtcaagaagagtatagatgagttaaattcagcatgggattctctaaataaagcttggaaagaccggattgacaaacttgaggaggcaatgcaggctgccgttcagtaccaggatggactgcaggcggtatttgactgggtagatattgcaggtggtaaattagcttcaatgtctccaattggaacagatctcgaaactgtcaagcagcagattgaagagctaaagcaatttaagtctgaggcctatcaacagcagatagaaatggaaagactgaatcatcaagcagagcttttgctaaagaaagtaacagaagagagtgacaaacacactgttcaagacccattaatggaactgaaattgatatgggatagcctggaggagagaatcatcaacagacagcataaactggagggtgctctattagccttgggtcagttccaacatgccctggatgagctcctggcatggctgacacacaccgagggcttgctaagtgagcagaaacctgttggaggagaccctaaagccattgaaattgaacttgccaagcatcatgtgctccaaaatgatgtattagcccatcagtccacagtggaagccgttaataaagcaggaaatgatctaattgaatcaagtgcaggagaagaagcaagcaaccttcagaacaagctagaggttttaaatcaacgctggcaaaatgttttggaaaaaacagaacaaaggaagcagcagctggatggtgccttgcgccaggccaaagggttccatggcgaaattgaggatttgcagcagtggctgactgacacggagcgtcatctgttggcatctaaaccgctgggaggtttaccggaaacagccaaggagcagcttaatgtccatatggaagtctgtgctgcctttgaagctaaagaagaaacatataagagtctgatgcagaaaggccagcagatgcttgcaagatgcccaaaatctgcagagacaaatattgaccaagacataaataacttgaaagaaaaatgggaatcggtggaaaccaaactcaatgaaaggaaaactaaactggaagaggctctcaacttggcaatggagttccacaattctctccaagacttcatcaactggcttactcaggctgaacagaccctaaatgtagcttctcggccaagtctcatcttggacacagtcttatttcaaattgacgaacacaaggtttttgccaatgaagtaaattctcatcgtgagcagataatagagctggacaaaactggaacccacctaaaatattttagtcagaaacaagatgttgttctaatcaagaatctacttatcagtgtacaaagtcgatgggaaaaagtggttcaacggttggtagagagaggaagatctttggatgatgcaaggaagagagccaagcagttccatgaagcttggagtaaacttatggagtggctagaagagtcagaaaagtctttggattctgaactggaaatcgcaaatgatccagacaaaataaaaacacaacttgcacaacataaggagtttcagaaatcactcggagccaagcattctgtctacgacaccaccaacaggactggacgttctctgaaggagaaaacctccctggctgatgacaacctgaaactggatgacatgctgagtgaactcagagacaaatgggataccatatgtggaaaatctgtggaaagacaaaacaaattggaggaagccctgttattttctggacaattcacagatgccctacaggctctcattgattggttatatagagttgaaccccagctggcagaagaccagcctgttcatggagacattgatttggtgatgaatctgatcgataatcacaaggccttccaaaaagagttggggaagaggaccagcagtgtgcaggccctgaagcgctcagcccgagaactcatagaaggcagtcgggatgactcctcctgggtcaaggtccagatgcaggaattaagcacacgctgggagaccgtgtgtgcactttctatatcaaagcaaacacggttagaagcagccctgcgtcaggcagaggaattccactcggtggtacatgccctcttggagtggctggctgaggcggagcaaaccctgcgtttccatggtgtcctcccagatgatgaggatgctctccggactctcattgatcagcataaagaattcatgaagaaactggaagaaaagagagctgaactaaataaagccaccactatgggcgacaccgttttggctatctgccaccccgactccatcactaccattaagcactggataacaatcatccgggcgaggtttgaggaggtgctggcctgggcaaagcaacatcagcagagattagcaagtgctctggctgggcttattgccaaacaggaattgttggaagctttgctggcttggttgcaatgggctgaaactacacttactgataaggataaagaagtcatcccccaggagatcgaagaggtgaaagcactcattgcagaacaccagaccttcatggaggaaatgaccagaaaacagcctgatgttgataaagtaacgaagacctataagaggagagctgctgatccttcctcattacaatcccatattccagtcttggataagggacgagcaggaagaaaacgctttccagcatcaagcttgtatccctctgggtcacagacacaaattgaaaccaaaaatcctagggtaaacttactggtgagcaaatggcagcaagtctggctcctggcgttggaaagaaggaggaaactcaatgatgccttggacagactagaggagctgagggaatttgctaactttgattttgatatttggcgcaaaaaatacatgcgatggatgaatcacaagaaatctcgagtgatggacttcttcaggagaattgataaagaccaggatgggaaaataacgcggcaggaatttattgatggaattctttcctcaaagtttccaaccagtcgcttggagatgagcgcagttgcagacatctttgacagagatggcgatggatatattgactactatgaatttgtagcagcccttcacccaaataaagatgcatataaacctatcacagatgccgacaaaatcgaagatgaggtgacaaggcaggtagctaagtgtaaatgtgcaaagcgatttcaagttgagcagattggtgataataaatacaggttcttcctgggaaatcagtttggagactcccagcaactgcgactggtccggatcctgcggagtactgtgatggttcgtgttggaggtggatggatggcacttgatgagttcttagtgaaaaatgatccttgcagggccaaaggaaggacaaacatggaactgcgtgagaagttcattttagcagatggtgccagccagggtatggctgctttccgaccccgaggccgaagatcccggccatcatcacgaggcgcttcacccaacagatccacttctgtgtccagtcaggctgcgcaggcggcctccccacaggtccctgccaccaccacacccaagattctccatcctttaacacgcaattatggtaaaccatggttgacaaacagcaaaatgtcaactccttgtaaagcagcagagtgctcagactttcccgtgccatctgcagagggaacgccaatacaaggaagcaagcttcgacttccaggatatttatcagggaaaggcttccactctggggaggacagtggcttgataacaactgcagctgccagagtccgaacacagtttgctgattccaagaagactcccagccgaccaggaagtcgagctggaagcaaagctggcagcagggccagcagccgccgaggcagtgatgcatcagactttgacatttcagaaatccagtccgtgtgctcagatgtggaaactgtcccccagacacacagacctacaccccgagcaggttctcggccatccacagcgaagccttcaaaaatccccacgccccagaggaaatcacctgccagcaaattggacaagtcctcaaagagatagtgcaattggttctaccaaggcccttccttgagcatttattatttaagtttgaacgatgtaaaatatggtgtagaaattcttgtgaaatattgcaagaggcgagtttaaaattctgcagatggccttatttgtgtatttgtctttttattttatctgtataattttttttgtcagatattctggggttaaagtcacatcatatgtgaggaggaaaagtttaacatgaactaacatttctgcactgtaacgtgccgggcacacactaaactcagttactgtacctacaggtaagtctacatcctctctgacagccacagcactacatcaatccctgacgttagggatacctcatgacattttcctgtttttatggaaactctgagaagctgaatgatacatgcaggggatattttttgagatgatttaaatgtaaaccaaaagatggaagacaaaaagacaaacacacccacacgcagtctttgcagtatctgacagagaactcacaggaagttacttcaagcacttgccagtactatgatattcaagtaccttgcagcatttctctgccattgctttcaatgaggccagaggcatcctggatattagacctattatactgtaagaatataagtataaagtgcgttcatatacatgtgaggttttcttttgcttgagtggacagtagcacctgtatcattgaactcattttgtatcagagcaattttgcttgcagaaagctatgaaataaaacacgtcccttaactgc

SEQ ID NO: 6 - protein, 2835 aa, Homo sapiens
WLVEKELMVSVLGPLSIDPNMLNTQRQQVQILLQEFATRKPQYEQLTAAGQGILSRPGEDPSLRGIVKEQLAAVTQKWDSLTGQLSDRCDWIDQAIVKSTQYQSLLRSLSDKLSDLDNKLSSSLAVSTHPDAMNQQLETAQKMKQEIQQEKKQIKVAQALCEDLSALVKEEYLKAELSRQLEGILKSFKDVEQKAENHVQHLQSACASSHQFQQMSRDFQAWLDTKKEEQNKSHPISAKLDVLESLIKDHKDFSKTLTAQSHMYEKTIAEGENLLLKTQGSEKAALQLQLNTIKTNWDTFNKQVKERENKLKESLEKALKYKEQVETLWPWIDKCQNNLEEIKFCLDPAEGENSIAKLKSLQKEMDQHFGMVELLNNTANSLLSVCEIDKEVVTDENKSLIQKVDMVTEQLHSKKFCLENMTQKFKEFQEVSKESKRQLQCAKEQLDIHDSLGSQAYSNKYLTMLQTQQKSLQALKHQVDLAKRLAQDLVVEASDSKGTSDVLLQVETIAQEHSTLSQQVDEKCSFLETKLQGIGHFQNTIREMFSQFAEFDDELDSMAPVGRDAETLQKQKETIKAFLKKLEALMASNDNANKTCKMMLATEETSPDLVGIKRDLEALSKQCNKLLDRAQAREEQVEGTIKRLEEFYSKLKEFSILLQKAEEHEESQGPVGMETETINQQLNMFKVFQKEEIEPLQGKQQDVNWLGQGLIQSAAKSTSTQGLEHDLDDVNARWKTLNKKVAQRAAQLQEALLHCGRFQDALESLLSWMVDTEELVANQKPPSAEFKVVKAQIQEQKLLQRLLDDRKSTVEVIKREGEKIATTAEPADKVKILKQLSLLDSRWEALLNKAETRNRQLEGISVVAQQFHETLEPLNEWLTTIEKRLVNCEPIGTQASKLEEQIAQHKVLQEDILLRKQNVDQALLNGLELLKQTTGDEVLIIQDKLEAIKARYKDITKLSTDVAKTLEQALQLARRLHSTHEELCTWLDKVEVELLSYETQVLKGEEASQAQMRPKELKKEAKNNKALLDSLNEVSSALLELVPWRAREGLEKMVAEDNERYRLVSDTITQKVEEIDAAILRSQQFDQAADAELSWITETEKKLMSLGDIRLEQDQTSAQLQVQKTFTMEILRHKDIIDDLVKSGHKIMTACSEEEKQSMKKKLDKVLKNYDTICQINSERYLQLERAQSLVNQFWETYEELWPWLTETQSIISQLPAPALEYETLRQQQEEHRQLRELIAEHKPHIDKMNKTGPQLLELSPGEGFSIQEKYVAADTLYSQIKEDVKKRAVALDEAISQSTQFHDKIDQILESLERIVERLRQPPSISAEVEKIKEQISENKNVSVDMEKLQPLYETLKQRGEEMIARSGGTDKDISAKAVQDKLDQMVFIWENIHTLVEEREAKLLDVMELAEKFWCDHMSLIVTIKDTQDFIRDLEDPGIDPSVVKQQQEAAETIREEIDGLQEELDIVINLGSELIAACGEPDKPIVKKSIDELNSAWDSLNKAWKDRIDKLEEAMQAAVQYQDGLQAVFDWVDIAGGKLASMSPIGTDLETVKQQIEELKQFKSEAYQQQIEMERLNHQAELLLKKVTEESDKHTVQDPLMELKLIWDSLEERIINRQHKLEGALLALGQFQHALDELLAWLTHTEGLLSEQKPVGGDPKAIEIELAKHHVLQNDVLAHQSTVEAVNKAGNDLIESSAGEEASNLQNKLEVLNQRWQNVLEKTEQRKQQLDGALRQAKGFHGEIEDLQQWLTDTERHLLASKPLGGLPETAKEQLNVHMEVCAAFEAKEETYKSLMQKGQQMLARCPKSAETNIDQDINNLKEKWESVETKLNERKTKLEEALNLAMEFHNSLQDFINWLTQAEQTLNVASRPSLILDTVLFQIDEHKVFANEVNSHREQIIELDKTGTHLKYFSQKQDVVLIKNLLISVQSRWEKVVQRLVERGRSLDDARKRAKQFHEAWSKLMEWLEESEKSLDSELEIANDPDKIKTQLAQHKEFQKSLGAKHSVYDTTNRTGRSLKEKTSLADDNLKLDDMLSELRDKWDTICGKSVERQNKLEEALLFSGQFTDALQALIDWLYRVEPQLAEDQPVHGDIDLVMNLIDNHKAFQKELGKRTSSVQALKRSARELIEGSRDDSSWVKVQMQELSTRWETVCALSISKQTRLEAALRQAEEFHSVVHALLEWLAEAEQTLRFHGVLPDDEDALRTLIDQHKEFMKKLEEKRAELNKATTMGDTVLAICHPDSITTIKHWITIIRARFEEVLAWAKQHQQRLASALAGLIAKQELLEALLAWLQWAETTLTDKDKEVIPQEIEEVKALIAEHQTFMEEMTRKQPDVDKVTKTYKRRAADPSSLQSHIPVLDKGRAGRKRFPASSLYPSGSQTQIETKNPRVNLLVSKWQQVWLLALERRRKLNDALDRLEELREFANFDFDIWRKKYMRWMNHKKSRVMDFFRRIDKDQDGKITRQEFIDGILSSKFPTSRLEMSAVADIFDRDGDGYIDYYEFVAALHPNKDAYKPITDADKIEDEVTRQVAKCKCAKRFQVEQIGDNKYRFFLGNQFGDSQQLRLVRILRSTVMVRVGGGWMALDEFLVKNDPCRAKGRTNMELREKFILADGASQGMAAFRPRGRRSRPSSRGASPNRSTSVSSQAAQAASPQVPATTTPKILHPLTRNYGKPWLTNSKMSTPCKAAECSDFPVPSAEGTPIQGSKLRLPGYLSGKGFHSGEDSGLITTAAARVRTQFADSKKTPSRPGSRAGSKAGSRASSRRGSDASDFDISEIQSVCSDVETVPQTHRPTPRAGSRPSTAKPSKIPTPQRKSPASKLDKSSKR

SEQ ID NO: 7 - DNA, 1058 bp, Homo sapiens
  CDS (1)..(927)
atgccgcgtcactgctccgccgccggctgctgcacacgggacacgcgcgagacgcgcaaccgcggcatctccttccacagacttcccaagaaggacaacccgaggcgaggcttgtggctggccaactgccagcggctggaccccagcggccagggcctgtgggacccggcatccgagtacatctacttctgctccaaacactttgaggaggactgctttgagctggtgggaatcagtggatatcacaggctaaaggagggggcagtccccaccatatttgagtctttctccaagttgcgccggacaaccaagaccaaaggacacagttacccacctggccccgctgaagtcagccggctcagacgatgcaggaagcgctgctccgagggccgagggcccacaactccattttctccacctccacctgctgatgtcacctgctttcctgtggaagaggcctcagcacctgccactttgccggcctccccagctgggaggctggagcctggccttagcagccccttttcagacctactgggccccttgggtgcccaggcagatgaagcaggctgcagcgcccagccttcaccagagcggcagccctcccctctcgaaccacggccagtctccccctcagcgtatatgctgcgcctgcccccacccgccggagcctacatccagaatgaacacagctaccaggtgggcagcgccttactctggaagcggcgagccgaggcagcccttgatgcccttgacaaggcccagcgccagctgcaggcctgcaagcggcgggagcagcggctgcggttgagactgaccaagctgcagcaggagcgggcacgggagaagcgggcacaggcagatgcccgccagactctgaaggagcatgtgcaggactttgccatgcagctgagcagcagcatggcctgaggggctgctggactgaccgaggggctgcccagcaagactgcagcctcttcctccctcagatcccaccagacccaccaggtgccataataaagcggattctagacggaaaaaaaaaaaaaaaaaaaaaa

SEQ ID NO: 8 - protein, 309 aa, Homo sapiens
MPRHCSAAGCCTRDTRETRNRGISFHRLPKKDNPRRGLWLANCQRLDPSGQGLWDPASEYIYFCSKHFEEDCFELVGISGYHRLKEGAVPTIFESFSKLRRTTKTKGHSYPPGPAEVSRLRRCRKRCSEGRGPTTPFSPPPPADVTCFPVEEASAPATLPASPAGRLEPGLSSPFSDLLGPLGAQADEAGCSAQPSPERQPSPLEPRPVSPSAYMLRLPPPAGAYIQNEHSYQVGSALLWKRRAEAALDALDKAQRQLQACKRREQRLRLRLTKLQQERAREKRAQADARQTLKEHVQDFAMQLSSSMA

SEQ ID NO: 9 - DNA, 8490 bp, Homo sapiens
  CDS (162)..(6584)
gagagaattgggtttgaggggattttggatagagcaaaagaggattgtttgtttttaattagtatagcagttgtaacctttttcctctctcagattttccccaagaacgctgtcccgtggtgctcgcaccacttttataccctgaaaaacgggacattctaatggacaggatcctgcctgattccggaggggtagctaaaaccatgatggagagctctttggctgatttcatgcaagaagtaggctatggcttttgtgcaagtattgaagaatgtcgcaatataatcgtgcagtttggtgttcgggaggtcacagctgcccaggttgcaagggttttgggaatgatggctcgaactcattcaggattaacagatggcattccattacagagtatttctgctccgggcagtgggatctggagtgatgggaaagataaaagtgatggagcacaggcacacacatggaatgtagaagtcttgattgacgttcttaaagaactgaatccaagtttgaatttcaaggaagtaacttatgaactggaccatcctggatttcaaattcgtgacagtaaaggacttcataatgtggtttatggcattcagaggggtttgggtatggaagtgttcccagtagacctcatatatagaccttggaaacatgctgaaggccagctctccttcattcaacattcccttataaatccagagatcttctgttttgctgactatccctgtcatactgttgccactgatattctgaaagcaccaccagaggatgacaatcgagaaattgccacatggaagagcttggatttgattgaatctctgctgaggcttgcagaggttgggcagtatgagcaagtcaaacagctcttcagcttccctatcaaacactgtccagacatgctggtattggccttactacaaattaacacctcttggcataccttgcgccatgaacttatctccactctgatgccaattttccttggaaaccatcctaactcagctattattttgcactatgcatggcatgggcagggacagtctccctcaattcgccaacttatcatgcatgcaatggcagaatggtacatgagaggggagcagtatgatcaggccaaattgtctcgaatacttgatgtggcccaggacttgaaggccttgtcaatgctgctaaatggtactccatttgcctttgttattgaccttgctgcacttgcttcacgtcgtgaatacctcaaacttgataagtggctcacagataaaattcgagagcatggggagccttttatccaggcgtgtatgacttttttaaagagacggtgtccttctattttgggcggacttgccccagaaaaagaccagcccaaaagtgctcaacttcctccagaaactttggcgacaatgttggcctgtctgcaagcttgtgcagggagtgtttctcaggagctatcagaaactatcctcaccatggtagccaattgcagtaatgttatgaataaggccagacaaccaccacctggagttatgccaaaaggacgtcctcctagtgctagcagcttagatgccatttctcctgttcagattgaccctcttgctggaatgacatctcttagtataggtggttcagctgcccctcacacccagagtatgcagggttttcctccaaatttgggttctgcattcagtacccctcagtcaccagcaaaagcatttccacccctttcaacccccaatcagaccactgcattcagtggtattggaggactttcatcacagcttccagtaggtggtcttggcacaggcagcctgactggtataggaactggtgctcttggactccctgcagtgaataacgacccttttgtacagaggaaactgggcacctctggactgaatcagcctacattccagcagagtaagatgaaaccttcggacttgtctcaggtgtggccagaggcaaaccagcactttagtaaagagatagatgatgaagcaaacagctatttccagcgaatatataatcatccaccacatccaaccatgtctgttgatgaggtattagaaatgctgcagagatttaaagactctactataaagagggaacgagaagtatttaactgtatgctaaggaacttgtttgaagaatatcgtttttttccccagtatcctgataaagagttacatataacagcctgcctatttggtggtataattgagaaaggactggtcacttacatggcactaggtctggctctacgatatgttcttgaagccttacgcaagccttttggatccaaaatgtattatttcgggattgctgcactagatagatttaaaaacagattgaaggactatccccagtattgtcagcatttggcttctatcagtcactttatgcaatttccacatcatttacaggagtatattgagtatggacagcagtctagagatcctcctgtgaaaatgcaaggctctatcacaacccctggaagtattgcactggctcaggcccaggctcaggcccaggttccagcaaaagctcctcttgctggtcaagttagcactatggtaaccacctcaacaactaccactgttgctaaaacggttacggtcaccaggccaactggagtcagctttaagaaagatgtgccaccttctattaatactacaaatatagatacgttgcttgtggccacagatcaaactgagagaattgtggagcccccagaaaatatccaggagaaaattgcttttattttcaataatctctcacagtcaaatatgacacaaaaggttgaagagctaaaggaaacggtgaaagaagaatttatgccttgggtttcacagtatctggttatgaagagagtcagtattgagccaaactttcatagcctgtattcaaacttccttgacacgctgaagaatcctgaatttaacaagatggttctgaatgagacctacagaaacattaaagtgctcctgacctctgataaagctgcagccaatttctcagatcgttctttgctgaagaacttgggacattggctaggaatgatcacattagctaaaaacaaacccatcttacacactgacttggatgtgaaatcattgctgctagaggcttatgttaaaggacaacaagaattgctctatgtagtgccctttgttgccaaagtcttagaatctagcattcgtagtgtggtttttaggccaccaaacccttggacaatggcaattatgaatgtattagctgagctacatcaggagcatgacttaaagttaaacttgaagtttgaaatcgaggttctctgcaagaaccttgcattagacatcaatgagctaaaacctggaaacctcctaaaggataaagatcgcctgaagaatttagatgagcaactctctgctccaaagaaagatgtcaagcagccagaagaactccctcccatcacaaccacaacaacttctactacaccagctaccaacaccacttgtacagccacggttccaccacagccacagtacagctaccacgacatcaatgtctattcccttgcgggcttggcaccacacattactctaaatccaacaattcccttgtttcaggcccatccacagttgaagcagtgtgtgcgtcaggcaattgaacgggctgtccaggagctggtccatcctgtggtggatcgatcaattaagattgccatgactacttgtgagcaaatagtcaggaaggattttgccctggattcggaggaatctcgaatgcgaatagcagctcatcacatgatgcgtaacttgacagctggaatggctatgattacatgcagggaacctttgctcatgagcatatctaccaacttaaaaaacagttttgcctcagcccttcgtactgcttccccacaacaaagagaaatgatggatcaggcagctgctcaattagctcaggacaattgtgagttggcttgctgttttattcagaagactgcagtagaaaaagcaggccctgagatggacaagagattagcaactgaatttgagctgagaaaacatgctaggcaagaaggacgcagatactgtgatcctgttgttttaacatatcaagctgaacggatgccagagcaaatcaggctgaaagttggtggtgtggacccaaagcagttggctgtttacgaagagtttgcacgcaatgttcctggcttcttacctacaaatgacttaagtcagcccacgggatttttagcccagcccatgaagcaagcttgggcaacagatgatgtagctcagatttatgataagtgtattacagaactggagcaacatctacatgccatcccaccaactttggccatgaaccctcaagctcaggctcttcgaagtctcttggaggttgtagttttatctcgaaactctcgggatgccatagctgctcttggattgctccaaaaggctgtagagggcttactagatgccacaagtggtgctgatgctgaccttctgctgcgctacagggaatgccacctcttggtcctaaaagctctgcaggatggccgggcatatgggtctccatggtgcaacaaacagatcacaaggtgcctaattgaatgtcgagatgaatataaatataatgtggaggctgtggagctgctaattcgcaatcatttggttaatatgcagcagtatgatcttcacctagcgcagtcaatggagaatggcttaaactacatggctgtggcatttgctatgcagttagtaaaaatcctgctggtggatgaaaggagtgttgctcatgttactgaggcagatctgttccacaccattgaaaccctcatgaggattaatgctcattccagaggcaatgctccagaaggattgccccagctgatggaagtagtgcgatccaactatgaagcaatgattgatcgtgctcatggaggcccaaactttatgatgcattctgggatctctcaagcctcagagtatgatgaccctccaggcctgagggagaaggcagagtatcttctgagggaatgggtgaatctctaccattcagcagcagctggccgcgacagtaccaaagctttctctgcatttgttggacagatgcaccagcaaggaatactgaagaccgatgatctcataacaaggttctttcgtctgtgtactgaaatgtgtgttgaaatcagttaccgtgctcaggctgagcagcagcacaatcctgctgccaatcccaccatgatccgagccaagtgctatcacaacctggatgcctttgttcgactcattgcactgctcgtgaaacactcaggggaggccaccaacactgtcacaaagattaatctgctgaacaaggtccttggtatagtagtgggagttctccttcaggatcatgatgttcgtcagagtgaatttcagcaacttccctaccatcgaatttttatcatgcttctcttggaactcaatgcacctgagcatgtgttggaaaccattaatttccagacacttacagctttctgcaatacattccacatcttgaggcctaccaaagctcctggctttgtatatgcctggcttgaactgatttcccatcggatatttattgcaagaatgctggcacatacgccacagcagaaggggtggcctatgtatgcacagctactgattgatttattcaaatatttagcgcctttccttagaaatgtggaactcaccaaacctatgcaaatcctctacaagggcactttaagagtgctgctggttcttttgcatgatttcccagagttcctttgtgattaccattatgggttctgtgatgtgatcccacctaattgtatccagttaagaaatttgatcctgagtgcctttccaagaaacatgaggctccccgacccattcactcctaatctaaaggtggacatgttgagtgaaattaacattgctccccggattctcaccaatttcactggagtaatgccacctcagttcaaaaaggatttggattcctatcttaaaactcgatcaccagtcactttcctgtctgatctgcgcagcaacctacaggtatccaatgaacctgggaatcgctacaacctccagctcatcaatgcactggtgctctatgtcgggactcaggccattgcgcacatccacaacaagggcagcacaccttcaatgagcaccatcactcactcagcacacatggatatcttccagaatttggctgtggacttggacactgagggtcgctatctctttttgaatgcaattgcaaatcagctccggtacccaaatagccacactcactacttcagttgcaccatgctgtacctttttgcagaggccaatacggaagccatccaagaacagatcacaagagttctcttggaacggttgattgtaaataggccacatccttggggtcttcttattaccttcattgagctgattaaaaacccagcgtttaagttctggaaccatgaatttgtacactgtgccccagaaatcgaaaagttattccagtcggtcgcacagtgctgcatgggacagaagcaggcccagcaagtaatggaagggacaggtgccagttagacgaaactgcatctctgttgtacgtgtcagtctagaggtctcactgcaccgagttcataaactgactgaagaatcctttcagctcttcctgactttcccagccctttggtttgtgggtatctgccccaactactgttgggatcagcctcctgtcttatgtgggcacgttccaaagtttaaatgcatttttttgactcttggccaaaatttagaagatgctgtgaatatcattttgaacttgtgtaatacatgaaagagaaaacctttgtctggaacttcttgggctttgtgcaagctgtgtccaaggcaagtacataaactggtaccttgtaatgaagaggcagctgatgccatgcacttgtctgagggcatagctccatgtcttctgacattcctggtgtcccaaagaatagcaaaaagccagtttgaatattatgtaacttatttttttaatgtggacaggggaccttgaaaatcactaagttattaaaaatgtggatgtgctagaattggatatgtccaggaacatgggaagggctcactattggaatcccatgagtttccattttgtctctacccaaacgtattccaaagctgactgcatttgtaccatcttatttcttttggggattatacacctcagccgcctgagatgggggtcagctctttatataaagggaaaccagaccaggcctaaagcccaccccctaccctcacccccacaatcctctcctgaaacttaaaaacagtgggaatataggaaagggaaccaaatctcattaattaattgttctcccccattaccccactgaatgaatggccatacaggctaagctgaataatgacaaagttgaaaggaccaatacagccccttttataaggattttgaatgttttgcaaatgtattggtccctgtgttgtattttgtagccttttcctgggcttcagctcccctacttcttgtatgtgtatgcatactgtagctaaccattaaagtcatgacacacacatgagtccactgtgcctttctcagtagcagcagccagtgctggtggtgaggaggaaaagtggacaatccagccctgcagaccttggggccatggggaaccaccaactaacttcttgctgaatgattgatttgattgattgattgataggtcattcctactactaagctggcatgtttaaggaaattgtatttttcttcctatttatttcaacactggacaaatgctggagcaggtttatctggttaagctgagtttaaaatacccagttttaatatccttttcccccaggtattttttttttttttaaagaaaatgagtagatacgtatttaaaaacttaacccacttaaaatttgccttacctttcatgactgtcaagttttatggccagagaggacaaaacagttcaaaattaaataattgaagtcctccttgagtgatgtcttagggtttattccctgagaggtggtttgtgccatctagactgaactttgggtaactatcgagtgccagttacacagcttattaaatccagagtcttttcaataaaggttaagtgacttcctcaaactagacttagatttaaaccaggggtctacctccaaagtctattattaaatgctgaaacacaacaagacttacttattactaccgtatgtccactggctttggttaaaactgagaacaaaacagctgtaacatgctttaagtaacaattagcagtgattcccaagtctgctgttctcaatcctaaaacacatcacccttacctgagttaatcctagcacaccaactttgctacttttcctcttggtgtttagtaagcaaaagaaagatccaaattttcctctttttcaaacatcaatgagtattagcttagaataaagttcttat

SEQ ID NO: 10 - protein, 2141 aa, Homo sapiens [sequence omitted]

SEQ ID NO: 11 - DNA, 20 bp, Homo sapiens
ttctctgccattttgaaacc

SEQ ID NO: 12 - DNA, 20 bp, Homo sapiens
ctctgccattttgaaaccct

SEQ ID NO: 13 - DNA, 20 bp, Homo sapiens
ctgccattttgaaaccctta

SEQ ID NO: 14 - DNA, 20 bp, Homo sapiens
ccagactccatggttctctg

SEQ ID NO: 15 - DNA, 20 bp, Homo sapiens
agactccatggttctctggg

SEQ ID NO: 16 - DNA, 20 bp, Homo sapiens
actccatggttctctgggga

SEQ ID NO: 17 - DNA, 20 bp, Homo sapiens
tgcttctccatttcttgagg

SEQ ID NO: 18 - DNA, 20 bp, Homo sapiens
cttctccatttcttgaggtt

SEQ ID NO: 19 - DNA, 20 bp, Homo sapiens
tctccatttcttgaggttga

SEQ ID NO: 20 - DNA, 20 bp, Homo sapiens
ttagccaccatggagagcaa

SEQ ID NO: 21 - DNA, 20 bp, Homo sapiens
agccaccatggagagcaatt

SEQ ID NO: 22 - DNA, 20 bp, Homo sapiens
ccaccatggagagcaattag

SEQ ID NO: 23 - DNA, 20 bp, Homo sapiens
ttcctcagcatagattttat

SEQ ID NO: 24 - DNA, 20 bp, Homo sapiens
cctcagcatagattttatga

SEQ ID NO: 25 - DNA, 20 bp, Homo sapiens
tcagcatagattttatgaga

SEQ ID NO: 26 - DNA, 20 bp, Homo sapiens
tcaacttccatctccctcag

SEQ ID NO: 27 - DNA, 20 bp, Homo sapiens
aacttccatctccctcagac

SEQ ID NO: 28 - DNA, 20 bp, Homo sapiens
cttccatctccctcagactc

SEQ ID NO: 29 - DNA, 20 bp, Homo sapiens
tggtcaggcatcactggacg

SEQ ID NO: 30 - DNA, 20 bp, Homo sapiens
gtcaggcatcactggacggt

SEQ ID NO: 31 - DNA, 20 bp, Homo sapiens
caggcatcactggacggtta

SEQ ID NO: 32 - DNA, 20 bp, Homo sapiens
gtctcctccatggccggcgc

SEQ ID NO: 33 - DNA, 20 bp, Homo sapiens
ctcctccatggccggcgccg

SEQ ID NO: 34 - DNA, 20 bp, Homo sapiens
cctccatggccggcgccgcc

SEQ ID NO: 35 - DNA, 20 bp, Homo sapiens
gaggagggtgccagggggaa

SEQ ID NO: 36 - DNA, 20 bp, Homo sapiens
cagggtcttcgtggctcacg

SEQ ID NO: 37 - DNA, 20 bp, Homo sapiens
ctgcgtgtagtggttgtgca

SEQ ID NO: 38 - DNA, 20 bp, Homo sapiens
accgactccattaccagccc

SEQ ID NO: 39 - DNA, 20 bp, Homo sapiens
cgactccattaccagcccct

SEQ ID NO: 40 - DNA, 20 bp, Homo sapiens
actccattaccagcccctga

SEQ ID NO: 41 - DNA, 20 bp, Homo sapiens
ttcttggacattgttccttt

SEQ ID NO: 42 - DNA, 20 bp, Homo sapiens
cttggacattgttcctttct

SEQ ID NO: 43 - DNA, 20 bp, Homo sapiens
tggacattgttcctttctaa

SEQ ID NO: 44 - DNA, 20 bp, Homo sapiens
gcttcctccatttttgccag

SEQ ID NO: 45 - DNA, 20 bp, Homo sapiens
ttcctccatttttgccagga

SEQ ID NO: 46 - DNA, 20 bp, Homo sapiens
cctccatttttgccaggaaa

SEQ ID NO: 47 - DNA, 20 bp, Homo sapiens
tgcgccgacatggtgctgct

SEQ ID NO: 48 - DNA, 20 bp, Homo sapiens
cgccgacatggtgctgctcc

SEQ ID NO: 49 - DNA, 20 bp, Homo sapiens
ccgacatggtgctgctccgc

SEQ ID NO: 50 - DNA, 20 bp, Homo sapiens
ctttttcagttcctttggtc

SEQ ID NO: 51 - DNA, 20 bp, Homo sapiens
cattttctcaagtccttctc

SEQ ID NO: 52 - DNA, 20 bp, Homo sapiens
taatccaggataactcagca

SEQ ID NO: 53 - DNA, 20 bp, Homo sapiens
ctactctccatgcctgcttc

SEQ ID NO: 54 - DNA, 20 bp, Homo sapiens
actctccatgcctgcttccc

SEQ ID NO: 55 - DNA, 20 bp, Homo sapiens
tctccatgcctgcttcccgg

SEQ ID NO: 56 - DNA, 20 bp, Homo sapiens
acctcagccatctgcaacaa

SEQ ID NO: 57 - DNA, 20 bp, Homo sapiens
ctcagccatctgcaacaagg

SEQ ID NO: 58 - DNA, 20 bp, Homo sapiens
cagccatctgcaacaaggct

SEQ ID NO: 59 - DNA, 20 bp, Homo sapiens
tgcgtctccatgcctctcag

SEQ ID NO: 60 - DNA, 20 bp, Homo sapiens
cgtctccatgcctctcagcc

SEQ ID NO: 61 - DNA, 20 bp, Homo sapiens
tctccatgcctctcagccgg

SEQ ID NO: 62 - DNA, 20 bp, Homo sapiens
actctgattggttcagacat

SEQ ID NO: 63 - DNA, 20 bp, Homo sapiens
cagtcacaccaagtttaaga

SEQ ID NO: 64 - DNA, 20 bp, Homo sapiens
ttgattacaacagggaatga

SEQ ID NO: 65 - DNA, 20 bp, Homo sapiens
gggacgctcatggtgcaggc

SEQ ID NO: 66 - DNA, 20 bp, Homo sapiens
gacgctcatggtgcaggccc

SEQ ID NO: 67 - DNA, 20 bp, Homo sapiens
cgctcatggtgcaggcccgg

SEQ ID NO: 68 - DNA, 20 bp, Homo sapiens
accaggaacatggctgcgac

SEQ ID NO: 69 - DNA, 20 bp, Homo sapiens
caggaacatggctgcgactc

SEQ ID NO: 70 - DNA, 20 bp, Homo sapiens
ggaacatggctgcgactcac

SEQ ID NO: 71 - DNA, 20 bp, Homo sapiens
ttaggctccatggccgacag

SEQ ID NO: 72 - DNA, 20 bp, Homo sapiens
aggctccatggccgacaggt

SEQ ID NO: 73 - DNA, 20 bp, Homo sapiens
gctccatggccgacaggtct

SEQ ID NO: 74 - DNA, 20 bp, Homo sapiens
aaggcaggcatcttggcatg

SEQ ID NO: 75 - DNA, 20 bp, Homo sapiens
ggcaggcatcttggcatgga

SEQ ID NO: 76 - DNA, 20 bp, Homo sapiens
caggcatcttggcatggatt

SEQ ID NO: 77 - DNA, 20 bp, Homo sapiens
gatgtcatcatttacagtat

SEQ ID NO: 78 - DNA, 20 bp, Homo sapiens
tgtcatcatttacagtatat

SEQ ID NO: 79 - DNA, 20 bp, Homo sapiens
tcatcatttacagtatatac

SEQ ID NO: 80 - DNA, 20 bp, Homo sapiens
cacaggtccattccgacgcc

SEQ ID NO: 81 - DNA, 20 bp, Homo sapiens
caggtccattccgacgcctc

SEQ ID NO: 82 - DNA, 20 bp, Homo sapiens
ggtccattccgacgcctctg

SEQ ID NO: 83 - DNA, 20 bp, Homo sapiens
tggaggctcataaataccac

SEQ ID NO: 84 - DNA, 20 bp, Homo sapiens
gaggctcataaataccacag

SEQ ID NO: 85 - DNA, 20 bp, Homo sapiens
ggctcataaataccacagct

SEQ ID NO: 86 - DNA, 20 bp, Homo sapiens
cttgtctccatgagggcacc

SEQ ID NO: 87 - DNA, 20 bp, Homo sapiens
tgtctccatgagggcaccag

SEQ ID NO: 88 - DNA, 20 bp, Homo sapiens
tctccatgagggcaccaggt

SEQ ID NO: 89 - DNA, 20 bp, Homo sapiens
aacactggcatcgcggcggc

SEQ ID NO: 90 - DNA, 20 bp, Homo sapiens
cactggcatcgcggcggcga

SEQ ID NO: 91 - DNA, 20 bp, Homo sapiens
ctggcatcgcggcggcgagc

SEQ ID NO: 92 - DNA, 20 bp, Homo sapiens
tcccgagacatgtctgtggt

SEQ ID NO: 93 - DNA, 20 bp, Homo sapiens
ccgagacatgtctgtggtca

SEQ ID NO: 94 - DNA, 20 bp, Homo sapiens
gagacatgtctgtggtcaag

SEQ ID NO: 95 - DNA, 20 bp, Homo sapiens
cctcggtggccgccatctcg

SEQ ID NO: 96 - DNA, 20 bp, Homo sapiens
tcggtggccgccatctcgcc

SEQ ID NO: 97 - DNA, 20 bp, Homo sapiens
aaattgtccatggtgcacaa

SEQ ID NO: 98 - DNA, 20 bp, Homo sapiens
attgtccatggtgcacaagt

SEQ ID NO: 99 - DNA, 20 bp, Homo sapiens
tgtccatggtgcacaagtct

SEQ ID NO: 100 - DNA, 20 bp, Homo sapiens
ctataactactactgtgcat

SEQ ID NO: 101 - DNA, 20 bp, Homo sapiens
gtactcttctttaaccagtg

SEQ ID NO: 102 - DNA, 20 bp, Homo sapiens
ggtctgtgtgtctgggggac

SEQ ID NO: 103 - DNA, 20 bp, Homo sapiens
agcgagttcatggtggcgct

SEQ ID NO: 104 - DNA, 20 bp, Homo sapiens
cgagttcatggtggcgctcg

SEQ ID NO: 105 - DNA, 20 bp, Homo sapiens
aaccaagacatgatggcagc

SEQ ID NO: 106 - DNA, 20 bp, Homo sapiens
ccaagacatgatggcagcag

SEQ ID NO: 107 - DNA, 20 bp, Homo sapiens
aagacatgatggcagcagga

SEQ ID NO: 108 - DNA, 20 bp, Homo sapiens
cacagtaccatgaacagtct

SEQ ID NO: 109 - DNA, 20 bp, Homo sapiens
cagtaccatgaacagtctct

SEQ ID NO: 110 - DNA, 20 bp, Homo sapiens
gtaccatgaacagtctctgg

SEQ ID NO: 111 - DNA, 20 bp, Homo sapiens
ggccgggacatggcgagcgc

SEQ ID NO: 112 - DNA, 20 bp, Homo sapiens
ccgggacatggcgagcgcgc

SEQ ID NO: 113 - DNA, 20 bp, Homo sapiens
gggacatggcgagcgcgcgg

SEQ ID NO: 114 - DNA, 20 bp, Homo sapiens
tcaaagctcatctcgcagtg

SEQ ID NO: 115 - DNA, 20 bp, Homo sapiens
aaagctcatctcgcagtgat

SEQ ID NO: 116 - DNA, 20 bp, Homo sapiens
agctcatctcgcagtgatac

SEQ ID NO: 117 - DNA, 20 bp, Homo sapiens
tgcacgaacatcccgccgcc

SEQ ID NO: 118 - DNA, 20 bp, Homo sapiens
cacgaacatcccgccgccgc

SEQ ID NO: 119 - DNA, 20 bp, Homo sapiens
cgaacatcccgccgccgccg

SEQ ID NO: 120 - DNA, 20 bp, Homo sapiens
actgtatctccttccaccat

SEQ ID NO: 121 - DNA, 20 bp, Homo sapiens
gattgactttctttacaaga

SEQ ID NO: 122 - DNA, 20 bp, Homo sapiens
gaacatctccagggcccccc

SEQ ID NO: 123 - DNA, 20 bp, Homo sapiens
agagttgccatattggactt

SEQ ID NO: 124 - DNA, 20 bp, Homo sapiens
agttgccatattggacttgg

SEQ ID NO: 125 - DNA, 20 bp, Homo sapiens
ttgccatattggacttggaa

SEQ ID NO: 126 - DNA, 20 bp, Homo sapiens
taggccggcatcttggcggc

SEQ ID NO: 127 - DNA, 20 bp, Homo sapiens
ggccggcatcttggcggcag

SEQ ID NO: 128 - DNA, 20 bp, Homo sapiens
ccggcatcttggcggcaggc

SEQ ID NO: 129 - DNA, 20 bp, Homo sapiens
ttttcttccatttcatctat

SEQ ID NO: 130 - DNA, 20 bp, Homo sapiens
ttcttccatttcatctattt

SEQ ID NO: 131 - DNA, 20 bp, Homo sapiens
cttccatttcatctatttta

SEQ ID NO: 132 - DNA, 20 bp, Homo sapiens
gacgaccccatagtggcacg

SEQ ID NO: 133 - DNA, 20 bp, Homo sapiens
cgaccccatagtggcacgtc

SEQ ID NO: 134 - DNA, 20 bp, Homo sapiens
accccatagtggcacgtcca

SEQ ID NO: 135 - DNA, 20 bp, Homo sapiens
tttttatccattcctcttcg

SEQ ID NO: 136 - DNA, 20 bp, Homo sapiens
tttatccattcctcttcgtc

SEQ ID NO: 137 - DNA, 20 bp, Homo sapiens
tatccattcctcttcgtcct

SEQ ID NO: 138 - DNA, 20 bp, Homo sapiens
tggagtgtcatcttggtgat

SEQ ID NO: 139 - DNA, 20 bp, Homo sapiens
gagtgtcatcttggtgatgc

SEQ ID NO: 140 - DNA, 20 bp, Homo sapiens
gtgtcatcttggtgatgcca

SEQ ID NO: 141 - DNA, 20 bp, Homo sapiens
cacatgggcatgctctgtgg

SEQ ID NO: 142 - DNA, 20 bp, Homo sapiens
catgggcatgctctgtgggc

SEQ ID NO: 143 - DNA, 20 bp, Homo sapiens
tgggcatgctctgtgggcct

SEQ ID NO: 144 - DNA, 20 bp, Homo sapiens
gtggaggccatggtgagttt

SEQ ID NO: 145 - DNA, 20 bp, Homo sapiens
ggaggccatggtgagtttgc

SEQ ID NO: 146 - DNA, 20 bp, Homo sapiens
aggccatggtgagtttgcct

SEQ ID NO: 147 - DNA, 20 bp, Homo sapiens
cggtcgcacatggttaccgt

SEQ ID NO: 148 - DNA, 20 bp, Homo sapiens
gtcgcacatggttaccgtgg

SEQ ID NO: 149 - DNA, 20 bp, Homo sapiens
cgcacatggttaccgtggag

SEQ ID NO: 150 - DNA, 20 bp, Homo sapiens
tcatcatccatggtgagctg

SEQ ID NO: 151 - DNA, 20 bp, Homo sapiens
atcatccatggtgagctggc

SEQ ID NO: 152 - DNA, 20 bp, Homo sapiens
catccatggtgagctggcgg

SEQ ID NO: 153 - DNA, 20 bp, Homo sapiens
caccctgacatgttggctac

SEQ ID NO: 154 - DNA, 20 bp, Homo sapiens
ccctgacatgttggctactg

SEQ ID NO: 155 - DNA, 20 bp, Homo sapiens
ctgacatgttggctactgct

SEQ ID NO: 156 - DNA, 20 bp, Homo sapiens
cgcagaggcatggtgcgtcg

SEQ ID NO: 157 - DNA, 20 bp, Homo sapiens
cagaggcatggtgcgtcggt

SEQ ID NO: 158 - DNA, 20 bp, Homo sapiens
gaggcatggtgcgtcggtcc

SEQ ID NO: 159 - DNA, 20 bp, Homo sapiens
gcaattctcatggtagtgag

SEQ ID NO: 160 - DNA, 20 bp, Homo sapiens
aattctcatggtagtgagtt

SEQ ID NO: 161 - DNA, 20 bp, Homo sapiens
ttctcatggtagtgagtttt

SEQ ID NO: 162 - DNA, 20 bp, Homo sapiens
atgttgtacatgacacttcc

SEQ ID NO: 163 - DNA, 20 bp, Homo sapiens
gttgtacatgacacttccaa

SEQ ID NO: 164 - DNA, 20 bp, Homo sapiens
tgtacatgacacttccaact

SEQ ID NO: 165 - DNA, 20 bp, Homo sapiens
gccgaagtcatgctccctct

SEQ ID NO: 166 - DNA, 20 bp, Homo sapiens
cgaagtcatgctccctctgt

SEQ ID NO: 167 - DNA, 20 bp, Homo sapiens
aagtcatgctccctctgtgg

SEQ ID NO: 168 - DNA, 20 bp, Homo sapiens
tagcacctgcatggttgctt

SEQ ID NO: 169 - DNA, 20 bp, Homo sapiens
gcacctgcatggttgcttcc

SEQ ID NO: 170 - DNA, 20 bp, Homo sapiens
agtcgccacatcccgccggc

SEQ ID NO: 171 - DNA, 20 bp, Homo sapiens
tcgccacatcccgccggcgg

SEQ ID NO: 172 - DNA, 20 bp, Homo sapiens
gccacatcccgccggcgggg

SEQ ID NO: 173 - DNA, 20 bp, Homo sapiens
gcggagcagtgacgcggcat

SEQ ID NO: 174 - DNA, 20 bp, Homo sapiens
gaggcctcttccacaggaaa

SEQ ID NO: 175 - DNA, 20 bp, Homo sapiens
atgctgctgctcagctgcat

SEQ ID NO: 176 - DNA, 20 bp, Homo sapiens
ggcctccaccattgcgtcga

SEQ ID NO: 177 - DNA, 20 bp, Homo sapiens
atggcctccaccattgcgtc

SEQ ID NO: 178 - DNA, 20 bp, Homo sapiens
ctatggcctccaccattgcg

SEQ ID NO: 179 - DNA, 20 bp, Homo sapiens
gatcctgtccattagaatgt

SEQ ID NO: 180 - DNA, 20 bp, Homo sapiens
aggatcctgtccattagaat

SEQ ID NO: 181 - DNA, 20 bp, Homo sapiens
gcaggatcctgtccattaga

SEQ ID NO: 182 - DNA, 20 bp, Homo sapiens
atagtctgccatggttgcac

SEQ ID NO: 183 - DNA, 20 bp, Homo sapiens
gaatagtctgccatggttgc

SEQ ID NO: 184 - DNA, 20 bp, Homo sapiens
ttgaatagtctgccatggtt

SEQ ID NO: 185 - DNA, 20 bp, Homo sapiens
cggggtcagcatggtgtggg

SEQ ID NO: 186 - DNA, 20 bp, Homo sapiens
ggcggggtcagcatggtgtg

SEQ ID NO: 187 - DNA, 20 bp, Homo sapiens
acggcggggtcagcatggtg

SEQ ID NO: 188 - DNA, 20 bp, Homo sapiens
gtttgaggccatgtccgcgc

SEQ ID NO: 189 - DNA, 20 bp, Homo sapiens
tcgtttgaggccatgtccgc

SEQ ID NO: 190 - DNA, 20 bp, Homo sapiens
aatcgtttgaggccatgtcc

SEQ ID NO: 191 - DNA, 20 bp, Homo sapiens
ccccggctccatggtgggcc

SEQ ID NO: 192 - DNA, 20 bp, Homo sapiens
gaccccggctccatggtggg

SEQ ID NO: 193 - DNA, 20 bp, Homo sapiens
cggaccccggctccatggtg

SEQ ID NO: 194 - DNA, 20 bp, Homo sapiens
cttcttgcccatatccgacc

SEQ ID NO: 195 - DNA, 20 bp, Homo sapiens
tgcttcttgcccatatccga

SEQ ID NO: 196 - DNA, 20 bp, Homo sapiens
tgtgcttcttgcccatatcc

SEQ ID NO: 197 - DNA, 20 bp, Homo sapiens
gagctcatctccaatcccat

SEQ ID NO: 198 - DNA, 20 bp, Homo sapiens
agcagggaaggggcccggga

SEQ ID NO: 199 - DNA, 20 bp, Homo sapiens
gggccagcactttgcccgct

SEQ ID NO: 200 - DNA, 20 bp, Homo sapiens
ttgagtagccattgtccacg

SEQ ID NO: 201 - DNA, 20 bp, Homo sapiens
gcttgagtagccattgtcca

SEQ ID NO: 202 - DNA, 20 bp, Homo sapiens
cagcttgagtagccattgtc

SEQ ID NO: 203 - DNA, 20 bp, Homo sapiens
gaacttcaacatctccccac

SEQ ID NO: 204 - DNA, 20 bp, Homo sapiens
ttgaacttcaacatctcccc

SEQ ID NO: 205 - DNA, 20 bp, Homo sapiens
atttgaacttcaacatctcc

SEQ ID NO: 206 - DNA, 20 bp, Homo sapiens
aaagaggctcattcccgtac

SEQ ID NO: 207 - DNA, 20 bp, Homo sapiens
tcaaagaggctcattcccgt

SEQ ID NO: 208 - DNA, 20 bp, Homo sapiens
gatcaaagaggctcattccc

SEQ ID NO: 209 - DNA, 20 bp, Homo sapiens
gggccggctcatgccgccac

SEQ ID NO: 210 - DNA, 20 bp, Homo sapiens
gggggccggctcatgccgcc

SEQ ID NO: 211 - DNA, 20 bp, Homo sapiens
gcgggggccggctcatgccg

SEQ ID NO: 212 - DNA, 20 bp, Homo sapiens
ctaccagtgcatccatgaca

SEQ ID NO: 213 - DNA, 20 bp, Homo sapiens
ttctaccagtgcatccatga

SEQ ID NO: 214 - DNA, 20 bp, Homo sapiens
tcttctaccagtgcatccat

SEQ ID NO: 215 - DNA, 20 bp, Homo sapiens
aggagtccccatcttgtgtt

SEQ ID NO: 216 - DNA, 20 bp, Homo sapiens
gcaggagtccccatcttgtg

SEQ ID NO: 217 - DNA, 20 bp, Homo sapiens
aagcaggagtccccatcttg

SEQ ID NO: 218 - DNA, 20 bp, Homo sapiens
gagggcgtacatagcaccgt

SEQ ID NO: 219 - DNA, 20 bp, Homo sapiens
aagagggcgtacatagcacc

SEQ ID NO: 220 - DNA, 20 bp, Homo sapiens
ggaagagggcgtacatagca

SEQ ID NO: 221 - DNA, 20 bp, Homo sapiens
cagggcgggcatcttctcgg

SEQ ID NO: 222 - DNA, 20 bp, Homo sapiens
cgcagggcgggcatcttctc

SEQ ID NO: 223 - DNA, 20 bp, Homo sapiens
ggcgcagggcgggcatcttc

SEQ ID NO: 224 - protein, 20 aa, Homo sapiens
PMDEPISSHPFHIEDEVDDI

SEQ ID NO: 225 - protein, 20 aa, Homo sapiens
TEPCWQYSDHHENKYCDLEC

SEQ ID NO: 226 - protein, 20 aa, Homo sapiens
LISKSVSQEKQEKGMANLAQ

SEQ ID NO: 227 - protein, 20 aa, Homo sapiens
GEFLFNKQLESIGIPQFHSP

SEQ ID NO: 228 - protein, 20 aa, Homo sapiens
VWRESEVNHYYEPKLIIDLS

SEQ ID NO: 229 - protein, 50 aa, Homo sapiens
QFSEHDWPVHNNFDIDEVQLDPRALSDVTDEEEVQVDPRKYLDGDREKYL

SEQ ID NO: 230 - protein, 50 aa, Homo sapiens
NSGEFLFNKQLESIGIPQFHSPVGSPLKSIQATLTPSAMKSSPQIPHQTY

SEQ ID NO: 231 - protein, 20 aa, Homo sapiens
MESGSTAASEEARSLRECEL

SEQ ID NO: 232 - protein, 20 aa, Homo sapiens
YVQKHNIQALLKDSIVQLCT

SEQ ID NO: 233 - protein, 20 aa, Homo sapiens
ARPERPMAFLREYFERLEKE

SEQ ID NO: 234 - protein, 20 aa, Homo sapiens
EAKQIQNLQKAGTRTDSRED

SEQ ID NO: 235 - protein, 20 aa, Homo sapiens
EISPPPPNPVVKGRRRRGAI

SEQ ID NO: 236 - protein, 50 aa, Homo sapiens
EAKQIQNLQKAGTRTDSREDEISPPPPNPVVKGRRRRGAISAEVYTEEDA

SEQ ID NO: 237 - protein, 50 aa, Homo sapiens
AASEEARSLRECELYVQKHNIQALLKDSIVQLCTARPERPMAFLREYFER

SEQ ID NO: 238 - protein, 20 aa, Homo sapiens
MEKHHVPSDFNVNVKVDTGP

SEQ ID NO: 239 - protein, 20 aa, Homo sapiens
REDLIKVLEDMRQEYELIIK

SEQ ID NO: 240 - protein, 20 aa, Homo sapiens
DEEDYTEPVADNETDYVEVM

SEQ ID NO: 241 - protein, 20 aa, Homo sapiens
MQAYDHLSYETPYETPQDEG

SEQ ID NO: 242 - protein, 20 aa, Homo sapiens
EITKPKRIRPSFKEAALRAY

SEQ ID NO: 243 - protein, 20 aa, Homo sapiens
VCGGAGGGLYGIDSMPDLRR

SEQ ID NO: 244 - protein, 20 aa, Homo sapiens
FKKTLQALIYPMSSTIPHNF

SEQ ID NO: 245 - protein, 50 aa, Homo sapiens
LYGIDSMPDLRRKKTLPIVRDVAMTLAARKSGLSLAMVIRTSLNNEELKM

SEQ ID NO: 246 - protein, 50 aa, Homo sapiens
LSYETPYETPQDEGYDGPADDMVSEEGLEPLNETSAEMEIREDENQNIPE

SEQ ID NO: 247 - protein, 20 aa, Homo sapiens
YLGLALDVDRIKKDQEEEED

SEQ ID NO: 248 - protein, 20 aa, Homo sapiens
DQGPPCPRLSRELLEVVEPE

SEQ ID NO: 249 - protein, 20 aa, Homo sapiens
LQDSLDRCYSTPSSCLEQPD

SEQ ID NO: 250 - protein, 20 aa, Homo sapiens
SCQPYGSSFYALEEKHVGFS

SEQ ID NO: 251 - protein, 50 aa, Homo sapiens
YLGLALDVDRIKKDQEEEEDQGPPCPRLSRELLEVVEPEVLQDSLDRCYS

SEQ ID NO: 252 - protein, 50 aa, Homo sapiens
RLSRELLEVVEPEVLQDSLDRCYSTPSSCLEQPDSCQPYGSSFYALEEKH

SEQ ID NO: 253 - protein, 20 aa, Homo sapiens
TPEQRGIFTISDHPAEQRGM

SEQ ID NO: 254 - protein, 20 aa, Homo sapiens
AVHGLELEALRLSLSNMHTA

SEQ ID NO: 255 - protein, 20 aa, Homo sapiens
HELELLREQHAREKEEVVLR

SEQ ID NO: 256 - protein, 20 aa, Homo sapiens
EDLQSEHGRCLEDLEFKFKE

SEQ ID NO: 257 - protein, 20 aa, Homo sapiens
LSELHEQLLARTSRVEDLEQ

SEQ ID NO: 258 - protein, 50 aa, Homo sapiens
ELEALRLSLSNMHTAQLELTQANLQKEKETALTELREMLNSRRAQELALL

SEQ ID NO: 259 - protein, 50 aa, Homo sapiens
KELSAKHQSEMEDLQNQFQKELAEQRAELEKIFQDKNQAERALRNLESHH

SEQ ID NO: 260 - protein, 20 aa, Homo sapiens
QQLEDMQKQLLHVQEKFYQI

SEQ ID NO: 261 - protein, 20 aa, Homo sapiens
ELDPGQFIDRARALIREQEM

SEQ ID NO: 262 - protein, 20 aa, Homo sapiens
SQVVDTVVKVFSAKPSRQVP

SEQ ID NO: 263 - protein, 20 aa, Homo sapiens
PLQIPQARFAVNGENHNFHT

SEQ ID NO: 264 - protein, 20 aa, Homo sapiens
TFGNVQMASSTDQTEALPLV

SEQ ID NO: 265 - protein, 50 aa, Homo sapiens
FSAKPSRQVPQVFPPLQIPQARFAVNGENHNFHTANQRLQCFGDVIIPNP

SEQ ID NO: 266 - protein, 50 aa, Homo sapiens
RESYRENKRKQKLPQQQQQSFQQLVSARKEQKREERRQLKQQLEDMQKQL

SEQ ID NO: 267 - protein, 20 aa, Homo sapiens
MEETQPPPQPKLPLCDSLMI

SEQ ID NO: 268 - protein, 20 aa, Homo sapiens
WLQTFNTASPCQDVKQLTSG

SEQ ID NO: 269 - protein, 20 aa, Homo sapiens
EELQEALAEKEELRQRCEEL

SEQ ID NO: 270 - protein, 20 aa, Homo sapiens
IDVLRATSDKANKLESTVEI

SEQ ID NO: 271 - protein, 20 aa, Homo sapiens
YRQKLQDLNDLRKQVKTLQE

SEQ ID NO: 272 - protein, 50 aa, Homo sapiens
EDVGDNWRIKASNVKKVLQGIMSYYHEFLGQQISEALIPDLNQITECSDP

SEQ ID NO: 273 - protein, 50 aa, Homo sapiens
RALEELQEALAEKEELRQRCEELDMQVTTLQDEKNSLVSENEMMNEKLDQ

SEQ ID NO: 274 - protein, 20 aa, Homo sapiens
SVFLFPPKPKDTLMISRTPE

SEQ ID NO: 275 - protein, 20 aa, Homo sapiens
VTCVVVDVSHEDPEVKFNWY

SEQ ID NO: 276 - protein, 20 aa, Homo sapiens
VDGVEVHNAKTKPREEQYNS

SEQ ID NO: 277 - protein, 20 aa, Homo sapiens
CLVKGFYPSDIAVEWESNGQ

SEQ ID NO: 278 - protein, 20 aa, Homo sapiens
PENNYKTTPPVLDSDGSFFL

SEQ ID NO: 279 - protein, 50 aa, Homo sapiens
TYRVVSVLTVLHQDWLNGKEYKCKVSNKALPAPIEKTISKAKGQPREPQV

SEQ ID NO: 280 - protein, 50 aa, Homo sapiens
DIAVEWESNGQPENNYKTTPPVLDSDGSFFLYSKLTVDKSRWQQGNVFSC

SEQ ID NO: 281 - protein, 20 aa, Homo sapiens
EQQLQAEAEHLRKELEGLAG

SEQ ID NO: 282 - protein, 20 aa, Homo sapiens
QLQAQVQDNEGLSRLNREQE

SEQ ID NO: 283 - protein, 20 aa, Homo sapiens
QAQVQDNEGLSRLNREQEER

SEQ ID NO: 284 - protein, 50 aa, Homo sapiens
EQQLQAEAEHLRKELEGLAGQLQAQVQDNEGLSRLNREQEERLLELERAA

SEQ ID NO: 285 - protein, 50 aa, Homo sapiens
AEHLRKELEGLAGQLQAQVQDNEGLSRLNREQEERLLELERAAELWGEQA

SEQ ID NO: 286 - protein, 20 aa, Homo sapiens
AEESELRFRQLTKEYQALQR

SEQ ID NO: 287 - protein, 20 aa, Homo sapiens
AYALLQEQTGGIIDAEREAK

SEQ ID NO: 288 - protein, 20 aa, Homo sapiens
AQEQLQAEVLRYKAKIEDLE

SEQ ID NO: 289 - protein, 20 aa, Homo sapiens
ATLAQKGQIEKQEAENHRLQ

SEQ ID NO: 290 - protein, 20 aa, Homo sapiens
QELQDARDQNELLEFRNLEL

SEQ ID NO: 291 - protein, 50 aa, Homo sapiens
AEESELRFRQLTKEYQALQRAYALLQEQTGGIIDAEREAKAQEQLQAEVL

SEQ ID NO: 292 - protein, 50 aa, Homo sapiens
RYKAKIEDLEATLAQKGQIEKQEAENHRLQQELQDARDQNELLEFRNLEL

SEQ ID NO: 293 - protein, 20 aa, Homo sapiens
MEEANIQPNRVTYQRLIASY

SEQ ID NO: 294 - protein, 20 aa, Homo sapiens
MENAENILTVMRDAGIEPGP

SEQ ID NO: 295 - protein, 20 aa, Homo sapiens
QTLEKVEKFELHLMDRDLLQ

SEQ ID NO: 296 - protein, 20 aa, Homo sapiens
VFGSFFLQHCVTMNTPVEKL

SEQ ID NO: 297 - protein, 20 aa, Homo sapiens
TDYCKKLKEVQMHSFPLQFT

SEQ ID NO: 298 - protein, 50 aa, Homo sapiens
MENAENILTVMRDAGIEPGPDTYLALLNAYAEKGDIDHVKQTLEKVEKFE

SEQ ID NO: 299 - protein, 50 aa, Homo sapiens
IIFSFSKAGYLSMSQKFWKKFTCERRYIPDAMNLILLLVTEKLEDVALQI

SEQ ID NO: 300 - protein, 20 aa, Homo sapiens
LTRVEVTEFEDIKSGYRIDF

SEQ ID NO: 301 - protein, 20 aa, Homo sapiens
YFDENPYFENKVLSKEFHLN

SEQ ID NO: 302 - protein, 20 aa, Homo sapiens
TKRSSQTQNKASRKRQHEEP

SEQ ID NO: 303 - protein, 20 aa, Homo sapiens
ESFFTWFTDHSDAGADELGE

SEQ ID NO: 304 - protein, 20 aa, Homo sapiens
VIKDDIWPNPLQYYLVPDMD

SEQ ID NO: 305 - protein, 50 aa, Homo sapiens
LTRVEVTEFEDIKSGYRIDFYFDENPYFENKVLSKEFHLNESGDPSSKST

SEQ ID NO: 306 - protein, 50 aa, Homo sapiens
EIKWKSGKDLTKRSSQTQNKASRKRQHEEPESFFTWFTDHSDAGADELGE

SEQ ID NO: 307 - protein, 20 aa, Homo sapiens
GVGSKHLMEGQKIITLDTAT

SEQ ID NO: 308 - protein, 20 aa, Homo sapiens
EIEGLSTGCKVYANIGEDTY

SEQ ID NO: 309 - protein, 20 aa, Homo sapiens
DIVIPVKDDPDEGEARLENE

SEQ ID NO: 310 - protein, 20 aa, Homo sapiens
HEIHHTGERRYQCLACGKSF

SEQ ID NO: 311 - protein, 20 aa, Homo sapiens
INYQFMSSHIKSVHSQDPSG

SEQ ID NO: 312 - protein, 50 aa, Homo sapiens
HDDHYELIVDGRVYYICIVCKRSYVCLTSLRRHFNIHSWEKKYPCRYCEK

SEQ ID NO: 313 - protein, 50 aa, Homo sapiens
YQCLACGKSFINYQFMSSHIKSVHSQDPSGDSKLYRLHPCRSLQIRQYAY

SEQ ID NO: 314 - protein, 20 aa, Homo sapiens
LSLKENSRLENELLENAEKL

SEQ ID NO: 315 - protein, 20 aa, Homo sapiens
AEYENLTNKLQRNLENVLAE

SEQ ID NO: 316 - protein, 20 aa, Homo sapiens
KFGDLDPSSAEFFLQEERLT

SEQ ID NO: 317 - protein, 20 aa, Homo sapiens
QMRNEYERQCRVLQDQVDEL

SEQ ID NO: 318 - protein, 20 aa, Homo sapiens
QSELEEYRAQGRVLRLPLKN

SEQ ID NO: 319 - protein, 50 aa, Homo sapiens
QVVREKEKLRSDLDKAEKLKSLMASEVDDHHAAIERRNEYNLRKLDGEYK

SEQ ID NO: 320 - protein, 50 aa, Homo sapiens
SEECNPLNMSIEAELVIEQMKEQHHRDICCLRLELEDKVRHYEKQLDETV

SEQ ID NO: 321 - protein, 20 aa, Homo sapiens
ILVELRDDIRDQVKEMSLIR

SEQ ID NO: 322 - protein, 20 aa, Homo sapiens
NTIMECQVCGFHEQRSHCSP

SEQ ID NO: 323 - protein, 20 aa, Homo sapiens
GPCPPGLQGNGTHCSDINEC

SEQ ID NO: 324 - protein, 20 aa, Homo sapiens
AHADPCFPGSSCINTMPGFH

SEQ ID NO: 325 - protein, 20 aa, Homo sapiens
CEACPRGYKGTQVSGVGIDY

SEQ ID NO: 326 - protein, 50 aa, Homo sapiens
AVTNALHSILGEQTKALVTQLTLFNQILVELRDDIRDQVKEMSLIRNTIM

SEQ ID NO: 327 - protein, 50 aa, Homo sapiens
INECAHADPCFPGSSCINTMPGFHCEACPRGYKGTQVSGVGIDYARASKQ

SEQ ID NO: 328 - protein, 20 aa, Homo sapiens
MSEPIRVLVTGAAGQIAYSL

SEQ ID NO: 329 - protein, 20 aa, Homo sapiens
AFKDLDVAILVGSMPRREGM

SEQ ID NO: 330 - protein, 20 aa, Homo sapiens
VIIWGNHSSTQYPDVNHAKV

SEQ ID NO: 331 - protein, 20 aa, Homo sapiens
VIIWGNHSSTQYPDVNHAKV

SEQ ID NO: 332 - protein, 20 aa, Homo sapiens
SSAMSAAKAICDHVRDIWFG

SEQ ID NO: 333 - protein, 50 aa, Homo sapiens
KKSVKVIVVGNPANTNCLTASKSAPSIPKENFSCLTRLDHNRAKAQIALK

SEQ ID NO: 334 - protein, 50 aa, Homo sapiens
QYPDVNHAKVKLQGKEVGVYEALKDDSWLKGEFVTTVQQRGAAVIKARKL

SEQ ID NO: 335 - protein, 20 aa, Homo sapiens
ESVVNSVVSLLLILEPDKQE

SEQ ID NO: 336 - protein, 20 aa, Homo sapiens
ALIESLCEKLVKFREGERPS

SEQ ID NO: 337 - protein, 20 aa, Homo sapiens
LTIFVSAKLASYVKFYQNNK

SEQ ID NO: 338 - protein, 20 aa, Homo sapiens
DFIDSLGLLHEQNMAKMRLL

SEQ ID NO: 339 - protein, 20 aa, Homo sapiens
LQIGADDVEAFVIDAVRTKM

SEQ ID NO: 340 - protein, 50 aa, Homo sapiens
MSVPAFIDISEEDQAAELRAYLKSKGAEISEENSEGGLHVDLAQIIEACD

SEQ ID NO: 341 - protein, 50 aa, Homo sapiens
LQIGADDVEAFVIDAVRTKMVYCKIDQTQRKVVVSHSTHRTFGKQRWQQL

SEQ ID NO: 342 - protein, 20 aa, Homo sapiens
RQFRRLFAQLAGDDMEVSAT

SEQ ID NO: 343 - protein, 20 aa, Homo sapiens
MNILNKVVTRHPDLKTDGFG

SEQ ID NO: 344 - protein, 20 aa, Homo sapiens
TCRSMVAVMDSDTTGKLGFE

SEQ ID NO: 345 - protein, 20 aa, Homo sapiens
FDTDRSGTICSSELPGAFEA

SEQ ID NO: 346 - protein, 20 aa, Homo sapiens
AGFHLNEHLYNMIIRRYSDE

SEQ ID NO: 347 - protein, 50 aa, Homo sapiens
EAAGFHLNEHLYNMIIRRYSDESGNMDFDNFISCLVRLDAMFRAFKSLDK

SEQ ID NO: 348 - protein, 50 aa, Homo sapiens
MNILNKVVTRHPDLKTDGFGIDTCRSMVAVMDSDTTGKLGFEEFKYLWNN

SEQ ID NO: 349 - protein, 20 aa, Homo sapiens
ERAPAQNPELTPGPAPAGPT

SEQ ID NO: 350 - protein, 20 aa, Homo sapiens
PAPETNAPNHPSPAEYVEVL

SEQ ID NO: 351 - protein, 20 aa, Homo sapiens
QELQRLESRLQPFLQRYYEV

SEQ ID NO: 352 - protein, 20 aa, Homo sapiens
ESSAEGAPPPGPAPPPATSH

SEQ ID NO: 353 - protein, 20 aa, Homo sapiens
PRVIRISHQSVEPVVMMHMN

SEQ ID NO: 354 - protein, 50 aa, Homo sapiens
ERAPAQNPELTPGPAPAGPTPAPETNAPNHPSPAEYVEVLQELQRLESRL

SEQ ID NO: 355 - protein, 50 aa, Homo sapiens
LGAAATTDYNNNHEGREEDQRLINLVGESLRLLGNTFVALSDLRCNLACT

SEQ ID NO: 356 - protein, 20 aa, Homo sapiens
GKTYHPNCFACTICKRPFPP

SEQ ID NO: 357 - protein, 20 aa, Homo sapiens
GDRVTFNGRDCLCQLCAQPM

SEQ ID NO: 358 - protein, 20 aa, Homo sapiens
SSSPKETTFSSNCAGCGRDI

SEQ ID NO: 359 - protein, 20 aa, Homo sapiens
DIKNGQALLALDKQWHLGCF

SEQ ID NO: 360 - protein, 50 aa, Homo sapiens
GKTYHPNCFACTICKRPFPPGDRVTFNGRDCLCQLCAQPMSSSPKETTFS

SEQ ID NO: 361 - protein, 50 aa, Homo sapiens
RDCLCQLCAQPMSSSPKETTFSSNCAGCGRDIKNGQALLALDKQWHLGCF

SEQ ID NO: 362 - protein, 20 aa, Homo sapiens
TTQHSSTLSTQQNTLSSSTS

SEQ ID NO: 363 - protein, 20 aa, Homo sapiens
HSSSSTFSTTSSTVSAPPPV

SEQ ID NO: 364 - protein, 20 aa, Homo sapiens
VSVSSSLNSGSSLGLSLGSN

SEQ ID NO: 365 - protein, 20 aa, Homo sapiens
NLPPGVPPLLPNPYIMAPGL

SEQ ID NO: 366 - protein, 20 aa, Homo sapiens
LHAYPPQVYGYDDLQMLQTR

SEQ ID NO: 367 - protein, 50 aa, Homo sapiens
TSVESEANLHSSSSTFSTTSSTVSAPPPVVSVSSSLNSGSSLGLSLGSNS

SEQ ID NO: 368 - protein, 50 aa, Homo sapiens
GLLHAYPPQVYGYDDLQMLQTRFPLDYYSIPFPTPTTPLTGRDGSLASNP

SEQ ID NO: 369 - protein, 20 aa, Homo sapiens
PEPAELSSGETEELQRIKWH

SEQ ID NO: 370 - protein, 20 aa, Homo sapiens
KEKELCIGRKKFNMDPAKGI

SEQ ID NO: 371 - protein, 20 aa, Homo sapiens
INLQVLQAFVDCHEFANLNL

SEQ ID NO: 372 - protein, 20 aa, Homo sapiens
STDTCYVLSFSIIMLNTSLH

SEQ ID NO: 373 - protein, 20 aa, Homo sapiens
DRMMEAFATRYCLCNPGVFQ

SEQ ID NO: 374 - protein, 50 aa, Homo sapiens
KFNMDPAKGIQYFIEHKLLTPDVQDIARFLYKGEGLNKTAIGTYLGERDP

SEQ ID NO: 375 - protein, 50 aa, Homo sapiens
VDCHEFANLNLVQALRQFLWSFRLPGEAQKIDRMMEAFATRYCLCNPGVF

SEQ ID NO: 376 - protein, 20 aa, Homo sapiens
RPKYAGLTFPKLFPDSLFPA

SEQ ID NO: 377 - protein, 20 aa, Homo sapiens
DSEHNKLKASQARDLLSKML

SEQ ID NO: 378 - protein, 20 aa, Homo sapiens
VNSSESLPPSSSVNDISSMS

SEQ ID NO: 379 - protein, 20 aa, Homo sapiens
TDQTLASDTDSSLEASAGPL

SEQ ID NO: 380 - protein, 20 aa, Homo sapiens
RNYVENRPKYAGLTFPKLFP

SEQ ID NO: 381 - protein, 50 aa, Homo sapiens
VWYDPAEVEAPPPQIYDKQLDEREHTIEEWKELIYKEVMNSEEKTKNGVV

SEQ ID NO: 382 - protein, 50 aa, Homo sapiens
KLFPDSLFPADSEHNKLKASQARDLLSKMLVIDPAKRISVDDALQHPYIN

SEQ ID NO: 383 - protein, 20 aa, Homo sapiens
AAVVEARAQASAAGILEEDL

SEQ ID NO: 384 - protein, 20 aa, Homo sapiens
RTARSALKLKNEEVESERER

SEQ ID NO: 385 - protein, 20 aa, Homo sapiens
AQALQEQGELKVAQGKALQE

SEQ ID NO: 386 - protein, 20 aa, Homo sapiens
LELEENHHKMECQQKLIKEL

SEQ ID NO: 387 - protein, 20 aa, Homo sapiens
AQGKALQENLALLTQTLAER

SEQ ID NO: 388 - protein, 50 aa, Homo sapiens
QKLTVQREQIRELEKDRETQRNVLEHQLLELEKKDQMIESQRGQVQDLKK

SEQ ID NO: 389 - protein, 50 aa, Homo sapiens
RGQIQELEKQREMQKAALELLSLDLKKRNQEVDLQQEQIQELEKCRSVLE

SEQ ID NO: 390 - protein, 20 aa, Homo sapiens
VTRLAKEVAKQCTDKRIRTN

SEQ ID NO: 391 - protein, 20 aa, Homo sapiens
LLQVCERIPTISTQLKILST

SEQ ID NO: 392 - protein, 20 aa, Homo sapiens
VKATMLGRTNISDEESEQAT

SEQ ID NO: 393 - protein, 20 aa, Homo sapiens
EMLVHNAQNLMQSVKETVRE

SEQ ID NO: 394 - protein, 20 aa, Homo sapiens
KIRTDAGFTLRWVRKTPWYQ

SEQ ID NO: 395 - protein, 50 aa, Homo sapiens
MSRLVRGGSGTKRALIQCAKDIAKASDEVTRLAKEVAKQCTDKRIRTNLL

SEQ ID NO: 396 - protein, 50 aa, Homo sapiens
EQATEMLVHNAQNLMQSVKETVREAEAASIKIRTDAGFTLRWVRKTPWYQ

SEQ ID NO: 397 - protein, 20 aa, Homo sapiens
VGNLPADITEDEFKRLFAKY

SEQ ID NO: 398 - protein, 20 aa, Homo sapiens
KPAARKAFERCSEGVFLLTT

SEQ ID NO: 399 - protein, 20 aa, Homo sapiens
AQHGTFEYEYSQRWKSLDEM

SEQ ID NO: 400 - protein, 20 aa, Homo sapiens
EKQQREQVEKNMKDAKDKLE

SEQ ID NO: 401 - protein, 20 aa, Homo sapiens
LSLLRRPGEKTYTQRCRLFG

SEQ ID NO: 402 - protein, 50 aa, Homo sapiens
QLRVRFATHAAALSVRNLSPYVSNELLEEAFSQFGPIERAVVIVDDRGRS

SEQ ID NO: 403 - protein, 50 aa, Homo sapiens
KPAARKAFERCSEGVFLLTTTPRPVIVEPLEQLDDEDGLPEKLAQKNPMY

SEQ ID NO: 404 - protein, 20 aa, Homo sapiens
IADILNQDAKNYHAWQHRQW

SEQ ID NO: 405 - protein, 20 aa, Homo sapiens
VIQEFKLWDNELQYVDQLLK

SEQ ID NO: 406 - protein, 20 aa, Homo sapiens
YFVISNTTGYNDRAVLEREV

SEQ ID NO: 407 - protein, 20 aa, Homo sapiens
QYTLEMIKLVPHNESAWNYL

SEQ ID NO: 408 - protein, 20 aa, Homo sapiens
KGILQDRGLSKYPNLLNQLL

SEQ ID NO: 409 - protein, 50 aa, Homo sapiens
YLKGILQDRGLSKYPNLLNQLLDLQPSHSSPYLIAFLVDIYEDMLENQCD

SEQ ID NO: 410 - protein, 50 aa, Homo sapiens
DILNQDAKNYHAWQHRQWVIQEFKLWDNELQYVDQLLKEDVRNNSVWNQR

SEQ ID NO: 411 - protein, 20 aa, Homo sapiens
NRNSDSAGPFAVPDHLRQDV

SEQ ID NO: 412 - protein, 20 aa, Homo sapiens
EALYDQHSNEYVVRNKKLWD

SEQ ID NO: 413 - protein, 20 aa, Homo sapiens
EALYDQHSNEYVVRNKKLWD

SEQ ID NO: 414 - protein, 20 aa, Homo sapiens
HGPLDMSNKMFSAEYEFFPE

SEQ ID NO: 415 - protein, 20 aa, Homo sapiens
ETRQILEKAGGLKPFLLGCP

SEQ ID NO: 416 - protein, 50 aa, Homo sapiens
EETRQILEKAGGLKPFLLGCPRFVVIDNCIALKKVASRLKKKRKKKNIKT

SEQ ID NO: 417 - protein, 50 aa, Homo sapiens
NRNSDSAGPFAVPDHLRQDVEEFEALYDQHSNEYVVRNKKLWDMNPKQKC

SEQ ID NO: 418 - protein, 20 aa, Homo sapiens
EFHNSKSSKELDFLREENHK

SEQ ID NO: 419 - protein, 20 aa, Homo sapiens
DLKRLSKDKDLEIDELQKRL

SEQ ID NO: 420 - protein, 20 aa, Homo sapiens
DWNMFVKLRSQECDWEEISV

SEQ ID NO: 421 - protein, 20 aa, Homo sapiens
RYVNKDMSIQELAVLVSGQK

SEQ ID NO: 422 - protein, 20 aa, Homo sapiens
VAVKREQRENHLRRSIVVIH

SEQ ID NO: 423 - protein, 50 aa, Homo sapiens
LREENHKLQLERQNLQLETRRLQSEINMAATETRDLRNMTVADSGTNHDS

SEQ ID NO: 424 - protein, 50 aa, Homo sapiens
RSQECDWEEISVKGPNGESSVIHDRKSGKKFSIEEALQSGRLTPAQYDRY

SEQ ID NO: 425 - protein, 20 aa, Homo sapiens
STEKNSLVFQLERLEQQMNS

SEQ ID NO: 426 - protein, 20 aa, Homo sapiens
DTETNLAGMYGKVRKAASSI

SEQ ID NO: 427 - protein, 20 aa, Homo sapiens
YPIARVFVIIYMALLHLWVM

SEQ ID NO: 428 - protein, 20 aa, Homo sapiens
VMIVLLTYTPEMHHDQPYGK

SEQ ID NO: 429 - protein, 20 aa, Homo sapiens
MSGIDNGEGTRLRNVPVLFN

SEQ ID NO: 430 - protein, 50 aa, Homo sapiens
STEKNSLVFQLERLEQQMNSASGSSSNGSSINMSGIDNGEGTRLRNVPVL

SEQ ID NO: 431 - protein, 50 aa, Homo sapiens
DTETNLAGMYGKVRKAASSIDQFSIRLGIFLRRYPIARVFVIIYMALLHL

SEQ ID NO: 432 - protein, 20 aa, Homo sapiens
RYPKLAALNPESNTAGLDIF

SEQ ID NO: 433 - protein, 20 aa, Homo sapiens
AKFSAYIKNSNPALNDNLEK

SEQ ID NO: 434 - protein, 20 aa, Homo sapiens
VDETSAEDEGVSQRKFLDGN

SEQ ID NO: 435 - protein, 20 aa, Homo sapiens
ELTLADCNLLPKLHIVQVVC

SEQ ID NO: 436 - protein, 20 aa, Homo sapiens
NAYAREEFASTCPDDEEIEL

SEQ ID NO: 437 - protein, 50 aa, Homo sapiens
ESNTAGLDIFAKFSAYIKNSNPALNDNLEKGLLKALKVLDNYLTSPLPEE

SEQ ID NO: 438 - protein, 50 aa, Homo sapiens
NLLPKLHIVQVVCKKYRGFTIPEAFRGVHRYLSNAYAREEFASTCPDDEE

SEQ ID NO: 439 - protein, 20 aa, Homo sapiens
GLGLGGGDCRVKIATKANPW

SEQ ID NO: 440 - protein, 20 aa, Homo sapiens
EETLHACQRLHQEGKFVELG

SEQ ID NO: 441 - protein, 20 aa, Homo sapiens
ELFPCLRHFGLRFYAYNPLA

SEQ ID NO: 442 - protein, 20 aa, Homo sapiens
FEAIALVEKALQAAYGASAP

SEQ ID NO: 443 - protein, 20 aa, Homo sapiens
TEEGPLEPAVVDAFNQAWHL

SEQ ID NO: 444 - protein, 50 aa, Homo sapiens
HQEGKFVELGLSNYASWEVAEICTLCKSNGWILPTVYQGMYNATTRQVET

SEQ ID NO: 445 - protein, 50 aa, Homo sapiens
LQAAYGASAPSVTSAALRWMYHHSQLQGAHGDAVILGMSSLEQLEQNLAA

SEQ ID NO: 446 - protein, 20 aa, Homo sapiens
SNRNSDSAGPFAVPDHLRQD

SEQ ID NO: 447 - protein, 20 aa, Homo sapiens
EALYDQHSNEYVVRNKKLWD

SEQ ID NO: 448 - protein, 20 aa, Homo sapiens
MNPKQKCSTLYDYFSQFLEE

SEQ ID NO: 449 - protein, 20 aa, Homo sapiens
HGPLDMSNKMFSAEYEFFPE

SEQ ID NO: 450 - protein, 20 aa, Homo sapiens
ETRQILEKAGGLKPFLLGCP

SEQ ID NO: 451 - protein, 50 aa, Homo sapiens; identical to SEQ ID NO: 416

SEQ ID NO: 452 - protein, 50 aa, Homo sapiens
FAVPDHLRQDVEEFALYDQHSNEYVVRNKKLWDMNPKQKCSTLYDYFSQF

SEQ ID NO: 453 - protein, 20 aa, Homo sapiens
GQSPTGSVCYERNGEVFYLT

SEQ ID NO: 454 - protein, 20 aa, Homo sapiens
KKQARCQGVVCAMKEAFGFI

SEQ ID NO: 455 - protein, 20 aa, Homo sapiens
VATDVRLLPQGTVIFEDISI

SEQ ID NO: 456 - protein, 20 aa, Homo sapiens
DKDTKSKVTLLEGDHVRFNI

SEQ ID NO: 457 - protein, 20 aa, Homo sapiens
HSHSDHRFLGTVEKEATFSN

SEQ ID NO: 458 - protein, 50 aa, Homo sapiens
GFIKCVDRDVRMFFHFSEILDGNQLHIADEVEFTVVPDMLSAQRNHAIRI

SEQ ID NO: 459 - protein, 50 aa, Homo sapiens
CAMKEAFGFIERGDVVKEIFFHYSEFKGDLETLQPGDDVEFTIKDRNGKE

SEQ ID NO: 460 - protein, 20 aa, Homo sapiens
HKLIHAASERVLSDARTILE

SEQ ID NO: 461 - protein, 20 aa, Homo sapiens
ENIQDQDVLLLIKKRAPSPL

SEQ ID NO: 462 - protein, 20 aa, Homo sapiens
PKMADVSAEEKKKQDQKAPD

SEQ ID NO: 463 - protein, 20 aa, Homo sapiens
KEAILRATANLPSYNMDRAA

SEQ ID NO: 464 - protein, 20 aa, Homo sapiens
VQTNMRDFQTELRKILVSLI

SEQ ID NO: 465 - protein, 50 aa, Homo sapiens
KSITHHKLIHAASERVLSDARTILEENIQDQDVLLLIKKRAPSPLPKMAD

SEQ ID NO: 466 - protein, 50 aa, Homo sapiens
MDRAAVQTNMRDFQTELRKILVSLIEVAQKLLALNPDAVELFKKANAMLD

SEQ ID NO: 467 - protein, 20 aa, Homo sapiens
SSQESLDISLCSTGSLGSLG

SEQ ID NO: 468 - protein, 20 aa, Homo sapiens
VVEIRSRAKIEKIRASLFNN

SEQ ID NO: 469 - protein, 20 aa, Homo sapiens
GLEDYFNDKSIKGEKLVPGA

SEQ ID NO: 470 - protein, 20 aa, Homo sapiens
QTPGNLLHLFFTNVRPPKKV

SEQ ID NO: 471 - protein, 20 aa, Homo sapiens
PKFDKSKYSKAGKEQHPVKV

SEQ ID NO: 472 - protein, 50 aa, Homo sapiens
EKIRASLFNNNDLIGLSSLDGEDELMEMSTEEILTVSVVNQSLFDTQGSP

SEQ ID NO: 473 - protein, 50 aa, Homo sapiens
SIKGEKLVPGAREVLTEIFKSCAHSEQTLSLTPAKPIRVSDIYLSKEQIN

SEQ ID NO: 474 - protein, 20 aa, Homo sapiens
TESRSSFSQHARTSGRVAVE

SEQ ID NO: 475 - protein, 20 aa, Homo sapiens
YRFPPKFTLKAGQVVTIWAA

SEQ ID NO: 476 - protein, 20 aa, Homo sapiens
RKLVRSVTVVEDDEDEDGDD

SEQ ID NO: 477 - protein, 20 aa, Homo sapiens
RLRSPGGRTHLLWLFCLQCH

SEQ ID NO: 478 - protein, 20 aa, Homo sapiens
LRGQSGHPLLPPGQLQPPNP

SEQ ID NO: 479 - protein, 50 aa, Homo sapiens
RSSFSQHARTSGRVAVEEVDEEGKFVRLRNKSNEDQSMGNWQIKRQNGDD

SEQ ID NO: 480 - protein, 50 aa, Homo sapiens
HLLWLFCLQCHGHSQLPQCGGQWGWQLRGQSGHPLLPPGQLQPPNPEPPE

SEQ ID NO: 481 - protein, 20 aa, Homo sapiens
PDFIMAKQVEQLEKEKKELQ

SEQ ID NO: 482 - protein, 20 aa, Homo sapiens
DMDLWEQQEEERITTMQLER

SEQ ID NO: 483 - protein, 20 aa, Homo sapiens
ERLAEERHNRLEERKRQRKE

SEQ ID NO: 484 - protein, 20 aa, Homo sapiens
ERRITYYREKEEEEQRRAEE

SEQ ID NO: 485 - protein, 20 aa, Homo sapiens
QMLKEREERERAERAKREEE

SEQ ID NO: 486 - protein, 50 aa, Homo sapiens
RITYYREKEEEEQRRAEEQMLKEREERERAERAKREEELREYQERVKKLE

SEQ ID NO: 487 - protein, 50 aa, Homo sapiens
MAKQVEQLEKEKKELQERLKNQEKKIDYFERAKRLEEIPLIKSAYEEQRI

SEQ ID NO: 488 - protein, 20 aa, Homo sapiens
QADNDFTNERLTALQENQTR

SEQ ID NO: 489 - protein, 20 aa, Homo sapiens
MDEQDLNEPLAKVSLLKDDL

SEQ ID NO: 490 - protein, 20 aa, Homo sapiens
EEERKAYRNQVEESTKQIQV

SEQ ID NO: 491 - protein, 20 aa, Homo sapiens
LHQAAAKVASERDTDIASLQ

SEQ ID NO: 492 - protein, 20 aa, Homo sapiens
EDQQREEATRLQGELEKLRK

SEQ ID NO: 493 - protein, 50 aa, Homo sapiens
LAKVSLLKDDLQGAQSEIEAKQEIQHLRKELIEAQELARTSKQKCFELQA

SEQ ID NO: 494 - protein, 50 aa, Homo sapiens
LHQAAAKVASERDTDIASLQEELKKVRAELERWRKAASEYEKEITSLQNS

SEQ ID NO: 495 - protein, 20 aa, Homo sapiens
LYSARDLQGLTVEHAIDSFR

SEQ ID NO: 496 - protein, 20 aa, Homo sapiens
EKNVELRKKKPDYLPYAEDE

SEQ ID NO: 497 - protein, 20 aa, Homo sapiens
SVDDLAQQKPRSILSKYDEE

SEQ ID NO: 498 - protein, 20 aa, Homo sapiens
LEGERPHSFRLEQGGTADGL

SEQ ID NO: 499 - protein, 20 aa, Homo sapiens
RERELEEIRAKLRLQAQSLS

SEQ ID NO: 500 - protein, 50 aa, Homo sapiens
LYSARDLQGLTVEHAIDSFREGETMILTLKDKGVLQEEEDVLVNVNLVDK

SEQ ID NO: 501 - protein, 50 aa, Homo sapiens
EIRAKLRLQAQSLSTVGPRLASEYLTPEEMVTFKKTKRRVKKIRKKEKEV

SEQ ID NO: 502 - protein, 20 aa, Homo sapiens
HKLIHAASERVLSDARTILE

SEQ ID NO: 503 - protein, 20 aa, Homo sapiens
ENIQDQDVLLLIKKRAPSPL

SEQ ID NO: 504 - protein, 20 aa, Homo sapiens
PKMADVSAEEKKKQDQKAPD

SEQ ID NO: 505 - protein, 20 aa, Homo sapiens
KEAILRATANLPSYNMDRAA

SEQ ID NO: 506 - protein, 20 aa, Homo sapiens
VQTNMRDFQTELRKILVSLI

SEQ ID NO: 507 - protein, 50 aa, Homo sapiens
ITHHKLIHAASERVLSDARTILEENIQDQDVLLLIKKRAPSPLPKMADVS

SEQ ID NO: 508 - protein, 50 aa, Homo sapiens
APDKEAILRATANLPSYNMDRAAVQTNMRDFQTELRKILVSLIEVAQKLL

SEQ ID NO: 509 - protein, 20 aa, Homo sapiens
TENYEQRIREQQEQLSLQQT

SEQ ID NO: 510 - protein, 20 aa, Homo sapiens
ETSARSLGSPLLHERGNIEK

SEQ ID NO: 511 - protein, 20 aa, Homo sapiens
NDSTFLHSTPERPGILSPAT

SEQ ID NO: 512 - protein, 20 aa, Homo sapiens
AIRGPQQPIWKPFPNQDSDS

SEQ ID NO: 513 - protein, 20 aa, Homo sapiens
VVLSGTDSELHIPRVCEFCQ

SEQ ID NO: 514 - protein, 50 aa, Homo sapiens
QLLLVNSTQDNNYGCVPLLEDSDTRKNTLTLAQPQDKVISGIAREKLPKV

SEQ ID NO: 515 - protein, 50 aa, Homo sapiens
QNLKTTDKTKPSNLVNTCIRTTLDRAACLPPGDHNALYVNSFPLLDPSDA

SEQ ID NO: 516 - protein, 20 aa, Homo sapiens
QLLLVNSTQDNNYGCVPLLE

SEQ ID NO: 517 - protein, 20 aa, Homo sapiens
ETSARSLGSPLLHERGNIEK

SEQ ID NO: 518 - protein, 20 aa, Homo sapiens
IQCTDKTDKQEALFTPQAKD

SEQ ID NO: 519 - protein, 20 aa, Homo sapiens
NDSTFLHSTPERPGILSPAT

SEQ ID NO: 520 - protein, 20 aa, Homo sapiens
QNLKTTDKTKPSNLVNTCIR

SEQ ID NO: 521 - protein, 50 aa, Homo sapiens
AIRGPQQPIWKPFPNQDSDSVVLSGTDSELHIPRVCEFCQAVFPPSITSR

SEQ ID NO: 522 - protein, 50 aa, Homo sapiens
QDNNYGCVPLLEDSDTRKNTLTLAQPQDKVISGIAREKLPKVRRQEVSSP

SEQ ID NO: 523 - protein, 20 aa, Homo sapiens
STQAALQAMEREAEQMGNEL

SEQ ID NO: 524 - protein, 20 aa, Homo sapiens
ALEKAARAELEMRLQNALNE

SEQ ID NO: 525 - protein, 20 aa, Homo sapiens
QRVEFATLQEALAHALTEKE

SEQ ID NO: 526 - protein, 20 aa, Homo sapiens
GKDQELAKLRGLEAAQIKEL

SEQ ID NO: 527 - protein, 20 aa, Homo sapiens
EAAQIKELEELRQTVKQLKE

SEQ ID NO: 528 - protein, 50 aa, Homo sapiens
AMEREAEQMGNELERLRAALMESQGQQQEERGQQEREVARLTQERGRAQA

SEQ ID NO: 529 - protein, 50 aa, Homo sapiens
VEFATLQEALAHALTEKEGKDQELAKLRGLEAAQIKELEELRQTVKQLKE

SEQ ID NO: 530 - protein, 20 aa, Homo sapiens
LEDEWQAISEIASTCNTILE

SEQ ID NO: 531 - protein, 20 aa, Homo sapiens
GTPKSDAEPEPGNLSEKVSH

SEQ ID NO: 532 - protein, 20 aa, Homo sapiens
LESMLRKLQEDLQKEKADRA

SEQ ID NO: 533 - protein, 20 aa, Homo sapiens
ALEEEVRSLRHNNRRLQAES

SEQ ID NO: 534 - protein, 20 aa, Homo sapiens
ESAATRLLLASKQLGSPTAD

SEQ ID NO: 535 - protein, 50 aa, Homo sapiens
LEDEWQAISEIASTCNTILESLSREGQPIPESGDPKGTPKSDAEPEPGNL

SEQ ID NO: 536 - protein, 50 aa, Homo sapiens
QKEKADRAALEEEVRSLRHNNRRLQAESESAATRLLLASKQLGSPTADLA

SEQ ID NO: 537 - protein, 20 aa, Homo sapiens
SLVSSYLQTQEMVSNFDLNS

SEQ ID NO: 538 - protein, 20 aa, Homo sapiens
QLEVREKQLQERMQQLDREN

SEQ ID NO: 539 - protein, 20 aa, Homo sapiens
QWEVTQATQNTVKELQTCLQ

SEQ ID NO: 540 - protein, 20 aa, Homo sapiens
KNQHLASFPGWLAMAQQKAD

SEQ ID NO: 541 - protein, 20 aa, Homo sapiens
NRQQSAQLEQLVKELQLKED

SEQ ID NO: 542 - protein, 50 aa, Homo sapiens
LKEDARASLERLVKEMAPLQEELSGKGQEADQLWRRLQELLAHTSSWEEE

SEQ ID NO: 543 - protein, 50 aa, Homo sapiens
AVSQQGEQLQTERERGRTAAEDNVRLTCLVAELQKQWEVTQATQNTVKEL

SEQ ID NO: 544 - protein, 20 aa, Homo sapiens
MCDRKAVIKNADMSEEMQQD

SEQ ID NO: 545 - protein, 20 aa, Homo sapiens
SVECATQALEKYNIEKDIAA

SEQ ID NO: 546 - protein, 20 aa, Homo sapiens
HIKKEFDKKYNPTWHCIVGR

SEQ ID NO: 547 - protein, 20 aa, Homo sapiens
NFGSYVTHETKHFIYFYLGQ

SEQ ID NO: 548 - protein, 50 aa, Homo sapiens
AHIKKEFDKKYNPTWHCIVGRNFGSYVTHETKHFIYFYLGQVAILLFKSG

SEQ ID NO: 549 - protein, 50 aa, Homo sapiens
MCDRKAVIKNADMSEEMQQDSVECATQALEKYNIEKDIAAHIKKEFDKKY

SEQ ID NO: 550 - protein, 20 aa, Homo sapiens
KRGILTLKYPIEHGIVTNWD

SEQ ID NO: 551 - protein, 20 aa, Homo sapiens
QIMFETFNTPAMYVAIQAVL

SEQ ID NO: 552 - protein, 20 aa, Homo sapiens
GRDLTDYLMKILTERGYSFT

SEQ ID NO: 553 - protein, 20 aa, Homo sapiens
AGRDLTDYLMKILTERGYSF

SEQ ID NO: 554 - protein, 20 aa, Homo sapiens
GGTTMYPGIADRMQKEITAL

SEQ ID NO: 555 - protein, 50 aa, Homo sapiens
IEHGIVTNWDDMEKIWHHTFYNELRVAPEEHPVLLTEAPLNPKANREKMT

SEQ ID NO: 556 - protein, 50 aa, Homo sapiens
KIKIIAPPERKYSVWIGGSILASLSTFQQMWISKQEYDESGPSIVHRKCF

SEQ ID NO: 557 - protein, 20 aa, Homo sapiens
FQDMMGHGSDYSLSEVLWVC

SEQ ID NO: 558 - protein, 20 aa, Homo sapiens
AKASRARTKAGDLRDTGIFL

SEQ ID NO: 559 - protein, 20 aa, Homo sapiens
DLLRKVRAKETRKRALSRLK

SEQ ID NO: 560 - protein, 20 aa, Homo sapiens
RTFNTSTGGLLLPSDTKRSQ

SEQ ID NO: 561 - protein, 20 aa, Homo sapiens
YLRPSLFVYPEESLVIGSST

SEQ ID NO: 562 - protein, 50 aa, Homo sapiens
DLMHLKKPGGFDISLFYRDIISIAEDEDLRVHFEESSKLEDLLRKVRAKE

SEQ ID NO: 563 - protein, 50 aa, Homo sapiens
ISVGIYNLVQKALKPPPIKLYRETNEPVKTKTRTFNTSTGGLLLPSDTKR

SEQ ID NO: 564 - protein, 20 aa, Homo sapiens
NQKAAESLADPTEYENLFPG

SEQ ID NO: 565 - protein, 20 aa, Homo sapiens
LKEAFVVEEWVKETHADLWP

SEQ ID NO: 566 - protein, 20 aa, Homo sapiens
AKQYPLVTPNEERNVMEEGK

SEQ ID NO: 567 - protein, 20 aa, Homo sapiens
DFQPSRSTAQQELDGKPASP

SEQ ID NO: 568 - protein, 20 aa, Homo sapiens
TPVIVASHTANKEEKSLLEL

SEQ ID NO: 569 - protein, 50 aa, Homo sapiens
VVKLWRENLSKVNQKAAESLADPTEYENLFPGLKEAFVVEEWVKETHADL

SEQ ID NO: 570 - protein, 50 aa, Homo sapiens
PASPTPVIVASHTANKEEKSLLELEVDLDNLELEDIDTTDINLDEDILDD

SEQ ID NO: 571 - protein, 20 aa, Homo sapiens
MRIAVICFCLLGITCAIPVK

SEQ ID NO: 572 - protein, 20 aa, Homo sapiens
QADSGSSEEKQLYNKYPDAV

SEQ ID NO: 573 - protein, 20 aa, Homo sapiens
PDAVATWLNPDPSQKQNLLA

SEQ ID NO: 574 - protein, 50 aa, Homo sapiens
MRIAVICFCLLGITCAIPVKQADSGSSEEKQLYNKYPDAVATWLNPDPSQ

SEQ ID NO: 575 - protein, 20 aa, Homo sapiens
NFLDGALVLEVDERDQDDEE

SEQ ID NO: 576 - protein, 20 aa, Homo sapiens
RAFYRIQATRLMTGAGNILK

SEQ ID NO: 577 - protein, 20 aa, Homo sapiens
NCGTVALTIIRRGGDLTNTV

SEQ ID NO: 578 - protein, 20 aa, Homo sapiens
DDDIFEEDENFLVHLSNVKV

SEQ ID NO: 579 - protein, 20 aa, Homo sapiens
TFEEPVTHVSESIGIMEVKV

SEQ ID NO: 580 - protein, 50 aa, Homo sapiens
RREMARILKELKQKHPDKEIEQLIELANYQVLSQQQKSRAFYRIQATRLM

SEQ ID NO: 581 - protein, 50 aa, Homo sapiens
TATVTIFDDDHAGIFTFEEPVTHVSESIGIMEVKVLRTSGARGNVIVPYK

SEQ ID NO: 582 - protein, 20 aa, Homo sapiens
AASVEQATQNFLEKGEQIAK

SEQ ID NO: 583 - protein, 20 aa, Homo sapiens
KKMVKLNYVAARRQQELKDP

SEQ ID NO: 584 - protein, 20 aa, Homo sapiens
PSLEERLESIISGAALMADS

SEQ ID NO: 585 - protein, 20 aa, Homo sapiens
FREHANKLVEVANLACSISN

SEQ ID NO: 586 - protein, 20 aa, Homo sapiens
SLCPQVINAALTLAARPQSK

SEQ ID NO: 587 - protein, 50 aa, Homo sapiens
GTMVRAARALLSAVTRLLILADMADVMRLLSHLKIVEEALEAVKNATNEQ

SEQ ID NO: 588 - protein, 50 aa, Homo sapiens
PLNIAIDKMTKKTRDLRRQLRKAVMDHISDSFLETNVPLLVLIEAAKSGN

SEQ ID NO: 589 - protein, 20 aa, Homo sapiens
PSLLRDVQLSGPGGQAQAEA

SEQ ID NO: 590 - protein, 20 aa, Homo sapiens
TMLKAICVDVDHGLLPREEW

SEQ ID NO: 591 - protein, 20 aa, Homo sapiens
QAKVAGSEENGTAETEEVED

SEQ ID NO: 592 - protein, 20 aa, Homo sapiens
ESASGELDLEAQFHLHFSSL

SEQ ID NO: 593 - protein, 20 aa, Homo sapiens
HHILMHLTEKAQEVTRKYQE

SEQ ID NO: 594 - protein, 50 aa, Homo sapiens
PSLLRDVQLSGPGGQAQAEAPDLYTYFTMLKAICVDVDHGLLPREEWQAK

SEQ ID NO: 595 - protein, 50 aa, Homo sapiens
ENGTAETEEVEDESASGELDLEAQFHLHFSSLHHILMHLTEKAQEVTRKY

SEQ ID NO: 596 - protein, 20 aa, Homo sapiens
LVKLAPDFDKIVESLSLLKD

SEQ ID NO: 597 - protein, 20 aa, Homo sapiens
DFFTSGSPEETAFRATDRGS

SEQ ID NO: 598 - protein, 20 aa, Homo sapiens
KELRKLVLQKDDKGIHHRKL

SEQ ID NO: 599 - protein, 20 aa, Homo sapiens
KTSVLEMIAQARIFPRGSGE

SEQ ID NO: 600 - protein, 20 aa, Homo sapiens
HEIELRMRKNVKEGCTVSPE

SEQ ID NO: 601 - protein, 50 aa, Homo sapiens
FRKVSDKEKIDQLQEELLHTQLKYQRILERLEKENKELRKLVLQKDDKGI

SEQ ID NO: 602 - protein, 50 aa, Homo sapiens
MTRSPVKVTLSEGPHHVALFKDSSREFDLTKEEDLAALRHEIELRMRKNV

SEQ ID NO: 603 - protein, 20 aa, Homo sapiens
TKGHSYPPGPAEVSRLRRCR

SEQ ID NO: 604 - protein, 20 aa, Homo sapiens
SAQPSPERQPSPLEPRPVSP

SEQ ID NO: 605 - protein, 20 aa, Homo sapiens
AALDALDKAQRQLQACKRRE

SEQ ID NO: 606 - protein, 20 aa, Homo sapiens
AREKRAQADARQTLKEHVQD

SEQ ID NO: 607 - protein, 20 aa, Homo sapiens
EKRAQADARQTLKEHVQDFA

SEQ ID NO: 608 - protein, 50 aa, Homo sapiens
PLGAQADEAGCSAQPSPERQPSPLEPRPVSPSAYMLRLPPPAGAYIQNEH

SEQ ID NO: 609 - protein, 50 aa, Homo sapiens; identical to SEQ ID NO: 608

SEQ ID NO: 610 - protein, 20 aa, Homo sapiens
AIVEFDYQAQHDDELTISVG

SEQ ID NO: 611 - protein, 20 aa, Homo sapiens
KKDPLTNKAPEKPLHEVPSG

SEQ ID NO: 612 - protein, 20 aa, Homo sapiens
VGDIIEVVGEVEEGWWEGVL

SEQ ID NO: 613 - protein, 20 aa, Homo sapiens
SESDGGDSSSTKSEGANGTV

SEQ ID NO: 614 - protein, 20 aa, Homo sapiens
EKTIGKKLPATTATPDSSKT

SEQ ID NO: 615 - protein, 50 aa, Homo sapiens
HDDELTISVGEIITNIRKEDGGWWEGQINGRRGLFPDNFVREIKKEMKKD

SEQ ID NO: 616 - protein, 50 aa, Homo sapiens
TKSEGANGTVATAAIQPKKVKGVGFGDIFKDKPIKLRPRSIEVENDFLPV

SEQ ID NO: 617 - protein, 24 aa, Homo sapiens
EHGEPFIQACMTFLKRRCPSILGG

SEQ ID NO: 618 - protein, 29 aa, Homo sapiens
QACAGSVSQELSETILTMVANCSNVMNKA

SEQ ID NO: 619 - protein, 27 aa, Homo sapiens
GMTSLSIGGSAAPHTQSMQGFPPNLGS

SEQ ID NO: 620 - protein, 29 aa, Homo sapiens
LPVGGLGTGSLTGIGTGALGLPAVNNDPF

SEQ ID NO: 621 - protein, 24 aa, Homo sapiens
ANQHFSKEIDDEANSYFQRIYNHP

SEQ ID NO: 622 - protein, 50 aa, Homo sapiens
FEEYRFFPQYPDKELHITACLFGGIIEKGLVTYMALGLALRYVLEALRKP

SEQ ID NO: 623 - protein, 50 aa, Homo sapiens
DTLLVATDQTERIVEPPENIQEKIAFIFNNLSQSNMTQKVEELKETVKEE

SEQ ID NO: 624 - protein, 28 aa, Homo sapiens
MADYSTVPPPSSGSAGGGGGGGGGGGVN

SEQ ID NO: 625 - protein, 20 aa, Homo sapiens
EQISRIQQESGCKIQIAPDS

SEQ ID NO: 626 - protein, 20 aa, Homo sapiens
EQISRIQQESGCKIQIAPDS

SEQ ID NO: 627 - protein, 20 aa, Homo sapiens
PYKVQQAKEMVLELIRDQGG

SEQ ID NO: 628 - protein, 20 aa, Homo sapiens
RGRGQGNWNMGPPGGLQEFN

SEQ ID NO: 629 - protein, 50 aa, Homo sapiens
MGPYNPAPYNPGPPGPAPHGPPAPYAPQGWGNAYPHWQQQAPPDPAKAGT

SEQ ID NO: 630 - protein, 50 aa, Homo sapiens
GGQKRPLEDGDQPDAKKVAPQNDSFGTQLPPMHQQQSRSVMTEEYKVPDG

SEQ ID NO: 631 - protein, 23 aa, Homo sapiens
EWLCLLSPSGPVCTCPNGKRLDN

SEQ ID NO: 632 - protein, 27 aa, Homo sapiens
NLQCFNGGSCFLNARRQPKCRCQPRYT

SEQ ID NO: 633 - protein, 25 aa, Homo sapiens
FTGPKCTQQVCAGYCANNSTCTVNQ

SEQ ID NO: 634 - protein, 29 aa, Homo sapiens
AADGSRQCRCTAYFEGSRCEVNKCSRCLE

SEQ ID NO: 635 - protein, 26 aa, Homo sapiens
CSNGGSCTMNSKMMPECQCPPHMTGP

SEQ ID NO: 636 - protein, 50 aa, Homo sapiens
VFWYKRRVQGAKGFQHQRMTNGAMNVEIGNPTYKMYEGGEPDDVGGLLDA

SEQ ID NO: 637 - protein, 50 aa, Homo sapiens
CFLNARRQPKCRCQPRYTGDKCELDQCWEHCRNGGTCAASPSGMPTCRCP

SEQ ID NO: 638 - protein, 20 aa, Homo sapiens
MASNDYTQQATQSYGAYPTQ

SEQ ID NO: 639 - protein, 20 aa, Homo sapiens
PGQGYSQQSSQPYGQQSYSG

SEQ ID NO: 640 - protein, 20 aa, Homo sapiens
PGQGYSQQSSQPYGQQSYSG

SEQ ID NO: 641 - protein, 20 aa, Homo sapiens
SQNTGYGTQSTPQGYGSTGG

SEQ ID NO: 642 - protein, 20 aa, Homo sapiens
YGSSQSSQSSYGQQSSYPGY

SEQ ID NO: 643 - protein, 50 aa, Homo sapiens
TGYGTQSTPQGYGSTGGYGSSQSSQSSYGQQSSYPGYGQQPAPSSTSGSY

SEQ ID NO: 644 - protein, 50 aa, Homo sapiens
MASNDYTQQATQSYGAYPTQPGQGYSQQSSQPYGQQSYSGYSQSTDTSGY

SEQ ID NO: 645 - protein, 20 aa, Homo sapiens
MASNDYTQQATQSYGAYPTQ

SEQ ID NO: 646 - protein, 20 aa, Homo sapiens
PGQGYSQQSSQPYGQQSYSG

SEQ ID NO: 647 - protein, 20 aa, Homo sapiens
YSQSTDTSGYGQSSYSSYGQ

SEQ ID NO: 648 - protein, 20 aa, Homo sapiens
SQNSYGTQSTPQGYGSTGGY

SEQ ID NO: 649 - protein, 20 aa, Homo sapiens
GSSQSSQSSYGQQSSYPGYG

SEQ ID NO: 650 - protein, 50 aa, Homo sapiens
NSYGTQSTPQGYGSTGGYGSSQSSQSSYGQQSSYPGYGQQPAPSSTSGSY

SEQ ID NO: 651 - protein, 50 aa, Homo sapiens
ASNDYTQQATQSYGAYPTQPGQGYSQQSSQPYGQQSYSGYSQSTDTSGYG

SEQ ID NO: 652 - protein, 20 aa, Homo sapiens
DTKFLCPLCMRSRRPRLETI

SEQ ID NO: 653 - protein, 20 aa, Homo sapiens
LLVALQRLPVRLPEGEALQC

SEQ ID NO: 654 - protein, 20 aa, Homo sapiens
PEEPPNYPAAPASDPLREGS

SEQ ID NO: 655 - protein, 20 aa, Homo sapiens
LPQLTGPVLELPEATRAPLE

SEQ ID NO: 656 - protein, 20 aa, Homo sapiens
EELMMEGDLLEVTLDENHSI

SEQ ID NO: 657 - protein, 50 aa, Homo sapiens
VRLPEGEALQCLTERAISWQGRARQALASEDVTALLGRLAELRQRLQAEP

SEQ ID NO: 658 - protein, 50 aa, Homo sapiens
DTKFLCPLCMRSRRPRLETILALLVALQRLPVRLPEGEALQCLTERAISW

SEQ ID NO: 659 - protein, 20 aa, Homo sapiens
QDYPYVMADSLLDVLTKGGH

SEQ ID NO: 660 - protein, 20 aa, Homo sapiens
MSLPEDEGHTRTLDTAKEME

SEQ ID NO: 661 - protein, 20 aa, Homo sapiens
IFQKKLDETTRLLRELQEAQ

SEQ ID NO: 662 - protein, 20 aa, Homo sapiens
PPNMICLLGPSYREMHLAEQ

SEQ ID NO: 663 - protein, 20 aa, Homo sapiens
TPGDIVSTYGVRKAMGISIP

SEQ ID NO: 664 - protein, 50 aa, Homo sapiens
MSLPEDEGHTRTLDTAKEMEITEVEPPGRLDSSTQDRLIALKAVTNFGVP

SEQ ID NO: 665 - protein, 50 aa, Homo sapiens
IFQKKLDETTRLLRELQEAQNERLSTRPPPNMICLLGPSYREMHLAEQVT

SEQ ID NO: 666 - protein, 20 aa, Homo sapiens
VAGGPRKTAAPVNALVSHLL

SEQ ID NO: 667 - protein, 20 aa, Homo sapiens
VEPEKLYAMPDPAGPDGHLP

SEQ ID NO: 668 - protein, 20 aa, Homo sapiens
AVATLCDLFDREIVVTISWA

SEQ ID NO: 669 - protein, 20 aa, Homo sapiens
KSIPGFSSLSLSDQMSVLQS

SEQ ID NO: 670 - protein, 20 aa, Homo sapiens
VWMEVLVLGVAQRSLPLQDE

SEQ ID NO: 671 - protein, 20 aa, Homo sapiens
VWMEVLVLGVAQRSLPLQDE

SEQ ID NO: 672 - protein, 50 aa, Homo sapiens
IPGFSSLSLSDQMSVLQSVWMEVLVLGVAQRSLPLQDELAFAEDLVLDEE

SEQ ID NO: 673 - protein, 20 aa, Homo sapiens
LHYGLPVVVKLLHPPSHWPL

SEQ ID NO: 674 - protein, 20 aa, Homo sapiens
VRAHQDTQRRTSMGGTQQQF

SEQ ID NO: 675 - protein, 20 aa, Homo sapiens
QLLYSPIENIQRVAAGVLCE

SEQ ID NO: 676 - protein, 20 aa, Homo sapiens
IEAEGATAPLTELLHSRNEG

SEQ ID NO: 677 - protein, 20 aa, Homo sapiens
GALHILARDVHNRIVIRGLN

SEQ ID NO: 678 - protein, 50 aa, Homo sapiens
QRRTSMGGTQQQFVEGVRMEEIVEGCTGALHILARDVHNRIVIRGLNTIP

SEQ ID NO: 679 - protein, 50 aa, Homo sapiens
CELAQDKEAAEAIEAEGATAPLTELLHSRNEGVATYAAAVLFRMSEDKPQ

SEQ ID NO: 680 - protein, 20 aa, Homo sapiens
SKIDWNNIRNSPPPFVPTLK

SEQ ID NO: 681 - protein, 20 aa, Homo sapiens
YSKALGILGRSESVVSGLDS

SEQ ID NO: 682 - protein, 20 aa, Homo sapiens
EVEAVLSQKEVELKASETQR

SEQ ID NO: 683 - protein, 20 aa, Homo sapiens
DIREQSRKLQEIKEQEYQAQ

SEQ ID NO: 684 - protein, 20 aa, Homo sapiens
RKATECQHKLLKAKDQGKPE

SEQ ID NO: 685 - protein, 20 aa, Homo sapiens
RKATECQHKLLKAKDQGKPE

SEQ ID NO: 686 - protein, 50 aa, Homo sapiens
IQTKSQQIQQMADKILELEEKHREAQVSAQHLEVHLKQKEQHYEEKIKVL

SEQ ID NO: 687 - protein, 20 aa, Homo sapiens
MSLFDLFRGFFGFPGPRSHR

SEQ ID NO: 688 - protein, 20 aa, Homo sapiens
FGFGFSFSPGGGIRFHDNFG

SEQ ID NO: 689 - protein, 20 aa, Homo sapiens
EGQTLRDSMLKYPDSHQPRI

SEQ ID NO: 690 - protein, 20 aa, Homo sapiens
TREDNDLDSQVSQEGLGPVL

SEQ ID NO: 691 - protein, 20 aa, Homo sapiens
TRHEADSSPRGDPESPRPPA

SEQ ID NO: 692 - protein, 50 aa, Homo sapiens
PKSYFKSISVTKITKPDGIVEERRTVVDSEGRTETTVTRHEADSSPRGDP

SEQ ID NO: 693 - protein, 50 aa, Homo sapiens
EEGGSWRRGNPRFHSPQHPPEEFGFGFSFSPGGGIRFHDNFGFDDLVRDF

SEQ ID NO: 694 - protein, 20 aa, Homo sapiens
SKQIQQLESNNRDLQDKNCL

SEQ ID NO: 695 - protein, 20 aa, Homo sapiens
LQDKNCLLETAKLKLEKEFI

SEQ ID NO: 696 - protein, 20 aa, Homo sapiens
LAKVELEKRQLQERFTDLEK

SEQ ID NO: 697 - protein, 20 aa, Homo sapiens
IYESIEEAKSEAMKEMEKKL

SEQ ID NO: 698 - protein, 20 aa, Homo sapiens
QKDVLNEDVRNLTLKIEQET

SEQ ID NO: 699 - protein, 50 aa, Homo sapiens
ERRDRTHGSEIINDLQGRICGLEEDLKNGKILLAKVELEKRQLQERFTDL

SEQ ID NO: 700 - protein, 50 aa, Homo sapiens
SQQKINELLKQKDVLNEDVRNLTLKIEQETQKRCLTQNDLKMQTQQVNTL

SEQ ID NO: 701 - protein, 20 aa, Homo sapiens
LKVLNEQLQSKEVELLQLRT

SEQ ID NO: 702 - protein, 20 aa, Homo sapiens
KECSDLKIELQKAKQTDPYQ

SEQ ID NO: 703 - protein, 20 aa, Homo sapiens
AELLRKLKTSTAIKKACAPV

SEQ ID NO: 704 - protein, 20 aa, Homo sapiens
PLPGDVKVLSEKAILQSWTD

SEQ ID NO: 705 - protein, 20 aa, Homo sapiens
AFGETKTKTLPLPNLPPLHY

SEQ ID NO: 706 - protein, 50 aa, Homo sapiens
ETQQVMRNLNPPSSNWEVEKLSCDLKIHGLEQELELMRKECSDLKIELQK

SEQ ID NO: 707 - protein, 50 aa, Homo sapiens
STKLHLMNFTATYTRHPPLLPNGKALCHTTSSPLPGDVKVLSEKAILQSW

SEQ ID NO: 708 - protein, 20 aa, Homo sapiens
YMQGLKELKESLSEVEEKYK

SEQ ID NO: 709 - protein, 20 aa, Homo sapiens
NEEKSKELERQKHMCSVLQH

SEQ ID NO: 710 - protein, 20 aa, Homo sapiens
LWKDKKIGALEKQKEYIACL

SEQ ID NO: 711 - protein, 20 aa, Homo sapiens
EPVAGAITVVSQEAAQVLES

SEQ ID NO: 712 - protein, 20 aa, Homo sapiens
EPVAGAITVVSQEAAQVLES

SEQ ID NO: 713 - protein, 50 aa, Homo sapiens
LKEGLRQRDELIEEKQRMQQKIDTMTKEVFDLQETLLWKDKKIGALEKQK

SEQ ID NO: 714 - protein, 50 aa, Homo sapiens
SAGEGPLDVRLRKLAGEKEELLSQIRKLKLQLEEERQKCSRNDGTVGDLA

SEQ ID NO: 715 - protein, 20 aa, Homo sapiens
SLQKHLAELNHQKQLESNKI

SEQ ID NO: 716 - protein, 20 aa, Homo sapiens
PQDGPRSKPQPKDNGDVCQD

SEQ ID NO: 717 - protein, 20 aa, Homo sapiens
CKNYISQYSEIAIQMMMHMQ

SEQ ID NO: 718 - protein, 20 aa, Homo sapiens
PIKKHEVPAKSDVYCEVCEF

SEQ ID NO: 719 - protein, 20 aa, Homo sapiens
NKTEKEILDAFDKMCSKLPK

SEQ ID NO: 720 - protein, 50 aa, Homo sapiens
MVTDIQTAVRTNSTFVQALVEHVKEECDRLGPGMADICKNYISQYSEIAI

SEQ ID NO: 721 - protein, 50 aa, Homo sapiens
QKQLESNKIPELDMTEVVAPFMANIPLLLYPQDGPRSKPQPKDNGDVCQD

SEQ ID NO: 722 - protein, 20 aa, Homo sapiens
MPALRPALLWALLALWLCCA

SEQ ID NO: 723 - protein, 20 aa, Homo sapiens
YCQHRDPCEKNRCQNGGTCV

SEQ ID NO: 724 - protein, 20 aa, Homo sapiens
CHMLSRDTYECTCQVGFTGK

SEQ ID NO: 725 - protein, 20 aa, Homo sapiens
TDVNECDIPGHCQHGGTCLN

SEQ ID NO: 726 - protein, 20 aa, Homo sapiens
VNGGTCRQTGDFTFECNCLP

SEQ ID NO: 727 - protein, 50 aa, Homo sapiens
TPAHALQCRDGYEPCVNEGMCVTYHNGTGYCKCPEGFLGEYCQHRDPCEK

SEQ ID NO: 728 - protein, 50 aa, Homo sapiens
SGFTGEDCQYSTSHPCFVSRPCLNGGTCHMLSRDTYECTCQVGFTGKECQ

(19) United States
(12) Patent Application Publication
(10) Pub. No.: US 2005/0112118 A1
(43) Pub. Date: May 26, 2005

(54) Compositions and methods for treating inflammatory disorders

(75) Inventors: Daniel Cimbora, Salt Lake City, UT (US); Karen Heichman, Salt Lake City, UT (US); Paul Bartel, Salt Lake City, UT (US); Kimberly Mauck, Sandy, UT (US); Angie Bush, Sandy, UT (US)

Correspondence Address: MYRIAD GENETICS INC.;INTELLECUTAL PROPERTY DEPARTMENT, 320 WAKARA WAY, SALT LAKE CITY, UT, 84108, US

(73) Assignee: Myriad Genetics, Incorporated, Salt Lake City, UT (US)

(21) Appl. No.: 10/690,276

(22) Filed: Oct. 20, 2003

Related U.S. Application Data
(63) Continuation-in-part of application No. 09/727,384, filed on Dec. 1, 2000, now abandoned.
(63) Continuation-in-part of application No. 10/035,344, filed on Jan. 4, 2002.
(63) Continuation-in-part of application No. 10/035,343, filed on Jan. 4, 2002, now abandoned.
(63) Continuation-in-part of application No. 10/099,924, filed on Mar. 14, 2002.
(63) Continuation-in-part of application No. 10/100,503, filed on Mar. 18, 2002.
(63) Continuation-in-part of application No. 10/014,814, filed on Dec. 14, 2001, now abandoned.
(63) Continuation-in-part of application No. 10/024,599, filed on Dec. 21, 2001.
(60) Provisional application No. 60/168,377, filed on Dec. 2, 1999.
(60) Provisional application No. 60/168,379, filed on Dec. 2, 1999.
(60) Provisional application No. 60/185,056, filed on Feb. 25, 2000.
(60) Provisional application No. 60/259,571, filed on Jan. 4, 2001.
(60) Provisional application No. 60/259,572, filed on Jan. 4, 2001.
(60) Provisional application No. 60/276,179, filed on Mar. 15, 2001.
(60) Provisional application No. 60/307,233, filed on Jul. 23, 2001.
(60) Provisional application No. 60/277,013, filed on Mar. 19, 2001.
(60) Provisional application No. 60/255,063, filed on Dec. 14, 2000.
(60) Provisional application No. 60/256,986, filed on Dec. 21, 2000.

(51) Int. Cl. A61K 39/395; A61K 38/17

(52) U.S. Cl. 424/143.1; 514/12

(57) ABSTRACT

Protein complexes are provided comprising at least one interacting pair of proteins. The protein complexes are useful in screening assays for identifying compounds effective in modulating the protein complexes, and in treating and/or preventing diseases and disorders associated with the protein complexes and/or their constituent interacting members.

### RELATED U.S. APPLICATIONS

This application is a continuation-in-part of U.S. patent application Ser. No. 09/727,384 filed Dec. 1, 2000, which is related to U.S. provisional patent application Ser. No. 60/168,377 filed 2 Dec. 1999, Ser. No. 60/168,379, filed 2 Dec. 1999, and Ser. No. 60/185,056 filed 5 Feb. 2000; and is a continuation-in-part of U.S. patent application Ser. No. 10/035,344 filed 4 Jan. 2002, which is related to U.S. provisional patent application Ser. No. 60/259,571, filed 4 Jan. 2001; and is a continuation-in-part of U.S. patent application Ser. No. 10/035,343 filed 4 Jan. 2002, which is related to U.S. provisional patent applications Ser. No. 60/259,571, filed 4 Jan. 2001; and is a continuation-in-part of U.S. patent application Ser. No. 10/099,924 filed 14 Mar. 2002, which is related to U.S. Provisional Application Ser. No. 60/276,179 filed Mar. 15, 2001, U.S. Provisional Application Ser. No. 60/307,233 filed Jul. 23, 2001, and U.S. Provisional Application Ser. No. 60/343,818 filed Oct. 25, 2001; and is a continuation-in-part of U.S. patent application Ser. No. 10/100,503 filed 18 Mar. 2002, which is related to U.S. provisional patent applications Ser. No. 60/277,013, filed 19 Mar. 2001; and is a continuation-in-part of U.S. patent application Ser. No. 10/014,814 filed 14 Dec. 2001, which is related to U.S. Provisional Application Ser. No. 60/255,063 filed 14 Dec. 2000; and is a continuation-in-part of U.S. patent application Ser. No. 10/024,599 filed 21 Dec. 2001, which is related to U.S. Provisional Application Ser. No. 60/256,986 filed 21 Dec. 2000, each of which is incorporated herein by reference.

### FIELD OF THE INVENTION

The present invention generally relates to methods and compositions for treating diseases, particularly to methods of using and modulating specific proteins and protein-protein interactions for purposes of drug screening and treatment of diseases.

### BACKGROUND OF THE INVENTION

Most drug discovery efforts today employ approaches to empirically identify small molecules that bind particular biological targets in vitro. These approaches generally involve “primary” high throughput screens designed to search vast combinatorial libraries of small molecules for “lead compounds” that often show a relatively weak affinity for the chosen target. However, once such lead compounds are identified in a “primary” high throughput screen, they can be subjected to further iterative rounds of chemical modification and testing by the process known to medicinal chemists as Structure Activity Relationship, or SAR. Generally, after several rounds of SAR-guided modification and in vitro screening, a set of optimized and related drug candidate compounds are subjected to the next phase of testing. This next phase generally involves the in vivo screening of the drug candidates in cell-based assays specifically designed to test the efficacy, toxicity and bioavailablity of the candidates. If the desired effects are obtained with reasonable dosages in these cell-based assays, animal studies are then initiated to determine whether the drug candidates have the desired activity in vivo. Only after careful study in well-defined animal models will a drug candidate be administered to humans in carefully regulated clinical trials.

The success or failure of a drug discovery program is heavily dependent on the identification and selection of druggable targets. In addition, once an appropriate drug target has been identified an efficient, preferably high throughput, screening assay needs to be established for drug screening against that particular drug target, which can be often be difficult to pragmatically achieve. The present invention provides novel drug targets for diseases such as inflammation and inflammatory disorders (e.g., asthma, rheumatoid arthritis, juvenile chronic arthritis, myositis, Chron's disease, gastritis, colitis, ulcerative colitis, inflammatory bowel disease, proctitis, pelvic inflammatory disease, systemic lupus erythematosus, rhinitis, conjunctivitis, scleritis, chronic inflammatory polyneuropathy, Tertiary Lyme disease, psoriasis, dermatitis, eczema, etc.) and discloses screening assays for identifying potential drugs that may be effective against the diseases through modulating the drug targets.

### SUMMARY OF THE INVENTION

The present invention is based on the discovery of novel interactions between pairs of proteins described in the tables below. The specific interactions lead to the identification of desirable novel drug targets. Specifically, the interactions implicate several newly discovered interactors in inflammation and inflammatory disorders and other disease pathways, and suggest that modulation of such interactors may lead to alleviation or treatment of the diseases. In addition, the interactions can lead to the formation of protein complexes both in vitro and in vivo. This enables novel approaches for drug screening to select not only drug candidates that modulate the well-known drug targets used as baits in the interaction discovery, but also modulators of the newly discovered interactors and protein-protein interactions. For example, screening assays can be established based on the interaction between a protein known to be involved in a disease pathway and one of its newly discovered protein interactors. Compounds that modulate or interact with the known target protein can be selected based on their ability either to compete with a newly discovered interactor for interaction with the target protein, or to promote the interaction between the target protein and the interactor.

Thus, in accordance with a first aspect of the present invention, isolated protein complexes are provided which are formed by the protein-protein interactions provided in the tables. In addition, homologues, derivatives, and fragments of the interacting proteins may also be used in forming protein complexes. In a specific embodiment, fragments of an interacting pair of proteins described in the tables containing regions responsible for the protein-protein interaction are used in forming a protein complex of the present invention. In another embodiment, at least one interacting protein member in a protein complex of the present invention is a fusion protein containing a protein in the tables or a homologue, derivative, or fragment thereof. In yet another embodiment, a protein complex is provided from a hybrid protein, which comprises, covalently linked together, directly or through a linker, a pair of interacting proteins described in the tables, or homologues, derivatives, or fragments thereof. In addition, nucleic acids encoding the hybrid protein are also provided.

In yet another aspect, the present invention also provides a method for making the protein complexes. The method includes the steps of providing the first protein and the second protein in the protein complexes of the present invention and contacting said first protein with said second protein. In addition, the protein complexes can be prepared by isolation or purification from tissues and cells or produced by recombinant expression of their protein members. The protein complexes can be incorporated into a protein microchip or microarray, which are useful in large-scale high throughput screening assays involving the protein complexes.

In accordance with a second aspect of the invention, antibodies are provided that are immunoreactive with a protein complex of the present invention. In one embodiment, an antibody is selectively immunoreactive with a protein complex of the present invention. In another embodiment, a bifunctional antibody is provided that has two different antigen binding sites, each being specific to a different interacting protein member in a protein complex of the present invention. The antibodies of the present invention can take various forms including polyclonal antibodies, monoclonal antibodies, chimeric antibodies, antibody fragments such as Fv fragments, single-chain Fv fragments (scFv), Fab′ fragments, and F(ab′)₂ fragments. Preferably, the antibodies are partially or fully humanized antibodies. The antibodies of the present invention can be readily prepared using procedures generally known in the art. For example, recombinant libraries such as phage display libraries and ribosome display libraries may be used to screen for antibodies with desirable specificities. In addition, various mutagenesis techniques such as site-directed mutagenesis and PCR diversification may be used in combination with the screening assays.

The present invention also provides detection methods for determining whether there is any aberration in a patient with respect to a protein complex formed by one or more interactions provided in accordance with this invention. In one embodiment, the method comprises detecting an aberrant concentration of the protein complexes of the present invention. Alternatively, the concentrations of one or more interacting protein members (at the protein or cDNA or mRNA level) of a protein complex of the present invention are measured. In addition, the cellular localization, or tissue or organ distribution of a protein complex of the present invention is determined to detect any aberrant localization or distribution of the protein complex. In another embodiment, mutations in one or more interacting protein members of a protein complex of the present invention can be detected. In particular, it is desirable to determine whether the interacting protein members have any mutations that will lead to, or are associated with, changes in the functional activity of the proteins or changes in their binding affinity to other interacting protein members in forming a protein complex of the present invention. In yet another embodiment, the binding constant of the interacting protein members of one or more protein complexes is determined. A kit may be used for conducting the detection methods of the present invention. Typically, the kit contains reagents useful in any of the above-described embodiments of the detection methods, including, e.g., antibodies specific to a protein complex of the present invention or interacting members thereof, and oligonucleotides selectively hybridizable to the cDNAs or mRNAs encoding one or more interacting protein members of a protein complex. The detection methods may be useful in diagnosing a disease or disorder such as inflammation and inflammatory disorders (e.g., asthma, rheumatoid arthritis, juvenile chronic arthritis, myositis, Chron's disease, gastritis, colitis, ulcerative colitis, inflammatory bowel disease, proctitis, pelvic inflammatory disease, systemic lupus erythematosus, rhinitis, conjunctivitis, scleritis, chronic inflammatory polyneuropathy, Tertiary Lyme disease, psoriasis, dermatitis, eczema, etc.), staging the disease or disorder, or identifying a predisposition to the disease or disorder.

The present invention also provides screening methods for selecting modulators of a protein complex provided according to the present invention. Screening methods are also provided for selecting modulators of the individual interacting proteins. The compounds identified in the screening methods of the present invention can be useful in modulating the functions or activities of the individual interacting proteins, or the protein complexes of the present invention. They may also be effective in modulating the cellular processes involving the proteins and protein complexes, and in preventing or ameliorating diseases or disorders such as inflammation and inflammatory disorders (e.g., asthma, rheumatoid arthritis, juvenile chronic arthritis, myositis, Chron's disease, gastritis, colitis, ulcerative colitis, inflammatory bowel disease, proctitis, pelvic inflammatory disease, systemic lupus erythematosus, rhinitis, conjunctivitis, scleritis, chronic inflammatory polyneuropathy, Tertiary Lyme disease, psoriasis, dermatitis, eczema, etc.).

Thus, test compounds may be screened in in vitro binding assays to identify compounds capable of binding a protein complex of the present invention, or its individual interacting protein members. The assays may include the steps of contacting the protein complex with a test compound and detecting the interaction between the interacting partners. In addition, in vitro dissociation assays may also be employed to select compounds capable of dissociating or destabilizing the protein complexes identified in accordance with the present invention. For example, the assays may entail (1) contacting the interacting members of a protein complex with each other in the presence of a test compound; and (2) detecting the interaction between the interacting members. An in vitro screening assay may also be used to identify compounds that trigger or initiate the formation of, or stabilize, a protein complex of the present invention.

In preferred embodiments, in vivo assays such as yeast two-hybrid assays and various derivatives thereof, preferably reverse two-hybrid assays, are utilized in identifying compounds that interfere with or disrupt the protein-protein interactions discovered according to the present invention. In addition, systems such as yeast two-hybrid assays are also useful in selecting compounds capable of triggering or initiating, enhancing or stabilizing the protein-protein interactions provided in the tables. In a specific embodiment, the screening method includes: (a) providing in a host cell a first fusion protein having a first protein of an interacting protein pair, or a homologue, derivative or fragment thereof, and a second fusion protein having the second protein of the pair, or a homologue, derivative or fragment thereof, wherein a DNA binding domain is fused to one of the first and second proteins while a transcription-activating domain is fused to the other of said first and second proteins; (b) providing in the host cell a reporter gene, wherein the transcription of the reporter gene is determined by the interaction between the first protein and the second protein; (c) allowing the first and second fusion proteins to interact with each other within the host cell in the presence of a test compound; and (d) determining the presence or absence of expression of the reporter gene.

In addition, the present invention also provides a method for selecting a compound capable of modulating a protein-protein interaction in accordance with the present invention, which comprises the steps of (1) contacting a test compound with an interacting protein disclosed in the tables, or a homologue, derivative or fragment thereof; and (2) determining whether said test compound is capable of binding said protein. In a preferred embodiment, the method further includes testing a selected test compound capable of binding said interacting protein for its ability to interfere with a protein-protein interaction according to the present invention involving said interacting protein, and optionally further testing the selected test compound for its ability to modulate cellular activities associated with said interacting protein and/or said protein-protein interaction.

The present invention also relates to a virtual screen method for providing a compound capable of modulating the interaction between the interacting members in a protein complex of the present invention. In one embodiment, the method comprises the steps of providing atomic coordinates defining a three-dimensional structure of a protein complex of the present invention, and designing or selecting, based on said atomic coordinates, compounds capable of interfering with the interaction between the interacting protein members of the protein complex. In another embodiment, the method comprises the steps of providing atomic coordinates defining a three-dimensional structure of an interacting protein described in the tables, and designing or selecting compounds capable of binding the interacting protein based on said atomic coordinates. In preferred embodiments, the method further includes testing a selected test compound for its ability to interfere with a protein-protein interaction provided in accordance with the present invention involving said interacting protein, and optionally further testing the selected test compound for its ability to modulate cellular activities associated with the interacting protein.

The present invention further provides a composition having two expression vectors. One vector contains a nucleic acid encoding a protein of an interacting protein pair according to the present invention, or a homologue, derivative or fragment thereof. Another vector contains the other protein of the interacting pair, or a homologue, derivative or fragment thereof. In addition, an expression vector is also provided containing (1) a first nucleic acid encoding one protein of an interacting protein pair of the present invention, or a homologue, derivative or fragment thereof; and (2) a second nucleic acid encoding the other protein of the interacting pair, or a homologue, derivative or fragment thereof.

Host cells are also provided containing the first and second nucleic acids or comprising the expression vector(s). In addition, the present invention also provides a host cell having two expression cassettes. One expression cassette includes a promoter operably linked to a nucleic acid encoding one protein of an interacting pair of the present invention, or a homologue, derivative or fragment thereof. Another expression cassette includes a promoter operably linked to a nucleic acid encoding the other protein of the interacting pair, or a homologue, derivative or fragment thereof. Preferably, the expression cassettes are chimeric expression cassettes with heterologous promoters included.

In specific embodiments of the host cells or expression vectors, one of the two nucleic acids is linked to a nucleic acid encoding a DNA binding domain, and the other is linked to a nucleic acid encoding a transcription-activation domain, whereby two fusion proteins can be encoded.

In accordance with yet another aspect of the present invention, methods are provided for modulating the functions and activities of a protein complex of the present invention, or interacting protein members thereof. The methods may be used in treating or preventing diseases and disorders such as inflammation and inflammatory disorders (e.g., asthma, rheumatoid arthritis, juvenile chronic arthritis, myositis, Chron's disease, gastritis, colitis, ulcerative colitis, inflammatory bowel disease, proctitis, pelvic inflammatory disease, systemic lupus erythematosus, rhinitis, conjunctivitis, scleritis, chronic inflammatory polyneuropathy, Tertiary Lyme disease, psoriasis, dermatitis, eczema, etc.). In one embodiment, the method comprises reducing a protein complex concentration and/or inhibiting the functional activities of the protein complex. Alternatively, the concentration and/or activity of one or more interacting members of a protein complex may be reduced or inhibited. Thus, the methods may include administering to a patient an antibody specific to a protein complex or an interacting protein member thereof, or an siRNA or antisense oligo or ribozyme selectively hybridizable to a gene or mRNA encoding an interacting member of the protein complex. Also useful is a compound identified in a screening assay of the present invention capable of disrupting the interaction between two interacting members of a protein complex, or inhibiting the activities of an interacting member of the protein complex. In addition, gene therapy methods may also be used in reducing the expression of the gene(s) encoding one or more interacting protein members of a protein complex.

In another embodiment, the methods for modulating the functions and activities of a protein complex of the present invention or interacting protein members thereof comprise increasing the protein complex concentration and/or activating the functional activities of the protein complex. Alternatively, the concentration and/or activity of one or more interacting members of a protein complex of the present invention may be increased. Thus, one or more interacting protein members of a protein complex of the present invention may be administered directly to a patient. Or, exogenous genes encoding one or more protein members of a protein complex of the present invention may be introduced into a patient by gene therapy techniques. In addition, a patient needing treatment or prevention may also be administered with compounds identified in a screening assay of the present invention capable of triggering or initiating, enhancing or stabilizing a protein-protein interaction of the present invention.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### DETAILED DESCRIPTION OF THE INVENTION

### 1. Definitions

The terms “polypeptide,” “protein,” and “peptide” are used herein interchangeably to refer to amino acid chains in which the amino acid residues are linked by peptide bonds or modified peptide bonds. The amino acid chains can be of any length of greater than two amino acids. Unless otherwise specified, the terms “polypeptide,” “protein,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, ubiquitinated forms, etc. Modifications also include intra-molecular crosslinking and covalent attachment to various moieties such as lipids, flavin, biotin, polyethylene glycol or derivatives thereof, etc. In addition, modifications may also include cyclization, branching and cross-linking. Further, amino acids other than the conventional twenty amino acids encoded by genes may also be included in a polypeptide.

The term “isolated polypeptide” as used herein is defined as a polypeptide molecule that is present in a form other than that found in nature. Thus, an isolated polypeptide can be a non-naturally occurring polypeptide. For example, an “isolated polypeptide” can be a “hybrid polypeptide.” An “isolated polypeptide” can also be a polypeptide derived from a naturally occurring polypeptide by additions or deletions or substitutions of amino acids. An isolated polypeptide can also be a “purified polypeptide” which is used herein to mean a specified polypeptide in a substantially homogeneous preparation substantially free of other cellular components, other polypeptides, viral materials, or culture medium, or when the polypeptide is chemically synthesized, chemical precursors or by-products associated with the chemical synthesis. A “purified polypeptide” can be obtained from natural or recombinant host cells by standard purification techniques, or by chemically synthesis, as will be apparent to skilled artisans.

The terms “hybrid protein,” “hybrid polypeptide,” “hybrid peptide,” “fusion protein,” “fusion polypeptide,” and “fusion peptide” are used herein interchangeably to mean a non-naturally occurring polypeptide or isolated polypeptide having a specified polypeptide molecule covalently linked to one or more other polypeptide molecules that do not link to the specified polypeptide in nature. Thus, a “hybrid protein” may be two naturally occurring proteins or fragments thereof linked together by a covalent linkage. A “hybrid protein” may also be a protein formed by covalently linking two artificial polypeptides together. Typically but not necessarily, the two or more polypeptide molecules are linked or “fused” together by a peptide bond forming a single non-branched polypeptide chain.

As used herein, the term “interacting” or “interaction” means that two protein domains, fragments or complete proteins exhibit sufficient physical affinity to each other so as to bring the two “interacting” protein domains, fragments or proteins physically close to each other. An extreme case of interaction is the formation of a chemical bond that results in continual and stable proximity of the two entities. Interactions that are based solely on physical affinities, although usually more dynamic than chemically bonded interactions, can be equally effective in co-localizing two proteins. Examples of physical affinities and chemical bonds include but are not limited to, forces caused by electrical charge differences, hydrophobicity, hydrogen bonds, van der Waals force, ionic force, covalent linkages, and combinations thereof. The state of proximity between the interaction domains, fragments, proteins or entities may be transient or permanent, reversible or irreversible. In any event, it is in contrast to and distinguishable from contact caused by natural random movement of two entities. Typically, although not necessarily, an “interaction” is exhibited by the binding between the interaction domains, fragments, proteins, or entities. Examples of interactions include specific interactions between antigen and antibody, ligand and receptor, enzyme and substrate, and the like.

An “interaction” between two protein domains, fragments or complete proteins can be determined by a number of methods. For example, an interaction is detectable by any commonly accepted approaches, including functional assays such as the two-hybrid systems. Protein-protein interactions can also be determined by various biophysical and biochemical approaches based on the affinity binding between the two interacting partners. Such biochemical methods generally known in the art include, but are not limited to, protein affinity chromatography, affinity blotting, immunoprecipitation, and the like. The binding constant for two interacting proteins, which reflects the strength or quality of the interaction, can also be determined using methods known in the art. See Phizicky and Fields, Microbiol. Rev., 59:94-123 (1995).

As used herein, the term “protein complex” means a composite unit that is a combination of two or more proteins formed by interaction between the proteins. Typically but not necessarily, a “protein complex” is formed by the binding of two or more proteins together through specific non-covalent binding affinities. However, covalent bonds may also be present between the interacting partners. For instance, the two interacting partners can be covalently crosslinked so that the protein complex becomes more stable.

The term “isolated protein complex” means a naturally occurring protein complex present in a composition or environment that is different from that found in its native or original cellular or biological environment in nature. An “isolated protein complex” may also be a protein complex that is not found in nature.

The term “protein fragment” as used herein means a polypeptide that represents a portion of a protein. When a protein fragment exhibits interactions with another protein or protein fragment, the two entities are said to interact through interaction domains that are contained within the entities.

As used herein, the term “domain” means a functional portion, segment or region of a protein, or polypeptide. “Interaction domain” refers specifically to a portion, segment or region of a protein, polypeptide or protein fragment that is responsible for the physical affinity of that protein, protein fragment or isolated domain for another protein, protein fragment or isolated domain.

The term “isolated” when used in reference to nucleic acids (which include gene sequences) of this invention is intended to mean that a nucleic acid molecule is present in a form other than that found in nature.

Thus, an isolated nucleic acid can be a non-naturally occurring nucleic acid. For example, the term “isolated nucleic acid” encompasses “recombinant nucleic acid” which is used herein to mean a hybrid nucleic acid produced by recombinant DNA technology having the specified nucleic acid molecule covalently linked to one or more nucleic acid molecules that are not the nucleic acids naturally flanking the specified nucleic acid in the naturally existing chromosome. One example of recombinant nucleic acid is a hybrid nucleic acid encoding a fusion protein. Another example is an expression vector having the specified nucleic acid inserted in a vector.

The term “isolated nucleic acid” also encompasses nucleic acid molecules that are present in a form other than that found in its original environment in nature with respect to its association with other molecules. In this respect, an “isolated nucleic acid” as used herein means a nucleic acid molecule having only a portion of the nucleic acid sequence in the chromosome but not one or more other portions present on the same chromosome. Thus, an isolated nucleic acid present in a form other than that found in its original environment in nature with respect to its association with other molecules typically includes no more than 10 kb of the naturally occurring nucleic acid sequences that immediately flank the gene in the naturally existing chromosome or genomic DNA. Thus, the term “isolated nucleic acid” encompasses the term “purified nucleic acid,” which means an isolated nucleic acid in a substantially homogeneous preparation substantially free of other cellular components, other nucleic acids, viral materials, or culture medium, or chemical precursors or by-products associated with chemical reactions for chemical synthesis of nucleic acids. Typically, a “purified nucleic acid” can be obtained by standard nucleic acid purification methods, as will be apparent to skilled artisans.

An isolated nucleic acid can be in a vector. However, it is noted that an “isolated nucleic acid” as used herein is distinct from a clone in a conventional library such as a genomic DNA library or a cDNA library in that the clones in a library are still in admixture with almost all the other nucleic acids from a chromosome or a cell.

The term “high stringency hybridization conditions,” when used in connection with nucleic acid hybridization, means hybridization conducted overnight at 42 degrees C. in a solution containing 50% formamide, 5×SSC (750 mM NaCl, 75 mM sodium citrate), 50 mM sodium phosphate, pH 7.6, 5× Denhardt's solution, 10% dextran sulfate, and 20 microgram/ml denatured and sheared salmon sperm DNA, with hybridization filters washed in 0.1×SSC at about 65° C. The term “moderate stringent hybridization conditions,” when used in connection with nucleic acid hybridization, means hybridization conducted overnight at 37 degrees C. in a solution containing 50% formamide, 5×SSC (750 mM NaCl, 75 mM sodium citrate), 50 mM sodium phosphate, pH 7.6, 5× Denhardt's solution, 10% dextran sulfate, and 20 microgram/ml denatured and sheared salmon sperm DNA, with hybridization filters washed in 1×SSC at about 50° C. It is noted that many other hybridization methods, solutions and temperatures can be used to achieve comparable stringent hybridization conditions as will be apparent to skilled artisans.

As used herein, the term “homologue,” when used in connection with a first native protein or fragment thereof that is discovered, according to the present invention, to interact with a second native protein or fragment thereof, means a polypeptide that exhibits a sufficient amino acid sequence homology (greater than 20%) and structural resemblance to the first native interacting protein, or to one of the interacting domains of the first native protein such that it is capable of interacting with the second native protein. Typically, a protein homologue of a native protein may have an amino acid sequence that is at least about 50%, 55%, 60%, 65% or 70%, preferably at least about 75%, more preferably at least about 80%, 85%, 86%, 87%, 88% or 89%, even more preferably at least 90%, 91%, 92%, 93% or 94%, and most preferably about 95%, 96%, 97%, 98% or 99% identical to the native protein. Examples of homologues may be the ortholog proteins of other species including animals, plants, yeast, bacteria, and the like. Homologues may also be selected by, e.g., mutagenesis in a native protein. For example, homologues may be identified by site-specific mutagenesis in combination with assays for detecting protein-protein interactions, e.g., the yeast two-hybrid system described below, as will be apparent to skilled artisans apprised of the present invention. Other techniques for detecting protein-protein interactions include, e.g., protein affinity chromatography, affinity blotting, in vitro binding assays, and the like.

For the purpose of comparing two different nucleic acid or polypeptide sequences, one sequence (test sequence) may be described to be a specific “percent identical to” another sequence (reference sequence) in the present disclosure. In this respect, the percentage identity is determined by the algorithm of Karlin and Altschul, Proc. Natl. Acad. Sci. USA, 90:5873-5877 (1993), which is incorporated into various BLAST programs. Specifically, the percentage identity is determined by the “BLAST 2 Sequences” tool, which is available at NCBI's website. See Tatusova and Madden, FEMS Microbiol. Lett., 174(2):247-250 (1999). For pairwise DNA-DNA comparison, the BLASTN 2.1.2 program is used with default parameters (Match: 1; Mismatch: −2; Open gap: 5 penalties; extension gap: 2 penalties; gap x_dropoff: 50; expect: 10; and word size: 11, with filter). For pairwise protein-protein sequence comparison, the BLASTP 2.1.2 program is employed using default parameters (Matrix: BLOSUM62; gap open: 11; gap extension: 1; x_dropoff: 15; expect: 10.0; and wordsize: 3, with filter). Percent identity of two sequences is calculated by aligning a test sequence with a reference sequence using BLAST 2.1.2., determining the number of amino acids or nucleotides in the aligned test sequence that are identical to amino acids or nucleotides in the same position of the reference sequence, and dividing the number of identical amino acids or nucleotides by the number of amino acids or nucleotides in the reference sequence. When BLAST 2.1.2 is used to compare two sequences, it aligns the sequences and yields the percent identity over defined, aligned regions. If the two sequences are aligned across their entire length, the percent identity yielded by the BLAST 2.1.1 is the percent identity of the two sequences. If BLAST 2.1.2 does not align the two sequences over their entire length, then the number of identical amino acids or nucleotides in the unaligned regions of the test sequence and reference sequence is considered to be zero and the percent identity is calculated by adding the number of identical amino acids or nucleotides in the aligned regions and dividing that number by the length of the reference sequence.

The term “derivative,” when used in connection with a first native protein (or fragment thereof) that is discovered, according to the present invention, to interact with a second native protein (or fragment thereof), means a modified form of the first native protein prepared by modifying the side chain groups of the first native protein without changing the amino acid sequence of the first native protein. The modified form, i.e., the derivative should be capable of interacting with the second native protein. Examples of modified forms include glycosylated forms, phosphorylated forms, myristylated forms, ribosylated forms, ubiquitinated forms, and the like. Derivatives also include hybrid or fusion proteins containing a native protein or a fragment thereof. Methods for preparing such derivative forms should be apparent to skilled artisans. The prepared derivatives can be easily tested for their ability to interact with the native interacting partner using techniques known in the art, e.g., protein affinity chromatography, affinity blotting, in vitro binding assays, yeast two-hybrid assays, and the like.

The term “antibody” as used herein encompasses both monoclonal and polyclonal antibodies that fall within any antibody classes, e.g., IgG, IgM, IgA, IgE, or derivatives thereof. The term “antibody” also includes antibody fragments including, but not limited to, Fab, F(ab′)₂, and conjugates of such fragments, and single-chain antibodies comprising an antigen recognition epitope. In addition, the term “antibody” also means humanized antibodies, including partially or fully humanized antibodies. An antibody may be obtained from an animal, or from a hybridoma cell line producing a monoclonal antibody, or obtained from cells or libraries recombinantly expressing a gene encoding a particular antibody.

The term “selectively immunoreactive” as used herein means that an antibody is reactive thus binds to a specific protein or protein complex, but not other similar proteins or fragments or components thereof.

The term “activity” when used in connection with proteins or protein complexes means any physiological or biochemical activities displayed by or associated with a particular protein or protein complex including but not limited to activities exhibited in biological processes and cellular functions, ability to interact with or bind another molecule or a moiety thereof, binding affinity or specificity to certain molecules, in vitro or in vivo stability (e.g., protein degradation rate, or in the case of protein complexes, the ability to maintain the form of a protein complex), antigenicity and immunogenicity, enzymatic activities, etc. Such activities may be detected or assayed by any of a variety of suitable methods as will be apparent to skilled artisans.

The term “compound” as used herein encompasses all types of organic or inorganic molecules, including but not limited proteins, peptides, polysaccharides, lipids, nucleic acids, small organic molecules, inorganic compounds, and derivatives thereof.

As used herein, the term “interaction antagonist” means a compound that interferes with, blocks, disrupts or destabilizes a protein-protein interaction; blocks or interferes with the formation of a protein complex; or destabilizes, disrupts or dissociates an existing protein complex.

The term “interaction agonist” as used herein means a compound that triggers, initiates, propagates, nucleates, or otherwise enhances the formation of a protein-protein interaction; triggers, initiates, propagates, nucleates, or otherwise enhances the formation of a protein complex; or stabilizes an existing protein complex.

Unless otherwise specified, the term “PRAK” as used herein means the human PRAK protein. The usage for naming other proteins should be similar unless otherwise specified in the present disclosure.

### 2. Protein Complexes

Novel protein-protein interactions have been discovered. The protein-protein interactions are provided in the tables below. Specific fragments capable of conferring interacting properties on the interacting proteins have also been identified. The GenBank reference numbers for the cDNA sequences encoding the interacting proteins are also noted in the tables.

TABLE 1
Binding regions of p38-regulated, mitogen-activated protein kinase
(PRAK) and extracellular signal-regulated kinase 3 (ERK3)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| PRAK | 304 | 471 | ERK3 | 36 | 502 |
| (GenBank | | | (GenBank | | |
| Accession No. | | | Accession No. | | |
| AF032437) | | | X80692) | | |

TABLE 2
Binding regions of p38-regulated, mitogen-activated
protein kinase (PRAK) and protein kinase, cAMP-dependent,
regulatory, type I, alpha (PRKAR1A)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| PRAK | 304 | 471 | PRKAR1A | 19 | 141 |
| (GenBank | | | (GenBank | | |
| Accession No. | | | Accession No. | | |
| AF032437) | | | A12295) | | |

TABLE 3
Binding regions of p38-regulated, mitogen-activated protein
kinase (PRAK) and keratin 23, isoform b (209) (KRT23(209))

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| PRAK | 3 | 304 | KRT23(209) | 1 | 43 |
| (GenBank | | | (GenBank | | |
| Accession No. | | | Accession No. | | |
| AF032437) | | | AL117538) | | |

TABLE 4
Binding regions of p38-regulated, mitogen-activated protein
kinase (PRAK) and novel protein PN7098 (PN7098)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| PRAK | 198 | 304 | PN7098 | 862 | 1177 |
| (GenBank | | | | | |
| Accession No. | | | | | |
| AF032437) | | | | | |

TABLE 5
Binding regions of p38-regulated, mitogen-activated
protein kinase (PRAK) and AL117237 (AL117237)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| PRAK | 3 | 304 | AL117237 | 401 | 488 |
| (GenBank | | | (GenBank | | |
| Accession No. | | | Accession No. | | |
| AF032437) | | | AL117237) | | |

TABLE 6
Binding regions of p38-regulated, mitogen-activated protein
kinase (PRAK) and pericentrin 2 (kendrin) (PCNT2)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| PRAK | 3 | 304 | PCNT2 | 191 | 568 |
| (GenBank | | | (GenBank | 191 | 571 |
| Accession No. | | | Accession No. | 191 | 574 |
| AF032437) | | | U52962) | | |

TABLE 7
Binding regions of p38-regulated, mitogen-activated protein
kinase (PRAK) and homeotic protein Prox 1 (PROXI)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| PRAK | 3 | 304 | PROXI | 203 | 465 |
| (GenBank | | | (GenBank | 204 | 450 |
| Accession No. | | | Accession No. | | |
| AF032437) | | | U44060) | | |

TABLE 8
Binding regions of p38-regulated, mitogen-activated
protein kinase (PRAK) and hook1 protein (HOOK1)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| PRAK | 3 | 304 | HOOK1 | 1 | 413 |
| (GenBank | | | (GenBank | | |
| Accession No. | | | Accession No. | | |
| AF032437) | | | AF044923) | | |

TABLE 9
Binding regions of p38-regulated, mitogen-activated
protein kinase (PRAK) and immunoglobulin gamma-1,
heavy chain, C region, 3′ end (IGHG1)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| PRAK | 3 | 304 | IGHG1 | 105 | 329 |
| (GenBank | | | (GenBank | | |
| Accession No. | | | Accession No. | | |
| AF032437) | | | J002287) | | |

TABLE 10
Binding regions of p38-regulated, mitogen-activated protein kinase
(PRAK) and golgi autoantigen, golgin subfamily a, 2 (GOLGA2)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| PRAK | 3 | 304 | GOLGA2 | 1 | 145 |
| (GenBank | | | (GenBank | 22 | 482 |
| Accession No. | | | Accession No. | 76 | 495 |
| AF032437) | | | L06147) | | |

TABLE 11
Binding regions of p38-regulated, mitogen-activated protein
kinase (PRAK) and hypothetical protein KIAA0555 (KIAA0555)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| PRAK | 3 | 304 | KIAA0555 | 228 | 538 |
| (GenBank | | | (GenBank | 461 | 583 |
| Accession No. | | | Accession No. | 462 | 724 |
| AF032437) | | | AB011127) | | |

TABLE 12
Binding regions of p38-regulated, mitogen-activated
protein kinase (PRAK) and leucine-rich PPR-motif
containing protein (LRPPRC)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| PRAK | 198 | 304 | LRPPRC | 31 | 263 |
| (GenBank | | | (GenBank | | |
| Accession No. | | | Accession No. | | |
| AF032437) | | | M92439) | | |

TABLE 13
Binding regions of mitogen-activated protein kinase-activated
protein kinase 2 (MAPKAP-K2) and extracellular
signal-regulated kinase 3 (ERK3)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| MAPKAP-K2 | 1 | 338 | ERK3 | 19 | 509 |
| (GenBank | | | (GenBank | | |
| Accession No. | | | Accession No. | | |
| U12779) | | | X80692) | | |

TABLE 14
Binding regions of mitogen-activated protein kinase-activated
protein kinase 2 (MAPKAP-K2) and leucine-rich PPR-motif
containing protein (LRPPRC)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| MAPKAP-K2 | 238 | 325 | LRPPRC | 31 | 263 |
| (GenBank | | | (GenBank | | |
| Accession No. | | | Accession No. | | |
| U12779) | | | M92439) | | |

TABLE 15
Binding regions of mitogen-activated protein kinase-activated
protein kinase 2 (MAPKAP-K2) and protein kinase, cAMP-dependent,
regulatory, type I, alpha (PRKAR1A)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| MAPKAP-K2 | 134 | 325 | PRKAR1A | 20 | 382 |
| (GenBank | | | (GenBank | | |
| Accession No. | | | Accession No. | | |
| U12779) | | | A12295) | | |

TABLE 16
Binding regions of mitogen-activated protein kinase-activated
protein kinase 2 (MAPKAP-K2) and SET translocation, myeloid
leukemia-associated, alt. transcript beta (277) (SET277)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| MAPKAP-K2 | 134 | 325 | SET(277) | 107 | 239 |
| (GenBank | | | (GenBank | | |
| Accession No. | | | Accession No. | | |
| U12779) | | | D45198) | | |

TABLE 17
Binding regions of mitogen-activated protein
kinase-activated protein kinase 2 (MAPKAP-K2)
and TL21 mRNA from LNCaP cell line (TL21)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| MAPKAP-K2 | 1 | 338 | TL21 | 2 | 92 |
| (GenBank | | | (GenBank | | |
| Accession No. | | | Accession No. | | |
| U12779) | | | X75692) | | |

TABLE 18
Binding regions of mitogen-activated protein kinase-activated
protein kinase 3 (MAPKAP-K3) and zinc finger transcription
factor KAISO (KAISO)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| MAPKAP-K3 | 1 | 304 | KAISO | 403 | 618 |
| (GenBank | | | (GenBank | | |
| Accession No. | | | Accession No. | | |
| U09578) | | | AC002086) | | |

TABLE 19
Binding regions of mitogen-activated protein kinase-activated
protein kinase 3 (MAPKAP-K3) and novel protein PN7771 (PN7771)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| MAPKAP-K3 | 114 | 304 | PN7771 | 384 | 687 |
| (GenBank | | | (GenBank | | |
| Accession No. | | | Accession No. | | |
| U09578) | | | NM_016350) | | |

TABLE 20
Binding regions of mitogen-activated protein kinase-activated
protein kinase 3 (MAPKAP-K3) and thrombospondin 3 (TSP3)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| MAPKAP-K3 | 1 | 304 | TSP3 | 215 | 366 |
| (GenBank | | | (GenBank | | |
| Accession No. | | | Accession No. | | |
| U09578) | | | L38969) | | |

TABLE 21
Binding regions of mitogen-activated protein
kinase-activated protein kinase 3 (MAPKAP-K3)
and malate dehydrogenase, cytoplasmic (MDH1)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| MAPKAP-K3 | 114 | 304 | MDH1 | 1 | 326 |
| (GenBank | | | (GenBank | | |
| Accession No. | | | Accession No. | | |
| U09578) | | | U20352) | | |

TABLE 22
Binding regions of mitogen-activated protein kinase-activated
protein kinase 3 (MAPKAP-K3) and GA17 (GA17)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| MAPKAP-K3 | 114 | 304 | GA17 | 1 | 363 |
| (GenBank | | | (GenBank | 6 | 374 |
| Accession No. | | | Accession No. | 14 | 374 |
| U09578) | | | AF064603) | | |

TABLE 23
Binding regions of Binding regions of mitogen-activated
protein kinase-activated protein kinase 3 (MAPKAP-K3)
and calpain 4, small subunit (CAPN4)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| MAPKAP-K3 | 114 | 304 | CAPN4 | 99 | 268 |
| (GenBank | | | (GenBank | 102 | 268 |
| Accession No. | | | Accession No. | | |
| U09578) | | | X04106) | | |

TABLE 24
Binding regions of Binding regions of mitogen-activated
protein kinase-activated protein kinase 3 (MAPKAP-K3)
and HLA-B-associated transcript 3 (BAT3)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| MAPKAP-K3 | 217 | 304 | BAT3 | 190 | 473 |
| (GenBank | | | (GenBank | | |
| Accession No. | | | Accession No. | | |
| U09578) | | | M33519) | | |

TABLE 25
Binding regions of protein kinase 1, mitogen-
and stress-activated (MSK1) and actin-binding
LIM protein 1, isoform a (778) (ABLIM1(778))

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| MSK1 | 426 | 686 | ABLIM1(778) | 174 | 251 |
| (GenBank | | | (GenBank | 197 | 251 |
| Accession No. | | | Accession No. | | |
| AF074393) | | | AF005654) | | |

TABLE 26
Binding regions of protein kinase 1, mitogen- and stress-activated
(MSK1) and NICE-4 protein (KIAA0144) (NICE-4)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| MSK1 | 426 | 686 | NICE-4 | 690 | 857 |
| (GenBank | | | (GenBank | | |
| Accession No. | | | Accession No. | | |
| AF074393) | | | D63478) | | |

TABLE 27
Binding regions of mitogen-activated protein kinase 14,
isoform 1 (360) (MAPK14(360)) and cytohesin-4 (CYT4)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| MAPK14(360) | 194 | 319 | CYT4 | 5 | 219 |
| (GenBank | | | (GenBank | | |
| Accession No. | | | Accession No. | | |
| L35253) | | | AF075458) | | |

TABLE 28
Binding regions of mitogen-activated protein kinase
14, isoform 1 (360) (MAPK14(360)) and c-Jun kinase
3, alt. transcript alpha2 (JNK3A2)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| MAPK14(360) | 1 | 130 | JNK3A2 | 295 | 465 |
| (GenBank | | | (GenBank | 371 | 464 |
| Accession No. | | | Accession No. | | |
| L35253) | | | U07620) | | |

TABLE 29
Binding regions of mitogen-activated protein kinase
14, isoform 1 (360) (MAPK14(360)) and centrosomal
Nek2-associated protein 1 (C-NAP1)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| MAPK14(360) | 1 | 130 | C-NAP1 | 1362 | 1579 |
| (GenBank | | | (GenBank | | |
| Accession No. | | | Accession No. | | |
| L35253) | | | AF049105) | | |

TABLE 30
Binding regions of mitogen-activated protein
kinase 14, isoform 1 (360) (MAPK14(360)) and
vinculin, alt. transcript (1066) (VCL(1066))

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| MAPK14(360) | 194 | 319 | VCL(1066) | 933 | 1067 |
| (GenBank | | | (GenBank | | |
| Accession No. | | | Accession No. | | |
| L35253) | | | M33308) | | |

TABLE 31
Binding regions of mitogen-activated protein kinase 14, isoform
1 (360) (MAPK14(360)) and splicing factor, PTB-associated (PSF)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| MAPK14(360) | 1 | 361 | PSF | 282 | 577 |
| (GenBank | | | (GenBank | | |
| Accession No. | | | Accession No. | | |
| L35253) | | | X70944) | | |

TABLE 32
Binding regions of v-akt murine thymoma viral oncogene homolog
1 (AKT1) and farnesyl transferase, CAAX box, alpha (FNTA)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| AKT1 | 1 | 150 | FNTA | 189 | 328 |
| (GenBank | | | (GenBank | | |
| Accession No. | | | Accession No. | | |
| M63167) | | | L10413) | | |

TABLE 33
Binding regions of v-akt murine thymoma viral oncogene homolog
1 (AKT1) and tetratricopeptide repeat domain 3 (TPRD)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| AKT1 | 1 | 150 | TPRD | 1058 | 1189 |
| (GenBank | | | (GenBank | | |
| Accession No. | | | Accession No. | | |
| M63167) | | | D84294) | | |

TABLE 34
Binding regions of v-akt murine thymoma viral oncogene
homolog 1 (AKT1) and novel protein PN9109 (PN9109)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| AKT1 | 1 | 118 | PN9109 | 2024 | 2239 |
| (GenBank | | | | | |
| Accession No. | | | | | |
| M63167) | | | | | |

TABLE 35
Binding regions of v-akt murine thymoma viral
oncogene homolog 1 (AKT1) and periplakin (PPL)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| AKT1 | 1 | 109 | PPL (GenBank | 1548 | 1756 |
| (GenBank | | | Accession No. | | |
| Accession No. | | | AF013717) | | |
| M63167) | | | | | |

TABLE 36
Binding regions of v-akt murine thymoma viral
oncogene homolog 1 (AKT1) and golgi autoantigen,
golgin, 84 kD protein (GOLGIN-84)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| AKT1 | 1 | 118 | GOLGIN-84 | 609 | 731 |
| (GenBank | | | (GenBank | | |
| Accession No. | | | Accession No. | | |
| M63167) | | | NM_005113) | | |

TABLE 37
Binding regions of v-akt murine thymoma viral oncogene
homolog 2, alt. transcript p55 (481) (AKT2(481)) and
chloride intracellular channel protein 1 (CLIC1)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| AKT2(481) | 1 | 108 | CLIC1 | 51 | 210 |
| (GenBank | | | (GenBank | 68 | 210 |
| Accession No. | | | Accession No. | | |
| M95936) | | | X87689) | | |

TABLE 38
Binding regions of v-akt murine thymoma viral oncogene
homolog 2, alt. transcript p55 (481) (AKT2(481)) and
aldo-keto reductase family 7, member A2 (AKR7A2)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| AKT2(481) | 1 | 108 | AKR7A2 | 82 | 330 |
| (GenBank | | | (GenBank | | |
| Accession No. | | | Accession No. | | |
| M95936) | | | NM_003689) | | |

TABLE 39
Binding regions of v-akt murine thymoma viral oncogene
homolog 2, alt. transcript p55 (481) (AKT2(481)) and
tetratricopeptide repeat domain 3 (TPRD)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| AKT2(481) | 1 | 108 | TPRD | 1058 | 1189 |
| (GenBank | | | (GenBank | | |
| Accession No. | | | Accession No. | | |
| M95936) | | | D84294) | | |

TABLE 40
Binding regions of ribosomal protein S6 kinase, 90 kDa, polypeptide
1 (RPS6KA1) (p90RSK) and Novel protein PN9109 (PN9109)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| RPS6KA1 | 600 | 736 | PN9109 | 2114 | 2239 |
| (GenBank | | | | | |
| Accession No. | | | | | |
| L07597) | | | | | |

TABLE 41
Binding regions of ribosomal protein S6 kinase,
90 kDa, polypeptide 1 (RPS6KA1) (p90RSK) and upstream
of N-ras, alt. transcript (798) (UNR(798))

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| (RPS6KA1) | 418 | 675 | UNR(798) | 110 | 528 |
| (GenBank | | | (GenBank | | |
| Accession No. | | | Accession No. | | |
| L07597) | | | L07597) | | |

TABLE 42
Binding regions of IkappaB kinase alpha (IKK-alpha) and glialblastoma
cell differentiation-related protein (GBDR1)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| IKK-alpha | 599 | 638 | GBDR1 | 55 | 185 |
| (GenBank | | | (GenBank | | |
| Accession No. | | | Accession No. | | |
| AF009225) | | | NM_016172) | | |

TABLE 43
Binding regions of IkappaB kinase beta (IKK-beta)
and hypothetical protein KIAA0614 (KIAA0614)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| IKK-beta | 301 | 602 | KIAA0614 | 2150 | 2232 |
| (GenBank | | | (GenBank | | |
| Accession No. | | | Accession No. | | |
| AF080158) | | | AB014514) | | |

TABLE 44
Binding regions of IkappaB kinase beta (IKK-beta)
and lactate dehydrogenase A (LDHM)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| IKK-beta | 301 | 602 | LDHM | 9 | 332 |
| (GenBank | | | (GenBank | | |
| Accession No. | | | Accession No. | | |
| AF080158) | | | X02152) | | |

TABLE 45
Binding regions of Binding regions of IkappaB kinase beta (IKK-beta)
and translation initiation factor 3, subunit 10 (EIF3S10)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| IKK-beta | 301 | 602 | EIF3S10 | 666 | 852 |
| (GenBank | | | (GenBank | | |
| Accession No. | | | Accession No. | | |
| AF080158) | | | NM_003750) | | |

TABLE 46
Binding regions of Binding regions of IkappaB kinase beta
(IKK-beta) and sarcolemmal associated protein-2 (SLAP-2)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| IKK-beta | 301 | 602 | SLAP-2 | 16 | 258 |
| (GenBank | | | (GenBank | | |
| Accession No. | | | Accession No. | | |
| AF080158) | | | AF100750) | | |

TABLE 47
Binding regions of Binding regions of IkappaB
kinase beta (IKK-beta) and SART-1 (SART-1)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| IKK-beta | 301 | 602 | SART-1 | 248 | 419 |
| (GenBank | | | (GenBank | | |
| Accession No. | | | Accession No. | | |
| AF080158) | | | AB006198) | | |

TABLE 48
Binding regions of Binding regions of IkappaB
kinase beta (IKK-beta) and glialblastoma cell
differentiation-related protein (GBDR1)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| IKK-beta | 301 | 602 | GBDR1 | 57 | 167 |
| (GenBank | | | (GenBank | | |
| Accession No. | | | Accession No. | | |
| AF080158) | | | NM_016172) | | |

TABLE 49
Binding regions of IkappaB kinase gamma (IKK-gamma)
and TRAF-interacting protein I-TRAF (ITRAF)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| IKK-gamma | 150 | 302 | ITRAF | 17 | 424 |
| (GenBank | | | (GenBank | | |
| Accession No. | | | Accession No. | | |
| AF074382) | | | U59863) | | |

TABLE 50
Binding regions of IkappaB kinase, inducible (IKK-i)
and TRAF-interacting protein I-TRAF (ITRAF)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| IKK-i (GenBank | 450 | 717 | ITRAF | 44 | 424 |
| Accession No. | | | (GenBank | | |
| AB016590) | | | Accession No. | | |
| | | | U59863) | | |

TABLE 51
Binding regions of IkappaB kinase, inducible (IKK-i)
and nuclear mitotic apparatus protein 1, 228 kD,
alt. transcript (2101) (NUMA1(2101))

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| IKK-i | 450 | 717 | NUMA1(2101) | 962 | 1092 |
| (GenBank | | | (GenBank | | |
| Accession No. | | | Accession No. | | |
| AB016590) | | | Z11583) | | |

TABLE 52
Binding regions of IkappaB kinase, inducible (IKK-i)
and GTPase-activating protein SPA-1 (SPA1)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| IKK-i | 450 | 717 | SPA1 | 925 | 1042 |
| (GenBank | | | (GenBank | | |
| Accession No. | | | Accession No. | | |
| AB016590) | | | AB005666) | | |

TABLE 53
Binding regions of IkappaB kinase, inducible (IKK-i) and
FYCO1: FYVE and coiled-coil domain containing 1 (PN13730)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| IKK-i | 450 | 717 | FYCO1 | 203 | 493 |
| (GenBank | | | (GenBank | | |
| Accession No. | | | Accession No. | | |
| AB016590) | | | AJ292348) | | |

TABLE 54
Binding regions of baculoviral IAP repeat-containing
5 (BIRC5) (survivin) and DNCL1: dynein, cytoplasmic,
light polypeptide (HDLC1)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| BIRC5 | 3 | 99 | DNCL1 | −20 | 89 |
| (GenBank | 47 | 143 | (GenBank | −20 | 89 |
| Accession No. | 89 | 143 | Accession No. | 1 | 90 |
| U75285) | | | U32944) | | |

TABLE 55
Binding regions of baculoviral IAP repeat-containing
5 (BIRC5) (survivin) and actin, beta (ACTB)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| BIRC5 | 3 | 99 | ACTB | 54 | 335 |
| (GenBank | | | (GenBank | 336 | 375 |
| Accession No. | | | Accession No. | | |
| U75285) | | | K00790) | | |

TABLE 56
Binding regions of baculoviral IAP repeat-containing
5 (BIRC5) (survivin) and DNA helicase II, ATP-dependent,
70 kD subunit (KU70)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| BIRC5 | 3 | 99 | KU70 | 131 | 404 |
| (GenBank | | | (GenBank | | |
| Accession No. | | | Accession No. | | |
| U75285) | | | NM_001469) | | |

TABLE 57
Binding regions of baculoviral IAP repeat-containing 5 (BIRC5)
(survivin) and coatomer beta-prime subunit (COPP)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| BIRC5 | 47 | 143 | COPP | 769 | 906 |
| (GenBank | | | (GenBank | | |
| Accession No. | | | Accession No. | | |
| U75285) | | | X70476) | | |

TABLE 58
Binding regions of baculoviral IAP repeat-containing 5 (BIRC5)
(survivin) and osteopontin, alt. transcript (OSTP)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| BIRC5 | 3 | 99 | OSTP | 1 | 56 |
| (GenBank | | | (GenBank | | |
| Accession No. | | | Accession No. | | |
| U75285) | | | X13694) | | |

TABLE 59
Binding regions of baculoviral IAP repeat-containing
5 (BIRC5) (survivin) and solute carrier family 8
(sodium/calcium exchanger), member 1 (SLC8A1)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| BIRC5 | 52 | 143 | SLC8A1 | 302 | 575 |
| (GenBank | | | (GenBank | | |
| Accession No. | | | Accession No. | | |
| U75285) | | | M91368) | | |

TABLE 60
Binding regions of baculoviral IAP repeat-containing
5 (BIRC5) (survivin) and catenin, alpha 2 (A2-CAT)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| BIRC5 | 52 | 143 | A2-CAT | 1 | 166 |
| (GenBank | | | (GenBank | 55 | 487 |
| Accession No. | | | Accession No. | | |
| U75285) | | | M94151) | | |

TABLE 61
Binding regions of cyclooxygenase 1 (COX1) and THRSP: thyroid
hormone responsive (SPOT14 homolog, rat) (THRSP)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| COX1 | 563 | 634 | THRSP | 30 | 146 |
| (GenBank | | | (GenBank | 34 | 146 |
| Accession No. | | | Accession No. | | |
| M59979) | | | Y08409) | | |

TABLE 62
Binding regions of cyclooxygenase 1 (cyclooxygenase1) and
OPA1: optic atrophy 1 (autosomal dominant) (KIAA0567)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| COX1 (GenBank | 563 | 600 | OPA1 | 162 | 421 |
| Accession No. | | | (GenBank | | |
| M59979) | | | Accession No. | | |
| | | | AB011139) | | |

TABLE 63
Binding regions of SET translocation, myeloid
leukemia-associated, alt. transcript beta (277)
(SET(277)) and Novel Protein 12218 (PN12218)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| SET(277) | 1 | 225 | PN12218 | 1 | 307 |
| (GenBank | | | | | |
| Accession No. | | | | | |
| D45198) | | | | | |

TABLE 64
Binding regions of novel protein PN12218 (PN12218)
and SET translocation, myeloid leukemia-associated,
alt. Transcript beta (277) (SET(277))

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| PN12218 | 105 | 305 | SET(277) | 4 | 241 |
| | | | (GenBank | −25 | 252 |
| | | | Accession No. | −3 | 234 |
| | | | D45198) | −1 | 251 |

TABLE 65
Binding regions of Zinc finger protein 36, C3H type
(ZFP36) and c-Cbl-interacting protein CIN85 (CIN85)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| ZFP36 | 223 | 327 | CIN85 | −40 | 458 |
| (GenBank | 150 | 327 | (GenBank | 4 | 264 |
| Accession No. | | | Accession No. | | |
| M92843) | | | AF230904) | | |

TABLE 66
Binding regions of Zinc finger protein 36, C3H
type (ZFP36) and Novel Protein 13734 (PN13734)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| ZFP36 | 223 | 327 | PN13734 | 372 | 901 |
| (GenBank | | | | | |
| Accession No. | | | | | |
| M92843) | | | | | |

TABLE 67
Binding regions of TIA1 cytotoxic granule-associated RNA
binding protein-like 1, isoform 1 (375) (TIAL1(375)) and
far upstream element-binding protein 1 (FUBP1)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| TIAL1 (375) | 1 | 376 | FUBP1 | 1 | 593 |
| (GenBank | | | (GenBank | | |
| Accession No. | | | Accession No. | | |
| M96954) | | | U05040) | | |

TABLE 68
Binding regions of chloride intracellular channel protein 1 (CLIC1)
and low density lipoprotein receptor-related protein 1 (LRP1)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| CLIC1 | 1 | 210 | LRP1 | 4157 | 4499 |
| (GenBank | | | (GenBank | | |
| Accession No. | | | Accession No. | | |
| X87689) | | | NM_002332) | | |

TABLE 69
Binding regions of Chloride intracellular channel protein
1 (CLIC1) and fusion, derived from t(12-16) malignant
liposarcoma, alt. transcript (525) (FUS(525))

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| CLIC1 | 1 | 210 | FUS(525) | −13 | 113 |
| (GenBank | | | (GenBank | | |
| Accession No. | | | Accession No. | | |
| X87689) | | | AF071213) | | |

TABLE 70
Binding regions of Chloride intracellular channel protein
1 (CLIC1) and Fusion, derived from t(12-16) malignant
liposarcoma, alt. transcript (526) (FUS(526))

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| CLIC1 | 1 | 210 | FUS(526) | −13 | 115 |
| (GenBank | | | (GenBank | | |
| Accession No. | | | Accession No. | | |
| X87689) | | | AF071213) | | |

TABLE 71
Binding regions of nuclear receptor coactivator
2 (NCOA2) and XE169 (XE169)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| NCOA2 | 366 | 624 | XE169 | 1239 | 1439 |
| (GenBank | | | (GenBank | | |
| Accession No. | | | Accession No. | | |
| X97674) | | | L25270) | | |

TABLE 72
Binding regions of nuclear receptor coactivator
2 (NCOA2) and NAG4 (NAG4)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| NCOA2 | 366 | 624 | NAG4 | 453 | 634 |
| (GenBank | | | (GenBank | | |
| Accession No. | | | Accession No. | | |
| X97674) | | | AF152604) | | |

TABLE 73
Binding regions of nuclear receptor coactivator 2 (NCOA2)
and estrogen-related receptor alpha (ESRRA)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| NCOA2 | 595 | 800 | ESRRA | 280 | 393 |
| (GenBank | | | (GenBank | | |
| Accession No. | | | Accession No. | | |
| X97674) | | | X51416) | | |

TABLE 74
Binding regions of nuclear receptor coactivator
2 (NCOA2) and beta catenin (CTNNB1)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| NCOA2 | 366 | 624 | CTNNB1 | 487 | 668 |
| (GenBank | | | (GenBank | | |
| Accession No. | | | Accession No. | | |
| X97674) | | | Z19054) | | |

TABLE 75
Binding regions of nuclear receptor coactivator
2 (NCOA2) and citron (CIT)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| NCOA2 | 1 | 368 | CIT | 360 | 790 |
| (GenBank | | | (GenBank | | |
| Accession No. | | | Accession No. | | |
| X97674) | | | AC002563) | | |

TABLE 76
Binding regions of nuclear receptor coactivator
2 (NCOA2) and HS1-binding protein (HS1BP1)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| NCOA2 | 366 | 624 | HS1BP1 | −25 | 279 |
| (GenBank | | | (GenBank | | |
| Accession No. | | | Accession No. | | |
| X97674) | | | U68566) | | |

TABLE 77
Binding regions of nuclear receptor coactivator
2 (NCOA2) and Rho-associated, coiled-coil
containing protein kinase 2 (ROCK2)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| NCOA2 | 366 | 624 | ROCK2 | 589 | 828 |
| (GenBank | | | (GenBank | | |
| Accession No. | | | Accession No. | | |
| X97674) | | | AB014519) | | |

TABLE 78
Binding regions of nuclear receptor coactivator
2 (NCOA2) and TILP (392) (TILP(392))

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| NCOA2 | 1 | 368 | TILP(392) | 170 | 392 |
| (GenBank | | | (GenBank | | |
| Accession No. | | | Accession No. | | |
| X97674) | | | AF044917) | | |

TABLE 79
Binding regions of nuclear receptor coactivator 2 (NCOA2) and
LRR FLI-I interacting protein 2, alt. transcript a (LRRFIP2a)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| NCOA2 | 1 | 368 | LRRFIP2a | 361 | 652 |
| (GenBank | | | (GenBank | | |
| Accession No. | | | Accession No. | | |
| X97674) | | | NM_006309) | | |

TABLE 80
Binding regions of nuclear receptor coactivator
2 (NCOA2) and prosaposin, alt. transcript (PSAP)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| NCOA2 | 366 | 624 | PSAP | 140 | 337 |
| (GenBank | | | (GenBank | | |
| Accession No. | | | Accession No. | | |
| X97674) | | | J03077) | | |

TABLE 81
Binding regions of estrogen receptor 1 (ESR1)
and notch (Drosophila) homolog 2 (NOTCH2)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| ESR1 (GenBank | 231 | 330 | NOTCH2 | 314 | 597 |
| Accession No. | | | (GenBank | | |
| X03635) | | | Accession No. | | |
| | | | AF315356) | | |

TABLE 82
Binding regions of estrogen receptor 2 (ER-beta)
and notch (Drosophila) homolog 2 (NOTCH2)

| BAIT PROTEIN | | | PREY PROTEIN | | |
|---|---|---|---|---|---|
| | Amino Acid | | | Amino Acid | |
| Name and | Coordinates | | Name and | Coordinates | |
| Accession No. | Start | Stop | Accession No. | Start | Stop |
| ER-beta | 1 | 148 | NOTCH2 | 61 | 250 |
| (GenBank | | | (GenBank | | |
| Accession No. | | | Accession No. | | |
| X99101) | | | AF315356) | | |

### 2.1. Biological Significance

Cellular events that are initiated by exposure to growth factors, cytokines and stress are propagated from the outside of the cell to the nucleus by means of several protein kinase signal transduction cascades. Mitogen-activated protein kinase 14 (MAPK14), also known as p38 kinase, is a member of the MAP kinase family of protein kinases. It is a key player in signal transduction pathways induced by the proinflammatory cytokines such as tumor necrosis factor (TNF), interleukin-1 (IL-1) and interleukin-6 (IL-6), and it also plays a critical role in the synthesis and release of the proinflammatory cytokines (Raingeaud et al., J. Biol. Chem. 270:7420-7426, 1995; Lee et al., J Leukoc. Biol. 59:152-157, 1996; Miyazawa et al., J. Biol. Chem. 273:24832-24838, 1998; Lee et al., Nature 372:739-746, 1994). Studies of inhibitors of MAPK14 have shown that blocking MAPK14 activity can cause anti-inflammatory effects, thus suggesting that this may be a mechanism of treating certain inflammatory diseases and disorders, such as asthma, rheumatoid arthritis, juvenile chronic arthritis, myositis, Chron's disease, gastritis, colitis, ulcerative colitis, inflammatory bowel disease, proctitis, pelvic inflammatory disease, systemic lupus erythematosus, rhinitis, conjunctivitis, scleritis, chronic inflammatory polyneuropathy, Tertiary Lyme disease, psoriasis, dermatitis, eczema, etc. Further, p38 kinase activity has been implicated in other human diseases such as atherosclerosis, cardiac hypertrophy and hypoxic brain injury (Grammer et al., J. Immunol. 161:1183-1193, 1998; Mach et al., Nature 394:200-203, 1998; Wang et al., J. Biol. Chem. 273:2161-2168, 1998; Nemoto et al., Mol. Cell. Biol. 18:3518-3526, 1998; Kawasaki et al., J. Biol. Chem. 272:18518-18521, 1997). Thus, by understanding p38 kinase function, one may gain novel insight into a cellular response mechanism that, in a number of tissues, leads to a large number of inflammatory diseases or disorders. Through such understanding, therapeutic targets and ultimately therapeutic compounds and compositions can be developed.

The search for the physiological substrates of MAPK14 has taken a number of approaches including a variety of biochemical and cell biological methods. There are four known human isoforms of MAPK14, or p38 kinase. In the older literature these are termed p38 kinase alpha, beta, gamma and delta. In the newer literature they are termed mitogen-activated protein kinase 14, isoforms 1, 2, 3 and 4. MAPK14(360) refers to the isoform that is 360 amino acids long, which corresponds to p39 kinase alpha.

The isoforms of MAPK14 (or p38 kinase) are thought to possess different physiological functions, likely because they have distinct substrate and tissue specificities. Some of the p38 kinase/MAPK14 substrates are known, and the list includes transcription factors and additional protein kinases that act downstream of p38 kinase/MAPK14. Four of the kinases that act downstream of p38 kinase/MAPK14-MAPKAP-K2, MAPKAP-K3, PRAK and MSK1—are currently being analyzed themselves and some of their substrates and regulators have been identified.

We have discovered several novel substrates and potential upstream regulators of the p38 kinases and their downstream effector kinases. Because inhibitors of p38 kinases can cause anti-inflammatory effects, modulating p38 kinases, p38 kinases interactions, or p38 kinase-interactors is believed to be capable of treating inflammatory diseases.

One novel substrate of p38 alpha/MAPK14(360) that we discovered is the guanine nucleotide-exchange protein cytohesin-4 (CYT4). CYT4 is a member of the PSCD proteins, consisting of an N-terminal coiled-coil motif, a central Sec7 homology domain, and a C-terminal pleckstrin homology (PH) domain. The coiled-coil motif is involved in homodimerization, the Sec7 domain contains guanine-nucleotide exchange protein (GEP) activity, and the PH domain interacts with phospholipids and is responsible for association of PSCD proteins with membranes. Members of this family appear to mediate the regulation of protein sorting and membrane trafficking. CYT4 exhibits GEP activity in vitro with ADP-ribosylation factors ARFI and ARF5 but is inactive with ARF6 (Ogasawara et al., J. Biol. Chem. 275:3221-3230, 2000). CYT4 may act as either a substrate or a regulator of p38 alpha kinase/MAPK14(360) in inflammation or other disease-related signal transduction pathways. Therefore, a modulator of CYT4 or MAPK14 or the interaction thereof may be used to treat inflammatory diseases.

We also identified interactions of the mitogen-activated MAP kinase activator 3pK (MAPKAP-K3). The first interactor, Kaiso (novel protein PN2012), bears similarity to the mouse transcription factor Kaiso (GenBank accession No. AF097416). Kaiso is a zinc-finger containing protein of the POZ-ZF variety; other related members of this family have been implicated in the developmental control and cancer (Daniel et al., Mol. Cell. Biol. 19:3614-3623, 1999). MAPKAP-K3 may phosphorylate this putative transcription factor, thereby altering its activity and affecting the transcription of a set of inflammation-related genes. In support of this hypothesis, Kaiso contains one MAPKAP consensus phosphorylation site. Because inhibitors of p38 kinases such as MAPKAP-K3 may cause anti-inflammatory effects, modulating p38 kinases, p38 kinases interactions, or p38 kinase-interactors is believed to be capable of treating inflammatory diseases. Therefore, a modulator of Kaiso or MAPKAP-K3 or interaction thereof may be used to treat inflammatory diseases.

The second interactor identified for MAPKAP-K3 is the novel protein PN7771. PN7771 is highly similar (greater than 90% amino acid identity) to Ninein. The nucleotide sequence encoding the novel protein PN7771 is provided as SEQ ID NO:3, and the amino acid sequence of the novel protein PN7771 is provided as SEQ ID NO:4. Ninein is a centrosome-associated protein that interacts with human glycogen synthase kinase 3beta (GSK-3beta) (Hong et al., Biochim. Biophys. Acta 1492:513-516, 2000), is localized to the pericentriolar matrix of the centrosomes, and reacts with centrosomal autoantibody sera (Mack et al., Arthritis Rheum. 41:551-558, 1998). PN7771 contains predicted calcium-binding EF hand motifs, a potential nuclear localization signal, a basic region-leucine zipper motif, a spectrin repeat, coiled-coil motifs, and Glu- and Gln-rich regions. The interaction with MAPKAP-K3 suggests PN7771 may be responsive to MAPK signaling pathways, perhaps serving as a substrate for MAPKAP-K3. In support of this, we find several MAPKAP consensus phosphorylation sites in PN7771. Therefore, a modulator of PN7771 or MAPKAP-K3 or interaction thereof may be used to treat inflammatory diseases.

The p38-regulated/activated kinase PRAK was also found to interact with the novel protein PN7098. The nucleotide sequence encoding the novel protein PN7098 is provided as SEQ ID NO:1, and the amino acid sequence of the novel protein PN7098 is provided as SEQ ID NO:2. PN7098 contains a PKC C1 (diacylglycerol/phorbol ester-binding) domain, several Ser-rich regions, and two potential nuclear localization signals. PN7098 is similar (86% amino acid identity) to the rat Munc13-3 protein (GenBank Accession No. U75361), which is involved in neurotransmitter release (Augustin et al., Biochem. J. 337(Pt. 3):363-371, 1999). PN7098 may function as either a regulator or a substrate of PRAK protein kinase activity. Therefore, a modulator of PN7098 or PRAK or interaction thereof may be used to treat inflammatory diseases.

Other interactors of p38 alpha kinase/MAPK14(360) were identified. The first of these, c-Jun kinase 3, alternate transcript alpha2 (JNK3A2), is also a serine/threonine protein kinase of the MAP kinase family that is involved in signal transduction (Gupta et al., EMBO J. 15:2760-2770, 1996). Like the p38 kinase pathway constituents, the JNK kinases are activated in response to extracellular stimulation by IL-1. The JNK kinases function by phosphorylating various transcription factors, thereby altering gene expression patterns. The interaction of p38 alpha kinase/MAPK14(360) and JNK3A2 suggests that JNK3A2 is either a substrate for p38 alpha kinase/MAPK14(360), and further identifies a potential link between JNK3 and the inflammatory response. In further support of such a link, we have subsequently identified interactions between p38 alpha kinase/MAPK14(360) and both JNK1 and JNK2. Therefore, modulating the activity of JNK3A2 or MAPK14(360) or interaction thereof may treat inflammatory diseases.

The second protein that interacts with p38 alpha kinase/MAPK14(360) is the large centrosomal protein C-NAP1. C-NAP1 is a 2,442 amino acid protein that was originally identified by its interaction with the Nek2 cell cycle-regulated protein kinase (Fry et al., J. Cell. Biol. 141:1563-1574, 1998). C-NAP contains multiple coiled-coil domains that are likely to be involved in protein-protein interactions. The finding that C-NAP1 interacts with p38 alpha kinase/MAPK14(360) suggests that it is a substrate of both Nek2 and p38 kinases. Thus, C-NAP1 may play a critical role in cellular growth control and in the cellular inflammatory response. Further, by inference, this result links p38 alpha kinase/MAPK14(360) to cellular growth control and Nek2 to inflammation. Therefore, modulating the activity of C-NAP1 or MAPK14(360) or interaction thereof may treat inflammatory diseases.

The third p38 alpha kinase/MAPK14(360)-interacting protein, vinculin, resides in the cytoplasmic side of adhesion plaques and may participate in actin microfilament attachment (Rudiger, Bioessays 20:733-740, 1998). Vinculin has been characterized as a tumor suppressor, suggesting that it may play a regulatory function in addition to a structural role in the cell. Vinculin is post-translationally modified by phosphorylation, suggesting it may be a s substrate for p38 kinase. Given the requirements for cytoskeletal rearrangement and changes in cell adhesion in the inflammatory response, our results suggest that phosphorylation of vinculin by p38 alpha kinase may be involved in cellular responses to inflammatory stimuli. This interaction is reminiscent of another interaction (see below) between a kinase downstream of p38 alpha kinase/MAPK14(360) (MSK1) and the actin-binding protein ABLIM. Therefore, modulating the activity of vinculin or MAPK14(360) or interaction thereof may treat inflammatory diseases.

The fourth p38 alpha kinase/MAPK14(360)-interacting protein was identified with a mutant p38 alpha kinase/MAPK14(360), in which lysine 53 was changed to a methionine (K53M), rendering the kinase catalytically inactive and presumably stabilizing transient protein-protein interactions. The K53M mutant was discovered to interact with the RNA splicing factor PSF. PSF is a nuclear protein that contains two RNA recognition motifs and has been found to form a complex with the polypyrimidine tract-binding protein PTB (Patton et al., Genes Dev. 7:393-406, 1993). Regulation of mRNA splicing is an effective way to modulate protein expression levels, and consequently the interaction of PSF and p38 alpha kinase/MAPK14(360) suggests that phosphorylation of the form by the latter may result in changes in the expression of proteins involved in the inflammatory response. Interestingly, PSF has been shown to bind to the protein phosphatase PP1 delta (Hirano et al., FEBS Lett. 389:191-194, 1996), suggesting a scenario in which PSF activity is controlled by the opposite actions of p38 alpha kinase/MAPK14(360) and PP1 delta phosphatase. Therefore, modulating the activity of PSF or MAPK14(360) or interaction thereof may treat inflammatory diseases.

MAPKAP-K2, a protein kinase that acts downstream of p38 kinase/MAPK14 in the same signal transduction pathway, was discovered to interact with five proteins. The first of these is a leucine-rich PPR-motif containing protein LRPPRC. LRPPRC, also known as L130, was identified by virtue of its high level of expression in hepatoblastoma cells (Hou et al., In Vitro Dev. Biol. Anim. 30A:111-114, 1994). The expression of LRPPRC in hepatoblastoma cells suggests a role in liver function or in the transformation of normal cells to malignant ones. Interestingly, this protein was also identified as an interactor of another highly related p38-activated protein kinase, PRAK (see below). LRPPRC interacts with the kinase domains of both MAPKAP-K2 and PRAK, suggesting it is a substrate for these kinases. Furthermore, the identification of LRPPRC as an interactor of two kinases involved in the same signaling pathway strongly suggests an important role for LRPPRC in the inflammatory response. Therefore, modulating the activity of LRPPRC or MAPKAP-K2 or interaction thereof may treat inflammatory diseases.

The second MAPKAP-K2 interactor, cAMP-dependent protein kinase (PKA) regulatory subunit type 1 alpha (PRKAR1A), is one component of the PKA serine/threonine protein kinase complex that plays a role in cellular signal transduction. Intracellular levels of cAMP increase in response to various chemical and hormonal stimuli, and PKA is in turn activated by binding to the second messenger cAMP (Francis et al., Crit. Rev. Clin. Lab Sci. 36:275-328, 1999). The regulatory subunit of PKA is phosphorylated, suggesting PRKARLA may serve as a substrate for MAPKAP-K2. Consistent with this, the region of MAPKAP-K2 that interacts with PRKAR1A includes the kinase domain. In addition, we find that this same subunit of PKA (PRKAR1A) can bind to another p38-activated protein kinase PRAK (see below). PRAK interacts with ERK3, another kinase involved in signal transduction, which also interacts directly with MAPKAP-K2 (see below). Taken together, these results suggest that PRKAR1A and PKA may be involved in the inflammatory response, perhaps as a substrate of the protein kinases. Therefore, modulating the activity of PRKAR1A or MAPKAP-K2 or interaction thereof may treat inflammatory diseases.

Another MAPKAP-K2 interactor involved in signal transduction, ERK3, was found using the MAPKAP-K2 K93M, T222D, T334D triple mutant protein. ERK3 (extracellular signal-regulated protein kinase 3) is a serine/threonine protein kinase (Cheng et al., J. Biol. Chem. 271:8951-8958, 1996). It is a nuclear protein present in several tissues and is expressed in response to a number of extracellular stimuli. ERK3 is likely part of the MAP kinase cascade initiated in response to pro-inflammatory stimuli. This role for ERK3 is supported by its interaction with the p38-regulated/activated kinase PRAK. Furthermore, the interactions of ERK3 with both MAPKAP-K2 and PRAK have been confirmed by in vitro assays. Therefore, modulating the activity of ERK3 or MAPKAP-K2 or interaction thereof may treat inflammatory diseases.

Another signal transduction protein that binds MAPKAP-K2 is the myeloid leukemia-associated protein SET, encoded by the SET translocation, myeloid leukemia-associated, alt. transcript beta (277). SET(277) may be involved in the generation of intracellular signaling events that lead to changes in transcriptional activity after binding of a ligand to HLA class II molecules (Vaesen et al., Biol. Chem. Hoppe-Seyler 375:113-126, 1994). SET is a strong inhibitor of protein phosphatase 2A (Li et al., J. Leukoc. Biol. 59:152-157, 1996), and appears to play a role in cell proliferation, as SET mRNA expression is markedly reduced in cells rendered quiescent by serum starvation, contact inhibition, or differentiation (Carlson et al., J. Am. Soc. Nephrol. 9:1873-1880, 1998). Consistent with a role for SET in growth control and differentiation, fusion of the SET protein with part of the CAN oncogenes as the result of a chromosome translocation results in leukemia (von Lindem et al., Mol. Cell. Biol. 12:3346-3355, 1992). SET is a ubiquitously expressed nuclear phosphoprotein that resembles members of the nucleosome assembly protein family. The SET protein is phosphorylated on serine and threonine residues (in addition to tyrosines), suggesting that SET may be a substrate of MAPKAP-K2. Therefore, modulating the activity of SET or MAPKAP-K2 or interaction thereof may treat inflammatory diseases.

The fourth MAPKAP-K2 interactor is the protein product of the TL21 transcript. In a study designed to examine cDNAs that differentially expressed between androgen-dependent and androgen-independent prostate carcinoma cell lines, TL21 was isolated as a transcript showing a marked increase in the androgen-dependent cell line (Blok et al., Prostate 26:213-224, 1995). The interaction of TL21 with MAPKAP-K2 suggests that TL21 may serve as a substrate or regulator of MAPKAP-K2 kinase activity. Therefore, a modulator of the activity of TL21 or MAPKAP-K2 or interaction thereof may be used to treat inflammatory diseases.

Five proteins were discovered to interact with p38-activated kinase, MAPKAP-K3. The first MAPKAP-K3 interactor is thrombospondin 3, an adhesive glycoprotein that is involved in cell-to-cell and cell-to-matrix interactions (Qabar et al., J. Biol. Chem. 269:1262-1269, 1994). Thereofore, modulating the activity of thrombospondin 3 or MAPKAP-K3 or interaction thereof may treat inflammatory diseases.

The second MAPKAP-K3 interactor is malate dehydrogenase 1 (MDH 1), a cytoplasmic enzyme that catalyzes an NAD-dependent reversible reaction of the citric acid cycle (Musrati et al., Gen. Physiol. Biophys. 17:193-210, 1998). The finding that MAPKAP-K3 interacts with this protein suggests that the protein kinase cascade that responds to inflammatory stimuli may affect cellular metabolism. Therefore, modulating the activity of MDH1 or MAPKAP-K3 or interaction thereof may treat inflammatory diseases.

The third MAPKAP-K3-interacting protein, GA17, has a PCI or PINT domain near the C-terminus. The PINT domain is found in proteasome subunits and proteins involved in translation initiation and intracellular signal transduction. GA17 is thought to function either upstream or downstream of MAPKAP-K3 in the inflammation response pathway. Therefore, a modulator of the activity of GA17 or MAPKAP-K3 or interaction thereof may be used to treat inflammatory diseases.

The fourth MAPKAP-K3 interactor is the small subunit of the calcium-dependent protease calpain (CAPN4). Calpain is a non-lysosomal calcium-activated thiol-protease composed of large and small subunits; the small subunit with which MAPKAP-K3 interacts possesses regulatory activity. Interestingly, calpain has been shown to interact with IL-2 receptor gamma chain, and is responsible for cleavage of this protein (Noguchi et al., Proc. Natl. Acad. Sci. USA 94:11534-11539, 1997). Furthermore, calpain inhibitors have been shown to interfere with NFκB activation (Kouba et al., J. Biol. Chem. 276:6214-6224, 2001, further implicating calpain in intracellular signaling in response to external stimuli. In light of these results, the interactions with MAPKAP-K3 suggest that calpain activity may be modulated by MAPKAP-K3 phosphorylation, and that this has an effect on signal transduction in response to inflammatory signals. Therefore, a modulator of the activity of CAPN4 or MAPKAP-K3 or interaction thereof may be used to treat inflammatory diseases.

The fifth MAPKAP-K3-interacting protein is BAT3. BAT3 is a large proline-rich protein that was identified as an HLA-B-associated transcript and was cloned from a human T-cell line (Banerji et al., Proc. Natl. Acad. Sci. USA 87:2374-2378, 1990). BAT3 is a large cytoplasmic protein that is very rich in proline and includes short tracts of polyproline, polyglycine, and charged amino acids. BAT3 transcripts are present in all adult tissues with the highest levels found in testis (Ozaki et al., DNA Cell Biol. 18:503-512, 1999). BAT3 was demonstrated to bind to candidate neuroblastoma tumor suppressor, DAN. DAN is a zinc-finger containing protein that may participate in the cell cycle regulation of DNA synthesis. Both DAN and BAT3 are down-regulated in transformed cells. The interaction with MAPKAP-K3 suggests they function either upstream or downstream of this kinase in the inflammatory response. Therefore, a modulator of the activity of BAT3 or MAPKAP-K3 or interaction thereof may be used to treat inflammatory diseases.

Another p38-activated protein kinase, MSK1, was discovered to interact with two proteins. The first, ABLIM, possesses two apparent functional domains: an actin-binding region and a LIM domain region that is likely involved in protein-protein kinase interactions (Roof et al., J. Cell Biol. 138:575-588, 1977). ABLIM may function by coupling the actin-based cytoskeleton to intracellular signaling pathways via its association with MSK1. This type of function is critical for cell differentiation and morphogenesis, events that occur in response to exposure to external stimuli. This interaction is reminiscent of the interaction between p38 alpha kinase and the cell adhesion/cytoskeleton related protein vinculin, suggesting that phosphorylation of cytoskeletal components may be an important response to inflammatory stimuli. Therefore, a modulator of ABLIM or MSK1 or interaction thereof may be used to treat inflammatory diseases.

MSK1 has also been demonstrated to interact with KIAA0144. KIAA0144 has Ser-, Pro- and Thr-rich regions. Analysis of homologous ESTs suggests expression in a large variety of tissues. Since the discovery of this interaction, KIAA0144 has been given the name NICE-4 by the NCBI. Interaction with MSK1 suggests NICE-4 could function either as a regulator or a substrate of MSK1. Therefore, a modulator of KIAA0144 or MSK1 or interaction thereof may be used to treat inflammatory diseases.

The p38 regulated/activated protein kinase PRAK was found to interact with eleven proteins. Two of these proteins, ERK3 and the cAMP-dependent protein kinase (PKA) regulatory subunit (PRKAR1A), are involved in signal transduction and have been described above as interactors of MAPKAP-K2. The interactions of ERK3 and PRKAR1A with both MAPKAP-K2 and PRAK strengthens the hypothesized role of PRKAR1A and ERK3 in the signal transduction cascades that result from inflammatory stimuli. Therefore, a modulator of ERK3, PRKAR1A, or PRAK or interaction thereof may be used to treat inflammatory diseases.

PRAK interacts with two proteins thought to be involved in vesicular transport. The first of these, HOOK1, was isolated based on sequence similarity to the Drosophila Hook protein. The Drosophila homologue is a cytoplasmic coiled-coil protein that functions in the endocytosis of transmembrane receptors and their ligands from the cell surface to the inside of the cell (Kramer et al., J. Cell Biol. 133:1205-1215, 1996). Human HOOK1 may participate in signal transduction by internalizing receptors or ligands involved in intercellular communication. Therefore, a modulator of HOOK1 or PRAK or interaction thereof may be used to treat inflammatory diseases.

The second PRAK interactor involved in intracellular protein transport is golgin-95, also known as golgi autoantigen, golgin subfamily a, 2, or GOLGA2. GOLGA2 is a coiled-coil protein that localizes to the Golgi apparatus (Fritzler et al., J. Exp. Med. 178:49-62, 1993; Barr, Curr. Biol. 9:381-384, 1999). Its precise function is unknown, but interestingly, it has been shown to cross-react with certain human autoimmune sera. The interaction of HOOK1 and GOLGA2 with PRAK suggests that these proteins may be substrates of PRAK protein kinase activity, and that PRAK may cause changes in intracellular transport in response to external signals by modulating the activity of these proteins. Therefore, a modulator of GOLGA2 or PRAK or interaction thereof may be used to treat inflammatory diseases.

PRAK also binds proteins that function in transcriptional regulation, immune response and mitosis. PRAK has been demonstrated to interact with the Prox1 transcription factor (PROX1). PROX1 is a homeobox-containing protein that has been well studied in mice, and it has been shown to be necessary for the development of the mouse lymphatic system (Wigle et al., Cell 98:769-778, 1999). PRAK may be capable of phosphorylating PROX1, thereby affecting its transcriptional function. PRAK has been shown to bind to the immunoglobulin gamma heavy chain constant region. Immunoglobulin molecules recognize antigens and are the first step of the immune response. Although immunoglobulin molecules normally reside outside of the cell, it is possible that PRAK or some other related protein kinase could phosphorylate them to affect their function. This interaction may serve as a direct tie between PRAK and the immune response. Therefore, a modulator of PROX1 or PRAK or interaction thereof may be used to treat inflammatory diseases.

PRAK has been found to interact with a large centrosomal protein known as pericentrin 2 (PCNT2), which is also called kendrin. PCNT2 forms a complex with gamma tubulin and the dynein motor, and likely plays a critical role in the organization of the mitotic spindle (Purohit et al., J. Cell Biol. 147:481-492, 1999). PRAK binding to PCNT2 suggests that PCNT2 is a substrate of PRAK; thus, PRAK may play an important function in the control of chromosome segregation at mitosis. This interaction is reminiscent of the interaction described above and formed between p38 alpha kinase/MAPK14(360) and the centrosomal protein C-NAP1, and may serve similar functions. Therefore, a modulator of PCNT2 or PRAK or interaction thereof may be used to treat inflammatory diseases.

PRAK has also been found to bind four other proteins. The first of these, KIAA0555, was isolated from brain, but analysis of homologous ESTs suggests it is expressed in a variety of tissues. KIAA0555 contains numerous predicted coiled-coil motifs, likely involved in protein-protein interactions, and it displays similarity (˜20% amino acid identity) to myosin heavy chains from a variety of organisms. We have subsequently identified an interaction between KIAA0555 and the protein 14-3-3 epsilon, which is a member of a large family of proteins involved in signal transduction. The domains of KIAA0555 with which PRAK and 14-3-3 interact overlap suggest that KIAA0555 may serve as a bridge between PRAK and 14-3-3-dependent signaling pathways. Therefore, a modulator of KIAA0555, 14-3-3 epsilon, or PRAK or interaction thereof may be used to treat inflammatory diseases.

The second PRAK interactor is the leucine-rich protein LRPPRC. LRPPRC was described above as an interactor of MAPKAP-K2. Both PRAK and MAPKAP-K2 interact with the same region of LRPPRC, supporting the assertion that LRPPRC plays a role in the inflammatory response. Therefore, a modulator of LRPPRC or PRAK or interaction thereof may be used to treat inflammatory diseases.

The final two PRAK interactors are the proteins corresponding to GenBank accession numbers, AL117237 and AL117538. AL117237 was isolated from adult uterus, and analysis of homologous ESTs suggests nearly ubiquitous expression. Analysis of the predicted protein sequence indicates the presence of a coiled-coil region, Arg- and Glu-rich regions, and several nuclear localization signals. AL117538 was isolated from adult testis, and analysis of homologous ESTs suggests expression in a variety of tissues. The predicted protein contains a spectrin repeat and a coiled-coil region. Since the discovery of the interaction formed between PRAK and AL117538, AL117538 has been recognized as an isoform of keratin. Consequently, AL117538 is now referred to as keratin 23, isoform b (209), or KRT23(209). The interaction of AL117237 and KRT23(209) with PRAK suggests that these two proteins may function either as substrates or regulators of the PRAK protein kinase activity and link these two proteins to the inflammatory response and to inflammation-associated diseases. Therefore, a modulator of AL117237, KRT23(290), or PRAK or interaction thereof may be used to treat inflammatory diseases.

Akt1 (AKT1) and Akt2 (AKT2) are serine/threonine protein kinases capable of phosphorylating a variety of known proteins. AKT1 and AKT2 are activated by platelet-derived growth factor (PDGF), a growth factor involved in the decision between cellular proliferation and apoptosis (Franke et al., Cell 81:727-736, 1995). AKT kinases are also activated by insulin-like growth factor (IGF1), and in this capacity are involved in survival of cerebellar neurons (Dudek et al., Science 275:661-665, 1997). Furthermore, AKT1 is involved in the activation of NFκB by tumor necrosis factor (TNF) (Ozes et al., Nature 401:82-85, 1999). AKT2 has been shown to be associated with pancreatic carcinomas (Cheng et al., Proc. Natl. Acad. Sci. USA 93:3636-3641, 1996). Akt kinases have been implicated in insulin-regulated glucose transport and the development of non-insulin dependent diabetes mellitus (Krook et al., Diabetes 47:1281-1286, 1998).

The p90/RSK kinase (also known as HU1 and ribosomal protein S6 kinase, 90 kDa, polypeptide 1, or RPS6KA1) is also involved in intracellular signaling cascades relevant to human disease. RPS6KA1 activity is regulated by growth factors, and the phosphorylation of two RPS6KA1 substrates, BAD and CREB, suppresses apoptosis in neurons (Bonni et al., Science 286:1358-1362, 1999). RPS6KA1 is also implicated in cell cycle control in response to Mos-MEK1 signaling (Bhatt and Ferrell, Science 286:1362-1365, 1999; Gross et al., Science 286:1365-1367, 1999).

Clearly, these kinases play varied and important roles in a number of intracellular signaling pathways, and are thus good starting points from which to identify novel protein interactions that define disease-related signal transduction pathways. To this end, AKT1 and AKT2 were used to identify Akt-interacting proteins that may be potential targets for drug intervention. Here, we describe new protein-protein interactions for AKT1, AKT2, and RPS6KA1. Modulating the activity of AKT1, AKT2, RPS6KA1, the newly discovered interactors of AKT1, AKT2, and RPS6KA1, or interactors thereof may be used to treat inflammatory disorders.

The first interactor for AKT1 is the alpha subunit of p21 (RAS) farnesyl transferase (FNTA). FNTA has been shown to bind to both the TGF-beta and activin receptors in the yeast two-hybrid assay (Ventura et al., J. Biol. Chem. 271:13931-13934, 1996; Wang et al., Science 271:1120-1122, 1996). Further, it has been shown that FNTA binds to the TGF-beta receptor in the absence of ligand, and that ligand binding causes the phosphorylation and release of FNTA. Presumably, FNTA is then free to interact with other cytoplasmic factors in the transmission of the TGF-beta signal. The finding that AKT1 interacts with FNTA suggests a direct connection between receptors at the cell surface and the intracellular signal transduction machinery involving AKT 1. Therefore, a modulator of FNTA or AKT1 or interaction thereof may be used to treat inflammatory diseases.

The second interactor for AKT1 is the periplakin protein (PPL). The plakins are cytoskeletal coiled-coil proteins that bind to intermediate filaments as well as actin and microtubule networks. Periplakin has been shown to bind to the intracellular portion of collagen type XVII in a yeast two-hybrid assay (Aho et al., Genomics 48:242-247, 1998). Periplakin appears to be highly expressed in tissues that are rich in epithelial cells. The interaction of periplakin with Akt1 suggests it may be a substrate of this kinase, and that its function may be modulated by phosphorylation. Alternatively, the subcellular localization of Akt1 may be altered by its interaction with periplakin. Therefore, a modulator of PPL or AKT1 or interaction thereof may be used to treat inflammatory diseases.

KIAA0728 (also known as PN9109) was found as an interactor of both AKT1 and RPS6KA1. The nucleotide sequence encoding the novel protein PN9109 is provided as SEQ ID NO:5, and the amino acid sequence of the novel protein PN9109 is provided as SEQ ID NO:6. PN9109 contains an EF hand calcium-binding motif, a nuclear localization sequence and six spectrin repeats. The AKT1- and RPS6KA1-interacting regions of PN9109 overlap, suggesting these proteins may bind the same domain of PN9109. The interaction of PN9109 with both AKT1 and RPS6KA1 suggests that it may act as a substrate for both enzymes, or alternatively that PN9109, by virtue of its spectrin repeats, may serve as a scaffold to link these two kinases together. Therefore, a modulator of PN9109 or AKT1 or interaction thereof may be used to treat inflammatory diseases.

AKT1 is found to interact with the integral membrane protein Golgin-84 (GOLGIN-84). Golgin-84 is a coiled-coil containing protein that was originally isolated as an interactor of the OCRL1 phosphatidylinositol(4,5)P2 5-phosphatase that is implicated in oculocerebrorenal syndrome (Bascom et al., J. Biol. Chem. 274:2953-2962, 1999). In vitro studies indicate that most of the golgin-84 protein is predicted to be cytoplasmic with only the most extreme C-terminus of the protein extending to the extracellular/vesicular side of membranes. Not surprisingly, the cytoplasmic portion of golgin-84 associates with AKT1. Therefore, a modulator of GOLGIN-83 or AKT1 or interaction thereof may be used to treat inflammatory diseases.

The TPR domain protein TPRD was found to interact with both AKT1 and AKT2. TPDR may play a major role in development since it is localized to the Down syndrome-critical region on human chromosome 21q22.2 (Ohirau et al., DNA Res. 3:9-16, 1996; Tsukahara et al., J. Biochem. (Tokyo) 120:820-827, 1996). Analysis of the amino acid sequence of TPRD reveals the presence of TPR repeats towards the N-terminus of the protein, a bipartite nuclear localization sequence, and a zinc finger. The region of TPRD that associates with the two Akts (amino acids 1058 to 1189) is located near the center of the protein and is distinct from any of the predicted structural domains. Therefore, a modulator of TPRD or AKT1 or interaction thereof may be used to treat inflammatory diseases.

AKT2 is found to interact with the aldehyde reductase AKR7A2 (aflatoxin B1-dialdehyde reductase or AFAR). AKR7A2 is an aldoketoreductase that resides in the cytoplasm of many if not all tissues. AKR7A2 appears to be highly regulated at the transcriptional level. Studies using rats have demonstrated that AKR7A2 mRNA and protein levels increase dramatically in the liver following exposure to dietary antioxidants (Ellis et al., Cancer Res. 56:2758-2766, 1996). The finding that AKR7A2 associates with AKT2 suggests that perhaps this enzyme is also regulated at the post-translational level by AKT2. Therefore, a modulator of AKR7A2 or AKT2 or interaction thereof may be used to treat inflammatory diseases.

The intracellular chloride channel protein CLIC1 was shown to interact with AKT2. CLIC1, also known as NCC27 (nuclear chloride channel-27), was first cloned from human U937 myelomonocytic cells and is the first member of the CLIC family of chloride channels (Valenzuela et al., J. Biol. Chem. 272:12575-12582, 1997). CLIC1 primarily localizes to the nuclear membrane and likely plays a role in the transport of chloride into the nucleus. The finding that CLIC1 and AKT2 associate with one another is rather intriguing, and it suggests that AKT2 may play a role in regulating nuclear ion transport. Interestingly, another related CLIC family member that localizes to the nuclear membrane, CLIC3, has been demonstrated to interact with a signal transduction protein, ERK7 (Qian et al., J. Biol. Chem. 274:1621-1627, 1999). Taken together, these results suggest that intracellular chloride channels may be intimately linked to transduction of extracellular signals. Therefore, a modulator of CLIC1 or AKT2 or interaction thereof may be used to treat inflammatory diseases.

Finally, the UNR (upstream of N-ras) protein was shown to associate with RPS6KA1. UNR contains several cold shock DNA-binding domains and two predicted peroxidase active sites. Transcription of UNR, which is located immediately upstream of the N-ras gene, interferes with transcription of N-ras (Boussadia et al., FEBS Lett. 420:20-24, 1997). Furthermore, the human and rat UNR genes appear to undergo exon skipping that is tissue-dependent (Boussadia et al., Biochim. Biophys. Acta 1172:64-72, 1993). Interestingly, one of the UNR protein products has been shown to interact with the protein product of the ALL-1 gene, which is involved in human chromosome translocations and other rearrangements in acute lymphocytic leukemia (Leshkowitz et al., Oncogene 13:2027-2031, 1996). ALL-1 is the human homolog of the Drosophila trithorax protein and plays a role in the regulation of homeotic genes involved in body segmentation. The finding that RPS6KA1 binds to UNR suggests that RSK may be capable of phosphorylating UNR, thereby affecting its function. Because UNR interacts with ALL-1, it seems likely that such regulation of UNR by RPS6KA1 might affect gene transcription. Therefore, a modulator of UNR or RPS6KA1, or the interaction thereof, may be used to treat inflammatory diseases.

Nuclear factor kappaB (NFκB) is an inducible transcription factor that regulates a large number of genes, particularly those involved in the inflammatory and immune responses (Barnes and Karin, New Engl. J. Med. 336:1066-1071, 1997; Baeuerle and Baichwal, Adv. Immunol. 65:111-137, 1997). NFκB has been demonstrated to be inappropriately regulated in a number of human inflammatory disorders, including rheumatoid and osteoarthritis, asthma, arteriosclerosis and inflammatory bowel disease, as well as some cancers (Luque and Gelinas, Semin. Cancer Biol. 8:103-111, 1997; Foxwell et al., Proc. Natl. Acad. Sci. USA 95:8211-8215, 1998; Barnes and Adcock, Eur. Respir. J. 12:221-234, 1998; Neurath et al., Gut 43:856-860, 1998; Hatada et al., Curr. Opin. Immunol. 12:52-58, 2000). Inhibiting NFκB activation has many potential applications in treating these diseases, and consequently is an area of intense interest for drug development. One mechanism by which steroids exert their broad-spectrum anti-inflammatory action is by inhibiting the activation of NFκB. By identifying non-steroidal means of inhibiting NFκB activation, it is hoped a class of novel immunosuppressive drugs that has the potency of steroids without their toxicity can be developed.

NFκB activity is controlled by protein-protein interactions that alter its subcellular localization (Karin and Ben-Neriah, Ann. Rev. Immunol. 18:621-663, 2000; Karin, J. Biol. Chem. 274:27339-27342, 1999; Mercurio and Manning, Curr. Opin. Cell Biol. 11:226-232, 1999). In unstimulated cells, NFκB is inactive and sequestered in the cytoplasm due to its interaction with IkappaB (IkB), which masks the NFκB nuclear localization signal. Upon stimulation, IkB is phosphorylated, which targets it for ubiquitination and proteasome-mediated degradation. Disruption of the IkB/NFκB complex frees NFκB to enter the nucleus and activate transcription of proinflammatory genes. A key step in NFκB activation is the initial phosphorylation of IkB; this is accomplished by IkB-kinase (IKK) family members, which are in turn responsive to signals from cell surface receptors for factors such as TNF-alpha and IL-1. Clearly, identifying all of the proteins involved in NFκB activation is necessary to understand the process by which extracellular signals are transduced into NFκB-mediated transcriptional responses. Furthermore, identification of these proteins will increase the repertoire of potential targets for therapeutic intervention in the treatment of diseases due to defects involving NFκB activation, such as arthritis, asthma, and cancer.

IkB kinases (IKKs) are responsible for signal-induced phosphorylation IkB, leading to IkB degradation and activation of NFκB. These proteins appear to function as a complex of IKK family members, and may interact with other cellular factors as well. Consequently, the IKKs and proteins with which they interact are potential targets of anti-inflammatory (and other) drugs. Four IKKs [IKK-alpha, IKK-beta, IKK-gamma, and inducible IKK (IKK-i)] have been identified (reviewed in Karin and Ben-Neriah, Ann. Rev. Immunol. 18:621-663, 2000; Karin, J. Biol. Chem. 274:27339-27342, 1999; Mercurio and Manning, Curr. Opin. Cell Biol. 11:226-232, 1999).

We have identified six new interactions for IKK-beta. The first is with the squamous cell carcinoma antigen SART-1. SART-1 was identified as an antigen on human squamous cell carcinoma cells that is recognized by cytotoxic T-lymphocytes. SART-1 does not have any recognizable structural domains that might give clues to its function. Interestingly, SART-1 has a high degree of homology to the mouse Haf protein (GenBank accession AF129931). Haf is described as a hypoxia associated factor that induces the expression of erythropoietin and VEGF. This similarity and the interaction with IKK-beta suggest SART-1 is involved in intracellular signaling both in response to, and leading to the production of, cell signaling factors. Therefore, a modulator of SART-1 or IKK-beta or interaction thereof may be used to treat inflammatory diseases.

The second IKK-beta interactor is a subunit of translation initiation factor 3 (EIF3S10). EIF3S10 is the largest subunit of the EIF3 complex. It contains a so-called PCI domain that is found in other proteins also found in large complexes, such as components of the COP9 signalosome (Scholler et al., DNA Cell Biol. 16:515-531, 1997). The interaction of EIF3S20 with IKK-beta suggests that phosphorylation of the translation machinery may be part of the inflammatory response. This possibility is further supported by our identification of interactions between MAPKAP-K3, a protein kinase involved in the inflammatory response, and the translation-associated proteins ERF-2, SUI1, and PAIP1. Therefore, a modulator of EIF33S10 or IKK-beta or interaction thereof may be used to treat inflammatory diseases.

The next IKK-beta interactor is the lactate dehydrogenase M chain (also known as LDH-A, or LDHM) was found to be an interactor. LDH is the last enzyme involved in anaerobic glycolysis, and resides in the cytosol. Although the significance of this interaction is not entirely clear, the demonstrated interaction with IKK-beta suggests that LDHM can act as a phosphorylation substrate of IKK-beta, and further suggests a link between NFκB activation and cellular metabolism. Therefore, a modulator of LDH or IKK-beta or interaction thereof may be used to treat inflammatory diseases.

IKK-beta is shown to interact with the sarcolemmal-associated protein SLAP-2. The SLAP proteins are a family of amphipathic alpha-helical proteins that associate with the membrane and form coiled-coil structures (Wigle et al., J. Biol. Chem. 272:32384-32394, 1997). We have previously identified an interaction between SLAP-2 and the insulin-regulated aminopeptidase IRAP, suggesting this protein functions both in insulin-dependent and inflammation-related signaling pathways. Therefore, a modulator of SLAP-2 or IKK-beta or interaction thereof may be used to treat inflammatory diseases.

We have identified an interaction between IKK-beta and the hypothetical protein KIAA0614. The function of KIAA0614 appears to be a putative HECT domain in the KIAA0614 protein sequence. The HECT domain is the consensus sequence found in ubiquitin transferases or so-called E3 ubiquitin ligases. IKK-beta contains a ubiquitin-like region that may be responsible for this interaction. In addition, KIAA0614 closely related to a protein described in the public databases as a protein phosphatase (GenBank accession AF174498), suggesting that KIAA0614 and IKK-beta may act together to control the phosphorylation status of cellular substrates such as IkB. Therefore, a modulator of KIAA0614 or IKK-beta or interaction thereof may be used to treat inflammatory diseases.

The next interactor, the glioblastoma cell differentiation-related protein GBDR1, was found to interact with both IKK-alpha and IKK-beta. The function of GBDR1 is not known but sequence analysis indicates the presence of two ubiquitin-associated domains. Consistent with this, the IKK-beta used to isolate GBDR1 contains a ubiquitin-like domain. In contrast, the fragment of IKK-alpha that associates with GBDR1 includes a helix-loop-helix domain rather than the ubiquitin-like domain. Nonetheless, the interaction of the same domain of GBDR1 with two different IKKs strongly suggests this protein is part of the signal transduction cascade that leads to NFκB activation. Therefore, modulating the activity of GBDR1 or IKK-(alpha or beta) or interaction thereof may treat inflammatory diseases.

One interactor for IKK-gamma (also known as NEMO) was identified. This interacting protein, ITRAF, is a known component of the NFκB activation cascade. ITRAF is known to bind to the conserved C-terminal domain of TRAF proteins and inhibit TRAF-mediated NF-kappa-B activation (Ling and Goeddel, J. Biol. Chem. 275:23853-23860, 2000). Phosphorylation of ITRAF results in its dissociation from TRAF and the subsequent activation of NFκB. We, and others, have found that another IKK—the inducible IkB kinase (IKK-i)—is able to interact with, and phosphorylate, ITRAF (Nomura et al., Genes Cells 5:191-202, 2000). The interaction with IKK-gamma may similarly result in modification of ITRAF. However, such a role for IKK-gamma is likely indirect, since IKK-gamma appears to be a non-catalytic IKK family member. This notion is consistent with the fact that the domain of IKK-i with which ITRAF interacts is a C-terminal (non-kinase) region of the protein. Therefore, a modulator of NEMO or IKK-gamma or interaction thereof may be used to treat inflammatory diseases.

The inducible IkB kinase (IKK-i) was found to interact with three proteins. The first of these is the signal-induced proliferation associated protein SPA1. SPA1 is over 90% identical to the murine homolog, which was originally isolated based on its inducible expression in lymphoid cells stimulated with IL-2; it was further shown that murine SPA1 hampers mitogen-induced cell cycle progression when abnormally or prematurely expressed (Hattori et al., Mol. Cell. Biol. 15:552-560, 1995). The N-terminal domains of both the human and murine SPAL proteins are highly homologous to the human Rap1 GTPase-activating protein (GAP). Human SPA1 exhibits GAP activity for Rap1 and Rap2, but not for Ras, Rho, or Ran (Kurachi et al., J. Biol. Chem. 272:28081-28088, 1997). In addition to the N-terminal GTPase activating domain, human SPA1 contains predicted coiled-coil, PDZ, and transmembrane domains. Human SPA1 is localized primarily to the perinuclear region and is widely expressed, with highest expression levels in lymphoid organs. The interaction with IKK-i suggests SPA-1 is involved in NFκB activation. Therefore, a modulator of SPA-1 or IKK-i or interaction thereof may be used to treat inflammatory diseases.

IKK-i is also found to interact with the nuclear mitotic apparatus protein NUMA1. NUMA1 is found in the nucleus during interphase and is associated with isolated nuclear matrices, and specifically localizes to the spindle apparatus during mitosis in a manner that suggests it is involved in the early steps of nuclear reassembly (Lydersen and Pettijohn, Cell 22 (2 Pt. 2):489-499, 1980). Analysis of the 2101 amino acid NUMA1 protein (NUMA1(2101)) reveals an unusually long central coiled-coil domain (>1400 amino acids). Interestingly, NUMA1 is one of a handful of proteins to which RAR-alpha can be fused in acute promyelocytic leukemia (APL). The most prevalent RAR-alpha fusion partner in APL is PML, and it has been proposed that disruption of PML organization is responsible for the APL phenotype. In rare cases of APL, the ligand- and DNA-binding domains of RAR-alpha are fused to the 5′ exons of NUMA1, resulting in a fusion protein that exists in sheet-like nuclear aggregates (Wells et al., Nat. Genet. 17:109-113, 1997). Wells et al. further demonstrate that PML organization is normal in cells expressing the RAR-alpha/NUMA1 fusion, suggesting that interference with retinoid signaling, and not disruption of PML organization, is essential to the APL phenotype and implicating an element of the mitotic apparatus in the molecular pathogenesis of human malignancy. The interaction of NUMA1 with an IKK suggests that cellular processes, such as mitosis and nuclear assembly, are under control of the same signaling pathways that activate NFκB. In support of this, we have previously found interactions between NUMA1 and the signaling proteins MAPKAP-K3, PRAK, AKT 1, and AKT2. Therefore, a modulator of NUMA1 or IKK-i or interaction thereof may be used to treat inflammatory diseases.

The final interaction for IKK-i is with FYCO1 (the novel protein PN13730). FYCO1 is a protein fragment 494 amino acids in length that contains predicted coiled-coil domains, a spectrin repeat, and regions similar to the leukemia inhibiting factor/oncostatin-M small cytokine signature and the syntaxin N-terminal motif. EST analysis suggests that FYCO1 is expressed in a number of tissues including breast, skin and ovary. The full length sequence of FYCO1 and along with the cDNA sequence is set forth in GenBank accession number AJ292348. FYCO1 corresponds to the N-terminus of AJ292348, which is known as FYVE and coiled-coil domain containing 1. Therefore, a modulator of FYCOL or IKK-i or interaction thereof may be used to treat inflammatory diseases.

Survivin, also known as baculoviral IAP repeat-containing protein 5, or BIRC5, has clearly been demonstrated to function as an anti-apoptotic factor and also appears to play a role in cytokinesis. Olie et al., Cancer Res., 60(11):2805-9 (2000); Chen et al., Neoplasia, 2(3):235-41 (2000). Survivin/BIRC5 has been shown to inhibit caspase function and to override the mitotic spindle checkpoint. Suzuki et al., Oncogene, 19(10):1346-53 (2000); Li et al., Nature, 396(6711):580-4 (1998). In addition, it has been shown that Survivin/BIRC5 has (positive) cell cycle effects that coincide with its movement from the cytoplasm to the nucleus where it interacts with the Cdk4/p16(INK4a) complex. Suzuki et al., Oncogene, 19(29):3225-34 (2000). This is followed by phosphorylation of Rb (anti-apoptotic).

Survivin/BIRC5 is one of few known proteins that integrate cell cycle progression and programmed cell death, and thus is involved in preserving homeostasis and developmental morphogenesis (reviewed in Altieri et al., Lab Invest., 79:1327-1333 (1999)). Survivin/BIRC5 is a 142 amino acid protein that contains a single Zn finger of the type found in other IAP (inhibitor of apoptosis) family members that is necessary for apoptosis inhibition, and a C-terminal RING finger thought to mediate protein-protein interaction (Cahill et al., Nature, 392:300-303 (1998)). In addition, survivin/BIRC5 contains numerous potential phosphorylation sites. Suppression of survivin/BIRC5 expression in HeLa cells results in increased apoptosis and inhibition of proliferation (Ambrosini et al., J. Biol. Chem., 273:11177-11182 (1998)). Furthermore, expression of a phosphorylation-defective (T34A) survivin/BIRC5 mutant protein in several human melanoma cell lines triggered apoptosis and enhanced sensitivity to the chemotherapeutic drug cisplatin, and either prevented tumor formation or retarded tumor growth in mice (Grossman et al., Proc. Natl. Acad. Sci. U.S.A., 98:635-640 (2001)).

Survivin/BIRC5 is expressed during G2/M phase of the cell cycle and associates with microtubules of the mitotic spindle during mitosis. Disruption of the association of survivin/BIRC5 with microtubules results in loss of anti-apoptotic activity and an increase in caspase-3 activity during mitosis (Li et al., Nature, 396:580-584 (1998). Survivin/BIRC5 interacts directly with, and inhibits, both caspase-3 and caspase-7, and therefore it has been proposed that the anti-apoptotic effects of survivin/BIRC5 are due to sequestration of these caspases in an inactive form on microtubules by survivin/BIRC5 (Shin et al., Biochemistry, 40:1117-1123 (2001)). Together, these results suggest that survivin/BIRC5 function counteracts a default apoptotic mechanism at the G2/M transition. Overexpression of survivin/BIRC5 in a variety of cancers (e.g. adenocarcinoma and high-grade lymphomas; (Cahill et al., Nature, 392:300-303 (1998)) may overcome an apoptotic checkpoint and favor aberrant cell cycle progression.

Survivin/BIRC5 interacts with dynein light chain 1 (HDLC1). The interacting region of surviving/BIRC5 is C-terminal to the BIR repeat (baculovirus inhibitor of apoptosis repeat), and it contains a coiled coil (aa 99-142), which is found in some structural proteins, such as myosins, and in some DNA-binding proteins as the so-called leucine-zipper. HDLC1 is an 89-amino acid protein, and the prey isolated here encodes the entire ORF as well as 20 “amino acids” of translated 5′-UTR. Dyneins are molecular motors that translocate along microtubules. Null mutations of Drosophila dlcl were lethal and caused embryonic degeneration and widespread apoptotic cell death. Recently, the proapoptotic Bcl-2 family member Bim was shown to be sequestered by LC8 DLC in healthy cells, and this interaction was disrupted by certain apoptotic stimuli. This freed Bim to translocate together with LC8 to Bcl-2 and to neutralize its antiapoptotic activity. Furthermore, it has been found that the 10-kD human DLC1 protein physically interacts with and inhibits the activity of neuronal nitric oxide synthase. Jaffrey and Snyder, Science, 274:774-7 (1996). In the brain, nitric oxide is responsible for the glutamate-linked enhancement of 3-prime, 5-prime cyclic guanosine monophosphate levels and may be involved in apoptosis, synaptogenesis, and neuronal development. It is thus not surprising that survivin/BIRC5 interacts with a protein having a central role in apoptosis. Accordingly, a modulator of DLC1 or survivin/BIRC5 or interaction thereof may be used to treat cancer and inflammatory diseases.

Survivin/BIRC5 is 142 amino acids long and was found to interact with cytoplasmic dynein light cha in 1 (HDLC1) at amino acids 2-99 and 47-142. Survivin/BIRC5 may be held in the cytoplasm in a complex with dynein light chain in the absence of an anti-apoptotic signal. This can occur either appropriately, such as during angiogenesis, or it can occur inappropriately, such as in many cancers. Tran et al., Biochem. Biophys. Res. Commun., 264(3):781-8 (1999); O'Conner et al., Am. J. Pathol., 156(2):393-8 1 (2000). Pathologically, survivin/BIRC5 expression correlates with a bad prognosis for cancer survival. Sarela et al., Gut, 46(5):645-50 (2000). Survivin/BIRC5 is an example of a more “cancer-specific” drug target, which may be useful in developing anticancer drugs that may have fewer cytotoxic side effects than do current chemotherapeutics. Buolamwini, Curr. Opin. Chem. Biol., 3(4):500-9 (1999).

Survivin/BIRC5 is a 142 amino acid protein that functions as an inhibitor of apoptosis (IAP). Survivin/BIRC5 is abundantly expressed in transformed cells of lymphoid and myeloid lineage, adenocarcinoma of the lung, pancreas, colon, breast, and prostate. Survivin/BIRC5 expression appears to be developmentally regulated; the protein is expressed in fetal tissues but is absent in adult, terminally differentiated tissues [Ambrosini et al. 1997 Nat Med 3:917]. Expression of survivin/BIRC5, a microtubule-associated protein, is specific to the G2/M phase of the cell cycle.

Survivin/BIRC5 acts to suppress apoptosis through inhibition of caspases 3 and 7. Unlike most IAP proteins, survivin/BIRC5 contains only a single BIR domain (baculovirus inhibitor of apoptosis repeat) and lacks a RING finger. Survivin/BIRC5 has the highest sequence homology to the yeast Bir1 protein that functions in cell division control and chromosome segregation [Li F et al. 2000 J Biol Chem 275:6707; Li F et al. 1998 Nature 396:580]. We have identified an interaction between the C-terminus of survivin/BIRC5 (aa89-142) and the full-length 89 aa protein cytoplasmic dynein light chain, HDLC1. The light chain is an accessory subunit of the cytoskeletal motor protein dynein that is responsible for microtubule-associated intracellular movement. Accordingly, a modulator of HDLC1 or survivin or interaction thereof may be used to treat cancer and inflammatory diseases.

The C-terminal portion of survivin/BIRC5 involved in this interaction is downstream of the BIR domain and contains a coiled-coil domain (aa99-142) that appears to be responsible for the interaction of survivin/BIRC5 with microtubules. A truncated survivin/BIRC5 mutant (M1-G99) binds minimally to polymerized microtubules and is not cytoprotective against taxol-induced apoptosis [Li F et al. 1998 Nature 396:580]. In addition, a point mutant (Cys84Ala) that alters a residue conserved throughout BIR domains, binds indistinguishably from wild type survivin/BIRC5 to microtubules but is not cytoprotective.

A functional BIR domain and localization to microtubules appear to be necessary for the inhibition of apoptosis by survivin/BIRC5. Inhibiting the interaction between HDLC1 and survivin/BIRC5 may result in a loss of survivin/BIRC5 from the microtubule and a negation of its function in inhibiting apoptosis. It has been demonstrated that gene targeting of survivin/BIRC5 with an antisense mRNA derived from EPR-1 in HeLa cells increased apoptosis and inhibited the growth of these transformed cells (Ambrosini et al. 1998 J Biol Chem 273: 11177).

In support of this approach are data derived with the Bcl-2 family member Bim. The pro-apoptotic activity of Bim appears to be regulated by its interaction with the dynein motor complex through the LC8 cytoplasmic dynein light chain (Puthalakath H 1999 Mol Cell 3:287). Apoptotic stimuli disrupt the interaction of LC8 with the dynein complex and result in the translocation of LC8 and Bim away from microtubules. It has been hypothesized that this release allows Bim to move to Bcl-2 to inhibit its anti-apoptotic effect.

Survivin/BIRC5 interacts with β-actin and DNA helicase II. The search of the brain library with survivin/BIRC5 (aa 3-99) identified β-actin and ATP-dependent DNA helicase II, 70 kD subunit (Ku70) as interactors. β-actin is a non-muscle cytoskeletal actin involved in cell motility. β-actin mRNA is differentially expressed in cells undergoing apoptosis, suggesting a link between β-actin and apoptosis, and therefore perhaps survivin/BIRC5. Naora and Naora, Biochem. Biophys. Res. Commun., 211(2):491-6 (1995). Actins in general have been linked to apoptosis as targets of caspases. Rossiter et al., Neuropathol. Appl. Neurobiol., 26(4):342-6 (2000). Actins have been shown to undergo rearrangement concomitant with apoptosis. Mashima et al., Oncogene, 18(15):2423-30 (1999); Suarez-Huerta et al., J Cell Physiol., 184(2):239-45 (2000). Interestingly, it has been shown that p53 binds to filamentous actin in a calcium-dependent manner. Metcalfe et al., Oncogene, 18(14):2351-5 (1999). Thus, it has been speculated that this may play a role in the transient and reversible nuclear to cytoplasmic shuttling which p53 undergoes. Metcalfe et al., Oncogene, 18(14):2351-5 (1999). For instance, during DNA synthesis, when non-pathological DNA strand breaks are present, a p53 response should not be triggered. A modulator of β-actin, DNA helicase II, or survivin or interaction thereof may be used to treat cancer and inflammatory diseases.

Another interactor, Ku70 (aa 131-403), is a single-stranded DNA- and ATP-dependent helicase thought to have a role in DNA repair (DNA damage sensor) and chromosomal translocation (double-strand break repair protein). It has been proposed that the presence or absence of Ku70 determines whether double-stranded breaks are repaired by nonhomologous end joining (DNA damage; Ku70 present) or by homologous recombination (meiosis; Ku70 absent). Goedecke et al., (Nat. Genet., 23(2):194-8 (1999). Li et al., Mol. Cell, 2(1):1-8 (1998) suggested that Ku70 is a candidate tumor suppressor gene since mice carrying a disruption of the Ku70 gene showed a propensity for malignant transformation (T-cell lymphomas). Autoantibodies against Ku70 are common in cases of systemic lupus erythematosis and autoimmune thyroiditis (Grave's disease), and this is how Ku70 was first identified. Interestingly, Takeda et al., J. Immunol., 163(11):6269-74 (1999) mentioned that caspase-cleaved proteins can elicit the generation of autoantibodies, because cleavage of self antigens may enhance their immunogenicity. In the context of the survivin/BIRC5-Ku70 interaction, it is possible that in the case of lupus, this represents a pathologic disorder of apoptosis in which Ku70 is inappropriately cleaved. Physiologically, it is likely that survivin/BIRC5 plays a role in targeting DNA metabolizing enzymes, such as the helicase Ku70, for proteolysis as one facet of the orchestrated process of programmed cell death. It has been disclosed that an ionizing radiation-induced Ku70-containing complex appears to regulate whether cells undergo apoptosis following a DNA insult. Yang et al., Proc. Natl. Acad. Sci. USA, 97(11):5907-12 (2000). In addition, it has been further suggested that Ku70 up-regulation (following ionization) serves to determine either a course of DNA repair or an arresting response, such as cell death. Brown et al., J. Biol. Chem., 275(9):6651-6 (2000). Therefore, a modulator of KU70 or survivin or interaction thereof may be used to treat cancer and inflammatory diseases.

The interactions between survivin/BIRC5 and proteins including HDLC1, beta-actin, DNA helicase II, COPP, OSTP, SLC8A1, A2-CAT suggest that these proteins are involved in common biological processes including, but not limited to, apoptosis, and disease pathways involving such cellular functions. Therefore, modulators of HDLC1, beta-actin, DNA helicase II, COPP, OSTP, SLC8A1, A2-CAT, or survivin or interaction thereof may be used to treat disease pathways such as cancer and inflammatory diseases.

Cyclooxygenases (Cox-1 and -2) catalyze the rate-limiting steps in prostanoid biosynthesis, and Cox-1 (COX1) is the target of nonsteroidal anti-inflammatory drugs (NSAIDS) such as aspirin. Prostanoids produced by the COX pathway signal via plasma membrane-localized, G-protein-coupled receptors as well as via nuclear receptors. Physiologically, various extracellular stimuli such as growth factors, cytokines and tumor promoters regulate the expression of COX-1 and -2 genes. COX-2 is over-expressed in rheumatoid arthritis, colorectal and breast cancer. NSAIDS treat arthritis and reduce the relative risk of colorectal cancer in humans. So inhibition of cyclooxygenase activity continues to be explored both for anti-inflammatory purposes as well as anti-neoplastic effects. Hla et al., Int. J. Biochem. Cell Biol., 31(5):551-7 (1999); DuBois, Aliment Pharmacol. Ther., 14 (Suppl. 1):64-7 (2000). Studies using COX-1- and COX-2-deficient mice confirm that both isoforms can contribute to the inflammatory response and that both isoforms have significant roles in carcinogenesis. Langenbach et al., Ann. N.Y. Acad. Sci., 889:52-61 (1999).

The cyclooxygenase 1 protein (COX1) was discovered to interact with the thyroid hormone responsive Spot 14 protein (THRSP). Cox-1 contains the short fragment carboxy-terminal to the catalytic domain. “Spot 14” is a nuclear protein induced in liver by hormones, such as thyroid hormone (T3), insulin, and glucagon, and by dietary substrates, such as carbohydrates (glucose) and polyunsaturated fatty acids. It is implicated in the transduction of these hormonal and dietary signals for increased lipid metabolism (synthesis) in hepatocytes, and this includes regulation of genes required for long-chain fatty acid synthesis. Kinlaw et al., J. Biol. Chem., 270(28):16615-8 (1995); Brown et al., J. Biol. Chem., 272(4):2163-6 (1997). Spot 14 is abundant only in lipogenic tissues (liver, adipose, lactating mammary) and is thought to function as a homodimeric transcriptional activator that mediates the switch of hepatic metabolism from the fasted to the fed state. Cunningham et al., Endocrinology, 138(12):5184-8 (1997). S14 antisense oligonucleotides inhibit both the intracellular production of lipids and their export as very low-density lipoprotein particles. The S14 gene is located in a region that is amplified in a subset of aggressive breast cancers. S14 is expressed in most breast cancer-derived cell lines and most breast cancer specimens but not in normal nonlactating mammary glands. S14 is associated with enhanced tumor lipogenesis, an established marker of poor prognosis. Cunningham et al., Thyroid, 8(9):815-25 (1998); Heemers et al., Biochem. Biophys. Res. Commun., 269(1):209-12 (2000). The metabolism of lipids is central to cell (and tumor) biology. It has been suggested that arachidonic acid and other polyunsaturated fats and/or their metabolites may not only promote tumor cell proliferation but that they may also be anti-apoptotic. Tang et al., Int. J. Cancer, 72(6):1078-87(1997). Therefore, it is thought that by modulating Cox-1, THRSP, or their interaction, tumor growth and/or inflammatory diseases may be countered or slowed.

Another interactor of Cox-1 was discovered to be KIAA0567 protein. KIAA0567 contains a coiled coil region and part of the dynamin GTPase catalytic domain. Dynamin GTPases are large GTPases that mediate vesicle trafficking. Dynamin participates in the endocytic uptake of receptors, associated ligands, and plasma membrane following an exocytic event. It has been shown that KIAA0567 appears to be the OPA1 gene, mutations in which give rise to the disease optic atrophy type 1. Delettre et al., Nat. Genet., 26(2):207-10 (2000). This autosomal dominant disease is the most prevalent hereditary optic neuropathy and results in progressive loss in visual acuity leading in many cases to legal blindness. Opa1 is a nuclear gene that is most abundantly expressed in retina, but it is also ubiquitously expressed, which is consistent with the finding that the Opa 1 (OPA1) protein is a component of the mitochondrial matrix. It has been hypothesized that dysfunction of the Opa1 protein affects mitochondrial integrity, resulting in an impairment of energy supply which is disastrous for optic nerve neurons which have a high energy demand. Alexander et al., Nat. Genet., 26(2):211-5 (2000). The two proteins are related by their involvement in lipid metabolism: Cox-1 by its metabolism of lipids to generate mediators of inflammation and OPA1 by its implicated role in mitochondrial energetics, central to which is the β-oxidation of fatty acids. The potential role of OPA1 in vesicle trafficking and membrane transport by virtue of its dynamin GTPase domain also makes it possible that Opa1 could play a role in the delivery of arachidonate to Cox-1. Therefore, a modulator of KIAA0567/OPA1 or Cox-1 or interaction thereof may be used to treat inflammatory diseases.

It is somewhat more notable to view the Cox-1-Opa1 interaction in combination with the Cox-1-Spot14 interaction described above. It is conspicuous that Cox-1, a metabolizer of certain fatty acids, associates with a protein involved in regulating fatty acid production (Spot 14) as well as with a protein involved with fatty acid utilization (Opa 1).

The interactions between COX1 and the COX1-interacting proteins suggest that these proteins are involved in common biological processes including, but not limited to, lipid metabolism, cell proliferation, apoptosis, optic neuropathy, and inflammatory response, and disease pathways involving such cellular functions.

The MAPKAP-K2 associated nuclear phosphatase inhibitor SET is a nuclear protein that is likely involved in signal transduction events in response to binding of a ligand to HLA class II molecules (Vaesen et al., Biol Chem Hoppe Seyler 375:113-126, 1994). It is a potent protein phosphatase 2A inhibitor and has some homology with the nucleosome assembly protein (NAP) family (Li et al., J. Biol. Chem. 271:11059-11062, 1996). We have previously found that MAPKAP-K2 interacts with SET, suggesting that SET plays a role in MAPK14 signaling and that a modulator of SET or MAPKAP-K2 or interaction thereof may be used to treat inflammatory diseases. SET was discovered to interact with PN12218. The nucleotide sequence encoding the novel protein PN12218 is provided as SEQ ID NO:7, and the amino acid sequence of the novel protein PN12218 is provided as SEQ ID NO:8. PN12218 contains a predicted coiled-coil motif. PN12218 displays sequence similarity to human cDNAs (GenBank AK025906 and AK024609). The interaction between SET and PN12218 suggests that these proteins may function together or sequentially in the MAPK14 signal transduction cascade. Therefore, a modulator of PN12218 or SET or interaction thereof may be used to treat inflammatory diseases.

The zinc finger protein ZFP36, which is involved in TNF-alpha regulations and inflammation, is a basic proline-rich protein that is localized to the nucleus and is thought to function as a transcriptional regulator. ZFP36 deficiency in mice results in a complex inflammatory syndrome in mice, and ZFP36-deficient macrophages exhibit increased production of TNF-alpha as a result of stabilization of TNF-alpha mRNA (Carballo et al., Science 281:1001-1005, 1998). These findings suggest that ZFP36 represents a potential target for anti-TNF-alpha therapies and that a modulator of ZFP36 may be used to treat inflammatory diseases. To further expand the number of potential targets for anti-inflammation therapy, ZFP36 was used to identify two ZFP36-interacting proteins. The first ZFP36 interactor is CIN85. CIN85 is an 85 kD protein that contains three SH3 domains and a predicted C-terminal coiled-coil domain, and displays homology to the adaptor proteins CMS (human) and CD2AP (mouse). CIN85 associates with c-Cbl, a substrate of protein tyrosine kinases that is rapidly phosphorylated upon stimulation of a variety of cell-surface receptors. This association is mediated by the second SH3 domain of CIN85, and was enhanced after EGF stimulation of 293 cells (Take et al., Biochem Biophys Res Commun 268:321-328, 2000). The association of CIN85 with c-Cbl correlated with its level of phosphorylation, suggesting a mechanism by which CIN85 may be responsive to MAPK14-dependent kinases. The association of CIN85 with TP suggests these protein may function together to control mRNA stability or gene transcription in response to inflammatory stimuli. Therefore, a modulator of CIN85 or ZFP36 or interaction thereof may be used to treat inflammatory diseases.

The second ZFP36 interactor is the novel protein PN13734. The nucleotide sequence encoding the novel protein PN13734 is provided as SEQ ID NO:9, and the amino acid sequence of the novel protein PN13734 is provided as SEQ ID NO:10. The PN13734 sequence predicts a 2,141 amino acid protein that contains several possible transmembrane domains and threonine-rich regions. The PN13734 sequence contains KIAA1007 (also know as AD-005, described as a novel adrenal gland protein fragment) and the hypothetical protein DKFZp434N241 (accession number AL117492), although these proteins represent only a small part of the PN13734 sequence. Homologous EST analysis suggests that PN13734 is highly expressed in a wide variety of tissues. Northern analysis performed by ProNet demonstrates the expression of an approximately 8.9 kb transcript in a variety of tissues (heart, brain, placenta, lung, liver, skeletal muscle, kidney, and pancreas), with the highest levels of expression in skeletal muscle and kidney. Therefore, a modulator of PN13734 or ZFP36 or interaction thereof may be used to treat inflammatory diseases.

Cytotoxic T-lymphocytes can induce target cells to apoptosis; a key step in this process is the activation of an endogenous endonuclease that degrades target cell DNA. The RNA-binding protein TIAL(375) is a nucleolysin that was isolated from an activated T-cell cDNA library on the basis of its similarity to TIA1. Both proteins are members of a family of RNA-binding proteins containing three RNA-binding motifs and a C-terminal auxiliary domain. TIAL(375) binds specifically to poly-A homopolymers and fragments DNA in permeabilized target cells (Kawakami et al., Proc. Natl. Acad. Sci. 89:8681-8685, 1992), suggesting it is a nuclease involved in T-cell induced apoptosis. Therefore, TIAL(375) is thought to be involved in apoptosis and cellular signaling. Therefore, a modulator of TIAL(375) may be used to treat cancer and/or inflammatory diseases.

TIAL(375) was discovered to interact with FUBP1 (FUSE binding protein 1). FUBP1 is present only in undifferentiated cells, and in these cells it binds to the FUSE (far upstream element) of the c-Myc gene and stimulates its expression (Duncan et al., Genes Dev. 8:465-480, 1994). FUBP1 appears to be required for c-Myc expression and cellular proliferation (He et al., EMBO J. 19:1034-1044, 2000), and interestingly, it binds preferentially to the FUSE sequence when it is in a single-stranded conformation. FUBP1 has been recently demonstrated to associate with the SMN1 (survival motor neuron) protein (Williams et al., FEBS Lett. 470:207-210, 2000), which is a nuclear protein we have previously identified as an interactor of the MAPK14-regulated kinase PRAK. SMN1 has been implicated in mRNA processing and is thought to play a key role in the biogenesis of small nuclear ribonucleoprotein particles (snRNPs). Thus, all three proteins (TIAL(375), SMN1 and FUBP1) appear to function by binding to single-stranded nucleotides and may coordinately function in transcriptional and/or post-transcriptional regulatory mechanisms that affect such key cellular players as TNF-alpha and c-Myc. Therefore, a modulator of FUBP1 or TIAL(375) or interaction thereof may be used to treat inflammatory diseases.

Akt1 and Akt2 are serine/threonine protein kinases capable of phosphorylating a variety of known proteins. Akt1 and Akt2 are activated by platelet-derived growth factor (PDGF), a growth factor involved in the decision between cellular proliferation and apoptosis (Franke et al., Cell 81:727-736, 1995). Akt kinases are also activated by insulin-like growth factor (IGF1), and in this capacity are involved in survival of cerebellar neurons (Dudek et al., Science 275:661-665, 1997). Furthermore, Akt1 is involved in the activation of NFkB by tumor necrosis factor (TNF) (Ozes et al., Nature 401:82-85, 1999). Akt kinases have been implicated in insulin-regulated glucose transport and the development of non-insulin dependent diabetes mellitus (Krook et al., Diabetes 47:1281-1286, 1998).

Clearly, Akt kinases play varied and important roles in a number of intracellular signaling pathways, and are thus good starting points form which to identify novel protein interactions that define disease-related signal transduction pathways. To this end, Akt1 and Akt2 were used to identify Akt-interacting proteins that may be potential targets for drug intervention. As a result of these studies, an interaction between Akt2 and intracellular chloride channel protein CLIC1 was identified. CLIC1, also known as NCC27 (nuclear chloride channel-27), was first cloned from human U937 myelomonocytic cells and is the first member of the CLIC family of chloride channels (Valenzuela et al., J. Biol. Chem. 272:12575-12582, 1997). CLIC1 primarily localizes to the nuclear membrane and likely plays a role in the transport of chloride into the nucleus. The finding that CLIC1 and Akt2 associate with one another is intriguing, suggesting that Akt2 may play a role in regulating nuclear ion transport. Interestingly, another related CLIC family member that localizes to the nuclear membrane, CLIC3, has been demonstrated to interact with a signal transduction protein, ERK7 (Qian et al., J. Biol. Chem. 274:1621-1627, 1999). Taken together, these results suggest that intracellular chloride channels may be intimately linked to transduction of extracellular signals. Here, we describe three new interactors of the chloride channel protein CLIC1.

The first interactors for CLIC1 are two isoforms of the RNA-binding protein TLS, termed TLSa (FUS(526), and TLSb (FUS(525)). TLS (also known as FUS) is fused to the transcription factor CHOP in malignant liposarcoma (Rabbitts et al., Nat. Genet. 4:175-180, 1993; Crozat et al., Nature 363:640-644, 1993), and to ERG in acute myeloid leukemia (Ichikawa et al., Cancer Res. 54:2865-2868, 1994; Panagopoulos et al., Genes Chromosomes Cancer 11:256-262, 1994). Furthermore, TLS/FUS is very similar to the EWS protein, which is often translocated in Ewing sarcoma. TLS (FUS) contains Arg-, Gln-, Ser-, and Gly-rich regions, an RNA recognition motif (RRM, a ˜90 amino acid domain found in known and putative RNA-binding proteins such as hnRNPs, snRNPs, and various regulatory proteins), and a RanBP-type zinc finger (found in Ran binding proteins involved in transport through the nuclear pore complex, and in Mdm2, which regulates p53 activity by binding to p53 and signaling its transport to the cytoplasm). The N-terminus of FUS has been shown to interact with RNA polymerase II, which the C-terminus interacts with SR (mRNA splicing) proteins (Yang et al., Mol. Cell Biol. 20:3345-3354, 2000). FUS was identified biochemically as a DNA-binding protein specifically induced by the tyrosine kinase activity of the oncoproteins BCR/ABL (Perrotti et al., EMBO J. 17:4442-4455, 1998). Suppression of TLS expression in myeloid precursor cells (by expression of an antisense construct) was shown to be associated with upregulation of the granulocyte colony-stimulating factor (GCSF) receptor expression and accelerated GCSF-stimulated differentiation, and downregulation of IL-3 receptor beta chain expression. These findings suggested that TLS may be involved in BCR/ABL leukemogenesis by controlling growth factor-dependent differentiation through the regulation of cytokine receptor expression. In support of this, disruption of the TLS homolog in mice demonstrates that TLS is essential for neonatal viability, influences lymphocyte development in a cell non-autonomous manner, is involved in B cell proliferative responses to mitogenic stimuli, and is required for maintenance of genome stability (Hicks et al., Nat. Genet. 24:175-179, 2000). The interaction of TLS with CLIC1 suggests that this putative chloride channel, located both within the nucleus as well as in the nuclear membrane, may mediate changes in transcription or mRNA processing in response to cellular signals. The amino acid sequences of the two TLS isoforms (a and b) are nearly identical, with only an S->T change at position 64 and an insertion of glycine at the next position distinguishing these proteins. Therefore, a modulator of TLS isoforms (a and/or b) or CLIC1 or interaction thereof may be used to treat inflammatory diseases.

The third interactor for CLIC1 is the low-density lipoprotein LRP1. LRP1 is a large (4,544 amino acid) protein that binds and internalizes a diverse set of ligands, making LRP the most multifunctional endocytic receptor known. LRP1 contains three clusters of putative ligand binding domains, each structurally comparable to the classical LDL receptor. In a mouse system, LRP1 functions as a receptor for alpha-2-macroglobulin (A2M), and it has been proposed that LRP1 acts as a sensor for necrotic cell death in tissues, leading to proinflammatory immune responses (Binder et al., Nature Immunology 2:151-155, 2000). LRP1 has also been shown to be involved in the uptake of apolipoprotein E-containing particles by neurons, and together with early linkage data this finding suggested a role for LRP1 in Alzheimer's disease. However, recent findings suggest that genetic variation in LRP1 is not a major risk factor in Alzheimer's disease (Scott et al., Neurogenetics 1: 179-183, 1998). The interaction of CLIC1 with LRP1 may be physiologically relevant, as CLIC1 is found at low abundance in the cytoplasm and cytoplasmic membrane. Therefore, a modulator of LRP1 or CLIC1 or interaction thereof may be used to treat inflammatory diseases.

Nuclear hormone receptors play important roles in development, reproduction, and physiology by altering gene transcription in response to hormonal signals (Whitfield et al., J. Biol. Chem. Suppl. 32-22:110-122, 1999; Klein-Hitpass et al., J. Mol. Med. 76:490-496, 1998). Misregulation of hormone receptor signaling pathways is responsible for a variety of diseases. For example, aldosterone and its receptor (the mineralocorticoid receptor, MCR) are involved in hypertension and congestive heart failure (Duprez et al., Curr. Hypertens. Rep. 2:327-334, 2000), and it has recently been shown that a missense mutation in MCR that alters its ligand specificity is responsible for pregnancy-exacerbated hypertension (Geller et al., Science 289:119-123, 2000). Lilkewise, glucocorticoids and the glucocorticoid receptor (GR) have been implicated in chronic inflammation and arthritis (Banres, P. J., Science 94:557-572, 1998), and the oxysterol liver receptor (LXR), farnesoid X receptor (FXR), and other nuclear receptors are involved in cholesterol homeostasis and atherogenesis (Schroepfer, G. J., Physiol. Rev. 80:361-554, 2000; Haynes et al., J Nucl. Cardiol. 7:500-508, 2000; Brown and Jessup, Atherosclerosis 142:1-28, 1999).

Collectively, the nuclear receptor superfamily is responsive to a wide variety of ligands. Nuclear hormone receptors share several important structural features, including a variable N-terminal region, a conserved central DNA-binding domain, a variable hinge region, and a conserved C-terminal ligand-binding domain (Moras and Gronemeyer, Curr. Op. Cell Biol. 10:384-291, 1998; Mangelsdorf et al., Cell 83:8350-839, 1995). Despite this conserved structural organization, interactions between ligands and receptors are remarkably specific. Hormone binding results in conformation changes in the receptor, allowing binding to specific DNA sequences (hormone response elements, HREs) in target gene promoters resulting in changes in target gene transcription. Interaction of nuclear hormone receptors with accessory proteins determines whether the receptor activates or represses transcription. Receptors can recruit coactivators that remodel chromatin and stabilize the RNA polymerase machinery, or alternatively can interact with factors that condense chromatin structure and inactivate gene expression (Wolffe et al., Cell Research 7:127-142, 1997). Furthermore, binding of a nuclear hormone receptor to other cellular proteins can alter the subcellular localization of the receptor and control its ability to bind hormone and HREs (DeFranco et al., J. Steroid Biochem. and Mol. Biol. 65:51-58, 1998). Clearly, identification of factors with which nuclear hormone receptors interact is vital to understanding the process by which hormonal signals are transduced into transcriptional responses. In addition, identification of receptor-interacting proteins will increase the repertoire of potential targets for therapeutic intervention in the treatment of diseases due to defects involving nuclear hormone signaling.

Nuclear receptor coactivator 2 (NCOA2, also known as glucocorticoid receptor-interacting protein 1 or GRIP1) is a transcriptional coactivator that mediates the stimulatory effect of nuclear hormone receptors on target gene transcription. NCOA2 was initially identified as a coactivator for glucocorticoid receptor, but in fact it is able to interact with many nuclear hormone receptors (Hong et al., Mol. Cell Biol. 17:2735-1744, 1997). NCOA2 is involved in the recruitment of transcriptional activators/chromatin remodeling factors such as CBP and PCAF to promoters involved in myogenesis (Chen et al., Genes Dev. 14:1209-1228, 2000). The interaction of NCOA2 with a variety of nuclear hormone receptors suggests that NCOA2 plays a role in multiple hormone-dependent signaling pathways, and consequently specificity in the responses is likely to be imparted by both the nuclear hormone receptor and other proteins with which NCOA2 interacts. Therefore, a modulator of NCOA2 may be used to treat inflammatory diseases.

The first three NCOA2-interacting proteins discovered are likely involved in transcriptional regulation. The first is the bromodomain protein NAG4. NAG4, also known as Celtix1 or BP465, is a human protein closely related to murine BP75, a novel bromodomain protein identified as an interactor of the PDZ domain in the BAS-like protein tyrosine phosphatase (PTP-BL) (Cuppen et al., FEBS Lett. 459:291-298, 1999). Bromodomains bind acetylated lysines, and bromodomain proteins are thought to be involved in the assembly of multiprotein complexes involved in transcriptional activation. The interaction of NCOA2 with a bromodomain protein is consistent with this hypothesized role, which is further strengthened by presence of two predicted bipartite nuclear localization signals near the N-terminus of NAG4, suggesting NAG4 may be a nuclear protein. Therefore, a modulator of NAG4 or NCOA2 or interaction thereof may be used to treat inflammatory diseases.

The second NCOA2-interactor is XE169, also known as SMCX. XE169 is encoded by an X-linked gene that, like its mouse homolog, escapes X inactivation (Wu et al., Hum. Mol. Genet. 3:153-160, 1994). Alternative splicing generates two distinct transcripts, either containing or missing 9 nucleotides, which in turn predict two XE169n protein isoforms of 1557 and 1560 amino acids, respectively. The NCOA2-interacting region of XE169 encompasses the region of this alternative splice, and appears to encode perhaps a third splice form; however, unlike the previously described 1557-residue isoform which lacks amino acids 1370-1372 (GKR), the region lacks amino acids 1371-1373 (KRD). The XE169 protein contains an ARID domain (AT-rich interacting domain) and two predicted PHD fingers; these domains are likely involved in positive and negative transcriptional regulation and chromatin remodeling. The presence of such domains makes the identified interaction with NCOA2 particularly intriguing. XE169 displays 50% amino acid identity over nearly 1600 amino acids to Rb-binding protein 2 (RBP2), suggesting a function in association with the Retinoblastoma protein. Therefore, a modulator of XE169 or NCOA2 or interaction thereof may be used to treat inflammatory diseases.

The third NCOA-2 interactor is the estrogen related receptor alpha (ERR-alpha). ERR-alpha is an orphan nuclear receptor that was initially identified by low stringency hybridization of a kidney cDNA library using a probe derived from the DNA-binding domain of estrogen receptor (Giguere et al., Nature 331:91-94, 1988). The DNA binding site preference for ERR-alpha has been characterized and termed the ERRE. Interestingly, the ERRE is found in the 5-prime-flanking region of the mitochondrial medium-chain acyl coenzyme A dehydrogenase gene that is involved in the metabolism of fat (Sladek et al., Mol. Cell Biol. 17:5400-5409, 1997). ERR-alpha is also thought to be involved in fat metabolism because the ERR-alpha gene is most highly expressed in tissues that preferentially utilize fatty acids such as kidney, heart and brown adipocytes. Furthermore, the association between ERR-alpha and NCOA2 has recently been reported using GST pull-down assays (Zhang et al., J. Biol. Chem. 275:20837-20846, 2000). Therefore, a modulator of ERR-alpha or NCOA2 or interaction thereof may be used to treat inflammatory diseases.

NCOA2 interacts with three kinase or kinase-associated protins involved in intracellular signal transduction. The first of these, KIAA0619 (also known as ROCK2), is a serine/threonine kinase that regulates cytokinesis, smooth muscle contraction, the formation of actin stress fibers and focal adhesions, and the activation of the c-fos serum response element, and is a target for the small GTPase Rho (Takahashi et al., Genomics 55:235-237, 1999). ROCK2/KIAA0619 is a 1388 amino acid protein that displays 65% identity over 1359 residues to p160/ROCK1, which is a Rho-associated kinase involved in cytoskeletal rearrangement that we have identified as an interactor of the farnesoid X-activated receptor (FXR). The interaction of two highly related Rho-associated kinases (i.e. FXR and NCOA2) strengthens the argument that these interactions are biologically relevant. ROCK2 contains an amino-terminal kinase domain, a C-terminal pleckstrin homology domain, and several predicted coiled-coil regions. Therefore, a modulator of KIAA0619 or NCOA2 or interaction thereof may be used to treat inflammatory diseases.

The second interaction for NCOA2 is with HAX1 (HS1BP1), which was originally identified by its association with HS1. HS1 (also known as HCLS1) is a protein that associates with protein tyrosine kinases and is involved in clonal expansion and deletion in lymphoid cells (Egashira et al., Cytogenet. Cell Genet. 72:175-176, 1996) and erythropoietin-induced differentiation of erythroid cells (Ingley et al., J. Biol. Chem. 275:7887-7893, 2000). Interaction of HS1BP1 with HS1 was confirmed by coimmunoprecipitation from transfected cells and by colocalization using confocal microscopy (Suzuki et al., J. Immunol. 158:2736-1744, 1997). HS1BP1 is a 279 amino acid protein that is expressed ubiquitously and is found in several subcellular compartments, including mitochondria, ER, and the nuclear envelope. Therefore, a modulator of HS1BP1 or NCOA2 or interaction thereof may be used to treat inflammatory diseases.

The final kinase-related NCOA2 interactor is TILP(392) (also known as PN12361). TILP(392) is similar to the protein product of the mouse AZ2 gene (GenBank accession AB007141). AZ2 is induced upon exposure 5-azacytidine, an inhibitor of DNA methyltransferase (Miyagawa et al., Gene 240:289-295, 1999). The AZ2 protein is primarily cytoplasmic and is found in the testis, brain and lung of mouse. The amino-terminus of the AZ2 protein is similar to ITRAF and TBK1, two proteins involved in the kinase-dependent signal transduction cascade leading to NFkappaB activation. IN fact, overexpression of AZ2 has been shown to inhibit TNF alpha-mediated activation of NFkappaB. Taken together, the finding the NCOA2 and TILP(392) can interact suggest that NCOA2 may be capable of influencing the activation of other transcription regulators such as NFkappaB. Therefore, a modulator of TILP(392) or NCOA2 or interaction thereof may be used to treat inflammatory diseases.

One NCOA2 interactor we have identified appears to play multiple roles in the cell, namely in cell adhesion/signaling and in transcriptional regulation. This interactor, beta-catenin (CTNNB1), is a component of the protein complex that anchors E-cadherins to the actin cytoskeleton, and is thus involved in the formation and maintenance of adherens junctions between epithelial cells. CTNNB1 also interacts with the APC (adenomatous polyposis of the colon) protein, which is localized to both the nucleus and cytoplasm and is a negative regulator of CTNNB1 activity. In the cytoplasm, the E-cadherin/CTNNB1/APC complex is thought to play a role in transmitting the contact inhibition signal into the cell, which is consistent with the hyperplasia phenotype of APC mutations. Interestingly, in APC mutants, CTNNB1 accumulates in the nucleus in a constitutively active complex with the transcription factor Tcf-4 (a component of the Wnt signaling pathway), and restoration of APC function dissociates these complexes (Korinek et al., Science 275:1784-1787, 1997, Morin et al., Science 275:1787-1790, 1997). Taken together, these results suggest that the anti-tumor activities of APC are related to its ability to suppress transcriptional activation by CTNNB1/Tcf-4 complexes. NCOA2/CTNNB1 complexes, if they form in vivo, may have properties similar to CTNNB1/Tcf-4. Therefore, a modulator of CTNNB1 or NCOA2 or interaction thereof may be used to treat inflammatory diseases.

The NCOA2 interactor LRRFIP2a [leucine-rich repeat (in FLII) interacting protein 2, splice variant a] has cellular functions that are not yet clear. LRRFIP2a and LRRFIP1 are a pair of proteins identified in a yeast two-hybrid assay as interactin with the leucine rich region of the human flightless-I (FLII) protein Fong et al., Genomics 58:146-157, 1999). Human FLII contains a gelsolin-like domain that is able to associate with actin, and although the biological role of human FLII is unknown, the deletion of one allele of FLII is associated with Smith-Magenis syndrome (SMS), the phenotypes of which include short stature, brachydactyly, developmental delay, dysmorphic features, sleep disturbances, and behavioral problems (Chen et al., Genes Dev. 14:1209-1228, 1995). LRRFIP1 exhibits sequence identity with the TRIP RNA-binding protein and GCF-2 transcriptional repressor, which are also related to the murine FLAP-1 gene. LRRFIP2a is a novel gene that shares sequence homology with LRRFIP1 and FLAP-1. A coiled-coil domain, conserved in LRRFIP1 and LRRFIP2a, serves as a potential interaction motif for the FLII leucine-rich repeats. Expression analyses suggest that the LRRFIP2a gene is active in heart and skeletal muscle (in which alternatively spliced forms appear to be expressed), pancreas, placenta, testis, and stomach. Therefore, a modulator of LRRFIP2a or NCOA2 or interaction thereof may be used to treat inflammatory diseases.

Another interaction for NCOA2 is with Prosaposin (PSAP). PSAP can either be targeted to lysosomes or secreted. In lysosomes, PSAP is proteolytically cleaved to yield four similar proteins (SAP-A, B, C, and D) that promote the degradation of glycosphingolipids by acidic hydrolases (Rorman and Grabowski, Genomics 5:486-492, 1989). When secreted, PSAP has neurite outgrowth activity (Qi et al., Biochemistry 38:6284-6291, 1999), can prevent cell death and increase ERK phosphorylation in Schwann cells (Hiraiwa et al., Proc. Natl. Acad. Sci 94:4778-4781, 1997), and acts to prevent degeneration of promote regeneration of injured peripheral nerves (Hiraiwa et al., Glia 26:353-360, 1999). Mutations in Prosaposin result in variants of metachromatic leukodystrophy and Gaucher's disease (glycocerebroside accumulation, hepatosplenomegaly, and regression of neurological maturation). NCOA2 interacts with amino acids 140-337 of PSAP; this region corresponds to SAP-B and part of SAP-C (after proteolytic processing), and includes the neurotropic region of the protein at the N-terminus of SAP-C. The significance of the interaction between PSAP and NCOA2 is not clear; from two-hybrid results it is not possible to determine which form of PSAP (intact vs. processed) interacts with NCOA2 in vivo, nor in which subcellular compartment the interaction takes place. Nonetheless, the involvement of PSAP in a variety of intra- and extracellular processes including cell signaling and growth control suggests the interaction with NCOA2 may be biologically relevant. Therefore, a modulator of PSAP or NCOA2 or interaction thereof may be used to treat inflammatory diseases.

The final interaction of NCOA2 is with CIT. CIT is a rho-associated serine/threonine kinase with close homologs in rat and mouse, contains both a pleckstrin homology domain and a phorbol ester/diacylglycerol binding domain. The prey sequence that interacts with NCOA2 includes the PE/DAG binding domain and most of the PH domain. EST analyses suggest a broad tissue distribution of CIT expression. Therefore, a modulator of CIT or NCOA2 or interaction thereof may be used to treat inflammatory diseases.

We have also identified interactions of NOTCH2 with ESR1 and ER-beta. ESR1 and ER-beta are nuclear hormone receptors that display sequence similarity to the glucocorticoid receptor (GR) and function as homodimers to regulate transcription in response to 17-beta-estradiol. Mutations in ESR1 have been implicated in the development and progression of breast cancer (Clark and McGuire, Semin. Oncol. 15(suppl. 1):20-25, 1988; McGuire et al., Molec. Endocr. 5:1571-1577,1991) and ESR1 and ER-beta are implicated in pituitary adenomas (Shupnik et al., J. Clin. Endocr. Metab. 83:3965-3972, 1998). ER activity appears to be modulated by phosphorylation at specific residues by the cyclin A-CDK2 complex (Rogatsky et al., J. Biol. Chem. 274:22296-22302, 1999) and by interaction with other cellular proteins such as rho GTPases (Su et al., J. Biol. Chem. 2000 November 1 [epub ahead of print], 2000; Knoblauch and Garabedian, Mol. Cell Biol. 19:3748-2759, 1999). Therefore, a modulator of ESR1, ER-beta, or NCOA2 or interaction thereof may be used to treat inflammatory diseases.

### 2.2. Protein Complexes

Accordingly, the present invention provides protein complexes formed by interacting pairs of proteins described in the tables. The present invention also provides protein complexes in which one or more of the interacting protein members are native proteins or homologues, derivatives or fragments of native proteins.

Thus, for example, one interacting partner in a protein complex can be a complete native PRAK, a PRAK homologue capable of interacting with, e.g., ERK3, a PRAK derivative, a derivative of the PRAK homologue, a PRAK fragment capable of interacting with ERK3 (PRAK fragment(s) containing the coordinates shown in Table 1), a homologue or derivative of the PRAK fragment, or a fusion protein containing (1) complete native PRAK, (2) a PRAK homologue capable of interacting with ERK3 or (3) a PRAK fragment capable of interacting with ERK3. Besides native ERK3, useful interacting partners for PRAK or a homologue or derivative or fragment thereof also include homologues of ERK3 capable of interacting with PRAK, derivatives of the native or homologue ERK3 capable of interacting with PRAK, fragments of the ERK3 capable of interacting with PRAK (e.g., a fragment containing the identified interacting regions shown in Table 1), derivatives of the ERK3 fragments, or fusion proteins containing (1) a complete ERK3, (2) a ERK3 homologue capable of interacting with PRAK or (3) a ERK3 fragment capable of interacting with PRAK.

ERK3 fragments capable of interacting with PRAK can be identified by the combination of molecular engineering of a ERK3-encoding nucleic acid and a method for testing protein-protein interaction. For example, the coordinates in Table 1 can be used as starting points and various ERK3 fragments falling within the coordinates can be generated by deletions from either or both ends of the coordinates. The resulting fragments can be tested for their ability to interact with PRAK using any methods known in the art for detecting protein-protein interactions (e.g., yeast two-hybrid method). Alternatively, various ERK3 fragments can be made by chemical synthesis. The ERK3 fragments can then be tested for their ability to interact with PRAK using any method known in the art for detecting protein-protein interactions. Examples of such methods include protein affinity chromatography, affinity blotting, in vitro binding assays, yeast two-hybrid assays, and the like. Likewise, PRAK fragments capable of interacting with ERK3 can also be identified in a similar manner.

Other protein complexes can be formed in a similar manner based on other interactions provided in the tables.

In a specific embodiment of the protein complex of the present invention, two or more interacting partners are directly fused together, or covalently linked together through a peptide linker, forming a hybrid protein having a single unbranched polypeptide chain. Thus, the protein complex may be formed by “intramolecular” interactions between two portions of the hybrid protein. Again, one or both of the fused or linked interacting partners in this protein complex may be a native protein or a homologue, derivative or fragment of a native protein.

The protein complexes of the present invention can also be in a modified form. For example, an antibody selectively immunoreactive with the protein complex can be bound to the protein complex. In another example, a non-antibody modulator capable of enhancing the interaction between the interacting partners in the protein complex may be included. Alternatively, the protein members in the protein complex may be cross-linked for purposes of stabilization. Various crosslinking methods may be used. For example, a bifunctional reagent in the form of R—S—S—R′ may be used in which the R and R′ groups can react with certain amino acid side chains in the protein complex forming covalent linkages. See e.g., Traut et al., in Creighton ed., Protein Function: A Practical Approach, IRL Press, Oxford, 1989; Baird et al., J. Biol. Chem., 251:6953-6962 (1976). Other useful crosslinking agents include, e.g., Denny-Jaffee reagent, a heterbiofunctional photoactivable moiety cleavable through an azo linkage (See Denny et al., Proc. Natl. Acad. Sci. USA, 81:5286-5290 (1984)), and ¹²⁵I-{S-[N-(3-iodo-4-azidosalicyl)cysteaminyl]-2-thiopyridine}, a cysteine-specific photocrosslinking reagent (see Chen et al., Science, 265:90-92 (1994)). The above-described protein complexes may further include any additional components, e.g., other proteins, nucleic acids, lipid molecules, monosaccharides or polysaccharides, ions, etc.

The present invention provides isolated nucleic acid molecules. The nucleic acid molecules can be in the form of DNA, RNA, or a chimera or hybrid thereof, and can be in any physical structures including single-stranded or double-stranded molecules, or in the form of a triple helix. In one embodiment, the isolated nucleic acid molecule has a sequence of SEQ ID NO:1, SEQ ID NO:3, SEQ ID NO:5, SEQ ID NO:7, or SEQ ID NO:9 or the complement thereof.

In addition, nucleic acid molecules are also contemplated, which are capable of specifically hybridizing, under stringent hybridization conditions, to a nucleic acid molecule having the sequence of SEQ ID NO:1, SEQ ID NO:3, SEQ ID NO:5, SEQ ID NO:7, or SEQ ID NO:9 or the coding sequence or complement thereof. Preferably, such nucleic acid molecules encode a polypeptide having the sequence of SEQ ID NO:2, SEQ ID NO:4, SEQ ID NO:6, SEQ ID NO:8, or SEQ ID NO:10 or fragment thereof.

In another embodiment, an isolated nucleic acid molecule is provided, which has a sequence that is at least 50%, preferably at least 60%, more preferably at least 75%, 80%, 82%, 85%, even more preferably at least 90%, 91%, 92%, 93%, 94%, 95%, 96%, 97%, 98%, or 99% identical to the sequence of SEQ ID NO:1, SEQ ID NO:3, SEQ ID NO:5, SEQ ID NO:7, or SEQ ID NO:9 or the coding sequence or complement thereof. Preferably, such nucleic acid molecules encode a polypeptide having the sequence of SEQ ID NO:2, SEQ ID NO:4, SEQ ID NO:6, SEQ ID NO:8, or SEQ ID NO:10.

As is apparent to skilled artisans, homologous nucleic acids or nucleic acids capable of hybridizing with a nucleic acid of the sequence of SEQ ID NO:1, SEQ ID NO:3, SEQ ID NO:5, SEQ ID NO:7, or SEQ ID NO:9 or the coding sequence thereof can be prepared by manipulating a nucleic acid molecule having a sequence of SEQ ID NO:1, SEQ ID NO:3, SEQ ID NO:5, SEQ ID NO:7, or SEQ ID NO:9. For example, various nucleotide substitutions, deletions or insertions can be incorporated into the nucleic acid molecule by standard molecular biology techniques. As will be apparent to skilled artisans, such nucleic acids are useful irrespective of whether they encode a functional protein. For example, they can be used as probes for isolating and/or detecting nucleic acids. Nevertheless, preferably the homologous nucleic acids or the nucleic acids capable of hybridizing with a nucleic acid of the sequence of SEQ ID NO:1, SEQ ID NO:3, SEQ ID NO:5, SEQ ID NO:7, or SEQ ID NO:9 encode a polypeptide having one or more activities of the polypeptides encoded by SEQ ID NO:1, SEQ ID NO:3, SEQ ID NO:5, SEQ ID NO:7, or SEQ ID NO:9.

In addition, nucleic acid molecules that encode the proteins having an amino acid sequence of SEQ ID NO:2, SEQ ID NO:4, SEQ ID NO:6, SEQ ID NO:8, or SEQ ID NO:10 are also intended to fall within the scope of the present invention. As will be immediately apparent to a skilled artisan, due to genetic code degeneracy, such nucleic acid molecules can be designed conveniently by nucleotide substitutions in the wild-type nucleotide sequence of SEQ ID NO:1, SEQ ID NO:3, SEQ ID NO:5, SEQ ID NO:7, or SEQ ID NO:9.

In addition, the present invention further encompasses nucleic acid molecules encoding a protein that has a sequence that is at least 75%, preferably at least 85%, 90%, 91%, 92%, 93%, or 94%, and more preferably at least 95%, 96%, 97%, 98%, or 99% identical to the sequence of SEQ ID NO:1, SEQ ID NO:3, SEQ ID NO:5, SEQ ID NO:7, or SEQ ID NO:9 or coding sequence thereof. The various nucleic acid molecules may be produced by chemical synthesis and/or recombinant techniques based on an isolated nucleic acid molecule having a sequence of SEQ ID NO:1, SEQ ID NO:3, SEQ ID NO:5, SEQ ID NO:7, or SEQ ID NO:9.

In another embodiment of the present invention, oligonucleotides are provided having a contiguous span of at least 10, 12, 15, 16, 17, 18, 19, 20, 21, 22, 23, 24, 25, 26, 27, 28, 29, 30, 35, 50, 75, 100, 200, 300, 400, 500, 600, 700, 800, 900, 1000, 1100, 1200, 1300, 1400, 1500, or 1585 nucleotides of the sequence of SEQ ID NO:1, SEQ ID NO:3, SEQ ID NO:5, SEQ ID NO:7, or SEQ ID NO:9, or the complement thereof. Preferably, the oligonucleotides are less than the full length of the sequence of SEQ ID NO:1, SEQ ID NO:3, SEQ ID NO:5, SEQ ID NO:7, or SEQ ID NO:9, more preferably no greater than 1200, 800, 600, 400, 200, 100, or 50 nucleotides in length. In a preferred embodiment, the oligonucleotides have a length of about 12-18, 19-25, 26-34, 35-50, or 51-100 nucleotides. In a specific embodiment, the oligonucleotide is a sequence encoding a contiguous span of at least 4, 5, 6, 7, 8, 9, 10, 11, 12, 13, 14, 15, 17, 20, 22, 25, 30, 35, 50, 100, 150, 200, 300, 400, or 500 amino acids of SEQ ID NO:1, SEQ ID NO:3, SEQ ID NO:5, SEQ ID NO:7, or SEQ ID NO:9. In another specific embodiment, the oligonucleotide is an antisense oligo as described in Section 11.2.2. In another specific embodiment, the oligonucleotide is a ribozyme molecule as described in Section 11.2.3. In yet another specific embodiment, the oligonucleotide can serve as a primer for nucleic acid amplification reactions, such as the Polymerase Chain Reaction (PCR).

The present invention further encompasses oligonucleotides that have a length of at least 10, 12, 15, 17, 18, 19, 20, 21, 22, 23, 24, 25, 30, 50, 75, 100, 200, 300, 400, 500, or 600 nucleotides and preferably no greater than 1500, 1300, 1100, 800, 600, 400, 200 or 100 nucleotides, and are at least 85%, 90%, 92% or 94%, and more preferably at least 95%, 96%, 97%, 98%, or 99% identical to a contiguous span of nucleotides of the sequence of SEQ ID NO:1, SEQ ID NO:3, SEQ ID NO:5, SEQ ID NO:7, or SEQ ID NO:9 or the complement thereof. The oligonucleotides can have a length of about 12-18, 19-25,26-34,35-50, 51-100, 101-250,251-500, 501-1000, 1000-1587 nucleotides. In a preferred embodiment, the oligonucleotides have a length of about 12-100, 15-75, 17-50, 21-50, or preferably 25-50 nucleotides. Preferably, the oligonucleotide is a sequence encoding a contiguous span of at least 4, 5, 6, 7, 8, 9, 10, 11, 12, 13, 14, 15, 17, 20, 22, 25, 30, 35, 50, 100, 150, 200, 250, 500, 600, 750, 900, 1150, 1300, or 1585 amino acids of SEQ ID NO:2, SEQ ID NO:4, SEQ ID NO:6, SEQ ID NO:8, or SEQ ID NO:10.

In addition, oligonucleotides are also contemplated having a length of at least 10, 12, 15, 17, 18, 19, 20, 21, 22, 23, 24, 25, 30, 50, 75, 100, 200, 300, 400, 500, or 600 nucleotides and preferably no greater than 1500, 1300, 1100, 800, 600, 400, 200 or 100 nucleotides, and capable of hybridizing to the nucleotide sequence of SEQ ID NO:1, SEQ ID NO:3, SEQ ID NO:5, SEQ ID NO:7, or SEQ ID NO:9, or the complement thereof, under stringent hybridization conditions. In a preferred embodiment, the oligonucleotides have a length of about 12-100, 15-75, 17-50,21-50, or preferably 25-50 nucleotides. In another preferred embodiment, the oligonucleotides capable of hybridizing to the nucleotide sequence of SEQ ID NO:1, SEQ ID NO:3, SEQ ID NO:5, SEQ ID NO:7, or SEQ ID NO:9 or the complement thereof encode a contiguous span of at least 7, 8, 9, 10, 11, 12, 13, 14, 15, 16, 17, 18, 19, 20, 25, 30, 50, 100, 150, 200, 300, 400, 500, 750, 1000, 1250, or 1500 amino acids of SEQ ID NO:2, SEQ ID NO:4, SEQ ID NO:6, SEQ ID NO:8, or SEQ ID NO:10.

As will be apparent to skilled artisans, the various oligonucleotides of the present invention are useful as probes for detecting nucleic acids in cells and tissues. They can also be used as primers for procedures including the amplification of nucleic acids or homologues thereof, sequencing nucleic acids, and the detection of mutations in nucleic acids or homologues thereof. In addition, the oligonucleotides may be used to encode a fragment, epitope or domain of proteins or a homologue thereof, which is useful in a variety of applications including use as antigenic epitopes for preparing antibodies against proteins.

It should be understood that the nucleic acid molecules of the present invention may be in standard forms with conventional nucleotide bases and backbones, but can also be in various modified forms, e.g., having therein modified nucleotide bases or backbones. Examples of modified nucleotide bases or backbones described in Section 11.2.2 in the context of modified antisense compounds should be equally applicable in this respect.

The present invention also provides isolated polypeptides. Tthe present invention also encompasses a polypeptide having an amino acid sequence that is at least 50%, preferably at least 60%, more preferably at least 75%, 80%, 85%, 90%, 91%, 92%, 93%, 94%, and even more preferably at least 95%, 96%, 97%, 98%, or 99% identical to the amino acid sequence of SEQ ID NO:2, SEQ ID NO:4, SEQ ID NO:6, SEQ ID NO:8, or SEQ ID NO:10. In a specific embodiment, the homologous polypeptide is a naturally occurring protein variant of a protein identified in a human population. Such a variant may be identified by assaying the nucleic acids or protein in a population, as is generally known in the art. In another embodiment, the present invention also provides an isolated polypeptide that is encoded by an isolated nucleic acid molecule that specifically hybridizes fully to the isolated nucleic acid molecule of SEQ ID NO:1, SEQ ID NO:3, SEQ ID NO:5, SEQ ID NO:7, or SEQ ID NO:9, or the complement thereof under moderately or highly stringent conditions.

The present invention further encompasses fragments proteins having a contiguous span of at least 6, 7, 8, 9, 10, 11, 12, 13, 14, 15, 16, 17, 18, 19, 20, 25, 50, 100, 150, 250, 500, 750, 1000, 1200, 1400, or at least 1585 amino acids of the sequence of SEQ ID NO:2, SEQ ID NO:4, SEQ ID NO:6, SEQ ID NO:8, or SEQ ID NO:10, but less than the full length of the sequence of SEQ ID NO:2, SEQ ID NO:4, SEQ ID NO:6, SEQ ID NO:8, or SEQ ID NO:10. For example, such fragments can be generated as a result of the deletion of a contiguous span of a certain number of amino acids from either or both of the amino and carboxyl termini of the protein having the sequence of SEQ ID NO:2, SEQ ID NO:4, SEQ ID NO:6, SEQ ID NO:8, or SEQ ID NO:10. In specific embodiments, a polypeptide is provided including a contiguous span of at least 4, 5, 6, 7, 8, 9, 10, 11, 12, 13, 14, 15, 18, 20, 25, 50, 150, 250, 300, 600, 850, 1100, 1350, or at least 1585 amino acids of the sequence of SEQ ID NO:2, SEQ ID NO:4, SEQ ID NO:6, SEQ ID NO:8, or SEQ ID NO:10. In other specific embodiments, the polypeptide fragments contain immunogenic or antigenic epitopes. Such epitopes can be readily determined by computer programs such as MacVector from International Biotechnologies, Inc. and Protean from DNAStar. In addition, epitopes can also be selected experimentally by any methods known in the art, e.g., in U.S. Pat. Nos. 4,833,092 and 5,194,392, both of which are incorporated herein by reference.

In addition, the present invention is also directed to polypeptides that are homologous to the foregoing polypeptide fragments. Such a homologous polypeptide may have the same length as one of the foregoing polypeptide fragments of the present invention (e.g., from 5 to 50, from 5 to 30, or from 7 to 25, or preferably 8 to 20 amino acids) but has an amino acid sequence that is at least 75%, 80%, 85%, 90%, preferably at least 95%, 96%, 97%, 98%, or, more preferably, at least 99% identical to the amino acid sequence of the corresponding polypeptide fragment.

Additionally, the present invention further relates to a hybrid polypeptide having any one of the foregoing polypeptides of the present invention covalently linked to another polypeptide. Such other polypeptide can also be one of the foregoing polypeptides of the present invention. Alternatively, such other polypeptide is not one of the foregoing polypeptides of the present invention. The covalent linkage in the hybrid polypeptide of the present invention can be merely a covalent bond between the two components of the hybrid polypeptide. Alternatively, any linker molecules may be used. For example, a peptide or a non-peptidic organic molecule may be used as a linker molecule.

### 2.3. Methods of Preparing Protein Complexes

The protein complex of the present invention can be prepared by a variety of methods. Specifically, a protein complex can be isolated directly from an animal tissue sample, preferably a human tissue sample containing the protein complex. Alternatively, a protein complex can be purified from host cells that recombinantly express the members of the protein complex. As will be apparent to a skilled artisan, a protein complex can be prepared from a tissue sample or recombinant host cells by coimmunoprecipitation using an antibody immunoreactive with an interacting protein partner, or preferably an antibody selectively immunoreactive with the protein complex as will be discussed in detail below.

The antibodies can be monoclonal or polyclonal. Coimmunoprecipitation is a commonly used method in the art for isolating or detecting bound proteins. In this procedure, generally a serum sample or tissue or cell lysate is admixed with a suitable antibody. The protein complex bound to the antibody is precipitated and washed. The bound protein complexes are then eluted.

Alternatively, immunoaffinity chromatography and immunoblotting techniques may also be used in isolating the protein complexes from native tissue samples or recombinant host cells using an antibody immunoreactive with an interacting protein partner, or preferably an antibody selectively immunoreactive with the protein complex. For example, in protein immunoaffinity chromatography, the antibody is covalently or non-covalently coupled to a matrix (e.g., Sepharose), which is then packed into a column. Extract from a tissue sample, or lysate from recombinant cells is passed through the column where it contacts the antibodies attached to the matrix. The column is then washed with a low-salt solution to wash away the unbound or loosely (non-specifically) bound components. The protein complexes that are retained in the column can be then eluted from the column using a high-salt solution, a competitive antigen of the antibody, a chaotropic solvent, or sodium dodecyl sulfate (SDS), or the like. In immunoblotting, crude proteins samples from a tissue sample extract or recombinant host cell lysate are fractionated by polyacrylamide gel electrophoresis (PAGE) and then transferred to a membrane, e.g., nitrocellulose. Components of the protein complex can then be located on the membrane and identified by a variety of techniques, e.g., probing with specific antibodies.

In another embodiment, individual interacting protein partners may be isolated or purified independently from tissue samples or recombinant host cells using similar methods as described above. The individual interacting protein partners are then combined under conditions conducive to their interaction thereby forming a protein complex of the present invention. It is noted that different protein-protein interactions may require different conditions. As a starting point, for example, a buffer having 20 mM Tris-HCl, pH 7.0 and 500 mM NaCl may be used. Several different parameters may be varied, including temperature, pH, salt concentration, reducing agent, and the like. Some minor degree of experimentation may be required to determine the optimum incubation condition, this being well within the capability of one skilled in the art once apprised of the present disclosure.

In yet another embodiment, the protein complex of the present invention may be prepared from tissue samples or recombinant host cells or other suitable sources by protein affinity chromatography or affinity blotting. That is, one of the interacting protein partners is used to isolate the other interacting protein partner(s) by binding affinity thus forming protein complexes. Thus, an interacting protein partner prepared by purification from tissue samples or by recombinant expression or chemical synthesis may be bound covalently or non-covalently to a matrix, e.g., Sepharose, which is then packed into a chromatography column. The tissue sample extract or cell lysate from the recombinant cells can then be contacted with the bound protein on the matrix. A low-salt solution is used to wash off the unbound or loosely bound components, and a high-salt solution is then employed to elute the bound protein complexes in the column. In affinity blotting, crude protein samples from a tissue sample or recombinant host cell lysate can be fractionated by polyacrylamide gel electrophoresis (PAGE) and then transferred to a membrane, e.g., nitrocellulose. The purified interacting protein member is then bound to its interacting protein partner(s) on the membrane forming protein complexes, which are then isolated from the membrane.

It will be apparent to skilled artisans that any recombinant expression methods may be used in the present invention for purposes of expressing the protein complexes or individual interacting proteins. Generally, a nucleic acid encoding an interacting protein member can be introduced into a suitable host cell. For purposes of forming a recombinant protein complex within a host cell, nucleic acids encoding two or more interacting protein members should be introduced into the host cell.

Typically, the nucleic acids, preferably in the form of DNA, are incorporated into a vector to form expression vectors capable of directing the production of the interacting protein member(s) once introduced into a host cell. Many types of vectors can be used for the present invention. Methods for the construction of an expression vector for purposes of this invention should be apparent to skilled artisans apprised of the present disclosure. See generally, Current Protocols in Molecular Biology, Vol. 2, Ed. Ausubel, et al., Greene Publish. Assoc. & Wiley Interscience, Ch. 13, 1988; Glover, DNA Cloning, Vol. II, IRL Press, Wash., D.C., Ch. 3, 1986; Bitter, et al., in Methods in Enzymology 153:516-544 (1987); The Molecular Biology of the Yeast Saccharomyces, Eds. Strathern et al., Cold Spring Harbor Press, Vols. I and II, 1982; and Sambrook et al., Molecular Cloning: A Laboratory Manual, Cold Spring Harbor Press, 1989.

Generally, the expression vectors include an expression cassette having a promoter operably linked to a DNA encoding an interacting protein member. The promoter can be a native promoter, i.e., the promoter found in naturally occurring cells to be responsible for the expression of the interacting protein member in the cells. Alternatively, the expression cassette can be a chimeric one, i.e., having a heterologous promoter that is not the native promoter responsible for the expression of the interacting protein member in naturally occurring cells. The expression vector may further include an origin of DNA replication for the replication of the vectors in host cells. Preferably, the expression vectors also include a replication origin for the amplification of the vectors in, e.g., E. coli, and selection marker(s) for selecting and maintaining only those host cells harboring the expression vectors. Additionally, the expression cassettes preferably also contain inducible elements, which function to control the transcription from the DNA encoding an interacting protein member. Other regulatory sequences such as transcriptional enhancer sequences and translation regulation sequences (e.g., Shine-Dalgarno sequence) can also be operably included in the expression cassettes. Termination sequences such as the polyadenylation signals from bovine growth hormone, SV40, lacZ and AcMNPV polyhedral protein genes may also be operably linked to the DNA encoding an interacting protein member in the expression cassettes. An epitope tag coding sequence for detection and/or purification of the expressed protein can also be operably linked to the DNA encoding an interacting protein member such that a fusion protein is expressed. Examples of useful epitope tags include, but are not limited to, influenza virus hemagglutinin (HA), Simian Virus 5 (V5), polyhistidine (6×His), c-myc, lacZ, GST, and the like. Proteins with polyhistidine tags can be easily detected and/or purified with Ni affinity columns, while specific antibodies immunoreactive with many epitope tags are generally commercially available. The expression vectors may also contain components that direct the expressed protein extracellularly or to a particular intracellular compartment. Signal peptides, nuclear localization sequences, endoplasmic reticulum retention signals, mitochondrial localization sequences, myristoylation signals, palmitoylation signals, and transmembrane sequences are examples of optional vector components that can determine the destination of expressed proteins. When it is desirable to express two or more interacting protein members in a single host cell, the DNA fragments encoding the interacting protein members may be incorporated into a single vector or different vectors.

The thus constructed expression vectors can be introduced into the host cells by any techniques known in the art, e.g., by direct DNA transformation, microinjection, electroporation, viral infection, lipofection, gene gun, and the like. The expression of the interacting protein members may be transient or stable. The expression vectors can be maintained in host cells in an extrachromosomal state, i.e., as self-replicating plasmids or viruses. Alternatively, the expression vectors can be integrated into chromosomes of the host cells by conventional techniques such as selection of stable cell lines or site-specific recombination. In stable cell lines, at least the expression cassette portion of the expression vector is integrated into a chromosome of the host cells.

The vector construct can be designed to be suitable for expression in various host cells, including but not limited to bacteria, yeast cells, plant cells, insect cells, and mammalian and human cells. Methods for preparing expression vectors for expression in different host cells should be apparent to a skilled artisan.

Homologues and fragments of the native interacting protein members can also be easily expressed using the recombinant methods described above. For example, to express a protein fragment, the DNA fragment incorporated into the expression vector can be selected such that it only encodes the protein fragment. Likewise, a specific hybrid protein can be expressed using a recombinant DNA encoding the hybrid protein. Similarly, a homologue protein may be expressed from a DNA sequence encoding the homologue protein. A homologue-encoding DNA sequence may be obtained by manipulating the native protein-encoding sequence using recombinant DNA techniques. For this purpose, random or site-directed mutagenesis can be conducted using techniques generally known in the art. To make protein derivatives, for example, the amino acid sequence of a native interacting protein member may be changed in predetermined manners by site-directed DNA mutagenesis to create or remove consensus sequences for, e.g., phosphorylation by protein kinases, glycosylation, ribosylation, myristolation, palmytoylation, ubiquitination, and the like. Alternatively, non-natural amino acids can be incorporated into an interacting protein member during the synthesis of the protein in recombinant host cells. For example, photoreactive lysine derivatives can be incorporated into an interacting protein member during translation by using a modified lysyl-tRNA. See, e.g., Wiedmann et al., Nature, 328:830-833 (1989); Musch et al., Cell, 69:343-352 (1992). Other photoreactive amino acid derivatives can also be incorporated in a similar manner. See, e.g., High et al., J. Biol. Chem., 368:28745-28751 (1993). Indeed, the photoreactive amino acid derivatives thus incorporated into an interacting protein member can function to cross-link the protein to its interacting protein partner in a protein complex under predetermined conditions.

In addition, derivatives of the native interacting protein members of the present invention can also be prepared by chemically linking certain moieties to amino acid side chains of the native proteins.

If desired, the homologues and derivatives thus generated can be tested to determine whether they are capable of interacting with their intended partners to form protein complexes. Testing can be conducted by e.g., the yeast two-hybrid system or other methods known in the art for detecting protein-protein interaction.

A hybrid protein as described above having any interacting pair of the proteins described in the tables, or a homologue, derivative, or fragment thereof covalently linked together by a peptide bond or a peptide linker can be expressed recombinantly from a chimeric nucleic acid, e.g., a DNA or mRNA fragment encoding the fusion protein. Accordingly, the present invention also provides a nucleic acid encoding the hybrid protein of the present invention. In addition, an expression vector having incorporated therein a nucleic acid encoding the hybrid protein of the present invention is also provided. The methods for making such chimeric nucleic acids and expression vectors containing them will be apparent to skilled artisans apprised of the present disclosure.

### 2.4. Protein Microchip

In accordance with another embodiment of the present invention, a protein microchip or microarray is provided having one or more of the protein complexes and/or antibodies selectively immunoreactive with the protein complexes of the present invention. Protein microarrays are becoming increasingly important in both proteomics research and protein-based detection and diagnosis of diseases. The protein microarrays in accordance with this embodiment of the present invention will be useful in a variety of applications including, e.g., large-scale or high-throughput screening for compounds capable of binding to the protein complexes or modulating the interactions between the interacting protein members in the protein complexes.

The protein microarray of the present invention can be prepared in a number of methods known in the art. An example of a suitable method is that disclosed in MacBeath and Schreiber, Science, 289:1760-1763 (2000). Essentially, glass microscope slides are treated with an aldehyde-containing silane reagent (SuperAldehyde Substrates purchased from TeleChem International, Cupertino, Calif.). Nanoliter volumes of protein samples in a phosphate-buffered saline with 40% glycerol are then spotted onto the treated slides using a high-precision contact-printing robot. After incubation, the slides are immersed in a bovine serum albumin (BSA)-containing buffer to quench the unreacted aldehydes and to form a BSA layer that functions to prevent non-specific protein binding in subsequent applications of the microchip. Alternatively, as disclosed in MacBeath and Schreiber, proteins or protein complexes of the present invention can be attached to a BSA-NHS slide by covalent linkages. BSA-NHS slides are fabricated by first attaching a molecular layer of BSA to the surface of glass slides and then activating the BSA with N,N′-disuccinimidyl carbonate. As a result, the amino groups of the lysine, aspartate, and glutamate residues on the BSA are activated and can form covalent urea or amide linkages with protein samples spotted on the slides. See MacBeath and Schreiber, Science, 289:1760-1763 (2000).

Another example of a useful method for preparing the protein microchip of the present invention is that disclosed in PCT Publication Nos. WO 00/4389A2 and WO 00/04382, both of which are assigned to Zyomyx and are incorporated herein by reference. First, a substrate or chip base is covered with one or more layers of thin organic film to eliminate any surface defects, insulate proteins from the base materials, and to ensure uniform protein array. Next, a plurality of protein-capturing agents (e.g., antibodies, peptides, etc.) are arrayed and attached to the base that is covered with the thin film. Proteins or protein complexes can then be bound to the capturing agents forming a protein microarray. The protein microchips are kept in flow chambers with an aqueous solution.

The protein microarray of the present invention can also be made by the method disclosed in PCT Publication No. WO 99/36576 assigned to Packard Bioscience Company, which is incorporated herein by reference. For example, a three-dimensional hydrophilic polymer matrix, i.e., a gel, is first dispensed on a solid substrate such as a glass slide. The polymer matrix gel is capable of expanding or contracting and contains a coupling reagent that reacts with amine groups. Thus, proteins and protein complexes can be contacted with the matrix gel in an expanded aqueous and porous state to allow reactions between the amine groups on the protein or protein complexes with the coupling reagents thus immobilizing the proteins and protein complexes on the substrate. Thereafter, the gel is contracted to embed the attached proteins and protein complexes in the matrix gel.

Alternatively, the proteins and protein complexes of the present invention can be incorporated into a commercially available protein microchip, e.g., the ProteinChip System from Ciphergen Biosystems Inc., Palo Alto, Calif. The ProteinChip System comprises metal chips having a treated surface, which interact with proteins. Basically, a metal chip surface is coated with a silicon dioxide film. The molecules of interest such as proteins and protein complexes can then be attached covalently to the chip surface via a silane coupling agent.

The protein microchips of the present invention can also be prepared with other methods known in the art, e.g., those disclosed in U.S. Pat. Nos. 6,087,102, 6,139,831, 6,087,103; PCT Publication Nos. WO 99/60156, WO 99/39210, WO 00/54046, WO 00/53625, WO 99/51773, WO 99/35289, WO 97/42507, WO 01/01142, WO 00/63694, WO 00/61806, WO 99/61148, WO 99/40434, all of which are incorporated herein by reference.

### 3. Antibodies

In accordance with another aspect of the present invention, an antibody immunoreactive against a protein complex of the present invention is provided. In one embodiment, the antibody is selectively immunoreactive with a protein complex of the present invention. Specifically, the phrase “selectively immunoreactive with a protein complex” as used herein means that the immunoreactivity of the antibody of the present invention with the protein complex is substantially higher than that with the individual interacting members of the protein complex so that the binding of the antibody to the protein complex is readily distinguishable from the binding of the antibody to the individual interacting member proteins based on the strength of the binding affinities. Preferably, the binding constants differ by a magnitude of at least 2 fold, more preferably at least 5 fold, even more preferably at least 10 fold, and most preferably at least 100 fold. In a specific embodiment, the antibody is not substantially immunoreactive with the interacting protein members of the protein complex.

The antibodies of the present invention can be readily prepared using procedures generally known in the art. See, e.g., Harlow and Lane, Antibodies: A Laboratory Manual, Cold Spring Harbor Press, 1988. Typically, the protein complex against which an immunoreactive antibody is desired is used as the antigen for producing an immune response in a host animal. In one embodiment, the protein complex used consists of the native proteins. Preferably, the protein complex includes only protein fragments containing interacting regions provided in the tables. As a result, a greater portion of the total antibodies may be selectively immunoreactive with the protein complexes. The interaction domains can be selected from, e.g., those regions summarized in Table 1. In addition, various techniques known in the art for predicting epitopes may also be employed to design antigenic peptides based on the interacting protein members in a protein complex of the present invention to increase the possibility of producing an antibody selectively immunoreactive with the protein complex. Suitable epitope-prediction computer programs include, e.g., MacVector from International Biotechnologies, Inc. and Protean from DNAStar.

In a specific embodiment, a hybrid protein as described above in Section 2.1 is used as an antigen which has a first protein that is any one of the proteins described in the tables, or a homologue, derivative, or fragment thereof covalently linked by a peptide bond or a peptide linker to a second protein which is the interacting partner of the first protein, or a homologue, derivative, or fragment of the second protein. In a preferred embodiment, the hybrid protein consists of two interacting domains selected from the regions identified in a table above, or homologues or derivatives thereof, covalently linked together by a peptide bond or a linker molecule.

The antibody of the present invention can be a polyclonal antibody to a protein complex of the present invention. To produce the polyclonal antibody, various animal hosts can be employed, including, e.g., mice, rats, rabbits, goats, guinea pigs, hamsters, etc. A suitable antigen which is a protein complex of the present invention or a derivative thereof as described above can be administered directly to a host animal to illicit immune reactions. Alternatively, it can be administered together with a carrier such as keyhole limpet hemocyanin (KLH), bovine serum albumin (BSA), ovalbumin, and Tetanus toxoid. Optionally, the antigen is conjugated to a carrier by a coupling agent such as carbodiimide, glutaraldehyde, and MBS. Any conventional adjuvants may be used to boost the immune response of the host animal to the protein complex antigen. Suitable adjuvants known in the art include but are not limited to Complete Freund's Adjuvant (which contains killed mycobacterial cells and mineral oil), incomplete Freund's Adjuvant (which lacks the cellular components), aluminum salts, MF59 from Chiron (Emeryville, Calif.), monophospholipid, synthetic trehalose dicorynomycolate (TDM) and cell wall skeleton (CWS) both from Corixa Corp. (Seattle, Wash.), non-ionic surfactant vesicles (NISV) from Proteus International PLC (Cheshire, U.K.), and saponins. The antigen preparation can be administered to a host animal by subcutaneous, intramuscular, intravenous, intradermal, or intraperitoneal injection, or by injection into a lymphoid organ.

The antibodies of the present invention may also be monoclonal. Such monoclonal antibodies may be developed using any conventional techniques known in the art. For example, the popular hybridoma method disclosed in Kohler and Milstein, Nature, 256:495-497 (1975) is now a well-developed technique that can be used in the present invention. See U.S. Pat. No. 4,376,110, which is incorporated herein by reference. Essentially, B-lymphocytes producing a polyclonal antibody against a protein complex of the present invention can be fused with myeloma cells to generate a library of hybridoma clones. The hybridoma population is then screened for antigen binding specificity and also for immunoglobulin class (isotype). In this manner, pure hybridoma clones producing specific homogenous antibodies can be selected. See generally, Harlow and Lane, Antibodies: A Laboratory Manual, Cold Spring Harbor Press, 1988. Alternatively, other techniques known in the art may also be used to prepare monoclonal antibodies, which include but are not limited to the EBV hybridoma technique, the human N-cell hybridoma technique, and the trioma technique.

In addition, antibodies selectively immunoreactive with a protein complex of the present invention may also be recombinantly produced. For example, cDNAs prepared by PCR amplification from activated B-lymphocytes or hybridomas may be cloned into an expression vector to form a cDNA library, which is then introduced into a host cell for recombinant expression. The cDNA encoding a specific desired protein may then be isolated from the library. The isolated cDNA can be introduced into a suitable host cell for the expression of the protein. Thus, recombinant techniques can be used to produce specific native antibodies, hybrid antibodies capable of simultaneous reaction with more than one antigen, chimeric antibodies (e.g., the constant and variable regions are derived from different sources), univalent antibodies that comprise one heavy and light chain pair coupled with the Fc region of a third (heavy) chain, Fab proteins, and the like. See U.S. Pat. No. 4,816,567; European Patent Publication No. 0088994; Munro, Nature, 312:597 (1984); Morrison, Science, 229:1202 (1985); Oi et al., BioTechniques, 4:214 (1986); and Wood et al., Nature, 314:446-449 (1985), all of which are incorporated herein by reference. Antibody fragments such as Fv fragments, single-chain Fv fragments (scFv), Fab′ fragments, and F(ab′)₂ fragments can also be recombinantly produced by methods disclosed in, e.g., U.S. Pat. No. 4,946,778; Skerra & Plückthun, Science, 240:1038-1041(1988); Better et al., Science, 240:1041-1043 (1988); and Bird, et al., Science, 242:423-426 (1988), all of which are incorporated herein by reference.

In a preferred embodiment, the antibodies provided in accordance with the present invention are partially or fully humanized antibodies. For this purpose, any methods known in the art may be used. For example, partially humanized chimeric antibodies having V regions derived from the tumor-specific mouse monoclonal antibody, but human C regions are disclosed in Morrison and Oi, Adv. Immunol., 44:65-92 (1989). In addition, fully humanized antibodies can be made using transgenic non-human animals. For example, transgenic non-human animals such as transgenic mice can be produced in which endogenous immunoglobulin genes are suppressed or deleted, while heterologous antibodies are encoded entirely by exogenous immunoglobulin genes, preferably human immunoglobulin genes, recombinantly introduced into the genome. See e.g., U.S. Pat. Nos. 5,530,101; 5,545,806; 6,075,181; PCT Publication No. WO 94/02602; Green et. al., Nat. Genetics, 7: 13-21 (1994); and Lonberg et al., Nature 368: 856-859 (1994), all of which are incorporated herein by reference. The transgenic non-human host animal may be immunized with suitable antigens such as a protein complex of the present invention or one or more of the interacting protein members thereof to illicit specific immune response thus producing humanized antibodies. In addition, cell lines producing specific humanized antibodies can also be derived from the immunized transgenic non-human animals. For example, mature B-lymphocytes obtained from a transgenic animal producing humanized antibodies can be fused to myeloma cells and the resulting hybridoma clones may be selected for specific humanized antibodies with desired binding specificities. Alternatively, cDNAs may be extracted from mature B-lymphocytes and used in establishing a library that is subsequently screened for clones encoding humanized antibodies with desired binding specificities.

In yet another embodiment, a bifunctional antibody is provided that has two different antigen binding sites, each being specific to a different interacting protein member in a protein complex of the present invention. The bifunctional antibody may be produced using a variety of methods known in the art. For example, two different monoclonal antibody-producing hybridomas can be fused together. One of the two hybridomas may produce a monoclonal antibody specific against an interacting protein member of a protein complex of the present invention, while the other hybridoma generates a monoclonal antibody immunoreactive with another interacting protein member of the protein complex. The thus formed new hybridoma produces different antibodies including a desired bifunctional antibody, i.e., an antibody immunoreactive with both of the interacting protein members. The bifunctional antibody can be readily purified. See Milstein and Cuello, Nature, 305:537-540 (1983).

Alternatively, a bifunctional antibody may also be produced using heterobifunctional crosslinkers to chemically link two different monoclonal antibodies, each being immunoreactive with a different interacting protein member of a protein complex. Therefore, the aggregate will bind to two interacting protein members of the protein complex. See Staerz et al, Nature, 314:628-631(1985); Perez et al, Nature, 316:354-356 (1985).

In addition, bifunctional antibodies can also be produced by recombinantly expressing light and heavy chain genes in a hybridoma that itself produces a monoclonal antibody. As a result, a mixture of antibodies including a bifunctional antibody is produced. See DeMonte et al, Proc. Natl. Acad. Sci., USA, 87:2941-2945 (1990); Lenz and Weidle, Gene, 87:213-218 (1990).

Preferably, a bifunctional antibody in accordance with the present invention is produced by the method disclosed in U.S. Pat. No. 5,582,996, which is incorporated herein by reference. For example, two different Fabs can be provided and mixed together. The first Fab can bind to an interacting protein member of a protein complex, and has a heavy chain constant region having a first complementary domain not naturally present in the Fab but capable of binding a second complementary domain. The second Fab is capable of binding another interacting protein member of the protein complex, and has a heavy chain constant region comprising a second complementary domain not naturally present in the Fab but capable of binding to the first complementary domain. Each of the two complementary domains is capable of stably binding to the other but not to itself. For example, the leucine zipper regions of c-fos and c-jun oncogenes may be used as the first and second complementary domains. As a result, the first and second complementary domains interact with each other to form a leucine zipper thus associating the two different Fabs into a single antibody construct capable of binding to two antigenic sites.

Other suitable methods known in the art for producing bifunctional antibodies may also be used, which include those disclosed in Holliger et al., Proc. Nat'l Acad. Sci. USA, 90:6444-6448 (1993); de Kruif et al., J. Biol. Chem., 271:7630-7634 (1996); Coloma and Morrison, Nat. Biotechnol., 15:159-163 (1997); Muller et al., FEBS Lett., 422:259-264 (1998); and Muller et al., FEBS Lett., 432:45-49 (1998), all of which are incorporated herein by reference.

### 4. Methods of Detecting Protein Complexes

Another aspect of the present invention relates to methods for detecting the protein complexes of the present invention, particularly for determining the concentration of a specific protein complex in a patient sample.

In one embodiment, the concentration of a protein complex of the present invention is determined in cells, tissue, or an organ of a patient. For example, the protein complex can be isolated or purified from a patient sample obtained from cells, tissue, or an organ of the patient and the amount thereof is determined. As described above, the protein complex can be prepared from cells, tissue or organ samples by coimmunoprecipitation using an antibody immunoreactive with an interacting protein member, a bifunctional antibody that is immunoreactive with two or more interacting protein members of the protein complex, or preferably an antibody selectively immunoreactive with the protein complex. When bifunctional antibodies or antibodies immunoreactive with only free interacting protein members are used, individual interacting protein members not complexed with other proteins may also be isolated along with the protein complex containing such individual proteins. However, they can be readily separated from the protein complex using methods known in the art, e.g., size-based separation methods such as gel filtration, or by subtracting the protein complex from the mixture using an antibody specific against another individual interacting protein member. Additionally, proteins in a sample can be separated in a gel such as polyacrylamide gel and subsequently immunoblotted using an antibody immunoreactive with the protein complex.

Alternatively, the concentration of the protein complex can be determined in a sample without separation, isolation or purification. For this purpose, it is preferred that an antibody selectively immunoreactive with the specific protein complex is used in an immunoassay. For example, immunocytochemical methods can be used. Other well known antibody-based techniques can also be used including, e.g., enzyme-linked immunosorbent assay (ELISA), radioimmunoassay (RIA), immunoradiometric assays (IRMA), fluorescent immunoassays, protein A immunoassays, and immunoenzymatic assays (IEMA). See e.g., U.S. Pat. Nos. 4,376,110 and 4,486,530, both of which are incorporated herein by reference.

In addition, since a specific protein complex is formed from its interacting protein members, if one of the interacting protein members is at a relatively low concentration in a patient, it may be reasonably expected that the concentration of the protein complex in the patient may also be low. Therefore, the concentration of an individual interacting protein member of a specific protein complex can be determined in a patient sample which can then be used as a reasonably accurate indicator of the concentration of the protein complex in the sample. For this purpose, antibodies against an individual interacting protein member of a specific complex can be used in any one of the methods described above. In a preferred embodiment, the concentration of each of the interacting protein members of a protein complex is determined in a patient sample and the relative concentration of the protein complex is then deduced.

In addition, the relative protein complex concentration in a patient can also be determined by determining the concentration of the mRNA encoding an interacting protein member of the protein complex. Preferably, each interacting protein member's mRNA concentration in a patient sample is determined. For this purpose, methods for determining mRNA concentration generally known in the art may all be used. Examples of such methods include, e.g., Northern blot assay, dot blot assay, PCR assay (preferably quantitative PCR assay), in situ hybridization assay, and the like.

As discussed above, each interaction between members of an interacting protein pair of the present invention suggests that the proteins and/or the protein complexes formed by such proteins may be involved in common biological processes and disease pathways. In addition, the interactions under physiological conditions may lead to the formation of protein complexes in vivo. The protein complexes are expected to mediate the functions and biological activities of the interacting members of the protein complexes. Thus, aberrations in the protein complexes or the individual proteins and the degree of the aberration may be indicators for the diseases or disorders. These aberrations may be used as parameters for classifying and/or staging one of the above-described diseases. In addition, they may also be indicators for patients' response to a drug therapy.

Association between a physiological state (e.g., physiological disorder, predisposition to the disorder, a disease state, response to a drug therapy, or other physiological phenomena or phenotypes) and a specific aberration in a protein complex of the present invention or an individual interacting member thereof can be readily determined by comparative analysis of the protein complex and/or the interacting members thereof in a normal population and an abnormal or affected population. Thus, for example, one can study the concentration, localization and distribution of a particular protein complex, mutations in the interacting protein members of the protein complex, and/or the binding affinity between the interacting protein members in both a normal population and a population affected with a particular physiological disorder described above. The study results can be compared and analyzed by statistical means. Any detected statistically significant difference in the two populations would indicate an association. For example, if the concentration of the protein complex is statistically significantly higher in the affected population than in the normal population, then it can be reasonably concluded that higher concentration of the protein complex is associated with the physiological disorder.

Thus, once an association is established between a particular type of aberration in a particular protein complex of the present invention or in an interacting protein member thereof and a physiological disorder or disease or predisposition to the physiological disorder or disease, then the particular physiological disorder or disease or predisposition to the physiological disorder or disease can be diagnosed or detected by determining whether a patient has the particular aberration.

Accordingly, the present invention also provides a method for diagnosing in a patient a disease or physiological disorder, or a predisposition to the disease or disorder, such as inflammation and inflammatory disorders (e.g., asthma, rheumatoid arthritis, juvenile chronic arthritis, myositis, Chron's disease, gastritis, colitis, ulcerative colitis, inflammatory bowel disease, proctitis, pelvic inflammatory disease, systemic lupus erythematosus, rhinitis, conjunctivitis, scleritis, chronic inflammatory polyneuropathy, Tertiary Lyme disease, psoriasis, dermatitis, eczema, etc.) by determining whether there is any aberration in the patient with respect to a protein complex identified according to the present invention. The same protein complex is analyzed in a normal individual and is compared with the results obtained in the patient. In this manner, any protein complex aberration in the patient can be detected. As used herein, the term “aberration” when used in the context of protein complexes of the present invention means any alterations of a protein complex including increased or decreased concentration of the protein complex in a particular cell or tissue or organ or the total body, altered localization of the protein complex in cellular compartments or in locations of a tissue or organ, changes in binding affinity of an interacting protein member of the protein complex, mutations in an interacting protein member or the gene encoding the protein, and the like. As will be apparent to a skilled artisan, the term “aberration” is used in a relative sense. That is, an aberration is relative to a normal condition.

As used herein, the term “diagnosis” means detecting a disease or disorder or determining the stage or degree of a disease or disorder. The term “diagnosis” also encompasses detecting a predisposition to a disease or disorder, determining the therapeutic effect of a drug therapy, or predicting the pattern of response to a drug therapy or xenobiotics. The diagnosis methods of the present invention may be used independently, or in combination with other diagnosing and/or staging methods known in the medical art for a particular disease or disorder.

Thus, in one embodiment, the method of diagnosis is conducted by detecting, in a patient, the concentrations of one or more protein complexes of the present invention using any one of the methods described above, and determining whether the patient has an aberrant concentration of the protein complexes.

The diagnosis may also be based on the determination of the concentrations of one or more interacting protein members (at the protein, cDNA or mRNA level) of a protein complex of the present invention. An aberrant concentration of an interacting protein member may indicate a physiological disorder or a predisposition to a physiological disorder.

In another embodiment, the method of diagnosis comprises determining, in a patient, the cellular localization, or tissue or organ distribution of a protein complex of the present invention and determining whether the patient has an aberrant localization or distribution of the protein complex. For example, immunocytochemical or immunohistochemical assays can be performed on a cell, tissue or organ sample from a patient using an antibody selectively immunoreactive with a protein complex of the present invention. Antibodies immunoreactive with both an individual interacting protein member and a protein complex containing the protein member may also be used, in which case it is preferred that antibodies immunoreactive with other interacting protein members are also used in the assay. In addition, nucleic acid probes may also be used in in situ hybridization assays to detect the localization or distribution of the mRNAs encoding the interacting protein members of a protein complex. Preferably, the mRNA encoding each interacting protein member of a protein complex is detected concurrently.

In yet another embodiment, the method of diagnosis of the present invention comprises detecting any mutations in one or more interacting protein members of a protein complex of the present invention. In particular, it is desirable to determine whether the interacting protein members have any mutations that will lead to, or are associated with, changes in the functional activity of the proteins or changes in their binding affinity to other interacting protein members in forming a protein complex of the present invention. Examples of such mutations include but are not limited to, e.g., deletions, insertions and rearrangements in the genes encoding the protein members, and nucleotide or amino acid substitutions and the like. In a preferred embodiment, the domains of the interacting protein members that are responsible for the protein-protein interactions, and lead to protein complex formation, are screened to detect any mutations therein. For example, genomic DNA or cDNA encoding an interacting protein member can be prepared from a patient sample, and sequenced. The thus obtained sequence may be compared with known wild-type sequences to identify any mutations. Alternatively, an interacting protein member may be purified from a patient sample and analyzed by protein sequencing or mass spectrometry to detect any amino acid sequence changes. Any methods known in the art for detecting mutations may be used, as will be apparent to skilled artisans apprised of the present disclosure.

In another embodiment, the method of diagnosis includes determining the binding constant of the interacting protein members of one or more protein complexes. For example, the interacting protein members can be obtained from a patient by direct purification or by recombinant expression from genomic DNAs or cDNAs prepared from a patient sample encoding the interacting protein members. Binding constants represent the strength of the protein-protein interaction between the interacting protein members in a protein complex. Thus, by measuring binding constants, subtle aberrations in binding affinity may be detected.

A number of methods known in the art for estimating and determining binding constants in protein-protein interactions are reviewed in Phizicky and Fields, et al., Microbiol. Rev., 59:94-123 (1995), which is incorporated herein by reference. For example, protein affinity chromatography may be used. First, columns are prepared with different concentrations of an interacting protein member, which is covalently bound to the columns. Then a preparation of an interacting protein partner is run through the column and washed with buffer. The interacting protein partner bound to the interacting protein member linked to the column is then eluted. A binding constant is then estimated based on the concentrations of the bound protein and the eluted protein. Alternatively, the method of sedimentation through gradients monitors the rate of sedimentation of a mixture of proteins through gradients of glycerol or sucrose. At concentrations above the binding constant, proteins can sediment as a protein complex. Thus, binding constant can be calculated based on the concentrations. Other suitable methods known in the art for estimating binding constant include but are not limited to gel filtration column such as nonequilibrium “small-zone” gel filtration columns (See e.g., Gill et al., J. Mol. Biol., 220:307-324 (1991)), the Hummel-Dreyer method of equilibrium gel filtration (See e.g., Hummel and Dreyer, Biochim. Biophys. Acta, 63:530-532 (1962)) and large-zone equilibrium gel filtration (See e.g., Gilbert and Kellett, J. Biol. Chem., 246:6079-6086 (1971)), sedimentation equilibrium (See e.g., Rivas and Minton, Trends Biochem., 18:284-287 (1993)), fluorescence methods such as fluorescence spectrum (See e.g., Otto-Bruc et al, Biochemistry, 32:8632-8645 (1993)) and fluorescence polarization or anisotropy with tagged molecules (See e.g., Weiel and Hershey, Biochemistry, 20:5859-5865 (1981)), solution equilibrium measured with immobilized binding protein (See e.g., Nelson and Long, Biochemistry, 30:2384-2390 (1991)), and surface plasmon resonance (See e.g., Panayotou et al., Mol. Cell. Biol., 13:3567-3576 (1993)).

In another embodiment, the diagnosis method of the present invention comprises detecting protein-protein interactions in functional assay systems such as the yeast two-hybrid system. Accordingly, to determine the protein-protein interaction between two interacting protein members that normally form a protein complex in normal individuals, cDNAs encoding the interacting protein members can be isolated from a patient to be diagnosed. The thus cloned cDNAs or fragments thereof can be subcloned into vectors for use in yeast two-hybrid systems. Preferably a reverse yeast two-hybrid system is used such that failure of interaction between the proteins may be positively detected. The use of yeast two-hybrid systems or other systems for detecting protein-protein interactions is known in the art and is described below in Section 5.3.1.

A kit may be used for conducting the diagnosis methods of the present invention. Typically, the kit should contain, in a carrier or compartmentalized container, reagents useful in any of the above-described embodiments of the diagnosis method. The carrier can be a container or support, in the form of, e.g., bag, box, tube, rack, and is optionally compartmentalized. The carrier may define an enclosed confinement for safety purposes during shipment and storage. In one embodiment, the kit includes an antibody selectively immunoreactive with a protein complex of the present invention. In addition, antibodies against individual interacting protein members of the protein complexes may also be included. The antibodies may be labeled with a detectable marker such as radioactive isotopes, or enzymatic or fluorescence markers. Alternatively secondary antibodies such as labeled anti-IgG and the like may be included for detection purposes. Optionally, the kit can include one or more of the protein complexes of the present invention prepared or purified from a normal individual or an individual afflicted with a physiological disorder associated with an aberration in the protein complexes or an interacting protein member thereof. In addition, the kit may further include one or more of the interacting protein members of the protein complexes of the present invention prepared or purified from a normal individual or an individual afflicted with a physiological disorder associated with an aberration in the protein complexes or an interacting protein member thereof. Suitable oligonucleotide primers useful in the amplification of the genes or cDNAs for the interacting protein members may also be provided in the kit. In particular, in a preferred embodiment, the kit includes a first oligonucleotide selectively hybridizable to the mRNA or cDNA encoding one member of an interacting pair of proteins and a second oligonucleotide selectively hybridizable to the mRNA or cDNA encoding the other of the interacting pair. Additional oligonucleotides hybridizing to a region of the genes encoding an interacting pair of proteins may also be included. Such oligonucleotides may be used as PCR primers for, e.g., quantitative PCR amplification of mRNAs encoding the interacting proteins, or as hybridizing probes for detecting the mRNAs. The oligonucleotides may have a length of from about 8 nucleotides to about 100 nucleotides, preferably from about 12 to about 50 nucleotides, and more preferably from about 15 to about 30 nucleotides. In addition, the kit may also contain oligonucleotides that can be used as hybridization probes for detecting the cDNAs or mRNAs encoding the interacting protein members. Preferably, instructions for using the kit or reagents contained therein are also included in the kit.

### 5. Use of Protein Complexes or Interacting Protein Members Thereof in Screening Assays for Modulators

The protein complexes of the present invention and interacting members thereof can also be used in screening assays to identify modulators of the protein complexes, and/or the interacting proteins. In addition, homologues, derivatives or fragments of the interacting proteins provided in this invention may also be used in such screening assays. As used herein, the term “modulator” encompasses any compounds that can cause any form of alteration of the biological activities or functions of the proteins or protein complexes, including, e.g., enhancing or reducing their biological activities, increasing or decreasing their stability, altering their affinity or specificity to certain other biological molecules, etc. In addition, the term “modulator” as used herein also includes any compounds that simply bind any of the proteins described in the tables, and/or the proteins complexes of the present invention. For example, a modulator can be an “interaction antagonist” capable of interfering with or disrupting or dissociating protein-protein interaction between an interacting pair of proteins identified in the tables, or homologues, fragments or derivatives thereof. A modulator can also be an “interaction agonist” that initiates or strengthens the interaction between the protein members of a protein complex of the present invention, or homologues, fragments or derivatives thereof.

In addition, the discovery of protein ligands of the present invention allows the use of screening assays to identify modulators of individual proteins of the protein complexes. Typical high-throughput screening assays involve measuring the modulation of the enzymatic activity of a protein. However, typical high-throughput screening assays are not applicable to proteins that exhibit little or no measurable enzymatic activity. The present discovery of novel ligands of proteins allows a screen to be setup that does not utilize enzymatic activity measurements. Consequently, the present invention enables a non-enzymatic high-throughput assay to be performed for modulators of individual proteins and/or protein complexes described in the tables.

Accordingly, the present invention provides screening methods for selecting modulators of any of the proteins described in the tables, or a mutant form thereof, or a protein-protein interaction between an interacting pair of proteins provided in the present invention, or homologues, fragments or derivatives thereof.

The selected compounds can be tested for their ability to modulate (interfere with or strengthen) the interaction between the interacting partners within the protein complexes of the present invention. In addition, the compounds can also be further tested for their ability to modulate (inhibit or enhance) cellular functions such as intracellular signaling in cells as well as their effectiveness in treating diseases such as inflammation and inflammatory disorders (e.g., asthma, rheumatoid arthritis, juvenile chronic arthritis, myositis, Chron's disease, gastritis, colitis, ulcerative colitis, inflammatory bowel disease, proctitis, pelvic inflammatory disease, systemic lupus erythematosus, rhinitis, conjunctivitis, scleritis, chronic inflammatory polyneuropathy, Tertiary Lyme disease, psoriasis, dermatitis, eczema, etc.).

The modulators selected in accordance with the screening methods of the present invention can be effective in modulating the functions or activities of individual interacting proteins, or the protein complexes of the present invention. For example, compounds capable of binding to the protein complexes may be capable of modulating the functions of the protein complexes. Additionally, compounds that interfere with, weaken, dissociate or disrupt, or alternatively, initiate, facilitate or stabilize the protein-protein interaction between the interacting protein members of the protein complexes can also be effective in modulating the functions or activities of the protein complexes. Thus, the compounds identified in the screening methods of the present invention can be made into therapeutically or prophylactically effective drugs for preventing or ameliorating diseases, disorders or symptoms caused by or associated with a protein complex or an interacting member thereof. Alternatively, they may be used as leads to aid the design and identification of therapeutically or prophylactically effective compounds for diseases, disorders or symptoms caused by or associated with the protein complex or interacting protein members thereof. The protein complexes and/or interacting protein members thereof in accordance with the present invention can be used in any of a variety of drug screening techniques. Drug screening can be performed as described herein or using well-known techniques, such as those described in U.S. Pat. Nos. 5,800,998 and 5,891,628, both of which are incorporated herein by reference.

### 5.1. Test Compounds

Any test compounds may be screened in the screening assays of the present invention to select modulators of the protein complexes or interacting members thereof. By the term “selecting” or “select” compounds it is intended to encompass both (a) choosing compounds from a group previously unknown to be modulators of a protein complex or interacting protein members thereof; and (b) testing compounds that are known to be capable of binding, or modulating the functions and activities of, a protein complex or interacting protein members thereof. Both types of compounds are generally referred to herein as “test compounds.” The test compounds may include, by way of example, proteins (e.g., antibodies, small peptides, artificial or natural proteins), nucleic acids, and derivatives, mimetics and analogs thereof, and small organic molecules having a molecular weight of no greater than 10,000 daltons, more preferably less than 5,000 daltons. Preferably, the test compounds are provided in library formats known in the art, e.g., in chemically synthesized libraries, recombinantly expressed libraries (e.g., phage display libraries), and in vitro translation-based libraries (e.g., ribosome display libraries).

For example, the screening assays of the present invention can be used in the antibody production processes described in Section 3 to select antibodies with desirable specificities. Various forms of antibodies or derivatives thereof may be screened, including but not limited to, polyclonal antibodies, monoclonal antibodies, bifunctional antibodies, chimeric antibodies, single chain antibodies, antibody fragments such as Fv fragments, single-chain Fv fragments (scFv), Fab′ fragments, and F(ab′)₂ fragments, and various modified forms of antibodies such as catalytic antibodies, and antibodies conjugated to toxins or drugs, and the like. The antibodies can be of any types such as IgG, IgE, IgA, or IgM. Humanized antibodies are particularly preferred. Preferably, the various antibodies and antibody fragments may be provided in libraries to allow large-scale high throughput screening. For example, expression libraries expressing antibodies or antibody fragments may be constructed by a method disclosed, e.g., in Huse et al., Science, 246:1275-1281 (1989), which is incorporated herein by reference. Single-chain Fv (scFv) antibodies are of particular interest in diagnostic and therapeutic applications. Methods for providing antibody libraries are also provided in U.S. Pat. Nos. 6,096,551; 5,844,093; 5,837,460; 5,789,208; and 5,667,988, all of which are incorporated herein by reference.

Peptidic test compounds may be peptides having L-amino acids and/or D-amino acids, phosphopeptides, and other types of peptides. The screened peptides can be of any size, but preferably have less than about 50 amino acids. Smaller peptides are easier to deliver into a patient's body. Various forms of modified peptides may also be screened. Like antibodies, peptides can also be provided in, e.g., combinatorial libraries. See generally, Gallop et al., J. Med. Chem., 37:1233-1251 (1994). Methods for making random peptide libraries are disclosed in, e.g., Devlin et al., Science, 249:404-406 (1990). Other suitable methods for constructing peptide libraries and screening peptides therefrom are disclosed in, e.g., Scott and Smith, Science, 249:386-390 (1990); Moran et al., J. Am. Chem. Soc., 117:10787-10788 (1995) (a library of electronically tagged synthetic peptides); Stachelhaus et al., Science, 269:69-72 (1995); U.S. Pat. Nos. 6,156,511; 6,107,059; 6,015,561; 5,750,344; 5,834,318; 5,750,344, all of which are incorporated herein by reference. For example, random-sequence peptide phage display libraries may be generated by cloning synthetic oligonucleotides into the gene III or gene VIII of an E. coli filamentous phage. The thus generated phage can propagate in E. coli. and express peptides encoded by the oligonucleotides as fusion proteins on the surface of the phage. Scott and Smith, Science, 249:368-390 (1990). Alternatively, the “peptides on plasmids” method may also be used to form peptide libraries. In this method, random peptides may be fused to the C-terminus of the E. coli. Lac repressor by recombinant technologies and expressed from a plasmid that also contains Lac repressor-binding sites. As a result, the peptide fusions bind to the same plasmid that encodes them.

Small organic or inorganic non-peptide non-nucleotide compounds are preferred test compounds for the screening assays of the present invention. They too can be provided in a library format. See generally, Gordan et al. J. Med. Chem., 37:1385-1401 (1994). For example, benzodiazepine libraries are provided in Bunin and Ellman, J. Am. Chem. Soc., 114:10997-10998 (1992), which is incorporated herein by reference. Methods for constructing and screening peptoid libraries are disclosed in Simon et al., Proc. Natl. Acad. Sci. USA, 89:9367-9371 (1992). Methods for the biosynthesis of novel polyketides in a library format are described in McDaniel et al, Science, 262:1546-1550 (1993) and Kao et al., Science, 265:509-512 (1994). Various libraries of small organic molecules and methods of construction thereof are disclosed in U.S. Pat. No. 6,162,926 (multiply-substituted fullerene derivatives); U.S. Pat. No. 6,093,798 (hydroxamic acid derivatives); U.S. Pat. No. 5,962,337 (combinatorial 1,4-benzodiazepin-2,5-dione library); U.S. Pat. No. 5,877,278 (Synthesis of N-substituted oligomers); U.S. Pat. No. 5,866,341 (compositions and methods for screening drug libraries); U.S. Pat. No. 5,792,821 (polymerizable cyclodextrin derivatives); U.S. Pat. No. 5,766,963 (hydroxypropylamine library); and U.S. Pat. No. 5,698,685 (morpholino-subunit combinatorial library), all of which are incorporated herein by reference.

Other compounds such as oligonucleotides and peptide nucleic acids (PNA), and analogs and derivatives thereof may also be screened to identify clinically useful compounds. Combinatorial libraries of oligonucleotides are also known in the art. See Gold et al., J. Biol. Chem., 270:13581-13584 (1995).

### 5.2. In Vitro Screening Assays

The test compounds may be screened in an in vitro assay to identify compounds capable of binding the protein complexes or interacting protein members thereof in accordance with the present invention. For this purpose, a test compound is contacted with a protein complex or an interacting protein member thereof under conditions and for a time sufficient to allow specific interaction between the test compound and the target components to occur, thereby resulting in the binding of the compound to the target, and the formation of a complex. Subsequently, the binding event is detected.

Various screening techniques known in the art may be used in the present invention. The protein complexes and the interacting protein members thereof may be prepared by any suitable methods, e.g., by recombinant expression and purification. The protein complexes and/or interacting protein members thereof (both are referred to as “target” hereinafter in this section) may be free in solution. A test compound may be mixed with a target forming a liquid mixture. The compound may be labeled with a detectable marker. Upon mixing under suitable conditions, the binding complex having the compound and the target may be co-immunoprecipitated and washed. The compound in the precipitated complex may be detected based on the marker on the compound.

In a preferred embodiment, the target is immobilized on a solid support or on a cell surface. Preferably, the target can be arrayed into a protein microchip in a method described in Section 2.3. For example, a target may be immobilized directly onto a microchip substrate such as glass slides or onto multi-well plates using non-neutralizing antibodies, i.e., antibodies capable of binding to the target but do not substantially affect its biological activities. To affect the screening, test compounds can be contacted with the immobilized target to allow binding to occur to form complexes under standard binding assay conditions. Either the targets or test compounds are labeled with a detectable marker using well-known labeling techniques. For example, U.S. Pat. No. 5,741,713 discloses combinatorial libraries of biochemical compounds labeled with NMR active isotopes. To identify binding compounds, one may measure the formation of the target-test compound complexes or kinetics for the formation thereof. When combinatorial libraries of organic non-peptide non-nucleic acid compounds are screened, it is preferred that labeled or encoded (or “tagged”) combinatorial libraries are used to allow rapid decoding of lead structures. This is especially important because, unlike biological libraries, individual compounds found in chemical libraries cannot be amplified by self-amplification. Tagged combinatorial libraries are provided in, e.g., Borchardt and Still, J. Am. Chem. Soc., 116:373-374 (1994) and Moran et al., J. Am. Chem. Soc., 117:10787-10788 (1995), both of which are incorporated herein by reference.

Alternatively, the test compounds can be immobilized on a solid support, e.g., forming a microarray of test compounds. The target protein or protein complex is then contacted with the test compounds. The target may be labeled with any suitable detection marker. For example, the target may be labeled with radioactive isotopes or fluorescence marker before binding reaction occurs. Alternatively, after the binding reactions, antibodies that are immunoreactive with the target and are labeled with radioactive materials, fluorescence markers, enzymes, or labeled secondary anti-Ig antibodies may be used to detect any bound target thus identifying the binding compound. One example of this embodiment is the protein probing method. That is, the target provided in accordance with the present invention is used as a probe to screen expression libraries of proteins or random peptides. The expression libraries can be phage display libraries, in vitro translation-based libraries, or ordinary expression cDNA libraries. The libraries may be immobilized on a solid support such as nitrocellulose filters. See e.g., Sikela and Hahn, Proc. Natl. Acad. Sci. USA, 84:3038-3042 (1987). The probe may be labeled with a radioactive isotope or a fluorescence marker. Alternatively, the probe can be biotinylated and detected with a streptavidin-alkaline phosphatase conjugate. More conveniently, the bound probe may be detected with an antibody.

In one embodiment, the proteins identified in the tables are used as targets in an assay to select modulators of the proteins in the tables. In a specific embodiment, a screening assay for modulators of PRAK is performed by using ERK3 as a ligand for PRAK. For example, in this screen, PRAK can be immobilized on a solid support and is contacted with test compounds. PROTIEN2 can be labeled with a detectable marker such as radioactive materials or fluorescence markers using label techniques known in the art. The labeled ERK3 is allowed to contact the immobilized PRAK and levels of PRAK-ERK3 protein complex formed are detected by washing away unbound ERK3. The ability of the test compounds to modulate PRAK is determined by comparing the level of PRAK-ERK3 complex formed when PRAK is contacted with test compounds to the level formed in the absence of test compounds. Alternatively, as will be apparent to skilled artisans, the ERK3 protein can be detected with labeled antibody against ERK3, or by an antibody specific to a polypeptide that is fused to ERK3.

In yet another embodiment, the protein complexes identified in the tables are used as a target in the assay. In a specific embodiment, a protein complex used in the screening assay includes a hybrid protein as described in Section 2.1, which is formed by fusion of two interacting protein members or fragments or interaction domains thereof. The hybrid protein may also be designed such that it contains a detectable epitope tag fused thereto. Suitable examples of such epitope tags include sequences derived from, e.g., influenza virus hemagglutinin (HA), Simian Virus 5 (V5), polyhistidine (6×His), c-myc, lacZ, GST, and the like.

In addition, a known ligand capable of binding to the target can be used in competitive binding assays. Complexes between the known ligand and the target can be formed and then contacted with test compounds. The ability of a test compound to interfere with the interaction between the target and the known ligand is measured. One exemplary ligand is an antibody capable of specifically binding the target. Particularly, such an antibody is especially useful for identifying peptides that share one or more antigenic determinants of the target protein complex or interacting protein members thereof.

In a specific preferred embodiment, the target is one member of an interacting pair of proteins disclosed according the present invention, or a homologue, derivative or fragment thereof, and the competitive ligand is the other member of the interacting pair of proteins, or a homologue, derivative or fragment thereof. Preferably, either the target or the ligand or both are labeled with or detectable marker. Alternatively, either the target or the ligand or both are fusion proteins that contain a detectable epitope tag having one or more sequences derived from, e.g., influenza virus hemagglutinin (HA), Simian Virus 5 (V5), polyhistidine (6×His), c-myc, lacZ, GST, and the like.

Thus, for example, the target can be immobilized to a solid support. The ligand can be a fusion protein having a fragment of an interactor of the target protein fused to an epitope tag, e.g., c-myc. The ligand can be contacted with the target in the presence or absence of one or more test compounds. Both ligand molecules associated with the immobilized target and ligand molecules not associated with the target can be detected with, e.g., an antibody against the c-myc tag. As a result, test compounds capable of binding the target or ligand, or disrupting the protein-protein interaction between the target and ligand can be identified or selected.

Test compounds may also be screened in an in vitro assay to identify compounds capable of dissociating the protein complexes identified in the tables above. Thus, for example, any one of the interacting pairs of proteins described in the tables above can be contacted with a test compound and the integrity of the protein complex can be assessed. Conversely, test compounds may also be screened to identify compounds capable of enhancing the interactions between the constituent members of the protein complexes formed by the interactions described in the tables. The assays can be conducted in a manner similar to the binding assays described above. For example, the presence or absence of a particular pair of interacting proteins can be detected by an antibody selectively immunoreactive with the protein complex formed by those two proteins. Thus, after incubation of the protein complex with a test compound, an immunoprecipitation assay can be conducted with the antibody. If the test compound disrupts the protein complex, then the amount of immunoprecipitated protein complex in this assay will be significantly less than that in a control assay in which the same protein complex is not contacted with the test compound. Similarly, two proteins—the interaction between which is to be enhanced—may be incubated together with a test compound. Thereafter, a protein complex formed by the two interacting proteins may be detected by the selectively immunoreactive antibody. The amount of protein complex may be compared to that formed in the absence of the test compound. Various other detection methods may be suitable in the dissociation assay, as will be apparent to a skilled artisan apprised of the present disclosure.

In another embodiment, fluorescent resonance energy transfer (FRET) is used to screen for modulators of interacting proteins of the protein complexes of the present invention. FRET assays measure the energy transfer of a fluorescent label to another fluorescent label. Fluorescent labels absorb light preferentially at one wavelength and emit light preferentially at a second wavelength. FRET assays utilize this characteristic by selecting a fluorescent label, called a donor fluorophore, that emits light preferentially at the wavelength a second label, called the acceptor fluorophore, preferentially absorbs light. The proximity of the donor and acceptor fluorophore can be determined by measuring the energy transfer from the donor fluorophore to the acceptor fluorophore. Measuring the energy transfer is performed by shining light on a solution containing acceptor and donor fluorophores at the wavelength the donor fluorophore absorbs light and measuring fluorescence at the wavelength the acceptor fluorophore emits light. The amount of fluorescence of the acceptor fluorophore indicates the proximity of the donor and acceptor fluorophores.

For example, FRET assays can be used to screen for modulators of PRAK by labeling PRAK or an antibody to PRAK with an acceptor fluorophore and labeling a PRAK substrate or interactor (e.g., ERK3) or an antibody to a PRAK substrate/interactor with an acceptor fluorophore. If the test compound is a PRAK modulator it will decrease the fluorescence of the acceptor fluorophore because the acceptor and donor fluorphore will not be as close to each other.

In a specific embodiment of a FRET assay, TP³⁺ is attached to an antibody to PRAK, and BODIPY-TMR is attached to an antibody to an interactor (e.g., ERK3). The fluorescently labeled antibodies, PRAK, and PRAK substrates are put in solution together. Light at the wavelength that TP³⁺ preferentially absorbs light is shined on the solution and the fluorescence of the solution is measured at the wavelength that BODIPY-TMR preferentially emits light. A test compound is then added to the solution and and light at the wavelength that TP³⁺ preferentially absorbs light is shined on the solution and the fluorescence of the solution is measured at the wavelength that BODIPY-TMR preferentially emits light. If the fluorescence of the solution with the test compound decreases compared to the fluorescence of the solution without the test compound then the test compound is a PRAK modulator.

### 5.3. In Vivo Screening Assays

Test compounds can also be screened in any in vivo assays to select modulators of the protein complexes or interacting protein members thereof in accordance with the present invention. For example, any in vivo assays known in the art to be useful in identifying compounds capable of strengthening or interfering with the stability of the protein complexes of the present invention may be used.

In a specific example, a screening assay for modulators of a PRAK is performed by using ERK3 as a ligand for PRAK. In this screen, PRAK is contacted with test compounds in the presence of ERK3 and the levels of PRAK-ERK3 protein complex formed when PRAK is contacted with the test compound in the presence of ERK3 is detected. The ability of the test compounds to modulate PRAK is determined by comparing the level of PRAK-ERK3 complex formed when PRAK is contacted with test compounds to the level formed in the absence of test compounds. If the level of PRAK-ERK3 protein complex formed when PRAK is contacted with the test compound then the test compound is a modulator of PRAK.

To screen peptidic compounds for modulators of PRAK, the two-hybrid systems described in Section 4 may be used in the screening assays in which the PRAK protein is expressed in, e.g., a bait fusion protein and the peptidic test compounds are expressed in, e.g., prey fusion proteins. Screening peptidic compounds for modulators of the proteins identified in the tables can also be performed using the two-hybrid systems described in Section 4 by expressing the proteins identified in the tables in, e.g., a bait fusion protein and expressing the peptidic test compounds in e.g., prey fusion proteins.

To screen for modulators of the protein-protein interaction between PRAK and a PRAK-interacting protein, the methods of the present invention typically comprise contacting the PRAK protein with the PRAK-interacting protein in the presence of a test compound, and determining the interaction between the PRAK protein and the PRAK-interacting protein. In a preferred embodiment, a two-hybrid system, e.g., a yeast two-hybrid system as described in detail in Section 4 is employed.

### 5.3.1. Two-Hybrid Assays

In a preferred embodiment, one of the yeast two-hybrid systems or their analogous or derivative forms is used. Examples of suitable two-hybrid systems known in the art include, but are not limited to, those disclosed in U.S. Pat. Nos. 5,283,173; 5,525,490; 5,585,245; 5,637,463; 5,695,941; 5,733,726; 5,776,689; 5,885,779; 5,905,025; 6,037,136; 6,057,101; 6,114,111; and Bartel and Fields, eds., The Yeast Two-Hybrid System, Oxford University Press, New York, N.Y., 1997, all of which are incorporated herein by reference.

Typically, in a classic transcription-based two-hybrid assay, two chimeric genes are prepared encoding two fusion proteins: one contains a transcription activation domain fused to an interacting protein member of a protein complex of the present invention or an interaction domain or fragment of the interacting protein member, while the other fusion protein includes a DNA binding domain fused to another interacting protein member of the protein complex or a fragment or interaction domain thereof. For the purpose of convenience, the two interacting protein members, fragments or interaction domains thereof are referred to as “bait fusion protein” and “prey fusion protein,” respectively. The chimeric genes encoding the fusion proteins are termed “bait chimeric gene” and “prey chimeric gene,” respectively. Typically, a “bait vector” and a “prey vector” are provided for the expression of a bait chimeric gene and a prey chimeric gene, respectively.

### 5.3.1.1. Vectors

Many types of vectors can be used in a transcription-based two-hybrid assay. Methods for the construction of bait vectors and prey vectors should be apparent to skilled artisans in the art apprised of the present disclosure. See generally, Current Protocols in Molecular Biology, Vol. 2, Ed. Ausubel, et al., Greene Publish. Assoc. & Wiley Interscience, Ch. 13, 1988; Glover, DNA Cloning, Vol. II, IRL Press, Wash., D.C., Ch. 3, 1986; Bitter, et al., in Methods in Enzymology 153:516-544 (1987); The Molecular Biology of the Yeast Saccharomyces, Eds. Strathem et al., Cold Spring Harbor Press, Vols. I and II, 1982; and Rothstein in DNA Cloning: A Practical Approach, Vol. 11, Ed. DM Glover, IRL Press, Wash., D.C., 1986.

Generally, the bait and prey vectors include an expression cassette having a promoter operably linked to a chimeric gene for the transcription of the chimeric gene. The vectors may also include an origin of DNA replication for the replication of the vectors in host cells and a replication origin for the amplification of the vectors in, e.g., E. coli, and selection marker(s) for selecting and maintaining only those host cells harboring the vectors. Additionally, the expression cassette preferably also contains inducible elements, which function to control the expression of a chimeric gene. Making the expression of the chimeric genes inducible and controllable is especially important in the event that the fusion proteins or components thereof are toxic to the host cells. Other regulatory sequences such as transcriptional enhancer sequences and translation regulation sequences (e.g., Shine-Dalgarno sequence) can also be included in the expression cassette. Termination sequences such as the bovine growth hormone, SV40, lacZ and AcMNPV polyhedral polyadenylation signals may also be operably linked to a chimeric gene in the expression cassette. An epitope tag coding sequence for detection and/or purification of the fusion proteins can also be operably linked to the chimeric gene in the expression cassette. Examples of useful epitope tags include, but are not limited to, influenza virus hemagglutinin (HA), Simian Virus 5 (V5), polyhistidine (6×His), c-myc, lacZ, GST, and the like. Proteins with polyhistidine tags can be easily detected and/or purified with Ni affinity columns, while specific antibodies to many epitope tags are generally commercially available. The vectors can be introduced into the host cells by any techniques known in the art, e.g., by direct DNA transformation, microinjection, electroporation, viral infection, lipofection, gene gun, and the like. The bait and prey vectors can be maintained in host cells in an extrachromosomal state, i.e., as self-replicating plasmids or viruses. Alternatively, one or both vectors can be integrated into chromosomes of the host cells by conventional techniques such as selection of stable cell lines or site-specific recombination.

The in vivo assays of the present invention can be conducted in many different host cells, including but not limited to bacteria, yeast cells, plant cells, insect cells, and mammalian cells. A skilled artisan will recognize that the designs of the vectors can vary with the host cells used. In one embodiment, the assay is conducted in prokaryotic cells such as Escherichia coli, Salmonella, Klebsiella, Pseudomonas, Caulobacter, and Rhizobium. Suitable origins of replication for the expression vectors useful in this embodiment of the present invention include, e.g., the ColE1, pSC101, and M13 origins of replication. Examples of suitable promoters include, for example, the T7 promoter, the lacZ promoter, and the like. In addition, inducible promoters are also useful in modulating the expression of the chimeric genes. For example, the lac operon from bacteriophage lambda plac5 is well known in the art and is inducible by the addition of IPTG to the growth medium. Other known inducible promoters useful in a bacteria expression system include pL of bacteriophage λ, the trp promoter, and hybrid promoters such as the tac promoter, and the like.

In addition, selection marker sequences for selecting and maintaining only those prokaryotic cells expressing the desirable fusion proteins should also be incorporated into the expression vectors. Numerous selection markers including auxotrophic markers and antibiotic resistance markers are known in the art and can all be useful for purposes of this invention. For example, the bla gene, which confers ampicillin resistance, is the most commonly used selection marker in prokaryotic expression vectors. Other suitable markers include genes that confer neomycin, kanamycin, or hygromycin resistance to the host cells. In fact, many vectors are commercially available from vendors such as Invitrogen Corp. of Carlsbad, Calif., Clontech Corp. of Palo Alto, Calif., and Stratagene Corp. of La Jolla, Calif., and Promega Corp. of Madison, Wis. These commercially available vectors, e.g., pBR322, pSPORT, pBluescriptIISK, pcDNAI, and pcDNAII all have a multiple cloning site into which the chimeric genes of the present invention can be conveniently inserted using conventional recombinant techniques. The constructed expression vectors can be introduced into host cells by various transformation or transfection techniques generally known in the art.

In another embodiment, mammalian cells are used as host cells for the expression of the fusion proteins and detection of protein-protein interactions. For this purpose, virtually any mammalian cells can be used including normal tissue cells, stable cell lines, and transformed tumor cells. Conveniently, mammalian cell lines such as CHO cells, Jurkat T cells, NIH 3T3 cells, HEK-293 cells, CV-1 cells, COS-1 cells, HeLa cells, VERO cells, MDCK cells, WI38 cells, and the like are used. Mammalian expression vectors are well known in the art and many are commercially available. Examples of suitable promoters for the transcription of the chimeric genes in mammalian cells include viral transcription promoters derived from adenovirus, simian virus 40 (SV40) (e.g., the early and late promoters of SV40), Rous sarcoma virus (RSV), and cytomegalovirus (CMV) (e.g., CMV immediate-early promoter), human immunodeficiency virus (HIV) (e.g., long terminal repeat (LTR)), vaccinia virus (e.g., 7.5K promoter), and herpes simplex virus (HSV) (e.g., thymidine kinase promoter). Inducible promoters can also be used. Suitable inducible promoters include, for example, the tetracycline responsive element (TRE) (See Gossen et al., Proc. Natl. Acad. Sci. USA, 89:5547-5551 (1992)), metallothionein IIA promoter, ecdysone-responsive promoter, and heat shock promoters. Suitable origins of replication for the replication and maintenance of the expression vectors in mammalian cells include, e.g., the Epstein Barr origin of replication in the presence of the Epstein Barr nuclear antigen (see Sugden et al., Mole. Cell. Biol., 5:410-413 (1985)) and the SV40 origin of replication in the presence of the SV40 T antigen (which is present in COS-1 and COS-7 cells) (see Margolskee et al., Mole. Cell. Biol., 8:2837 (1988)). Suitable selection markers include, but are not limited to, genes conferring resistance to neomycin, hygromycin, zeocin, and the like. Many commercially available mammalian expression vectors may be useful for the present invention, including, e.g., pCEP4, pcDNAI, pIND, pSecTag2, pVAX1, pcDNA3.1, and pBI-EGFP, and pDisplay. The vectors can be introduced into mammalian cells using any known techniques such as calcium phosphate precipitation, lipofection, electroporation, and the like. The bait vector and prey vector can be co-transformed into the same cell or, alternatively, introduced into two different cells which are subsequently fused together by cell fusion or other suitable techniques.

Viral expression vectors, which permit introduction of recombinant genes into cells by viral infection, can also be used for the expression of the fusion proteins. Viral expression vectors generally known in the art include viral vectors based on adenovirus, bovine papilloma virus, murine stem cell virus (MSCV), MFG virus, and retrovirus. See Sarver, et al., Mol. Cell. Biol., 1: 486 (1981); Logan & Shenk, Proc. Natl. Acad. Sci. USA, 81:3655-3659 (1984); Mackett, et al., Proc. Natl. Acad. Sci. USA, 79:7415-7419 (1982); Mackett, et al., J. Virol., 49:857-864 (1984); Panicali, et al., Proc. Natl. Acad. Sci. USA, 79:4927-4931 (1982); Cone & Mulligan, Proc. Natl. Acad. Sci. USA, 81:6349-6353 (1984); Mann et al., Cell, 33:153-159 (1993); Pear et al., Proc. Natl. Acad. Sci. USA, 90:8392-8396 (1993); Kitamura et al., Proc. Natl. Acad. Sci. USA, 92:9146-9150 (1995); Kinsella et al., Human Gene Therapy, 7:1405-1413 (1996); Hofinann et al., Proc. Natl. Acad. Sci. USA, 93:5185-5190 (1996); Choate et al., Human Gene Therapy, 7:2247 (1996); WO 94/19478; Hawley et al., Gene Therapy, 1:136 (1994) and Rivere et al., Genetics, 92:6733 (1995), all of which are incorporated by reference.

Generally, to construct a viral vector, a chimeric gene according to the present invention can be operably linked to a suitable promoter. The promoter-chimeric gene construct is then inserted into a non-essential region of the viral vector, typically a modified viral genome. This results in a viable recombinant virus capable of expressing the fusion protein encoded by the chimeric gene in infected host cells. Once in the host cell, the recombinant virus typically is integrated into the genome of the host cell. However, recombinant bovine papilloma viruses typically replicate and remain as extrachromosomal elements.

In another embodiment, the detection assays of the present invention are conducted in plant cell systems. Methods for expressing exogenous proteins in plant cells are well known in the art. See generally, Weissbach & Weissbach, Methods for Plant Molecular Biology, Academic Press, NY, 1988; Grierson & Corey, Plant Molecular Biology, 2d Ed., Blackie, London, 1988. Recombinant virus expression vectors based on, e.g., cauliflower mosaic virus (CaMV) or tobacco mosaic virus (TMV) can all be used. Alternatively, recombinant plasmid expression vectors such as Ti plasmid vectors and Ri plasmid vectors are also useful. The chimeric genes encoding the fusion proteins of the present invention can be conveniently cloned into the expression vectors and placed under control of a viral promoter such as the 35S RNA and 19S RNA promoters of CaMV or the coat protein promoter of TMV, or of a plant promoter, e.g., the promoter of the small subunit of RUBISCO and heat shock promoters (e.g., soybean hsp17.5-E or hsp17.3-B promoters).

In addition, the in vivo assay of the present invention can also be conducted in insect cells, e.g., Spodoptera frugiperda cells, using a baculovirus expression system. Expression vectors and host cells useful in this system are well known in the art and are generally available from various commercial vendors. For example, the chimeric genes of the present invention can be conveniently cloned into a non-essential region (e.g., the polyhedrin gene) of an Autographa californica nuclear polyhedrosis virus (AcNPV) vector and placed under control of an AcNPV promoter (e.g., the polyhedrin promoter). The non-occluded recombinant viruses thus generated can be used to infect host cells such as Spodoptera frugiperda cells in which the chimeric genes are expressed. See U.S. Pat. No. 4,215,051.

In a preferred embodiment of the present invention, the fusion proteins are expressed in a yeast expression system using yeasts such as Saccharomyces cerevisiae, Hansenula polymorpha, Pichia pastoris, and Schizosaccharomyces pombe as host cells. The expression of recombinant proteins in yeasts is a well-developed field, and the techniques useful in this respect are disclosed in detail in The Molecular Biology of the Yeast Saccharomyces, Eds. Strathem et al., Vols. I and II, Cold Spring Harbor Press, 1982; Ausubel et al., Current Protocols in Molecular Biology, New York, Wiley, 1994; and Guthrie and Fink, Guide to Yeast Genetics and Molecular Biology, in Methods in Enzymology, Vol. 194, 1991, all of which are incorporated herein by reference. Sudbery, Curr. Opin. Biotech., 7:517-524 (1996) reviews the successes in the art of expressing recombinant proteins in various yeast species; the entire content and references cited therein are incorporated herein by reference. In addition, Bartel and Fields, eds., The Yeast Two-Hybrid System, Oxford University Press, New York, N.Y., 1997 contains extensive discussions of recombinant expression of fusion proteins in yeasts in the context of various yeast two-hybrid systems, and cites numerous relevant references. These and other methods known in the art can all be used for purposes of the present invention. The application of such methods to the present invention should be apparent to a skilled artisan apprised of the present disclosure.

Generally, each of the two chimeric genes is included in a separate expression vector (bait vector and prey vector). Both vectors can be co-transformed into a single yeast host cell. As will be apparent to a skilled artisan, it is also possible to express both chimeric genes from a single vector. In a preferred embodiment, the bait vector and prey vector are introduced into two haploid yeast cells of opposite mating types, e.g., a-type and α-type, respectively. The two haploid cells can be mated at a desired time to form a diploid cell expressing both chimeric genes.

Generally, the bait and prey vectors for recombinant expression in yeast include a yeast replication origin such as the 2μ origin or the ARSH4 sequence for the replication and maintenance of the vectors in yeast cells. Preferably, the vectors also have a bacteria origin of replication (e.g., ColE1) and a bacteria selection marker (e.g., amp^{R} marker, i.e., bla gene). Optionally, the CEN6 centromeric sequence is included to control the replication of the vectors in yeast cells. Any constitutive or inducible promoters capable of driving gene transcription in yeast cells may be employed to control the expression of the chimeric genes. Such promoters are operably linked to the chimeric genes. Examples of suitable constitutive promoters include but are not limited to the yeast ADH1, PGK1 TEF2, GPD1, HIS3, and CYC1 promoters. Examples of suitable inducible promoters include but are not limited to the yeast GAL1 (inducible by galactose), CUP 1 (inducible by Cu⁺⁺), and FUS1 (inducible by pheromone) promoters; the AOX/MOX promoter from H. polymorpha and P. pastoris (repressed by glucose or ethanol and induced by methanol); chimeric promoters such as those that contain LexA operators (inducible by LexA-containing transcription factors); and the like. Inducible promoters are preferred when the fusion proteins encoded by the chimeric genes are toxic to the host cells. If it is desirable, certain transcription repressing sequences such as the upstream repressing sequence (URS) from SPO13 promoter can be operably linked to the promoter sequence, e.g., to the 5′ end of the promoter region. Such upstream repressing sequences function to fine-tune the expression level of the chimeric genes.

Preferably, a transcriptional termination signal is operably linked to the chimeric genes in the vectors. Generally, transcriptional termination signal sequences derived from, e.g., the CYC1 and ADH1 genes can be used.

Additionally, it is preferred that the bait vector and prey vector contain one or more selectable markers for the selection and maintenance of only those yeast cells that harbor one or both chimeric genes. Any selectable markers known in the art can be used for purposes of this invention so long as yeast cells expressing the chimeric gene(s) can be positively identified or negatively selected. Examples of markers that can be positively identified are those based on color assays, including the lacZ gene (which encodes β-galactosidase), the firefly luciferase gene, secreted alkaline phosphatase, horseradish peroxidase, the blue fluorescent protein (BFP), and the green fluorescent protein (GFP) gene (see Cubitt et al., Trends Biochem. Sci., 20:448-455 (1995)). Other markers allowing detection by fluorescence, chemiluminescence, UV absorption, infrared radiation, and the like can also be used. Among the markers that can be selected are auxotrophic markers including, but not limited to, URA3, HIS3, TRP1, LEU2, LYS2, ADE2, and the like. Typically, for purposes of auxotrophic selection, the yeast host cells transformed with bait vector and/or prey vector are cultured in a medium lacking a particular nutrient. Other selectable markers are not based on auxotrophies, but rather on resistance or sensitivity to an antibiotic or other xenobiotic. Examples of such markers include but are not limited to chloramphenicol acetyl transferase (CAT) gene, which confers resistance to chloramphenicol; CAN1 gene, which encodes an arginine permease and thereby renders cells sensitive to canavanine (see Sikorski et al., Meth. Enzymol., 194:302-318 (1991)); the bacterial kanamycin resistance gene (kan^{R}), which renders eukaryotic cells resistant to the aminoglycoside G418 (see Wach et al., Yeast, 10:1793-1808 (1994)); and CYH2 gene, which confers sensitivity to cycloheximide (see Sikorski et al., Meth. Enzymol., 194:302-318 (1991)). In addition, the CUP1 gene, which encodes metallothionein and thereby confers resistance to copper, is also a suitable selection marker. Each of the above selection markers may be used alone or in combination. One or more selection markers can be included in a particular bait or prey vector. The bait vector and prey vector may have the same or different selection markers. In addition, the selection pressure can be placed on the transformed host cells either before or after mating the haploid yeast cells.

As will be apparent, the selection markers used should complement the host strains in which the bait and/or prey vectors are expressed. In other words, when a gene is used as a selection marker gene, a yeast strain lacking the selection marker gene (or having mutation in the corresponding gene) should be used as host cells. Numerous yeast strains or derivative strains corresponding to various selection markers are known in the art. Many of them have been developed specifically for certain yeast two-hybrid systems. The application and optional modification of such strains with respect to the present invention will be apparent to a skilled artisan apprised of the present disclosure. Methods for genetically manipulating yeast strains using genetic crossing or recombinant mutagenesis are well known in the art. See e.g., Rothstein, Meth. Enzymol., 101:202-211 (1983). By way of example, the following yeast strains are well known in the art, and can be used in the present invention upon necessary modifications and adjustment:
L40 strain which has the genotype MATa his3Δ200 trp1-901 leu2-3,112 ade2 LYS2::(lexAop)4-HIS3 URA3::(lexAop)8-lacZ;
EGY48 strain which has the genotype MATα trp1 his3 ura3 6ops-LEU2; and
MaV103 strain which has the genotype MATα ura3-52 leu2-3,112 trp1-901 his3Δ200 ade2-101 gal4Δ gal80Δ SPAL10::URA3 GAL1::HIS3::lys2 (see Kumar et al., J. Biol. Chem. 272:13548-13554 (1997); Vidal et al., Proc. Natl. Acad. Sci. USA, 93:10315-10320 (1996)). Such strains are generally available in the research community, and can also be obtained by simple yeast genetic manipulation. See, e.g., The Yeast Two-Hybrid System, Bartel and Fields, eds., pages 173-182, Oxford University Press, New York, N.Y., 1997.

In addition, the following yeast strains are commercially available:
Y190 strain which is available from Clontech, Palo Alto, Calif. and has the genotype MATα gal4 gal80 his3Δ200 trp1-901 ade2-101 ura3-52 leu2-3, 112 URA3::GAL1-lacZ LYS2::GAL1-HIS3 cyh^{r}; and
YRG-2 Strain which is available from Stratagene, La Jolla, Calif. and has the genotype MATα ura3-52 his3-200 ade2-101 lys2-801 trp1-901 leu2-3, 112 gal4-542 gal80-538 LYS2::GAL1-HIS3 URA3::GAL1/CYC1-lacZ.

In fact, different versions of vectors and host strains specially designed for yeast two-hybrid system analysis are available in kits from commercial vendors such as Clontech, Palo Alto, Calif. and Stratagene, La Jolla, Calif., all of which can be modified for use in the present invention.

### 5.3.1.2. Reporters

Generally, in a transcription-based two-hybrid assay, the interaction between a bait fusion protein and a prey fusion protein brings the DNA-binding domain and the transcription-activation domain into proximity forming a functional transcriptional factor that acts on a specific promoter to drive the expression of a reporter protein. The transcription activation domain and the DNA-binding domain may be selected from various known transcriptional activators, e.g., GAL4, GCN4, ARD1, the human estrogen receptor, E. coli LexA protein, herpes simplex virus VP16 (Triezenberg et al., Genes Dev. 2:718-729 (1988)), the E. coli B42 protein (acid blob, see Gyuris et al., Cell, 75:791-803 (1993)), NF-kB p65, and the like. The reporter gene and the promoter driving its transcription typically are incorporated into a separate reporter vector. Alternatively, the host cells are engineered to contain such a promoter-reporter gene sequence in their chromosomes. Thus, the interaction or lack of interaction between two interacting protein members of a protein complex can be determined by detecting or measuring changes in the assay system's reporter. Although the reporters and selection markers can be of similar types and used in a similar manner in the present invention, the reporters and selection markers should be carefully selected in a particular detection assay such that they are distinguishable from each other and do not interfere with each other's function.

Many different types of reporters are useful in the screening assays. For example, a reporter protein may be a fusion protein having an epitope tag fused to a protein. Commonly used and commercially available epitope tags include sequences derived from, e.g., influenza virus hemagglutinin (HA), Simian Virus 5 (V5), polyhistidine (6×His), c-myc, lacZ, GST, and the like. Antibodies specific to these epitope tags are generally commercially available. Thus, the expressed reporter can be detected using an epitope-specific antibody in an immunoassay.

In another embodiment, the reporter is selected such that it can be detected by a color-based assay. Examples of such reporters include, e.g., the lacZ protein (β-galactosidase), the green fluorescent protein (GFP), which can be detected by fluorescence assay and sorted by flow-activated cell sorting (FACS) (See Cubitt et al., Trends Biochem. Sci., 20:448-455 (1995)), secreted alkaline phosphatase, horseradish peroxidase, the blue fluorescent protein (BFP), and luciferase photoproteins such as aequorin, obelin, mnemiopsin, and berovin (See U.S. Pat. No. 6,087,476, which is incorporated herein by reference).

Alternatively, an auxotrophic factor is used as a reporter in a host strain deficient in the auxotrophic factor. Thus, suitable auxotrophic reporter genes include, but are not limited to, URA3, HIS3, TRP1, LEU2, LYS2, ADE2, and the like. For example, yeast cells containing a mutant URA3 gene can be used as host cells (Ura⁻ phenotype). Such cells lack URA3-encoded functional orotidine-5′-phosphate decarboxylase, an enzyme required by yeast cells for the biosynthesis of uracil. As a result, the cells are unable to grow on a medium lacking uracil. However, wild-type orotidine-5′-phosphate decarboxylase catalyzes the conversion of a non-toxic compound 5-fluoroorotic acid (5-FOA) to a toxic product, 5-fluorouracil. Thus, yeast cells containing a wild-type URA3 gene are sensitive to 5-FOA and cannot grow on a medium containing 5-FOA. Therefore, when the interaction between the interacting protein members in the fusion proteins results in the expression of active orotidine-5′-phosphate decarboxylase, the Ura⁻ (Foa^{R}) yeast cells will be able to grow on a uracil deficient medium (SC-Ura plates). However, such cells will not survive on a medium containing 5-FOA. Thus, protein-protein interactions can be detected based on cell growth.

Additionally, antibiotic resistance reporters can also be employed in a similar manner. In this respect, host cells sensitive to a particular antibiotic are used. Antibiotic resistance reporters include, for example, the chloramphenicol acetyl transferase (CAT) gene and the kan^{R} gene, which confer resistance to G418 in eukaryotes, and kanamycin in prokaryotes, respectively.

### 5.3.1.3. Screening Assays for Interaction Antagonists

The screening assays of the present invention are useful for identifying compounds capable of interfering with, disrupting, or dissociating the protein-protein interactions formed between members of the interacting protein pairs disclosed in the tables above, or between mutant and wild type, or mutant and mutant forms of these proteins. Since the protein complexes of the present invention are associated with inflammation and inflammatory disorders (e.g., asthma, rheumatoid arthritis, juvenile chronic arthritis, myositis, Chron's disease, gastritis, colitis, ulcerative colitis, inflammatory bowel disease, proctitis, pelvic inflammatory disease, systemic lupus erythematosus, rhinitis, conjunctivitis, scleritis, chronic inflammatory polyneuropathy, Tertiary Lyme disease, psoriasis, dermatitis, eczema, etc.) (either directly through their known cellular roles or functions or through the association of mutant forms of these proteins with the disease, or indirectly—through their interactions with other proteins known to be linked to inflammation and inflammatory disorders (e.g., asthma, rheumatoid arthritis, juvenile chronic arthritis, myositis, Chron's disease, gastritis, colitis, ulcerative colitis, inflammatory bowel disease, proctitis, pelvic inflammatory disease, systemic lupus erythematosus, rhinitis, conjunctivitis, scleritis, chronic inflammatory polyneuropathy, Tertiary Lyme disease, psoriasis, dermatitis, eczema, etc.)), disruption or dissociation of particular protein-protein interactions may be desirable to ameliorate the disease condition, or to alleviate disease symptoms. Alternatively, if the disease or disorder is associated with increased expression of any of the proteins presented in the tables, or with expression of a mutant form, or forms, of these proteins, then the disease or disorder may be ameliorated, or symptoms reduced, by weakening or dissociating the interaction between the interacting proteins in patients. Also, if a disease or disorder is associated with a mutant form of an interacting protein that form stronger protein-protein interactions with its protein partner than its wild type counterpart, then the disease or disorder may be treated with a compound that weakens, disrupts or interferes with the interaction between the mutant protein and its interacting partner.

In a screening assay for an interaction antagonist, a first protein, which is a protein selected from any of the protein pairs described in the tables (or a homologue, fragment or derivative thereof), or a mutant form of the first protein (or a homologue, fragment or derivative thereof), and a second protein, which is the interacting partner of the first protein identified in the tables above (or a homologue, fragment or derivative thereof), or a mutant form of the second protein (or a homologue, fragment or derivative thereof), are used as test proteins expressed in the form of fusion proteins as described above for purposes of a two-hybrid assay. The fusion proteins are expressed in a host cell and allowed to interact with each other in the presence of one or more test compounds.

In a preferred embodiment, a counterselectable marker is used as a reporter such that a detectable signal (e.g., appearance of color or fluorescence, or cell survival) is present only when the test compound is capable of interfering with the interaction between the two test proteins. In this respect, the reporters used in various “reverse two-hybrid systems” known in the art may be employed. Reverse two-hybrid systems are disclosed in, e.g., U.S. Pat. Nos. 5,525,490; 5,733,726; 5,885,779; Vidal et al., Proc. Natl. Acad. Sci. USA, 93:10315-10320 (1996); and Vidal et al., Proc. Natl. Acad. Sci. USA, 93:10321-10326 (1996), all of which are incorporated herein by reference.

Examples of suitable counterselectable reporters useful in a yeast system include the URA3 gene (encoding orotidine-5′-decarboxylase, which converts 5-fluroorotic acid (5-FOA) to the toxic metabolite 5-fluorouracil), the CANI gene (encoding arginine permease, which transports the toxic arginine analog canavanine into yeast cells), the GAL1 gene (encoding galactokinase, which catalyzes the conversion of 2-deoxygalactose to toxic 2-deoxygalactose-1-phosphate), the LYS2 gene (encoding α-aminoadipate reductase, which renders yeast cells unable to grow on a medium containing α-aminoadipate as the sole nitrogen source), the MET15 gene (encoding O-acetylhomoserine sulfhydrylase, which confers on yeast cells sensitivity to methyl mercury), and the CYH2 gene (encoding L29 ribosomal protein, which confers sensitivity to cycloheximide). In addition, any known cytotoxic agents including cytotoxic proteins such as the diphtheria toxin (DTA) catalytic domain can also be used as counterselectable reporters. See U.S. Pat. No. 5,733,726. DTA causes the ADP-ribosylation of elongation factor-2 and thus inhibits protein synthesis and causes cell death. Other examples of cytotoxic agents include ricin, Shiga toxin, and exotoxin A of Pseudomonas aeruginosa.

For example, when the URA3 gene is used as a counterselectable reporter gene, yeast cells containing a mutant URA3 gene can be used as host cells (Ura⁻Foa^{R} phenotype) for the in vivo assay. Such cells lack URA3-encoded functional orotidine-5′-phosphate decarboxylase, an enzyme required for the biosynthesis of uracil. As a result, the cells are unable to grow on media lacking uracil. However, because of the absence of a wild-type orotidine-5′-phosphate decarboxylase, the yeast cells cannot convert non-toxic 5-fluoroorotic acid (5-FOA) to a toxic product, 5-fluorouracil. Thus, such yeast cells are resistant to 5-FOA and can grow on a medium containing 5-FOA. Therefore, for example, to screen for a compound capable of disrupting interactions between PRAK (or a homologue, fragment or derivative thereof), or a mutant form of PRAK (or a homologue, fragment or derivative thereof), and ERK3 (or a homologue, fragment or derivative thereof), or a mutant form of ERK3 (or a homologue, fragment or derivative thereof), PRAK (or a homologue, fragment or derivative thereof) is expressed as a fusion protein with a DNA-binding domain of a suitable transcription activator while ERK3 (or a homologue, fragment or derivative thereof) is expressed as a fusion protein with a transcription activation domain of a suitable transcription activator. In the host strain, the reporter URA3 gene may be operably linked to a promoter specifically responsive to the association of the transcription activation domain and the DNA-binding domain. After the fusion proteins are expressed in the Ura⁻Foa^{R} yeast cells, an in vivo screening assay can be conducted in the presence of a test compound with the yeast cells being cultured on a medium containing uracil and 5-FOA. If the test compound does not disrupt the interaction between PRAK and ERK3, active URA3 gene product, i.e., orotidine-5′-decarboxylase, which converts 5-FOA to toxic 5-fluorouracil, is expressed. As a result, the yeast cells cannot grow. On the other hand, when the test compound disrupts the interaction between PRAK and ERK3, no active orotidine-5′-decarboxylase is produced in the host yeast cells. Consequently, the yeast cells will survive and grow on the 5-FOA-containing medium. Therefore, compounds capable of interfering with or dissociating the interaction between PRAK and ERK3 can thus be identified based on colony formation.

As will be apparent, the screening assay of the present invention can be applied in a format appropriate for large-scale screening. For example, combinatorial technologies can be employed to construct combinatorial libraries of small organic molecules or small peptides. See generally, e.g., Kenan et al., Trends Biochem. Sc., 19:57-64 (1994); Gallop et al., J. Med. Chem., 37:1233-1251 (1994); Gordon et al., J. Med. Chem., 37:1385-1401 (1994); Ecker et al., Biotechnology, 13:351-360 (1995). Such combinatorial libraries of compounds can be applied to the screening assay of the present invention to isolate specific modulators of particular protein-protein interactions. In the case of random peptide libraries, the random peptides can be co-expressed with the fusion proteins of the present invention in host cells and assayed in vivo. See e.g., Yang et al., Nucl. Acids Res., 23:1152-1156 (1995). Alternatively, they can be added to the culture medium for uptake by the host cells.

Conveniently, yeast mating is used in an in vivo screening assay. For example, haploid cells of a-mating type expressing one fusion protein as described above are mated with haploid cells of α-mating type expressing the other fusion protein. Upon mating, the diploid cells are spread on a suitable medium to form a lawn. Drops of test compounds can be deposited onto different areas of the lawn. After culturing the lawn for an appropriate period of time, drops containing a compound capable of modulating the interaction between the particular test proteins in the fusion proteins can be identified by stimulation or inhibition of growth in the vicinity of the drops.

The screening assays of the present invention for identifying compounds capable of modulating protein-protein interactions can also be fine-tuned by various techniques to adjust the thresholds or sensitivity of the positive and negative selections. Mutations can be introduced into the reporter proteins to adjust their activities. The uptake of test compounds by the host cells can also be adjusted. For example, yeast high uptake mutants such as the erg6 mutant strains can facilitate yeast uptake of the test compounds. See Gaber et al., Mol. Cell. Biol., 9:3447-3456 (1989). Likewise, the uptake of the selection compounds such as 5-FOA, 2-deoxygalactose, cycloheximide, α-aminoadipate, and the like can also be fine-tuned.

Generally, a control assay is performed in which the above screening assay is conducted in the absence of the test compound. The result of this assay is then compared with that obtained in the presence of the test compound.

### 5.3.1.4. Screening Assays for Interaction Agonists

The screening assays of the present invention can also be used to identify compounds that trigger or initiate, enhance or stabilize the protein-protein interactions formed between members of the interacting protein pairs disclosed in the tables above, or between combinations of mutant and wild type forms of such proteins, or pairs of mutant proteins. For example, if a disease or disorder is associated with the decreased expression of any one of the individual proteins, or one of the protein pairs selected from the tables, then the disease or disorder may be treated by strengthening or stabilizing the interactions between the interacting partner proteins in patients. Alternatively, if a disease or disorder is associated with a mutant form, or forms, of the interacting proteins that exhibit weakened or abolished interactions with their binding partner(s), then the disease or disorder may be treated with a compound that initiates or stabilizes the interaction between the mutant form, or forms, of the interacting proteins.

Thus, a screening assay can be performed in the same manner as described above, except that a positively selectable marker is used. For example, a first protein, which is any protein selected from the proteins described in the tables (or a homologue, fragment, or derivative thereof), or a mutant form of the first protein (or a homologue, fragment, or derivative thereof), and a second protein, which is an interacting partner of the first protein (or a homologue, fragment, or derivative thereof), or a mutant form of the second protein (or a homologue, fragment, or derivative thereof), are used as test proteins expressed in the form of fusion proteins as described above for purposes of a two-hybrid assay. The fusion proteins are expressed in host cells and are allowed to interact with each other in the presence of one or more test compounds.

A gene encoding a positively selectable marker such as β-galatosidase may be used as a reporter gene such that when a test compound enables, enhances or strengthens the interaction between a first protein, (or a homologue, fragment, or derivative thereof), or a mutant form of the first protein (or a homologue, fragment, or derivative thereof), and a second protein (or a homologue, fragment, or derivative thereof), or a mutant form of the second (or a homologue, fragment, or derivative thereof), β-galatosidase is expressed. As a result, the compound may be identified based on the appearance of a blue color when the host cells are cultured in a medium containing X-Gal.

Generally, a control assay is performed in which the above screening assay is conducted in the absence of the test compound. The result of this assay is then compared with that obtained in the presence of the test compound.

### 5.4. Optimization of the Identified Compounds

Once test compounds are selected that are capable of modulating the interaction between the interacting protein pairs of proteins described in the tables, or modulating the activity or intracellular levels of their constituent proteins, a secondary assay can be performed to confirm the specificity and effect of the compounds selected in the primary screens. Exemplary secondary assays are cell-based assays or animal based assays.

For example, anti-inflammatory compounds can be identified in a cell-based assay by their ability to inhibit the secretion of cytokines from activated T cells. T cells play a central role in raising an inflammatory response upon stimulation by specific antigens. The human Jurkat T leukemia cell line can be used as a model system. T cell receptor activation in this cell line, as measured by the secretion of the cytokines TNF-α, IL-2, and IFN-γ, can be achieved in vitro by combined stimulation with anti CD3 and anti CD28 antibodies. Alternatively, activation can also be achieved by a combination of phorbol ester and calcium ionophore believed to stimulate protein kinase C and calcineurin, respectively (Iñiguez et al., J. Immunol. 163:111-119 (1999)).

Anti-inflammatory compounds can be identified in the mouse carrageenan-induced foot paw edema model (See Winter et al., Proc. Soc. Exp. Biol. Med. 111:544-547 (1962)). In this assay a seaweed-derived sulfated polysaccharide, carrageenan, is used as an antigen/irritant. Carrageenan is injected into the paw of mice, which results in swelling due to inflammation. The degree of swelling is a measure of the inflammatory response. Thus, the anti-inflammatory effect of a compound can be tested by administering a compound to a mouse and measuring a reduction in carrageenan-induced paw swelling compared to the carrageenan-induced paw swelling of control mice that do not have the compound administered to them.

Anti-inflammatory compounds can also be identified in the rat adjuvant induced arthritis assay (Jaffee et al., Agents Actions 27:344-346 (1988)). In this assay arthritis is induced in rats by e.g. injection of an adjuvant such as Mycobacterium butyricum. After a period following injection of the adjuvant, an increase in paw volume of the rats is measured. Rats with a substantial increase in paw volume are randomly separated into two groups. Group 1 rats are treated with a test compound for a dosing period and at the end of the dosing period paw volume of Group 1 rats are compared to Group 2 rats. A reduction in paw volume of Group 1 rats compared to Group 2 rats indicates that the administered compound has an anti-inflammatory effect.

In addition, once test compounds are selected that are capable of modulating the proteins in the tables or the interaction between the interacting protein pairs of proteins described in the tables, or modulating the activity or intracellular levels of their constituent proteins, a data set including data defining the identity or characteristics of the test compounds can be generated. The data set may include information relating to the properties of a selected test compound, e.g., chemical structure, chirality, molecular weight, melting point, etc. Alternatively, the data set may simply include assigned identification numbers understood by the researchers conducting the screening assay and/or researchers receiving the data set as representing specific test compounds. The data or information can be cast in a transmittable form that can be communicated or transmitted to other researchers, particularly researchers in a different country. Such a transmittable form can vary and can be tangible or intangible. For example, the data set defining one or more selected test compounds can be embodied in texts, tables, diagrams, molecular structures, photographs, charts, images or any other visual forms. The data or information can be recorded on a tangible media such as paper or embodied in computer-readable forms (e.g., electronic, electromagnetic, optical or other signals). The data in a computer-readable form can be stored in a computer usable storage medium (e.g., floppy disks, magnetic tapes, optical disks, and the like) or transmitted directly through a communication infrastructure. In particular, the data embodied in electronic signals can be transmitted in the form of email or posted on a website on the Internet or Intranet. In addition, the information or data on a selected test compound can also be recorded in an audio form and transmitted through any suitable media, e.g., analog or digital cable lines, fiber optic cables, etc., via telephone, facsimile, wireless mobile phone, Internet phone and the like.

Thus, the information and data on a test compound selected in a screening assay described above or by virtual screening as discussed below can be produced anywhere in the world and transmitted to a different location. For example, when a screening assay is conducted offshore, the information and data on a selected test compound can be generated and cast in a transmittable form as described above. The data and information in a transmittable form thus can be imported into the U.S. or transmitted to any other countries, where the data and information may be used in further testing the selected test compound and/or in modifying and optimizing the selected test compound to develop lead compounds for testing in clinical trials.

Compounds can also be selected based on structural models of the target protein or protein complex and/or test compounds. In addition, once an effective compound is identified, structural analogs or mimetics thereof can be produced based on rational drug design with the aim of improving drug efficacy and stability, and reducing side effects. Methods known in the art for rational drug design can be used in the present invention. See, e.g., Hodgson et al., Bio/Technology, 9:19-21 (1991); U.S. Pat. Nos. 5,800,998 and 5,891,628, all of which are incorporated herein by reference. An example of rational drug design is the development of HIV protease inhibitors. See Erickson et al., Science, 249:527-533 (1990).

In this respect, structural information on the target protein or protein complex is obtained. Preferably, atomic coordinates defining a three-dimensional structure of the target protein or protein complex can be obtained. For example, each of the interacting pairs can be expressed and purified. The purified interacting protein pairs are then allowed to interact with each other in vitro under appropriate conditions. Optionally, the interacting protein complex can be stabilized by crosslinking or other techniques. The interacting complex can be studied using various biophysical techniques including, e.g., X-ray crystallography, NMR, computer modeling, mass spectrometry, and the like. Likewise, structural information can also be obtained from protein complexes formed by interacting proteins and a compound that initiates or stabilizes the interaction of the proteins. Methods for obtaining such atomic coordinates by X-ray crystallography, NMR, and the like are known in the art and the application thereof to the target protein or protein complex of the present invention should be apparent to skilled persons in the art of structural biology. See Smyth and Martin, Mol. Pathol., 53:8-14 (2000); Oakley and Wilce, Clin. Exp. Pharmacol. Physiol., 27(3):145-151 (2000); Ferentz and Wagner, Q. Rev. Biophys., 33:29-65 (2000); Hicks, Curr. Med. Chem., 8(6):627-650 (2001); and Roberts, Curr. Opin. Biotechnol., 10:42-47 (1999).

In addition, understanding of the interaction between the proteins of interest in the presence or absence of a modulator can also be derived by mutagenic analysis using a yeast two-hybrid system or other methods for detecting protein-protein interactions. In this respect, various mutations can be introduced into the interacting proteins and the effect of the mutations on protein-protein interaction examined by a suitable method such as the yeast two-hybrid system.

Various mutations including amino acid substitutions, deletions and insertions can be introduced into a protein sequence using conventional recombinant DNA technologies. Generally, it is particularly desirable to decipher the protein binding sites. Thus, it is important that the mutations introduced only affect protein-protein interactions and cause minimal structural disturbances. Mutations are preferably designed based on knowledge of the three-dimensional structure of the interacting proteins. Preferably, mutations are introduced to alter charged amino acids or hydrophobic amino acids exposed on the surface of the proteins, since ionic interactions and hydrophobic interactions are often involved in protein-protein interactions. Alternatively, the “alanine scanning mutagenesis” technique is used. See Wells, et al., Methods Enzymol., 202:301-306 (1991); Bass et al., Proc. Natl. Acad. Sci. USA, 88:4498-4502 (1991); Bennet et al., J. Biol. Chem., 266:5191-5201 (1991); Diamond et al., J. Virol., 68:863-876 (1994). Using this technique, charged or hydrophobic amino acid residues of the interacting proteins are replaced by alanine, and the effect on the interaction between the proteins is analyzed using e.g., the yeast two-hybrid system. For example, the entire protein sequence can be scanned in a window of five amino acids. When two or more charged or hydrophobic amino acids appear in a window, the charged or hydrophobic amino acids are changed to alanine using standard recombinant DNA techniques. The thus-mutated proteins are used as “test proteins” in the above-described two-hybrid assays to examine the effect of the mutations on protein-protein interaction. Preferably, the mutational analyses are conducted both in the presence and in the absence of an identified modulator compound. In this manner, the domains or residues of the proteins important to protein-protein interaction and/or the interaction between the modulator compound and the interacting proteins can be identified.

Based on the information obtained, structural relationships between the interacting proteins, as well as between the identified modulators and the interacting proteins are elucidated. For example, for the identified modulators (i.e., lead compounds), the three-dimensional structure and chemical moieties critical to their modulating effect on the interacting proteins are revealed. Using this information and various techniques known in the art of molecular modeling (i.e., simulated annealing), medicinal chemists can then design analog compounds that might be more effective modulators of the protein-protein interactions of the present invention. For example, the analog compounds might show more specific or tighter binding to their targets, and thereby might exhibit fewer side effects, or might have more desirable pharmacological characteristics (e.g., greater solubility).

In addition, if the lead compound is a peptide, it can also be analyzed by the alanine scanning technique and/or the two-hybrid assay to determine the domains or residues of the peptide important to its modulating effect on particular protein-protein interactions. The peptide compound can be used as a lead molecule for rational design of small organic molecules or peptide mimetics. See Huber et al., Curr. Med. Chem., 1:13-34 (1994).

The domains, residues or moieties critical to the modulating effect of the identified compound constitute the active region of the compound known as its “pharmacophore.” Once the pharmacophore has been elucidated, a structural model can be established by a modeling process that may incorporate data from NMR analysis, X-ray diffraction data, alanine scanning, spectroscopic techniques and the like. Various techniques including computational analysis (e.g., molecular modeling and simulated annealing), similarity mapping and the like can all be used in this modeling process. See e.g., Perry et al., in OSAR: Quantitative Structure-Activity Relationships in Drug Design, pp. 189-193, Alan R. Liss, Inc., 1989; Rotivinen et al., Acta Pharmaceutical Fennica, 97:159-166 (1988); Lewis et al., Proc. R. Soc. Lond., 236:125-140 (1989); McKinaly et al., Annu. Rev. Pharmacol. Toxiciol., 29:111-122 (1989). Commercial molecular modeling systems available from Polygen Corporation, Waltham, Mass., include the CHARMm program, which performs energy minimization and molecular dynamics functions, and QUANTA program, which performs construction, graphic modeling and analysis of molecular structure. Such programs allow interactive construction, modification, and visualization of molecules. Other computer modeling programs are also available from BioDesign, Inc. (Pasadena, Calif.), Hypercube, Inc. (Cambridge, Ontario), and Allelix, Inc. (Mississauga, Ontario, Canada).

A template can be formed based on the established model. Various compounds can then be designed by linking various chemical groups or moieties to the template. Various moieties of the template can also be replaced. In addition, in the case of a peptide lead compound, the peptide or mimetics thereof can be cyclized, e.g., by linking the N-terminus and C-terminus together, to increase its stability. These rationally designed compounds are further tested. In this manner, pharmacologically acceptable and stable compounds with improved efficacy and reduced side effects can be developed. The compounds identified in accordance with the present invention can be incorporated into a pharmaceutical formulation suitable for administration to an individual.

In addition, the structural models or atomic coordinates defining a three-dimensional structure of the target protein or protein complex can also be used in virtual screen to select compounds capable of modulating the target protein or protein complex. Various methods of computer-based virtual screen using atomic coordinates are generally known in the art. For example, U.S. Pat. No. 5,798,247 (which is incorporated herein by reference) discloses a method of identifying a compound (specifically, an interleukin converting enzyme inhibitor) by determining binding interactions between an organic compound and binding sites of a binding cavity within the target protein. The binding sites are defined by atomic coordinates.

The compounds designed or selected based on rational drug design or virtual screen can be tested for their ability to modulate (interfere with or strengthen) the interaction between the interacting partners within the protein complexes of the present invention. In addition, the compounds can also be further tested for their ability to modulate (inhibit or enhance) cellular functions such as intracellular signaling in cells as well as their effectiveness in treating diseases such as inflammation and inflammatory disorders (e.g., asthma, rheumatoid arthritis, juvenile chronic arthritis, myositis, Chron's disease, gastritis, colitis, ulcerative colitis, inflammatory bowel disease, proctitis, pelvic inflammatory disease, systemic lupus erythematosus, rhinitis, conjunctivitis, scleritis, chronic inflammatory polyneuropathy, Tertiary Lyme disease, psoriasis, dermatitis, eczema, etc.).

Following the selection of desirable compounds according to the methods disclosed above, the methods of the present invention further provide for the manufacture of the selected compounds. Compounds found to desirably modulate the interaction between the interacting protein pairs of proteins of the present invention, or to desirably modulate the activity or intracellular levels of their constituent proteins, can be manufactured for further experimental studies, or for therapeutic use.

### 6. Therapeutic Applications

As described above, the interactions between the interacting pairs of proteins of the present invention suggest that these proteins and/or the protein complexes formed by them may be involved in common biological processes and disease pathways. The protein complexes may mediate the functions of the individual proteins of each interacting protein pair, or of the interacting pairs themselves, in the biological processes or disease pathways. Thus, one may modulate such biological processes or treat diseases by modulating the functions and activities of any of the individual proteins described in the tables, and/or a protein complex comprising some combination of these proteins. As used herein, modulating a protein selected from the tables, or a protein complex comprising some combination of these proteins means altering (enhancing or reducing) the intracellular concentrations or activities of the proteins or protein complexes, e.g., increasing the concentrations of a particular protein described in the tables, or a protein complex comprising some combination of these proteins, enhancing or reducing their biological activities, increasing or decreasing their stability, altering their affinity or specificity to certain other biological molecules, etc. For example, a pair of interacting proteins listed in the tables may be involved in intracellular signaling. Thus, assays such as those described in Section 4 may be used in determining the effect of an aberration in a particular protein complex or an interacting member thereof on intracellular signaling. In addition, it is also possible to determine, using the same assay methods, the presence or absence of an association between a protein complex of the present invention or an interacting member thereof and a physiological disorder or disease such as inflammation and inflammatory disorders (e.g., asthma, rheumatoid arthritis, juvenile chronic arthritis, myositis, Chron's disease, gastritis, colitis, ulcerative colitis, inflammatory bowel disease, proctitis, pelvic inflammatory disease, systemic lupus erythematosus, rhinitis, conjunctivitis, scleritis, chronic inflammatory polyneuropathy, Tertiary Lyme disease, psoriasis, dermatitis, eczema, etc.) or predisposition to a physiological disorder or disease.

Once such associations are established, the diagnostic methods as described in Section 4 can be used in diagnosing the disease or disorder, or a patient's predisposition to it. In addition, various in vitro and in vivo assays may be employed to test the therapeutic or prophylactic efficacies of the various therapeutic approaches described in Sections 6.2 and 6.3 that are aimed at modulating the functions and activities of a particular protein complex of the present invention, or an interacting member thereof. Similar assays can also be used to test whether the therapeutic approaches described in Sections 6.2 and 6.3 result in the modulation of intracellular signaling. The cell model or transgenic animal model described in Section 7 may be employed in the in vitro and in vivo assays.

In accordance with this aspect of the present invention, methods are provided for modulating (promoting or inhibiting) a protein complex of the present invention formed by the interactions described in the tables. The human cells can be in in vitro cell or tissue cultures. The methods are also applicable to human cells in a patient.

In one embodiment, the concentration of a protein complex formed by the interactions described in the tables is reduced in the cells. Various methods can be employed to reduce the concentration of the protein complex. For example, the protein complex concentration can be reduced by interfering with the interactions between the interacting protein partners. Hence, compounds capable of interfering with interactions between interacting pairs of proteins identified in the tables can be administered to the cells in vitro or in vivo in a patient. Such compounds can be compounds capable of binding specific proteins listed in the tables. They can also be antibodies immunoreactive with specific proteins identified in the tables. Also, the compounds can be small peptides derived from a first interacting protein of the present invention, or a mimetic thereof, that are capable of binding a second protein of the present invention, the second protein being a binding partner of the first protein as shown in the tables above.

In another embodiment, the method of modulating the protein complex includes inhibiting the expression of any of the individual proteins described in the tables. The inhibition can be at the transcriptional, translational, or post-translational level. For example, antisense compounds and ribozyme compounds can be administered to human cells in cultures or in human bodies. In addition, RNA interference technologies may also be employed to administer to cells double-stranded RNA or RNA hairpins capable of “knocking down” the expression of any of the interacting proteins of the present invention.

In the various embodiments described above, preferably the concentrations or activities of both partners in an interacting pair of proteins of the present invention are reduced or inhibited, or the concentration or activitie of a single constituent protein of a protein complex formed by the interactions described in the tables is reduced or inhibited.

In yet another embodiment, an antibody selectively immunoreactive with a pair of interacting proteins identified in the tables is administered to cells in vitro or in human bodies to inhibit the protein complex activities and/or reduce the concentration of the protein complex in the cells or patient.

Further provided by the present invention is a method of treatment of a disease or disorder comprising identifying a patient that has a particular disease or disorder, shows symptoms of having a particular disease or disorder, is predisposed to, or at risk of developing a particular disease or disorder, and treating the disease or disorder by modulating a protein or protein-protein interaction according to the present invention.

### 6.1. Applicable Diseases

The methods for modulating the functions and activities of a protein complex of the present invention, or an interacting member thereof, may be employed to modulate intracellular signaling. In addition, the methods may also be used in the treatment or prevention of diseases and disorders such as inflammation and inflammatory disorders (e.g., asthma, rheumatoid arthritis, juvenile chronic arthritis, myositis, Chron's disease, gastritis, colitis, ulcerative colitis, inflammatory bowel disease, proctitis, pelvic inflammatory disease, systemic lupus erythematosus, rhinitis, conjunctivitis, scleritis, chronic inflammatory polyneuropathy, Tertiary Lyme disease, psoriasis, dermatitis, eczema, etc.). The methods may also be useful for treating or preventing other diseases such as cancer, Alzheimer's disease, cardiovascular diseases such as atherosclerosis, and coronary heart disease.

### 6.2. Inhibiting Protein Complex or Interacting Protein Members Thereof

In one aspect of the present invention, methods are provided for reducing in cells or tissue the concentration and/or activity of a protein complex identified in accordance with the present invention that comprises one or more of the interacting pairs of proteins described in the tables. In addition, methods are also provided for reducing in cells or tissue the concentration and/or activity of any of the individual proteins identified in the tables. By reducing the concentration of a protein complex and/or one or more of the protein constituents of the protein complex and/or inhibiting the functional activities of the protein complex and/or one or more of the protein constituents of the protein complex, the diseases involving such a protein complex or protein constituents of the protein complex may be treated or prevented.

### 6.2.1. Antibody Therapy

In one embodiment, an antibody may be administered to cells or tissue in vitro or to patients. The antibody administered may be immunoreactive with any of the individual proteins described in the tables, or with one of the protein complexes of the present invention. Suitable antibodies may be monoclonal or polyclonal that fall within any antibody class, e.g., IgG, IgM, IgA, IgE, etc. The antibody suitable for this invention may also take a form of various antibody fragments including, but not limited to, Fab and F(ab′)₂, single-chain fragments (scFv), and the like. In another embodiment, an antibody selectively immunoreactive with the protein complex formed from at least one of the interacting pairs of proteins described in the tables, is administered to cells or tissue in vitro or in to patient. In yet another embodiment, an antibody specific to an individual protein selected from any of the tables is administered to cells or tissue in vitro or in a patient. Methods for making the antibodies of the present invention should be apparent to a person of skill in the art, especially in view of the discussions in Section 3 above. The antibodies can be administered in any suitable form via any suitable route as described in Section 8 below. Preferably, the antibodies are administered in a pharmaceutical composition together with a pharmaceutically acceptable carrier.

Alternatively, the antibodies may be delivered by a gene-therapy approach. That is, nucleic acids encoding the antibodies, particularly single-chain fragments (scFv), may be introduced into cells or tissue in vitro or in a patient such that desirable antibodies may be produced recombinantly in vivo from the nucleic acids. For this purpose, the nucleic acids with appropriate transcriptional and translation regulatory sequences can be directly administered into the patient. Alternatively, the nucleic acids can be incorporated into a suitable vector as described in Sections 2.2 and 5.3.1.1 and delivered into cells or tissue in vitro or in a patient along with the vector. The expression vector containing the nucleic acids can be administered directly to cells or tissue in vitro or in a patient. It can also be introduced into cells, preferably cells derived from a patient to be treated, and subsequently delivered into the patient by cell transplantation. See Section 6.3.2 below.

### 6.2.2. siRNA Therapy

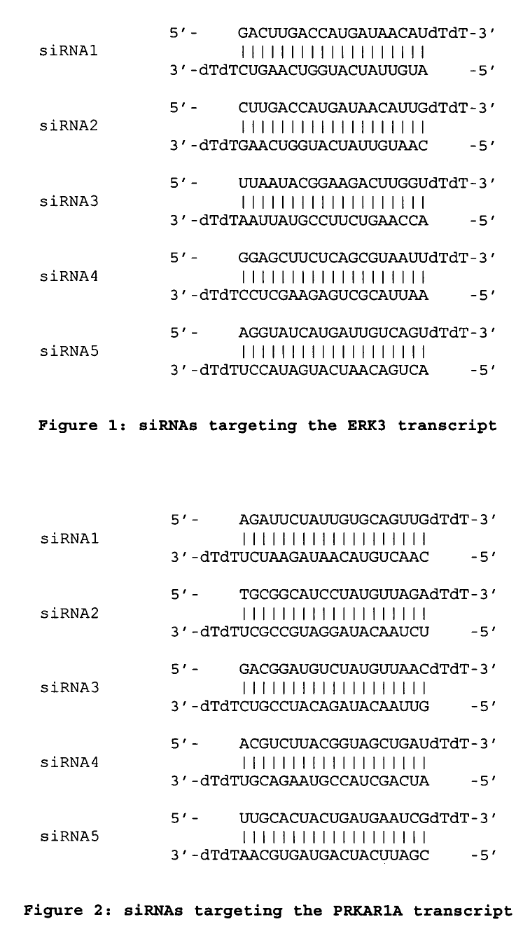

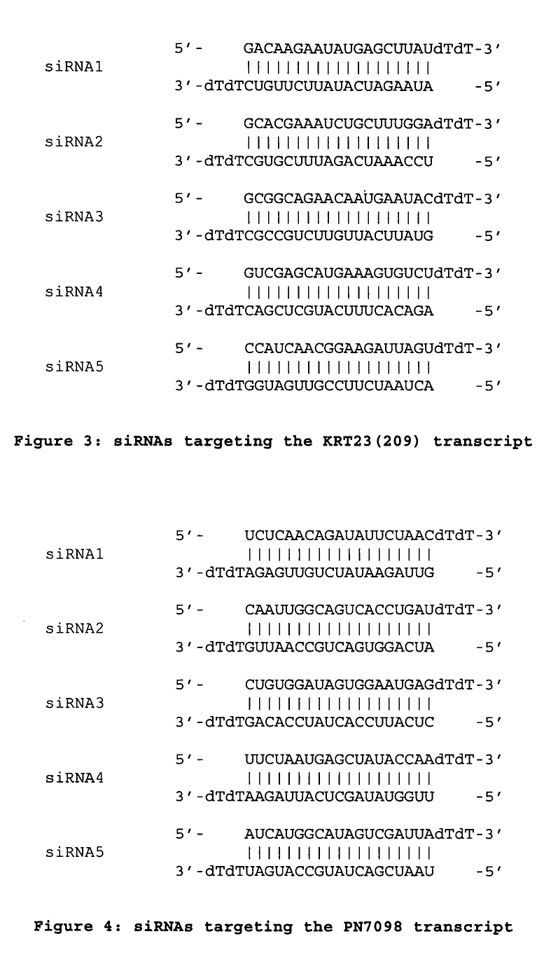

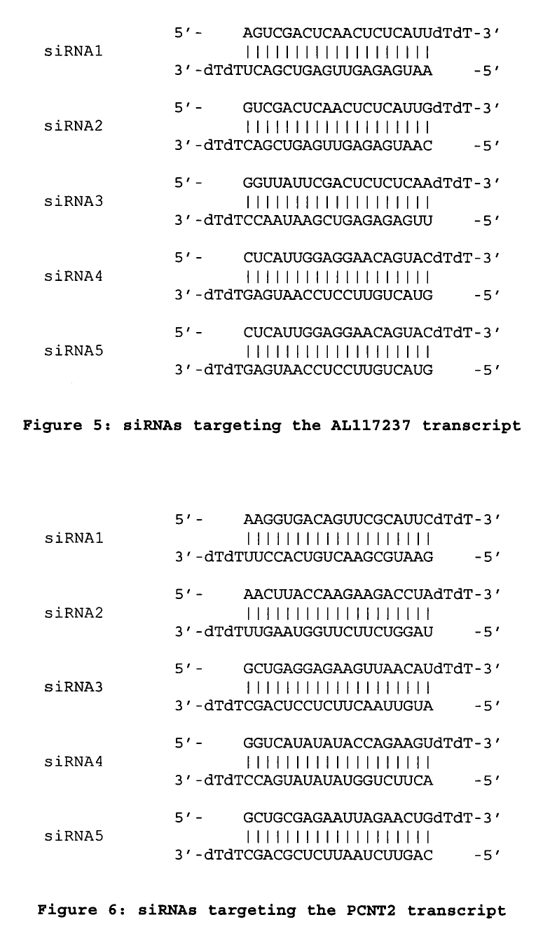

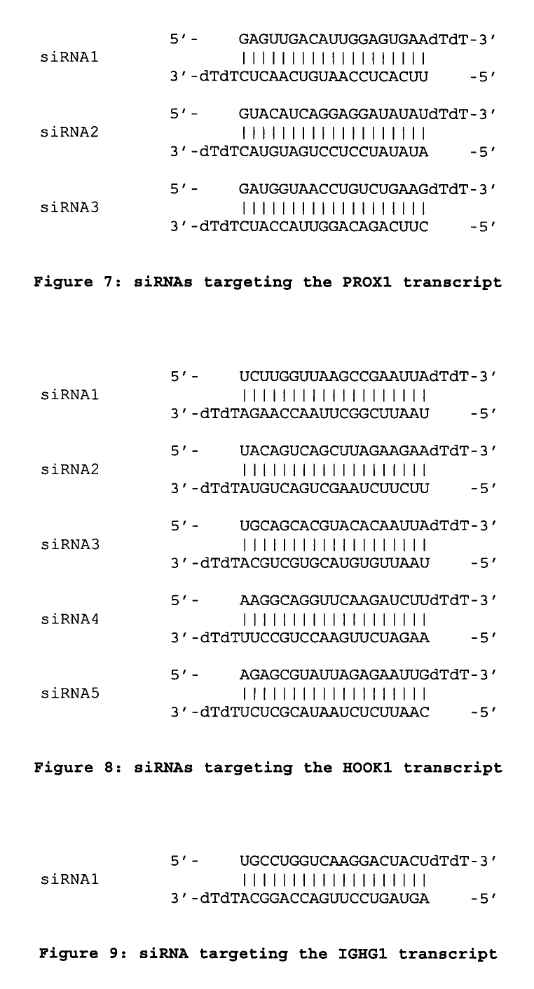

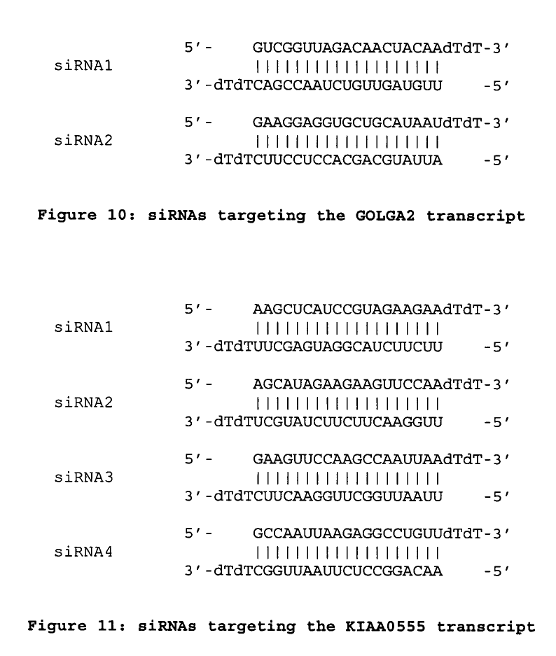

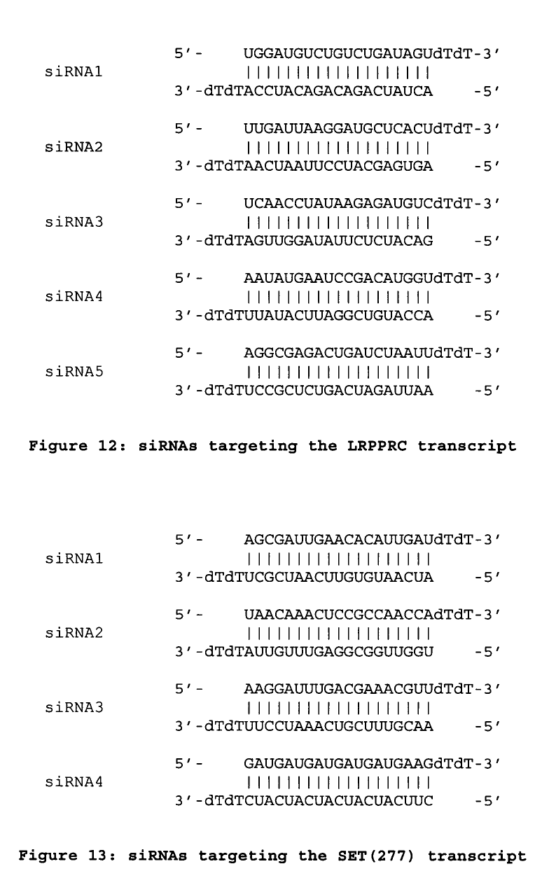

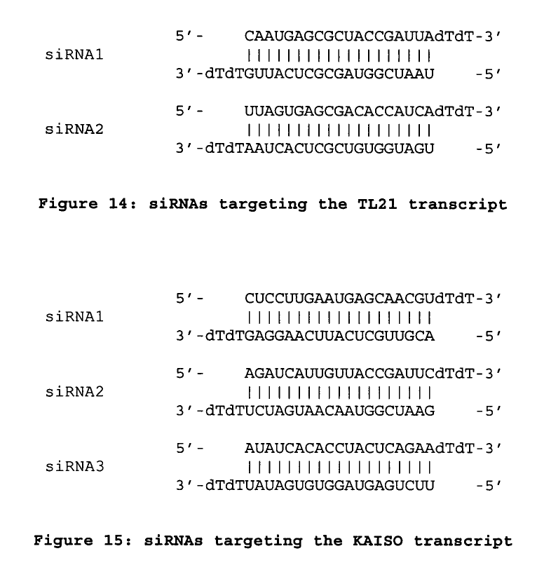

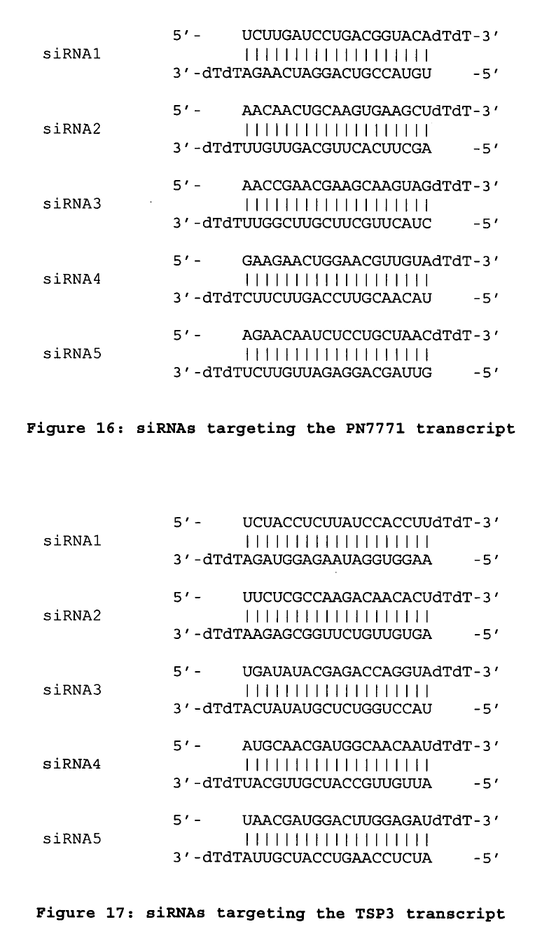

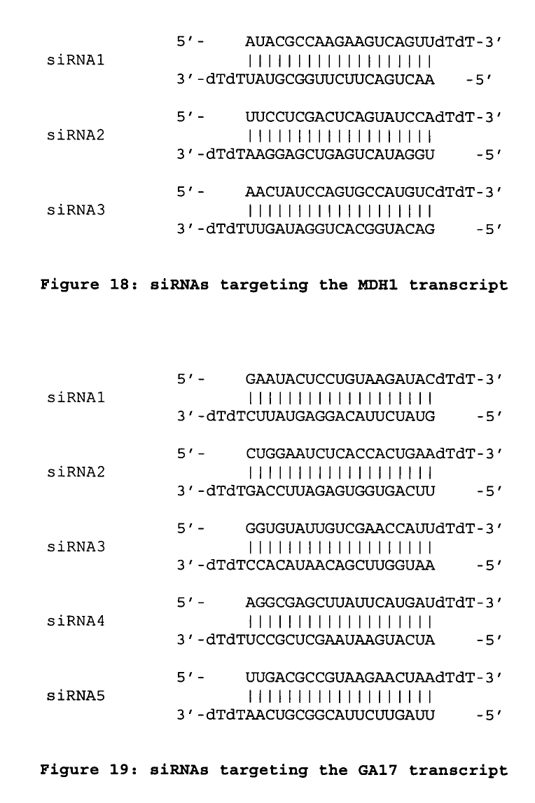

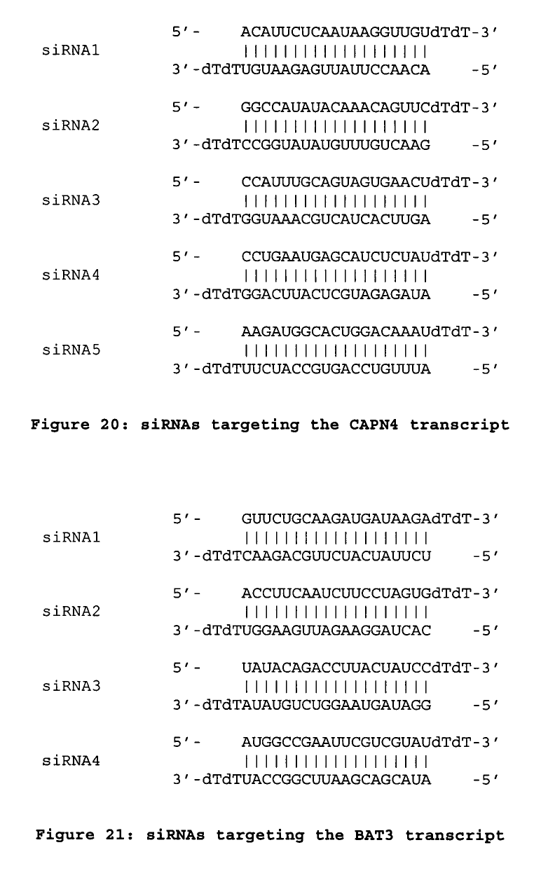

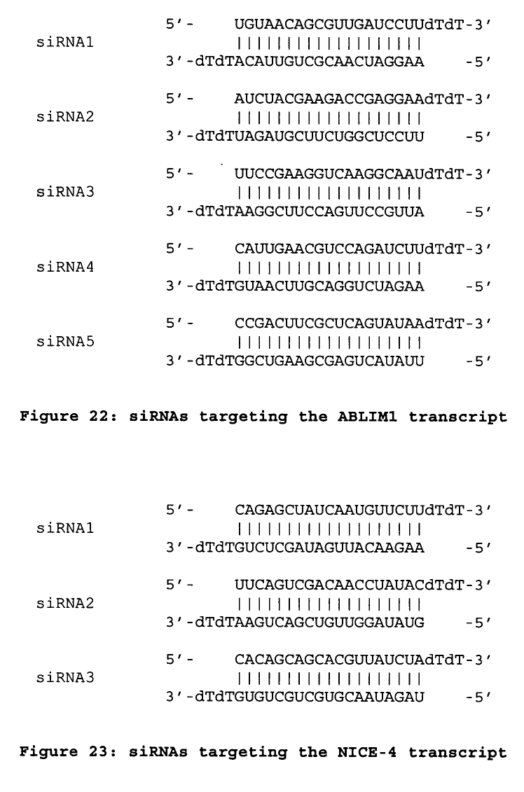

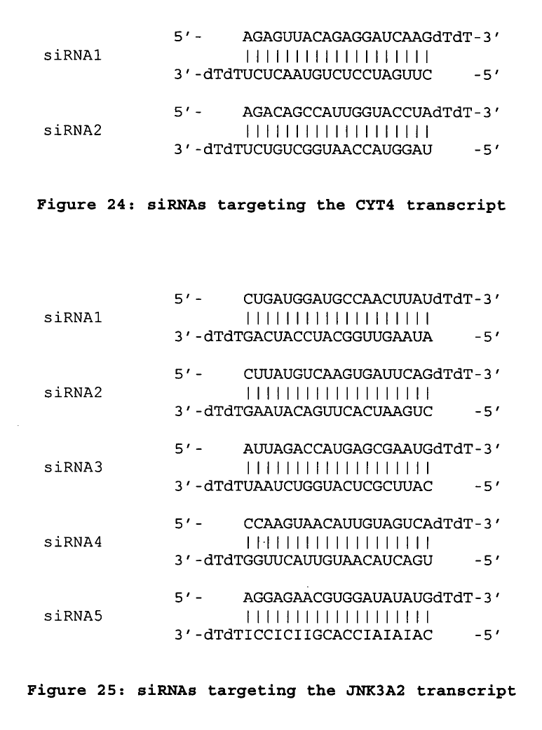

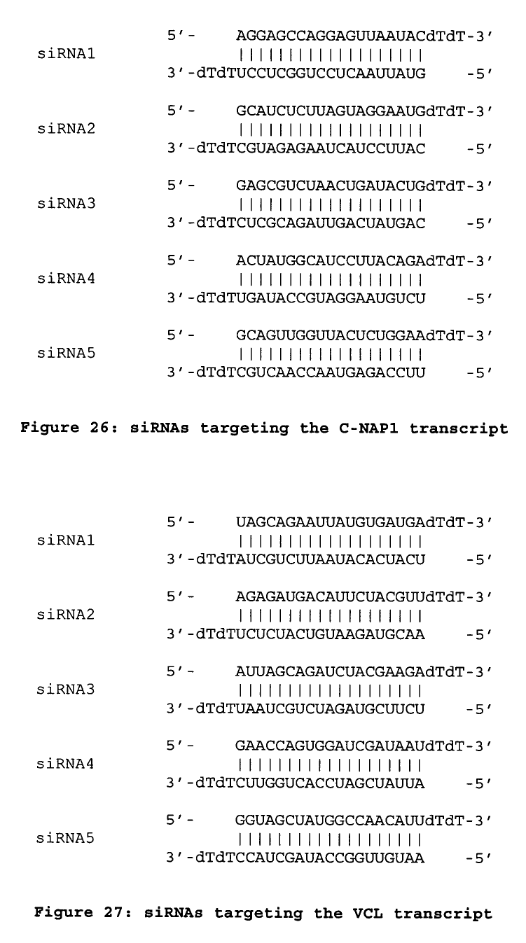

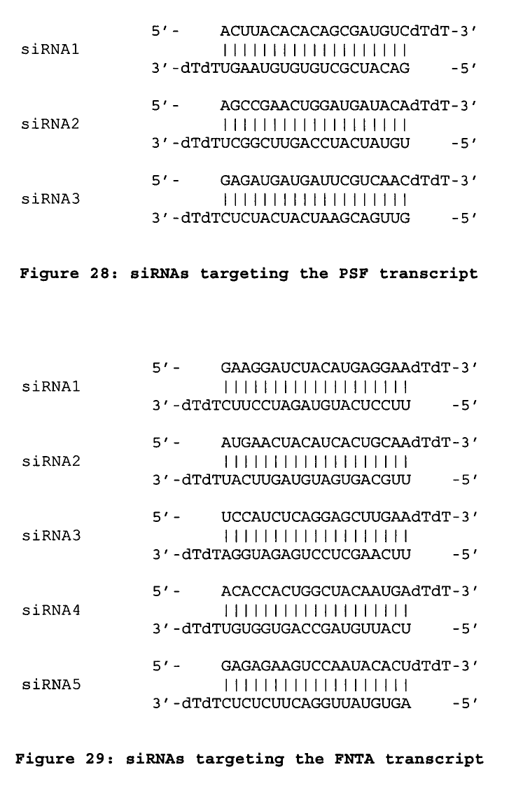

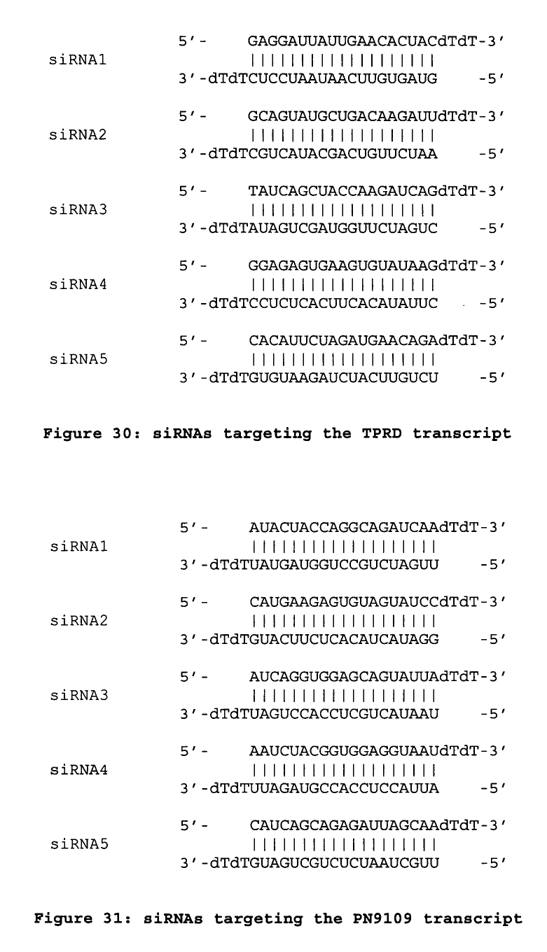

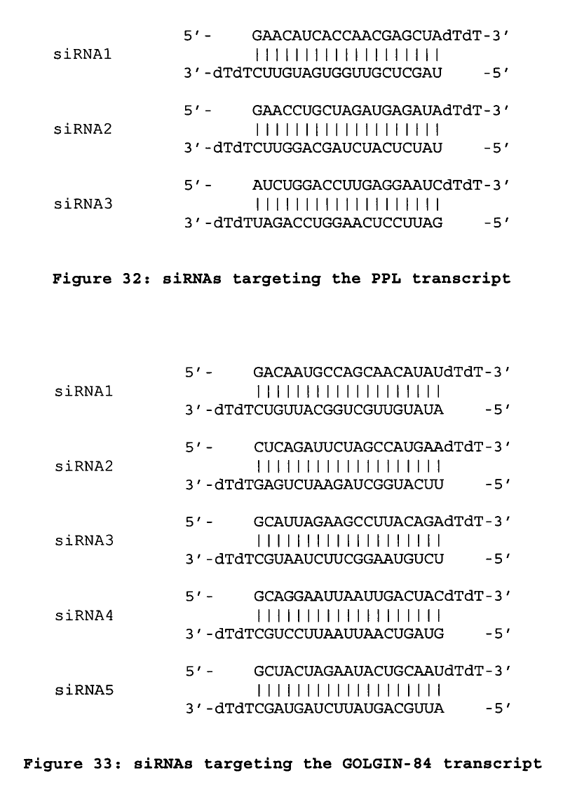

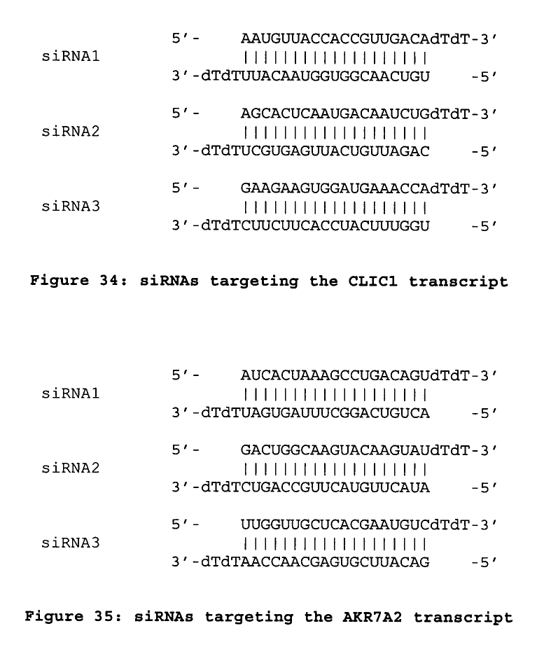

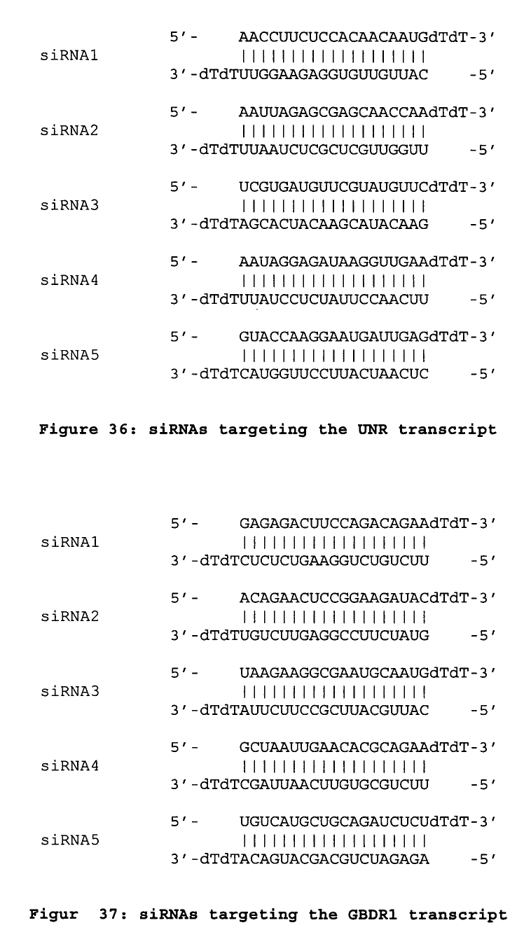

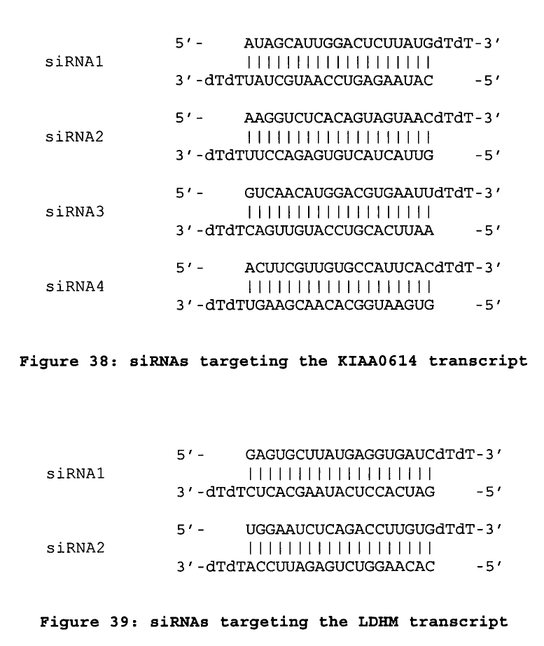

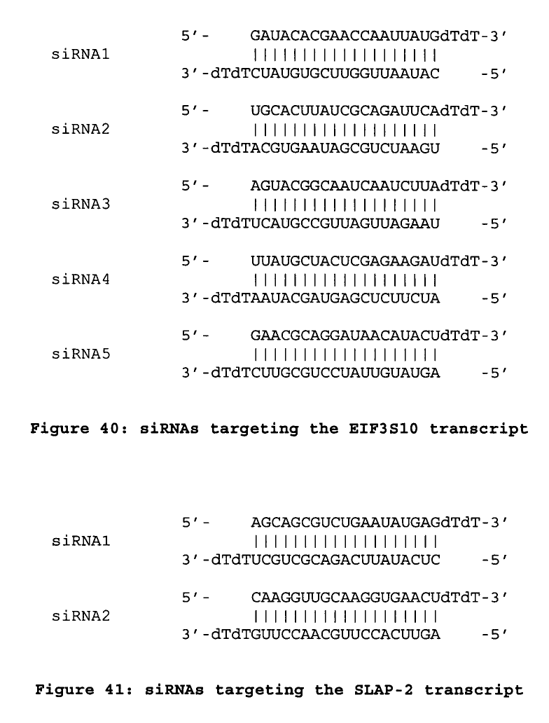

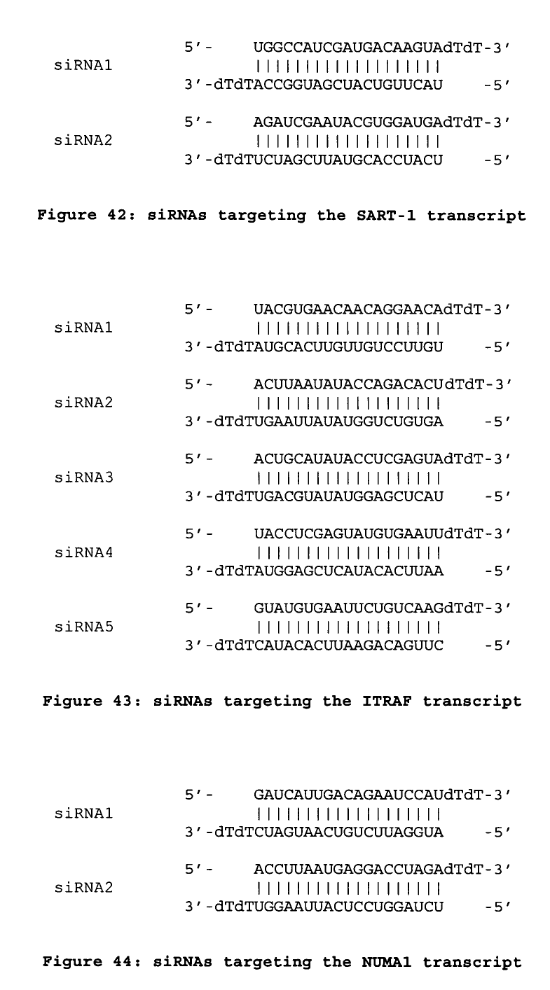

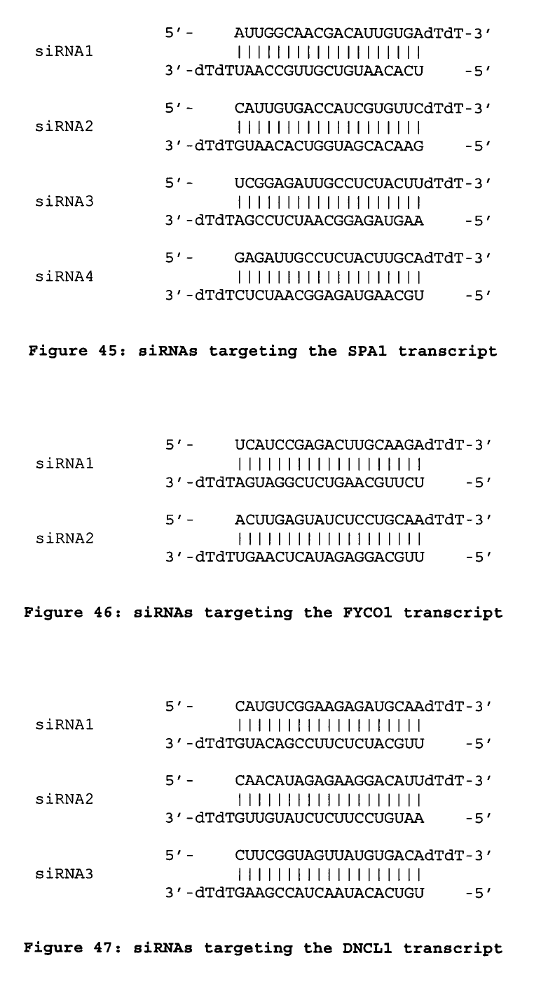

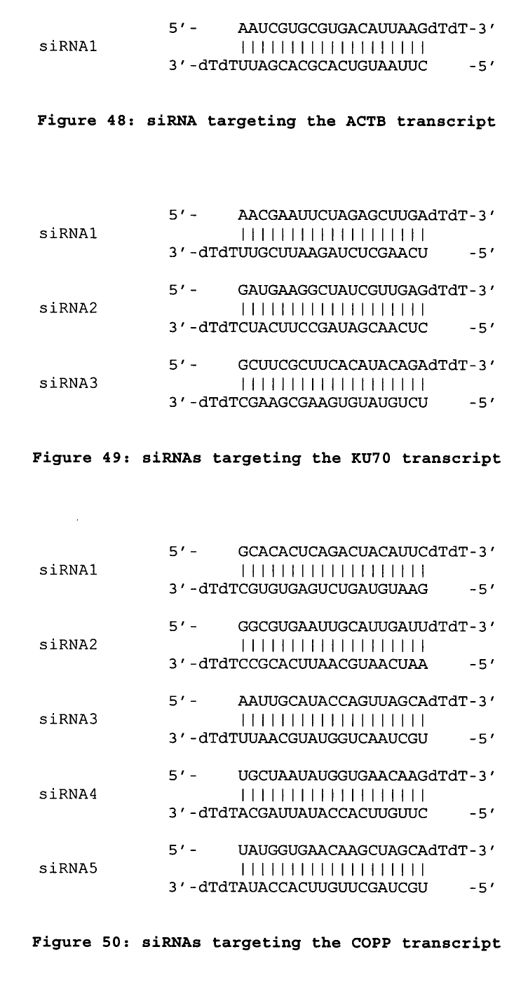

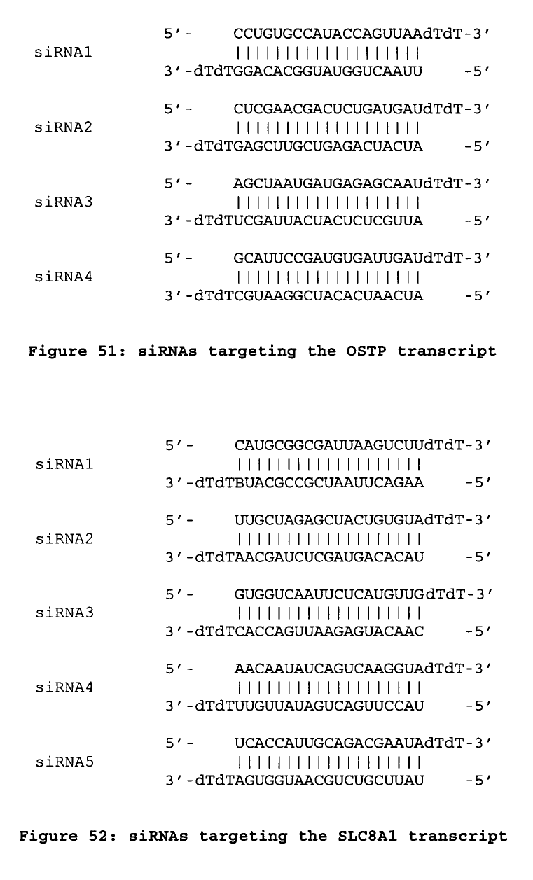

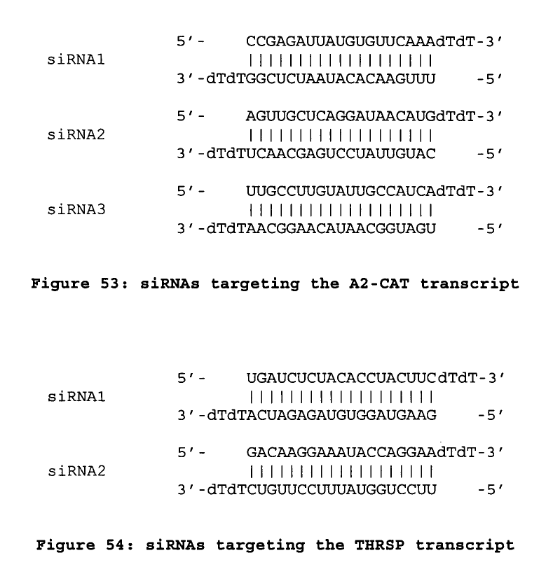

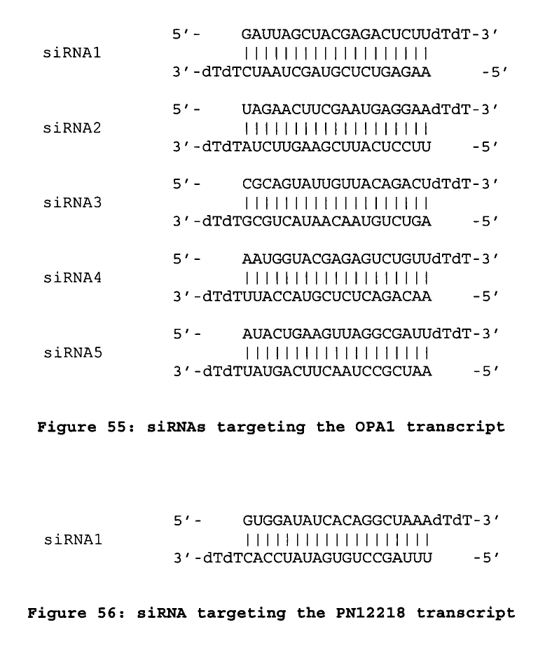

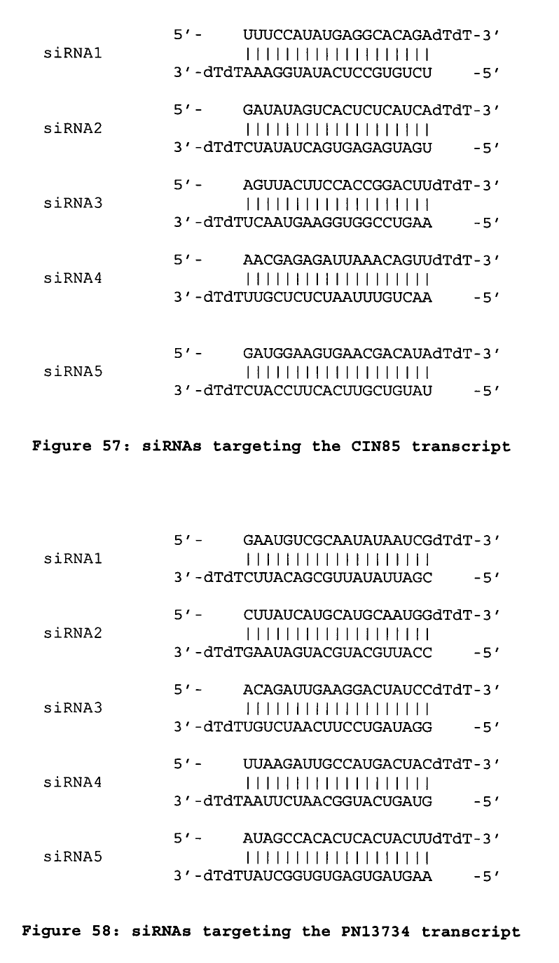

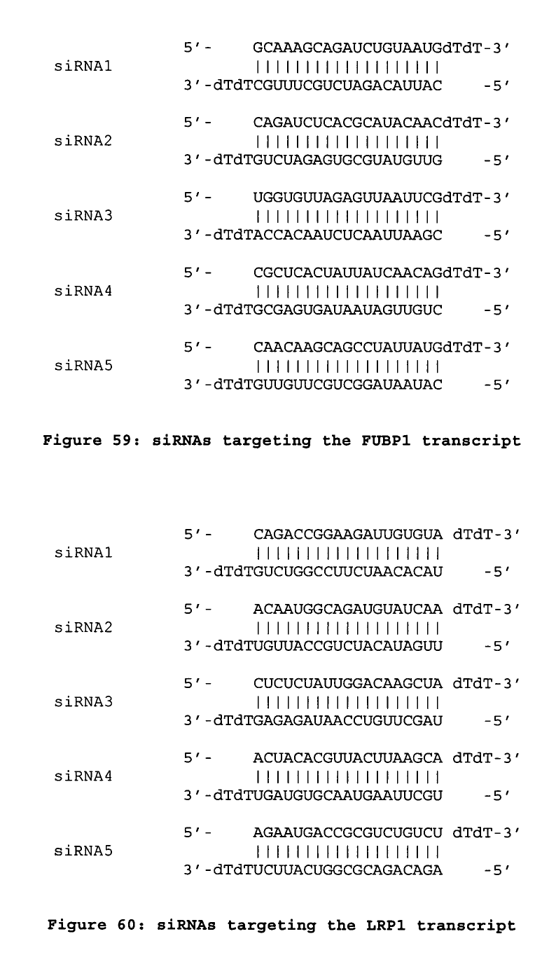

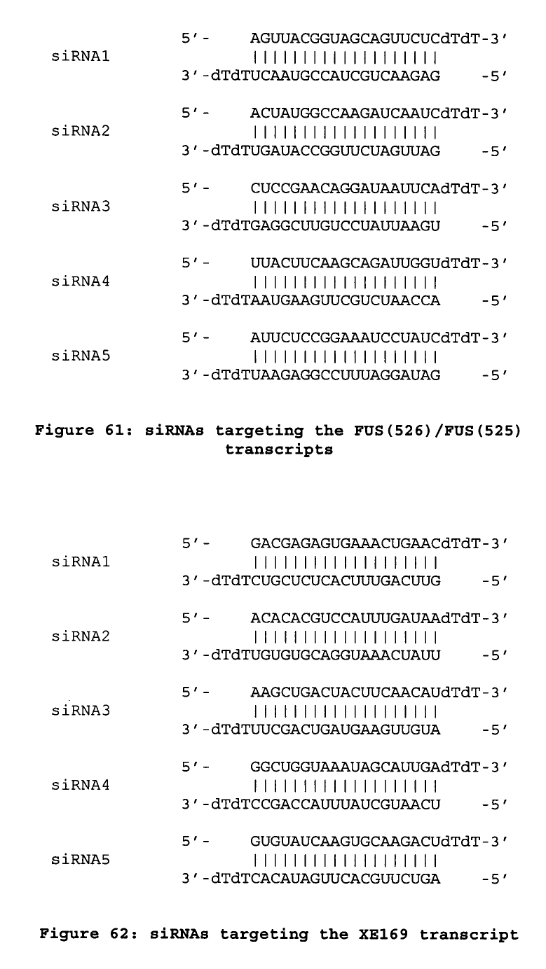

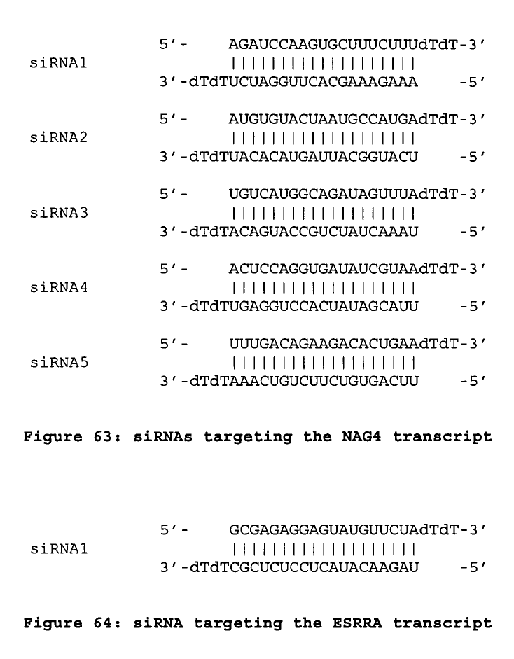

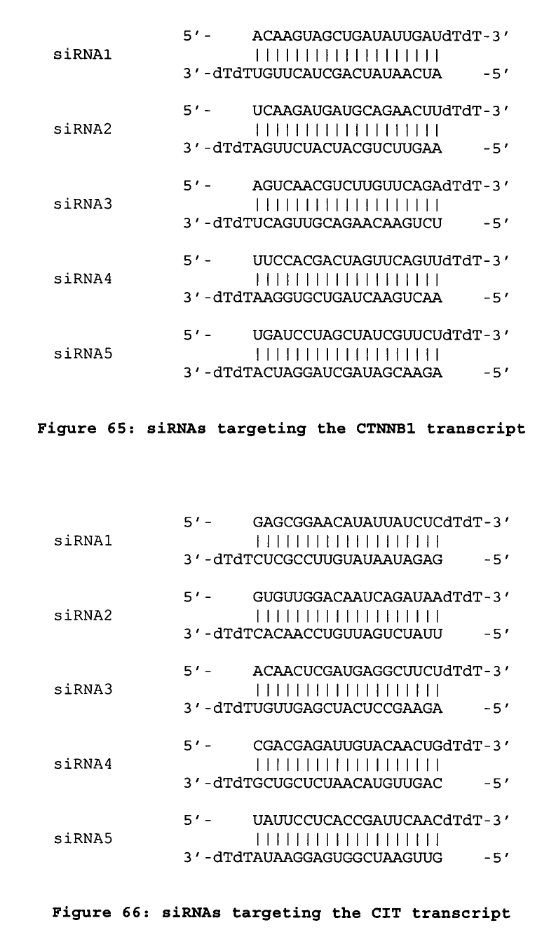

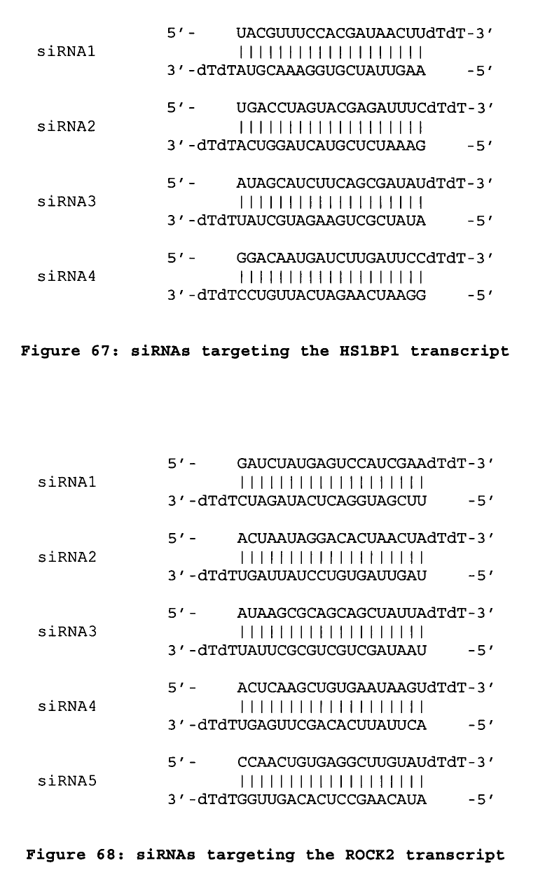

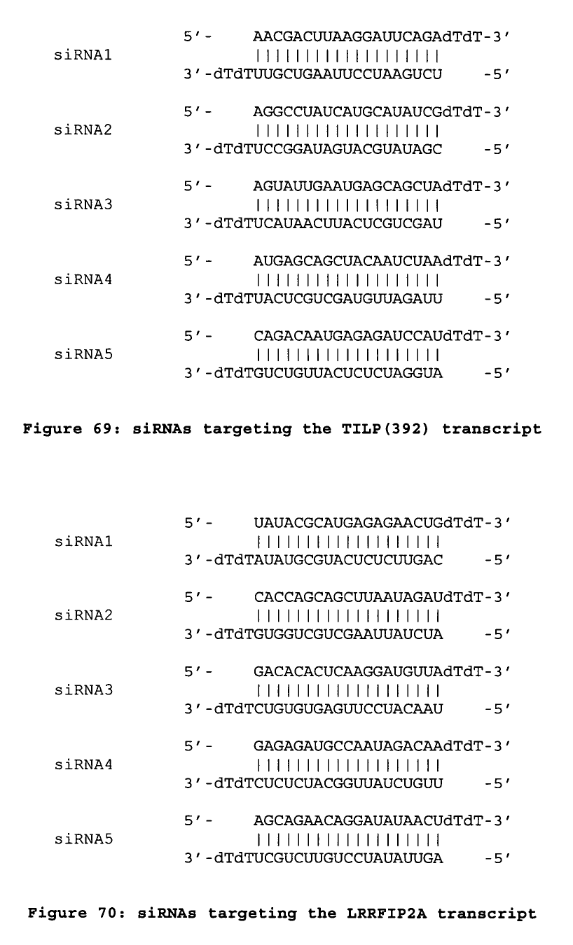

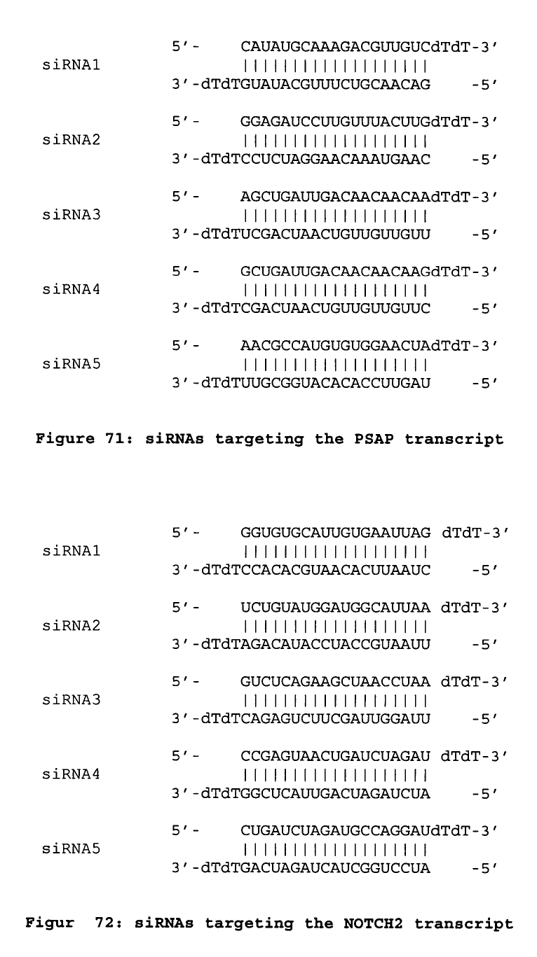

In another embodiment, double-stranded small interfering RNA (siRNA) compounds specific to nucleic acids encoding one or more interacting protein members of a protein complex identified in the present invention are administered to cells or tissue in vitro or in a patient to be therapeutically or prophylactically treated. FIGS. 1-72 depict the structures of siRNA compounds designed to reduce the expression of specific proteins that comprise the protein complexes of the present invention.

As is generally known in the art now, siRNA compounds are RNA duplexes comprising two complementary single-stranded RNAs of 21 nucleotides that form 19 base pairs and possess 3′ overhangs of two nucleotides. See Elbashir et al., Nature 411:494-498 (2001); and PCT Publication Nos. WO 00/44895; WO 01/36646; WO 99/32619; WO 00/01846; WO 01/29058; WO 99/07409; and WO 00/44914. When appropriately targeted via its nucleotide sequence to a specific mRNA in cells, an siRNA can specifically suppress gene expression through a process known as RNA interference (RNAi). See e.g., Zamore & Aronin, Nature Medicine, 9:266-267 (2003). siRNAs can reduce the cellular level of specific mRNAs, and decrease the level of proteins coded by such mRNAs. siRNAs utilize sequence complementarity to target an mRNA for destruction, and are sequence-specific. Thus, they can be highly target-specific, and in mammals have been shown to target mRNAs encoded by different alleles of the same gene. Because of this precision, side effects typically associated with traditional drugs can be reduced or eliminated. In addition, they are relatively stable, and like antisense and ribozyme molecules, they can also be modified to achieve improved pharmaceutical characteristics, such as increased stability, deliverability, and ease of manufacture. Moreover, because siRNA molecules take advantage of a natural cellular pathway, i.e., RNA interference, they are highly efficient in destroying targeted mRNA molecules. As a result, it is relatively easy to achieve a therapeutically effective concentration of an siRNA compound in patients. Thus, siRNAs are a promising new class of drugs being actively developed by pharmaceutical companies.

Indeed, in vivo inhibition of specific gene expression by RNAi has been achieved in various organisms including mammals. For example, Song et al., Nature Medicine, 9:347-351 (2003) discloses that intravenous injection of Fas siRNA compounds into laboratory mice with autoimmune hepatitis specifically reduced Fas mRNA levels and expression of Fas protein in mouse liver cells. The gene silencing effect persisted without diminution for 10 days after the intravenous injection. The injected siRNA was effective in protecting the mice from liver failure and fibrosis. Song et al., Nature Medicine, 9:347-351 (2003). Several other approaches for delivery of siRNA into animals have also proved to be successful. See e.g., McCaffery et al., Nature, 418:38-39 (2002); Lewis et al., Nature Genetics, 32:107-108 (2002); and Xia et al., Nature Biotech., 20:1006-1010 (2002).

The siRNA compounds provided according to the present invention can be synthesized using conventional RNA synthesis methods. For example, they can be chemically synthesized using appropriately protected ribonucleoside phosphoramidites and a conventional DNA/RNA synthesizer. Various applicable methods for RNA synthesis are disclosed in, e.g., Usman et al., J. Am. Chem. Soc., 109:7845-7854 (1987) and Scaringe et al., Nucleic Acids Res., 18:5433-5441 (1990). Custom siRNA synthesis services are available from commercial vendors such as Ambion (Austin, Tex., USA), Dharmacon Research (Lafayette, Colo., USA), Pierce Chemical (Rockford, Ill., USA), ChemGenes (Ashland, Mass., USA), Proligo (Hamburg, Germany), and Cruachem (Glasgow, UK).

The siRNA compounds can also be various modified equivalents of the siRNA structures. As used herein, “modified equivalent” means a modified form of a particular siRNA compound having the same target-specificity (i.e., recognizing the same mRNA molecules that complement the unmodified particular siRNA compound). Thus, a modified equivalent of an unmodified siRNA compound can have modified ribonucleotides, that is, ribonucleotides that contain a modification in the chemical structure of an unmodified nucleotide base, sugar and/or phosphate (or phospodiester linkage). As is known in the art, an “unmodified ribonucleotide” has one of the bases adenine, cytosine, guanine, and uracil joined to the 1′ carbon of beta-D-ribo-furanose.

Preferably, modified siRNA compounds contain modified backbones or non-natural internucleoside linkages, e.g., modified phosphorous-containing backbones and non-phosphorous backbones such as morpholino backbones; siloxane, sulfide, sulfoxide, sulfone, sulfonate, sulfonamide, and sulfamate backbones; formacetyl and thioformacetyl backbones; alkene-containing backbones; methyleneimino and methylenehydrazino backbones; amide backbones, and the like.

Examples of modified phosphorous-containing backbones include, but are not limited to phosphorothioates, phosphorodithioates, chiral phosphorothioates, phosphotriesters, aminoalkylphosphotriesters, alkyl phosphonates, thionoalkylphosphonates, phosphinates, phosphoramidates, thionophosphoramidates, thionoalkylphosphotriesters, and boranophosphates and various salt forms thereof. See e.g., U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Examples of the non-phosphorous containing backbones described above are disclosed in, e.g., U.S. Pat. Nos. 5,034,506; 5,185,444; 5,214,134; 5,216,141; 5,235,033; 5,264,562; 5,264,564; 5,405,938; 5,434,257; 5,470,967; 5,489,677; 5,541,307; 5,561,225; 5,596,086; 5,610,289; 5,602,240; 5,608,046; 5,610,289; 5,618,704; 5,623,070; 5,663,312; 5,677,437; and 5,677,439, each of which is herein incorporated by reference.

Modified forms of siRNA compounds can also contain modified nucleosides (nucleoside analogs), i.e., modified purine or pyrimidine bases, e.g., 5-substituted pyrimidines, 6-azapyrimidines, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), 2-thiouridine, 4-thiouridine, 5-(carboxyhydroxymethyl)uridine, 5′-carboxymethylaminomethyl-2-thiouridine, 5-carboxymethylaminomethyluridine, 5-methoxyaminomethyl-2-thiouridine, 5-methylaminomethyluridine, 5-methylcarbonyhnethyluridine, 5-methyloxyuridine, 5-methyl-2-thiouridine, 4-acetylcytidine, 3-methylcytidine, propyne, quesosine, wybutosine, wybutoxosine, beta-D-galactosylqueosine, N-2, N-6 and O-substituted purines, inosine, 1-methyladenosine, 1-methylinosine, 2,2-dimethylguanosine, 2-methyladenosine, 2-methylguanosine, N6-methyladenosine, 7-methylguanosine, 2-methylthio-N-6-isopentenyladenosine, beta-D-mannosylqueosine, uridine-5-oxyacetic acid, 2-thiocytidine, threonine derivatives, and the like. See e.g., U.S. Pat. Nos. 3,687,808; 4,845,205; 5,130,302; 5,175,273; 5,367,066; 5,432,272; 5,459,255; 5,484,908; 5,502,177; 5,525,711; 5,587,469; 5,594,121; 5,596,091; 5,681,941; and 5,750,692, PCT Publication No. WO 92/07065; PCT Publication No. WO 93/15187; and Limbach et al., Nucleic Acids Res., 22:2183 (1994), each of which is incorporated herein by reference in its entirety.

In addition, modified siRNA compounds can also have substituted or modified sugar moieties, e.g., 2′-O-methoxyethyl sugar moieties. See e.g., U.S. Pat. Nos. 4,981,957; 5,118,800; 5,319,080; 5,393,878; 5,446,137; 5,466,786; 5,514,785; 5,567,811; 5,576,427; 5,591,722; 5,610,300; 5,627,0531 5,639,873; 5,646,265; 5,658,873; 5,670,633; and 5,700,920, each of which is herein incorporated by reference.

Modified siRNA compounds may be synthesized by the methods disclosed in, e.g., U.S. Pat. No. 5,652,094; International Publication Nos. WO 91/03162; WO 92/07065 and WO 93/15187; European Patent Application No. 92110298.4; Perrault et al., Nature, 344:565 (1990); Pieken et al., Science, 253:314 (1991); and Usman and Cedergren, Trends in Biochem. Sci., 17:334 (1992).

Preferably, the 3′ overhangs of the siRNAs of the present invention are modified to provide resistance to cellular nucleases. In one embodiment the 3′ overhangs comprise 2′-deoxyribonucleotides. In preferred embodiments (depicted in FIGS. 1-72) these 3′ overhangs comprise a dinucleotide made of two 2′-deoxythymine residues (i.e., dTdT) linked by a 5′-3′ phosphodiester linkage.

siRNA compounds may be administered to mammals by various methods through different routes. For example, they can be administered by intravenous injection. See Song et al., Nature Medicine, 9:347-351 (2003). They can also be delivered directly to a particular organ or tissue by any suitable localized administration methods. Several other approaches for delivery of siRNA into animals have also proved to be successful. See e.g., McCaffery et al., Nature, 418:38-39 (2002); Lewis et al., Nature Genetics, 32:107-108 (2002); and Xia et al., Nature Biotech., 20:1006-1010 (2002). Alternatively, they may be delivered encapsulated in liposomes, by iontophoresis, or by incorporation into other vehicles such as hydrogels, cyclodextrins, biodegradable nanocapsules, and bioadhesive microspheres.

In addition, they may also be delivered by a gene therapy approach, using a DNA vector from which siRNA compounds in, e.g., small hairpin form (shRNA), can be transcribed directly. Recent studies have demonstrated that while double-stranded siRNAs are very effective at mediating RNAi, short, single-stranded, hairpin-shaped RNAs can also mediate RNAi, presumably because they fold into intramolecular duplexes that are processed into double-stranded siRNAs by cellular enzymes. Sui et al., Proc. Natl. Acad. Sci. U.S.A., 99:5515-5520 (2002); Yu et al., Proc. Natl. Acad. Sci. U.S.A., 99:6047-6052 (2002); and Paul et al., Nature Biotech., 20:505-508 (2002)). This discovery has significant and far-reaching implications, since the production of such shRNAs can be readily achieved in vivo by transfecting cells or tissues with DNA vectors bearing short inverted repeats separated by a small number of (e.g., 3 to 9) nucleotides that direct the transcription of such small hairpin RNAs. Additionally, if mechanisms are included to direct the integration of the transcription cassette into the host cell genome, or to ensure the stability of the transcription vector, the RNAi caused by the encoded shRNAs, can be made stable and heritable. Not only have such techniques been used to “knock down” the expression of specific genes in mammalian cells, but they have now been successfully employed to knock down the expression of exogenously expressed transgenes, as well as endogenous genes in the brain and liver of living mice. See generally Hannon, Nature. 418:244-251 (2002) and Shi, Trends Genet., 19:9-12 (2003); see also Xia et al., Nature Biotech., 20:1006-1010 (2002).

Additional siRNA compounds targeted at different sites of the nucleic acids encoding one or more interacting protein members of a protein complex identified in the present invention may also be designed and synthesized according to general guidelines provided herein and generally known to skilled artisans. See e.g., Elbashir, et al. (Nature 411: 494-498 (2001). For example, guidelines have been compiled into “The siRNA User Guide” which is available at the website of The Rockefeller University, New York, N.Y.

Additionally, to assist in the design of siRNAs for the efficient RNAi-mediated silencing of any target gene, several siRNA supply companies maintain web-based design tools that utilize these general guidelines for “picking” siRNAs when presented with the mRNA or coding DNA sequence of the target gene. Examples of such tools can be found at the web sites of Dharmacon, Inc. (Lafayette, Colo.), Ambion, Inc. (Austin, Tex.), and Qiagen, Inc. (Valencia, Calif.), among others. Generally speaking, when provided with an mRNA or coding DNA sequence, these design tools scan the sequence for potential siRNA targets, using several distinct criteria. For example, the design tools may scan for an open reading frame and limit further scanning to that region of sequence. They may then scan for a particular dinucleotide, the most desirable of which being AA, or alternatively CA, GA or TA. Upon finding one of these dinucleotides, they will then examine the dinucleotide and the 19 nucleotides immediately 3′ of it for G/C content, nucleotide triplets (esp. GGG & CCC), and, using a BLAST algorithm search, for whether or not the 19 nucleotide sequence is unique to a specific target gene in the human genome. The features that make for an “ideal” target sequence are: (1) a 5′-most dinucleotide sequence of AA, or, less preferably, CA, GA or TA; (2) a G/C content of approximately 30-50%; (3) lack of trinucleotide repeats, especially GGG and CCC, and (4) being unique to the target gene (i.e., sequences that share no significant homology with genes other than the one being targeted), so that other genes are not inadvertently targeted by the same siRNA designed for this particular target sequence. Another criteria to be considered is whether or not the target sequence includes a known polymorphic site. If so, siRNAs designed to target one particular allele may not effectively target another allele, since single base mismatches between the target sequence and its complementary strand in a given siRNA can greatly reduce the effectiveness of RNAi induced by that siRNA. Given that target sequence and such design tools and design criteria, an ordinarily skilled artisan apprised of the present disclosure should be able to design and synthesized additional siRNA compounds useful in reducing the mRNA level and therefore protein level of one or more interacting protein members of a protein complex identified in the present invention.

### 6.2.3. Antisense Therapy

In another embodiment, antisense compounds specific to nucleic acids encoding one or more interacting protein members of a protein complex identified in the present invention are administered to cells or tissue in vitro or in a patient to be therapeutically or prophylactically treated. The antisense compounds should specifically inhibit the expression of the one or more interacting protein members. Examples of antisense compounds specific to nucleic acids encoding individual proteins in the tables above are provided in SEQ ID NOs:11-223.

As is known in the art, antisense drugs generally act by hybridizing to a particular target nucleic acid thus blocking gene expression. Methods for designing antisense compounds and using such compounds in treating diseases are well known and well developed in the art. For example, the antisense drug Vitravene® (fomivirsen), a 21-base long oligonucleotide, has been successfully developed and marketed by Isis Pharmaceuticals, Inc. for treating cytomegalovirus (CMV)-induced retinitis.

Any methods for designing and making antisense compounds may be used for the purpose of the present invention. See generally, Sanghvi et al., eds., Antisense Research and Applications, CRC Press, Boca Raton, 1993. Typically, antisense compounds are oligonucleotides designed based on the nucleotide sequence of the mRNA or gene of one or more target proteins, e.g., the interacting protein members of a particular protein complex of the present invention. In particular, antisense compounds can be designed to specifically hybridize to a particular region of the gene sequence or mRNA of one or more of the interacting protein members to modulate (increase or decrease) replication, transcription, or translation. As used herein, the term “specifically hybridize” or paraphrases thereof means a sufficient degree of complementarity or pairing between an antisense oligo and a target DNA or mRNA such that stable and specific binding occurs therebetween. In particular, 100% complementary or pairing is not required. Specific hybridization takes place when sufficient hybridization occurs between the antisense compound and its intended target nucleic acids in the substantial absence of non-specific binding of the antisense compound to non-target sequences under predetermined conditions, e.g., for purposes of in vivo treatment, preferably under physiological conditions. Preferably, specific hybridization results in the interference with normal expression of the target DNA or mRNA.

For example, antisense oligonucleotides can be designed to specifically hybridize to target genes, in regions critical for regulation of transcription; to pre-mRNAs, in regions critical for correct splicing of nascent transcripts; and to mature mRNAs, in regions critical for translation initiation or mRNA stability and localization.

As is generally known in the art, commonly used oligonucleotides are oligomers or polymers of ribonucleotides or deoxyribonucleotides, that are composed of a naturally-occurring nitrogenous base, a sugar (ribose or deoxyribose) and a phosphate group. In nature, the nucleotides are linked together by phosphodiester bonds between the 3′ and 5′ positions of neighboring sugar moieties. However, it is noted that the term “oligonucleotides” also encompasses various non-naturally occurring mimetics and derivatives, i.e., modified forms, of naturally occurring oligonucleotides as described below. Typically an antisense compound of the present invention is an oligonucleotide having from about 6 to about 200, and preferably from about 8 to about 30 nucleoside bases.

The antisense compounds preferably contain modified backbones or non-natural internucleoside linkages, including but not limited to, modified phosphorous-containing backbones and non-phosphorous backbones such as morpholino backbones; siloxane, sulfide, sulfoxide, sulfone, sulfonate, sulfonamide, and sulfamate backbones; formacetyl and thioformacetyl backbones; alkene-containing backbones; methyleneimino and methylenehydrazino backbones; amide backbones, and the like.

Examples of modified phosphorous-containing backbones include, but are not limited to phosphorothioates, phosphorodithioates, chiral phosphorothioates, phosphotriesters, aminoalkylphosphotriesters, alkyl phosphonates, thionoalkylphosphonates, phosphinates, phosphoramidates, thionophosphoramidates, thionoalkylphosphotriesters, and boranophosphates and various salt forms thereof. See e.g., U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Examples of the non-phosphorous containing backbones described above are disclosed in, e.g., U.S. Pat. Nos. 5,034,506; 5,185,444; 5,214,134; 5,216,141; 5,235,033; 5,264,562; 5,264,564; 5,405,938; 5,434,257; 5,470,967; 5,489,677; 5,541,307; 5,561,225; 5,596,086; 5,610,289; 5,602,240; 5,608,046; 5,610,289; 5,618,704; 5,623,070; 5,663,312; 5,677,437; and 5,677,439, each of which is herein incorporated by reference.

Another useful modified oligonucleotide is peptide nucleic acid (PNA), in which the sugar-backbone of an oligonucleotide is replaced with an amide containing backbone, e.g., an aminoethylglycine backbone. See U.S. Pat. Nos. 5,539,082 and 5,714,331; and Nielsen et al., Science, 254, 1497-1500 (1991), all of which are incorporated herein by reference. PNA antisense compounds are resistant to RNase H digestion and thus exhibit longer half-life. In addition, various modifications may be made in PNA backbones to impart desirable drug profiles such as better stability, increased drug uptake, higher affinity to target nucleic acid, etc.

Alternatively, the antisense compounds are oligonucleotides containing modified nucleosides, i.e., modified purine or pyrimidine bases, e.g., 5-substituted pyrimidines, 6-azapyrimidines, and N-2, N-6 and O-substituted purines, and the like. See e.g., U.S. Pat. Nos. 3,687,808; 4,845,205; 5,130,302; 5,175,273; 5,367,066; 5,432,272; 5,459,255; 5,484,908; 5,502,177; 5,525,711; 5,587,469; 5,594,121; 5,596,091; 5,681,941; and 5,750,692, each of which is incorporated herein by reference in its entirety.

In addition, oligonucleotides with substituted or modified sugar moieties may also be used. For example, an antisense compound may have one or more 2′-O-methoxyethyl sugar moieties. See e.g., U.S. Pat. Nos. 4,981,957; 5,118,800; 5,319,080; 5,393,878; 5,446,137; 5,466,786; 5,514,785; 5,567,811; 5,576,427; 5,591,722; 5,610,300; 5,627,0531 5,639,873; 5,646,265; 5,658,873; 5,670,633; and 5,700,920, each of which is herein incorporated by reference.

Other types of oligonucleotide modifications are also useful including linking an oligonucleotide to a lipid, phospholipid or cholesterol moiety, cholic acid, thioether, aliphatic chain, polyamine, polyethylene glycol (PEG), or a protein or peptide. The modified oligonucleotides may exhibit increased uptake into cells, and improved stability, i.e., resistance to nuclease digestion and other biodegradations. See e.g., U.S. Pat. No. 4,522,811; Burnham, Am. J. Hosp. Pharm., 15:210-218 (1994).

Antisense compounds can be synthesized using any suitable methods known in the art. In fact, antisense compounds may be custom made by commercial suppliers. Alternatively, antisense compounds may be prepared using DNA synthesizers available commercially from various vendors, e.g., Applied Biosystems Group of Norwalk, Conn.

The antisense compounds can be formulated into a pharmaceutical composition with suitable carriers and administered into cells or tissue in vitro or in a patient using any suitable route of administration. Alternatively, the antisense compounds may also be used in a “gene-therapy” approach. That is, the oligonucleotide is subcloned into a suitable vector and transformed into human cells. The antisense oligonucleotide is then produced in vivo through transcription. Methods for gene therapy are disclosed in Section 6.3.2 below.

### 6.2.4. Ribozyme Therapy

In another embodiment, an enzymatic RNA or ribozyme is designed to target the nucleic acids encoding one or more of the interacting protein members of the protein complexes of the present invention. Ribozymes are RNA molecules possessing enzymatic activity. One class of ribozymes is capable of repeatedly cleaving other separate RNA molecules into two or more pieces in a nucleotide base sequence specific manner. See Kim et al., Proc. Natl. Acad. of Sci. USA, 84:8788 (1987); Haseloff and Gerlach, Nature, 334:585 (1988); and Jefferies et al., Nucleic Acid Res., 17:1371 (1989). Such ribozymes typically have two functional domains: a catalytic domain and a binding sequence that guides the binding of ribozymes to a target RNA through complementary base-pairing. Once a specifically-designed ribozyme is bound to a target mRNA, it enzymatically cleaves the target mRNA, typically reducing its stability and destroying its ability to direct translation of an encoded protein. After a ribozyme has cleaved its RNA target, it is released from that target RNA and thereafter can bind and cleave another target. That is, a single ribozyme molecule can repeatedly bind and cleave new targets. Therefore, one advantage of ribozyme treatment is that a lower amount of exogenous RNA is required as compared to conventional antisense therapies. In addition, ribozymes exhibit less affinity to mRNA targets than DNA-based antisense oligonucleotides, and therefore are less prone to bind to unintended targets.

In accordance with the present invention, a ribozyme may target any portion of the mRNA encoding one or more interacting protein members of the protein complexes formed by the interactions described in the tables. Methods for selecting a ribozyme target sequence and designing and making ribozymes are generally known in the art. See e.g., U.S. Pat. Nos. 4,987,071; 5,496,698; 5,525,468; 5,631,359; 5,646,020; 5,672,511; and 6,140,491, each of which is incorporated herein by reference in its entirety. For example, suitable ribozymes may be designed in various configurations such as hammerhead motifs, hairpin motifs, hepatitis delta virus motifs, group I intron motifs, or RNase P RNA motifs. See e.g., U.S. Pat. Nos. 4,987,071; 5,496,698; 5,525,468; 5,631,359; 5,646,020; 5,672,511; and 6,140,491; Rossi et al., AIDS Res. Human Retroviruses 8:183 (1992); Hampel and Tritz, Biochemistry 28:4929 (1989); Hampel et al., Nucleic Acids Res., 18:299 (1990); Perrotta and Been, Biochemistry 31:16 (1992); and Guerrier-Takada et al., Cell, 35:849 (1983).

Ribozymes can be synthesized by the same methods used for normal RNA synthesis. For example, such methods are disclosed in Usman et al., J. Am. Chem. Soc., 109:7845-7854 (1987) and Scaringe et al., Nucleic Acids Res., 18:5433-5441 (1990). Modified ribozymes may be synthesized by the methods disclosed in, e.g., U.S. Pat. No. 5,652,094; International Publication Nos. WO 91/03162; WO 92/07065 and WO 93/15187; European Patent Application No. 92110298.4; Perrault et al., Nature, 344:565 (1990); Pieken et al., Science, 253:314 (1991); and Usman and Cedergren, Trends in Biochem. Sci., 17:334 (1992).

Ribozymes of the present invention may be administered to cells by any known methods, e.g., disclosed in International Publication No. WO 94/02595. For example, they can be administered directly to cells or tissue in vitro or in a patient through any suitable route, e.g., intravenous injection. Alternatively, they may be delivered encapsulated in liposomes, by iontophoresis, or by incorporation into other vehicles such as hydrogels, cyclodextrins, biodegradable nanocapsules, and bioadhesive microspheres. In addition, they may also be delivered by a gene therapy approach, using a DNA vector from which the ribozyme RNA can be transcribed directly. Gene therapy methods are disclosed in detail below in Section 6.3.2.

### 6.2.5. Other Methods

The in-patient concentrations and activities of the protein complexes and interacting proteins of the present invention may also be altered by other methods. For example, compounds identified in accordance with the methods described in Section 5 that are capable of interfering with or dissociating protein-protein interactions between the interacting protein members of a protein complex may be administered to cells or tissue in vitro or in a patient. Compounds identified in in vitro binding assays described in Section 5.2 that bind to the protein complexes of the present invention, or the interacting members thereof, may also be used in the treatment. Compounds identified in in vitro binding assays described in Section 5.2 that bind to the protein complexes of the present invention, or the interacting members thereof, may also be used in the treatment.

In addition, potentially useful agents also include incomplete proteins, i.e., fragments of the interacting protein members that are capable of binding to their respective binding partners in a protein complex but are defective with respect to their normal cellular functions. For example, binding domains of the interacting member proteins of a protein complex may be used as competitive inhibitors of the activities of the protein complex. As will be apparent to skilled artisans, derivatives or homologues of the binding domains may also be used. Binding domains can be easily identified using molecular biology techniques, e.g., mutagenesis in combination with yeast two-hybrid assays. Preferably, the protein fragment used is a fragment of an interacting protein member having a length of less than 90%, 80%, more preferably less than 75%, 65%, 50%, or less than 40% of the full length of the protein member. Examples of protein fragments of the proteins in the tables above that are potentially useful agents are provided by SEQ ID NOs:224-728.

In one embodiment, a fragment of a protein identified in the tables above is administered. In a specific embodiment, one or more of the interaction domains of a protein identified in the tables, within the regions listed in the tables, is administered to cells or tissue in vitro, or are administered to a patient in need of such treatment. For example, suitable protein fragments can include polypeptides having a contiguous span of 4, 5, 6, 7, 8, 9, 10, 11, 12, 13, 14, 15, 18, 20 or 25, preferably from 4 to 30, 40 or 50 amino acids or more of the sequence of a first protein identified in the tables, that are capable of interacting with a second protein described in the tables. Also, suitable protein fragments can include peptides capable of binding one or more of the proteins described in the tables, and having an amino acid sequence of from 4 to 30 amino acids that is at least 75%, 80%, 82%, 85%, 87%, 90%, 95% or more identical to a contiguous span of amino acids of a protein described in the tables. Alternatively, a polypeptide capable of interacting with a first protein of an interacting pair of proteins of the present invention, and having a contiguous span of 4, 5, 6, 7, 8, 9, 10, 11, 12, 13, 14, 15, 18, 20 or 25, preferably from 4 to 30, 40 or 50 or more amino acids of the amino acid sequence of a second protein of the same interacting pair of proteins, may be administered. Also, other examples of suitable compounds include a peptide capable of binding a first interacting partner of a pair of interacting proteins of the present invention and having an amino acid sequence of from 4 to 30, 40, 50 or more amino acids that is at least 75%, 80%, 82%, 85%, 87%, 90%, 92%, 95% or more identical to a contiguous span of amino acids from a second interacting partner of a pair of interacting proteins of the present invention. In addition, the administered compounds can be an antibody or antibody fragment, preferably a single-chain antibody immunoreactive with any of the proteins listed in the tables, or a protein complex of the present invention.

The protein fragments suitable as competitive inhibitors can be delivered into cells by direct cell internalization, receptor mediated endocytosis, or via a “transporter.” It is noted that when the target proteins or protein complexes to be modulated reside inside cells, the compound administered to cells in vitro or in vivo in the method of the present invention preferably is delivered into the cells in order to achieve optimal results. Thus, preferably, the compound to be delivered is associated with a transporter capable of increasing the uptake of the compound by cells harboring the target protein or protein complex. As used herein, the term “transporter” refers to an entity (e.g., a compound or a composition or a physical structure formed from multiple copies of a compound or multiple different compounds) that is capable of facilitating the uptake of a compound of the present invention by animal cells, particularly human cells. That is, the cell uptake of a compound of the present invention in the presence of a “transporter” is at least 50% higher than the cell uptake of the compound in the absence of the “transporter.” Preferably, a “transporter” is selected such that the cell uptake of a compound of the present invention in the presence of a “transporter” is at least 75% higher, preferably at least 100% or 200% higher, and more preferably at least 300%, 400% or 500% higher than the cell uptake of the compound in the absence of the “transporter.” Methods of assaying cell uptake of a compound should be apparent to skilled artisans. For example, the compound to be delivered can be labeled with a radioactive isotope or another detectable marker (e.g., a fluorescence marker), and added to cultured cells in the presence or absence of a transporter, and incubated for a time period sufficient to allow maximal uptake. Cells can then be separated from the culture medium and the detectable signal (e.g., radioactivity) caused by the compound inside the cells can be measured. The result obtained in the presence of a transporter can be compared to that obtained in the absence of a transporter.

Many molecules and structures known in the art can be used as “transporters.” In one embodiment, a penetratin is used as a transporter. For example, the homeodomain of Antennapedia, a Drosophila transcription factor, can be used as a transporter to deliver a compound of the present invention. Indeed, any suitable member of the penetratin class of peptides can be used to carry a compound of the present invention into cells. Penetratins are disclosed in, e.g., Derossi et al., Trends Cell Biol., 8:84-87 (1998), which is incorporated herein by reference. Penetratins transport molecules attached thereto across cytoplasmic membranes or nuclear membranes efficiently, in a receptor-independent, energy-independent, and cell type-independent manner. Methods for using a penetratin as a carrier to deliver oligonucleotides and polypeptides are also disclosed in U.S. Pat. No. 6,080,724; Pooga et al., Nat. Biotech., 16:857 (1998); and Schutze et al., J. Immunol., 157:650 (1996), all of which are incorporated herein by reference. U.S. Pat. No. 6,080,724 defines the minimal requirements for a penetratin peptide as a peptide of 16 amino acids with 6 to 10 of which being hydrophobic. The amino acid at position 6 counting from either the N- or C-terminus is tryptophan, while the amino acids at positions 3 and 5 counting from either the N- or C-terminus are not both valine. Preferably, the helix 3 of the homeodomain of Drosophila Antennapedia is used as a transporter. More preferably, a peptide having a sequence of amino acid residues 43-58 of the homeodomain Antp is employed as a transporter. In addition, other naturally occurring homologs of the helix 3 of the homeodomain of Drosophila Antennapedia can be used. For example, homeodomains of Fushi-tarazu and Engrailed have been shown to be capable of transporting peptides into cells. See Han et al., Mol. Cells, 10:728-32 (2000). As used herein, the term “penetratin” also encompasses peptoid analogs of the penetratin peptides. Typically, the penetratin peptides and peptoid analogs thereof are covalently linked to a compound to be delivered into cells thus increasing the cellular uptake of the compound.

In another embodiment, the HIV-1 tat protein or a derivative thereof is used as a “transporter” covalently linked to a compound according to the present invention. The use of HIV-1 tat protein and derivatives thereof to deliver macromolecules into cells has been known in the art. See Green and Loewenstein, Cell, 55:1179 (1988); Frankel and Pabo, Cell, 55:1189 (1988); Vives et al., J. Biol. Chem., 272:16010-16017 (1997); Schwarze et al., Science, 285:1569-1572 (1999). It is known that the sequence responsible for cellular uptake consists of the highly basic region, amino acid residues 49-57. See e.g., Vives et al., J. Biol. Chem., 272:16010-16017 (1997); Wender et al., Proc. Nat'l Acad. Sci. USA, 97:13003-13008 (2000). The basic domain is believed to target the lipid bilayer component of cell membranes. It causes a covalently linked protein or nucleic acid to cross cell membrane rapidly in a cell type-independent manner. Proteins ranging in size from 15 to 120 kD have been delivered with this technology into a variety of cell types both in vitro and in vivo. See Schwarze et al., Science, 285:1569-1572 (1999). Any HIV tat-derived peptides or peptoid analogs thereof capable of transporting macromolecules such as peptides can be used for purposes of the present invention. For example, any native tat peptides having the highly basic region, amino acid residues 49-57 can be used as a transporter by covalently linking it to the compound to be delivered. In addition, various analogs of the tat peptide of amino acid residues 49-57 can also be useful transporters for purposes of this invention. Examples of various such analogs are disclosed in Wender et al., Proc. Nat'l Acad. Sci. USA, 97:13003-13008 (2000) (which is incorporated herein by reference) including, e.g., d-Tat₄₉₋₅₇, retro-inverso isomers of l- or d-Tat₄₉₋₅₇ (i.e., l-Tat₅₇₋₄₉ and d-Tat₅₇₋₄₉), L-arginine oligomers, D-arginine oligomers, L-lysine oligomers, D-lysine oligomers, L-histine oligomers, D-histine oligomers, L-ornithine oligomers, D-ornithine oligomers, and various homologues, derivatives (e.g., modified forms with conjugates linked to the small peptides) and peptoid analogs thereof. Preferably, arginine oligomers are preferred to the other oligomers, since arginine oligomers are much more efficient in promoting cellular uptake. As used herein, the term “oligomer” means a molecule that includes a covalently linked chain of amino acid residues of the same amino acids having a large enough number of such amino acid residues to confer transporter activities on the molecule. Typically, an oligomer contains at least 6, preferably at least 7, 8, or 9 such amino acid residues. In one embodiment, the transporter is a peptide that includes at least six contiguous amino acid residues that are a combination of two or more of L-arginine, D-arginine, L-lysine, D-lysine, L-histidine, D-histine, L-ornithine, and D-ornithine.

Other useful transporters known in the art include, but are not limited to, short peptide sequences derived from fibroblast growth factor (See Lin et al., J. Biol. Chem., 270:14255-14258 (1998)), Galparan (See Pooga et al., FASEB J 12:67-77 (1998)), and HSV-1 structural protein VP22 (See Elliott and O'Hare, Cell, 88:223-233 (1997)).

As the above-described various transporters are generally peptides, fusion proteins can be conveniently made by recombinant expression to contain a transporter peptide covalently linked by a peptide bond to a competitive protein fragment. Alternatively, conventional methods can be used to chemically synthesize a transporter peptide or a peptide of the present invention or both.

The hybrid peptide can be administered to cells or tissue in vitro or to a patient in a suitable pharmaceutical composition as provided in Section 8.

In addition to peptide-based transporters, various other types of transporters can also be used, including but not limited to cationic liposomes (see Rui et al., J. Am. Chem. Soc., 120:11213-11218 (1998)), dendrimers (Kono et al., Bioconjugate Chem., 10:1115-1121 (1999)), siderophores (Ghosh et al., Chem. Biol., 3:1011-1019 (1996)), etc. In a specific embodiment, the compound according to the present invention is encapsulated into liposomes for delivery into cells.

Additionally, when a compound according to the present invention is a peptide, it can be administered to cells by a gene therapy method. That is, a nucleic acid encoding the peptide can be administered to in vitro cells or to cells in vivo in a human or animal body. Any suitable gene therapy methods may be used for purposes of the present invention. Various gene therapy methods are well known in the art and are described in Section 6.3.2. below. Successes in gene therapy have been reported recently. See e.g., Kay et al., Nature Genet., 24:257-61 (2000); Cavazzana-Calvo et al., Science, 288:669 (2000); and Blaese et al., Science, 270: 475 (1995); Kantoff, et al., J. Exp. Med., 166:219 (1987).

In yet another embodiment, the gene therapy methods discussed in Section 6.3.2 below are used to “knock out” the gene encoding an interacting protein member of a protein complex, or to reduce the gene expression level. For example, the gene may be replaced with a different gene sequence or a non-functional sequence or simply deleted by homologous recombination. In another gene therapy embodiment, the method disclosed in U.S. Pat. No. 5,641,670, which is incorporated herein by reference, may be used to reduce the expression of the genes for the interacting protein members. Essentially, an exogenous DNA having at least a regulatory sequence, an exon and a splice donor site can be introduced into an endogenous gene encoding an interacting protein member by homologous recombination such that the regulatory sequence, the exon and the splice donor site present in the DNA construct become operatively linked to the endogenous gene. As a result, the expression of the endogenous gene is controlled by the newly introduced exogenous regulatory sequence. Therefore, when the exogenous regulatory sequence is a strong gene expression repressor, the expression of the endogenous gene encoding the interacting protein member is reduced or blocked. See U.S. Pat. No. 5,641,670.

### 6.3. Activation of Protein Complex or Interacting Protein Members Thereof

The present invention also provides methods for increasing in cells or tissue in vitro or in a patient the concentration and/or activity of a protein complex, or of an individual protein member thereof, identified in accordance with the present invention. Such methods can be particularly useful in instances where a reduced concentration and/or activity of a protein complex, or a protein member thereof, is associated with a particular disease or disorder to be treated, or where an increased concentration and/or activity of a protein complex, or a protein member thereof, would be beneficial to the improvement of a cellular function or disease state. By increasing the concentration of the protein complex, or a protein member thereof, and/or stimulating the functional activities of the protein complex or a protein member thereof, the disease or disorder may be treated or prevented.

### 6.3.1. Administration of Protein Complex or Protein Members Thereof

Where the concentration or activity of a particular protein complex of the present invention, or any individual protein constituent of a protein complex in cells or tissue in vitro or in a patient is determined to be low or is desired to be increased, the protein complex, or an individual constituent protein of the protein complex may be administered directly to the patient to increase the concentration and/or activity of the protein complex, or the individual constituent protein. For this purpose, protein complexes prepared by any one of the methods described in Section 2.2 may be administered to the patient, preferably in a pharmaceutical composition as described below. Alternatively, one or more individual interacting protein members of the protein complex may also be administered to the patient in need of treatment. For example, one or more of the individual proteins or the interacting pairs of proteins described in the tables may be given to cells or tissue in vitro or to a patient. Proteins isolated or purified from normal individuals or recombinantly produced can all be used in this respect. Preferably, two or more interacting protein members of a protein complex are administered. The proteins or protein complexes may be administered to a patient needing treatment using any of the methods described in Section 8.

### 6.3.2. Gene Therapy

In another embodiment, the concentration and/or activity of a particular protein complex comprising one or more of the interacting pairs of proteins described in the tables or an individual constituent protein of a protein complex of the present invention is increased or restored in patients, tissue or cells by a gene therapy approach. For example, nucleic acids encoding one or more protein members of a protein complex of the present invention, or portions or fragments thereof are introduced into patients, tissue, or cells such that the protein(s) are expressed from the introduced nucleic acids. For these purposes, nucleic acids encoding one or more of the proteins described in the tables, or fragments, homologues or derivatives thereof can be used in the gene therapy in accordance with the present invention. For example, if a disease-causing mutation exists in one of the protein members in cells or tissue in vitro or in a patient, then a nucleic acid encoding a wild-type protein can be introduced into tissue cells of the patient. The exogenous nucleic acid can be used to replace the corresponding endogenous defective gene by, e.g., homologous recombination. See U.S. Pat. No. 6,010,908, which is incorporated herein by reference. Alternatively, if the disease-causing mutation is a recessive mutation, the exogenous nucleic acid is simply used to express a wild-type protein in addition to the endogenous mutant protein. In another approach, the method disclosed in U.S. Pat. No. 6,077,705 may be employed in gene therapy. That is, the patient is administered both a nucleic acid construct encoding a ribozyme and a nucleic acid construct comprising a ribozyme resistant gene encoding a wild type form of the gene product. As a result, undesirable expression of the endogenous gene is inhibited and a desirable wild-type exogenous gene is introduced. In yet another embodiment, if the endogenous gene is of wild-type and the level of expression of the protein encoded thereby is desired to be increased, additional copies of wild-type exogenous genes may be introduced into the patient by gene therapy, or alternatively, a gene activation method such as that disclosed in U.S. Pat. No. 5,641,670 may be used.

Various gene therapy methods are well known in the art. Successes in gene therapy have been reported recently. See e.g., Kay et al., Nature Genet., 24:257-61 (2000); Cavazzana-Calvo et al., Science, 288:669 (2000); and Blaese et al., Science, 270: 475 (1995); Kantoff, et al., J. Exp. Med. 166:219 (1987).

Any suitable gene therapy methods may be used for the purposes of the present invention. Generally, a nucleic acid encoding a desirable protein (e.g., one selected from any of the tables) is incorporated into a suitable expression vector and is operably linked to a promoter in the vector. Suitable promoters include but are not limited to viral transcription promoters derived from adenovirus, simian virus 40 (SV40) (e.g., the early and late promoters of SV40), Rous sarcoma virus (RSV), and cytomegalovirus (CMV) (e.g., CMV immediate-early promoter), human immunodeficiency virus (HIV) (e.g., long terminal repeat (LTR)), vaccinia virus (e.g., 7.5K promoter), and herpes simplex virus (HSV) (e.g., thymidine kinase promoter). Where tissue-specific expression of the exogenous gene is desirable, tissue-specific promoters may be operably linked to the exogenous gene. In addition, selection markers may also be included in the vector for purposes of selecting, in vitro, those cells that contain the exogenous gene. Various selection markers known in the art may be used including, but not limited to, e.g., genes conferring resistance to neomycin, hygromycin, zeocin, and the like.

In one embodiment, the exogenous nucleic acid (gene) is incorporated into a plasmid DNA vector. Many commercially available expression vectors may be useful for the present invention, including, e.g., pCEP4, pcDNAI, pIND, pSecTag2, pVAX1, pcDNA3.1, and pBI-EGFP, and pDisplay.

Various viral vectors may also be used. Typically, in a viral vector, the viral genome is engineered to eliminate the disease-causing capability of the virus, e.g., the ability to replicate in the host cells. The exogenous nucleic acid to be introduced into cells or tissue in vitro or in a patient may be incorporated into the engineered viral genome, e.g., by inserting it into a viral gene that is non-essential to the viral infectivity. Viral vectors are convenient to use as they can be easily introduced into cells, tissues and patients by way of infection. Once in the host cell, the recombinant virus typically is integrated into the genome of the host cell. In rare instances, the recombinant virus may also replicate and remain as extrachromosomal elements.

A large number of retroviral vectors have been developed for gene therapy. These include vectors derived from oncoretroviruses (e.g., MLV), lentiviruses (e.g., HIV and SIV) and other retroviruses. For example, gene therapy vectors have been developed based on murine leukemia virus (See, Cepko, et al., Cell, 37:1053-1062 (1984), Cone and Mulligan, Proc. Natl. Acad. Sci. U.S.A., 81:6349-6353 (1984)), mouse mammary tumor virus (See, Salmons et al., Biochem. Biophys. Res. Commun.,159:1191-1198 (1984)), gibbon ape leukemia virus (See, Miller et al., J Virology, 65:2220-2224 (1991)), HIV, (See Shimada et al., J. Clin. Invest., 88:1043-1047 (1991)), and avian retroviruses (See Cosset et al., J Virology, 64:1070-1078 (1990)). In addition, various retroviral vectors are also described in U.S. Pat. Nos. 6,168,916; 6,140,111; 6,096,534; 5,985,655; 5,911,983; 4,980,286; and 4,868,116, all of which are incorporated herein by reference.

Adeno-associated virus (AAV) vectors have been successfully tested in clinical trials. See e.g., Kay et al., Nature Genet. 24:257-61 (2000). AAV is a naturally occurring defective virus that requires other viruses such as adenoviruses or herpes viruses as helper viruses. See Muzyczka, Curr. Top. Microbiol. Immun., 158:97 (1992). A recombinant AAV virus useful as a gene therapy vector is disclosed in U.S. Pat. No. 6,153,436, which is incorporated herein by reference.

Adenoviral vectors can also be useful for purposes of gene therapy in accordance with the present invention. For example, U.S. Pat. No. 6,001,816 discloses an adenoviral vector, which is used to deliver a leptin gene intravenously to a mammal to treat obesity. Other recombinant adenoviral vectors may also be used, which include those disclosed in U.S. Pat. Nos. 6,171,855; 6,140,087; 6,063,622; 6,033,908; and 5,932,210, and Rosenfeld et al., Science, 252:431-434 (1991); and Rosenfeld et al., Cell, 68:143-155 (1992).

Other useful viral vectors include recombinant hepatitis viral vectors (See, e.g., U.S. Pat. No. 5,981,274), and recombinant entomopox vectors (See, e.g., U.S. Pat. Nos. 5,721,352 and 5,753,258).

Other non-traditional vectors may also be used for purposes of this invention. For example, International Publication No. WO 94/18834 discloses a method of delivering DNA into mammalian cells by conjugating the DNA to be delivered with a polyelectrolyte to form a complex. The complex may be microinjected into or taken up by cells.

The exogenous gene fragment or plasmid DNA vector containing the exogenous gene may also be introduced into cells by way of receptor-mediated endocytosis. See e.g., U.S. Pat. No. 6,090,619; Wu and Wu, J. Biol. Chem., 263:14621 (1988); Curiel et al., Proc. Natl. Acad. Sci. USA, 88:8850 (1991). For example, U.S. Pat. No. 6,083,741 discloses introducing an exogenous nucleic acid into mammalian cells by associating the nucleic acid to a polycation moiety (e.g., poly-L-lysine having 3-100 lysine residues), which is itself coupled to an integrin receptor binding moiety (e.g., a cyclic peptide having the sequence Arg-Gly-Asp).

Alternatively, the exogenous nucleic acid or vectors containing it can also be delivered into cells via amphiphiles. See e.g., U.S. Pat. No. 6,071,890. Typically, the exogenous nucleic acid or a vector containing the nucleic acid forms a complex with the cationic amphiphile. Mammalian cells contacted with the complex can readily take it up.

The exogenous gene can be introduced into cells or tissue in vitro or in a patient for purposes of gene therapy by various methods known in the art. For example, the exogenous gene sequences alone or in a conjugated or complex form described above, or incorporated into viral or DNA vectors, may be administered directly by injection into an appropriate tissue or organ of a patient. Alternatively, catheters or like devices may be used to deliver exogenous gene sequences, complexes, or vectors into a target organ or tissue. Suitable catheters are disclosed in, e.g., U.S. Pat. Nos. 4,186,745; 5,397,307; 5,547,472; 5,674,192; and 6,129,705, all of which are incorporated herein by reference.

In addition, the exogenous gene or vectors containing the gene can be introduced into isolated cells using any known techniques such as calcium phosphate precipitation, microinjection, lipofection, electroporation, biolystics, receptor-mediated endocytosis, and the like. Cells expressing the exogenous gene may be selected and redelivered back to the patient by, e.g., injection or cell transplantation. The appropriate amount of cells delivered to a patient will vary with patient conditions, and desired effect, which can be determined by a skilled artisan. See e.g., U.S. Pat. Nos. 6,054,288; 6,048,524; and 6,048,729. Preferably, the cells used are autologous, i.e., cells obtained from the patient being treated.

### 6.4. Small Organic Compounds

Diseases or disorders in cells or tissue in vitro, or in a patient, associated with the decreased concentration or activity of a protein complex of the present invention, or an individual protein constituent of a protein complex identified in accordance with the present invention, can also be ameliorated by administering to the patient a compound identified by the methods described in Sections 5.3.1.4, 5.2, and Section 5.4, which is capable of modulating the functions or intracellular levels of the protein complex or a constituent protein, e.g., by triggering or initiating, enhancing or stabilizing protein-protein interaction between the interacting protein members of the protein complex, or the mutant forms of such interacting protein members found in the patient.

For example the ATPase activity of DNCL1, in known to be inhibited by erythro-9-[3-(2-hydroxynonyl)] adenine (EHNA) (Schilwa et al., Proc. Natl. Acad. Sci. USA 81:6044-60488 (1984); Ekstrom and Kanje J. Neurochem. 43:1342-1345 (1984); and Penningroth Methods Enzymol. 134:477-487 (1986)). As described above, DNCL1 interacts with survivin/BIRC5, and is therefore implicated in modulating the antiapoptotic activity of survivin/BIRC5. Treatment of patients with EHNA may therefore have desirable effects in certain circumstances, and may be useful for the treatment of inflammatory disorders and diseases, where apoptotic pathways are activated.

Additionally, as described above, CAPN4 has been shown to interact MAPKAP-K3. Alpha-mercaptoacrylate derivatives (exemplified by the compound PD150606) with potent and selective inhibitor actions against calpains, have been identified (Wang et al., Adv. Exp. Med. Biol. 389:95-101 (1996)). In particular, PD150606 has been shown to inhibit calpain activity in intact cells when applied in the low micromolar range (Wang et al., Proc. Natl. Acad. Sci. USA 93:6687-6692 (1996)). Subsequently, inhibition of calpain I has been shown to reduce the development of acute and chronic inflammation in animal models (Cuzzocrea et al., Am. J. Pathol. 157:2065-2079 (2000)). Hence, specific inhibition of CAPN4 by PD 150606, and related compounds, and/or the disruption of the interactions occurring between CAPN4—MAPKAP-K3, may provide a novel therapeutic approach for the treatment of inflammation and inflammatory disorders and diseases.

### 7. Cell and Animal Models

In another aspect of the present invention, cell and animal models are provided in which one or more of the constituent proteins of the interacting pairs of proteins described in the tables, exhibit aberrant function, activity, or concentration when compared with wild type cells and animals (e.g., increased or decreased concentration, altered interactions between protein complex constituents due to mutations in interaction domains, and/or altered distribution or localization of the proteins in organs, tissues, cells, or cellular compartments). Such cell and animal models are useful tools for studying cellular functions and biological processes associated with the proteins identified in the tables. Such cell and animal models are also useful tools for studying disorders and diseases associated with the proteins identified in the tables, and for testing various methods for modulating the cellular functions, and for treating the diseases and disorders, associated with aberrations in these proteins. For example, a cell or animal model may be used to determine if PRAK exhibits aberrant function, activity, or concentration when compared with wild type cells or animals. In another example, a cell or animal model may be used to determine if ERK3 exhibits aberrant function, activity, or concentration when compared with wild type cells or animals.

### 7.1. Cell Models

Cell models having an aberrant form of one or more of the proteins or protein complexes identified in the tables are provided in accordance with the present invention.

The cell models may be established by isolating, from a patient, cells having an aberrant form of one or more of the protein complexes of the present invention. The isolated cells may be cultured in vitro as a primary cell culture. Alternatively, the cells obtained from the primary cell culture or directly from the patient may be immortalized to establish a human cell line. Any methods for constructing immortalized human cell lines may be used in this respect. See generally Yeager and Reddel, Curr. Opini. Biotech., 10:465-469 (1999). For example, the human cells may be immortalized by transfection of plasmids expressing the SV40 early region genes (See e.g., Jha et al., Exp. Cell Res., 245:1-7 (1998)), introduction of the HPV E6 and E7 oncogenes (See e.g., Reznikoff et al., Genes Dev., 8:2227-2240 (1994)), and infection with Epstein-Barr virus (See e.g., Tahara et al., Oncogene, 15:1911-1920 (1997)). Alternatively, the human cells may be immortalized by recombinantly expressing the gene for the human telomerase catalytic subunit hTERT in the human cells. See Bodnar et al., Science, 279:349-352 (1998).

In alternative embodiments, cell models are provided by recombinantly manipulating appropriate host cells. The host cells may be bacteria cells, yeast cells, insect cells, plant cells, animal cells, and the like. Preferably, the cells are derived from mammals, most preferably humans. The host cells may be obtained directly from an individual, or a primary cell culture, or preferably an immortal stable human cell line. In a preferred embodiment, human embryonic stem cells or pluripotent cell lines derived from human stem cells are used as host cells. Methods for obtaining such cells are disclosed in, e.g., Shamblott, et al., Proc. Natl. Acad. Sci. USA, 95:13726-13731 (1998) and Thomson et al., Science, 282:1145-1147 (1998).

In one embodiment, a cell model is provided by recombinantly expressing one or more of the proteins or protein complexes identified in the tables in cells that do not normally express such protein complexes. For example, cells that do not contain a particular protein or protein complex may be engineered to express the protein or protein complex. In a specific embodiment, a particular human protein complex is expressed in non-human cells. The cell model may be prepared by introducing into host cells nucleic acids encoding all interacting protein members required for the formation of a particular protein complex, and expressing the protein members in the host cells. For this purpose, the recombinant expression methods described in Section 2.2 may be used. In addition, the methods for introducing nucleic acids into host cells disclosed in the context of gene therapy in Section 6.3.2 may also be used.

In another embodiment, a cell model over-expressing one or more of the proteins or protein complexes identified in the tables. The cell model may be established by increasing the expression level of one or more of the interacting protein members of the protein complexes. In a specific embodiment, all interacting protein members of a particular protein complex are over-expressed. The over-expression may be achieved by introducing into host cells exogenous nucleic acids encoding the proteins to be over-expressed, and selecting those cells that over-express the proteins. The expression of the exogenous nucleic acids may be transient or, preferably stable. The recombinant expression methods described in Section 2.2, and the methods for introducing nucleic acids into host cells disclosed in the context of gene therapy in Section 6.3.2 may be used. Alternatively, the gene activation method disclosed in U.S. Pat. No. 5,641,670 can be used. Any host cells may be employed for establishing the cell model. Preferably, human cells lacking a protein or protein complex to be over-expressed, or having a normal concentration of the protein or protein complex, are used as host cells. The host cells may be obtained directly from an individual, or a primary cell culture, or preferably a stable immortal human cell line. In a preferred embodiment, human embryonic stem cells or pluripotent cell lines derived from human stem cells are used as host cells. Methods for obtaining such cells are disclosed in, e.g., Shamblott, et al., Proc. Natl. Acad. Sci. USA, 95:13726-13731 (1998), and Thomson et al., Science, 282:1145-1147 (1998).

In yet another embodiment, a cell model expressing an abnormally low level of one or more of the proteins or protein complexes identified in the tables is provided. Typically, the cell model is established by genetically manipulating cells that express a normal and detectable level of a protein or protein complex identified in the tables. Generally the expression level of one or more of the interacting protein members of the protein complex is reduced by recombinant methods. In a specific embodiment, the expression of all interacting protein members of a particular protein complex is reduced. The reduced expression may be achieved by “knocking out” the genes encoding one or more interacting protein members. Alternatively, mutations that can cause reduced expression level (e.g., reduced transcription and/or translation efficiency, and decreased mRNA stability) may also be introduced into the gene by homologous recombination. A gene encoding a ribozyme, antisense, or siRNA compound specific to the mRNA encoding an interacting protein member may also be introduced into the host cells, preferably stably integrated into the genome of the host cells. In addition, a gene encoding an antibody or fragment thereof specific to an interacting protein member may also be introduced into the host cells. The recombinant expression methods described in Sections 2.2, 6.1 and 6.2 can all be used for purposes of manipulating the host cells.

In a specific embodiment, an siRNA compound specific to the mRNA encoding PRAK is introduced into a host cell in order to decrease the expression level of PRAK. In another specific embodiment, an siRNA compound specific to the mRNA encoding ERK3 is introduced into a host cell in order to decrease the expression level of ERK3.

The present invention also contemplates a cell model provided by recombinant DNA techniques that exhibits aberrant interactions between the interacting protein members of a protein complex identified in the present invention. For example, variants of the interacting protein members of a particular protein complex exhibiting altered protein-protein interaction properties and the nucleic acid variants encoding such variant proteins may be obtained by random or site-directed mutagenesis in combination with a protein-protein interaction assay system, particularly the yeast two-hybrid system described in Section 5.3.1. Essentially, the genes encoding one or more interacting protein members of a particular protein complex may be subject to random or site-specific mutagenesis and the mutated gene sequences are used in yeast two-hybrid system to test the protein-protein interaction characteristics of the protein variants encoded by the gene variants. In this manner, variants of the interacting protein members of the protein complex may be identified that exhibit altered protein-protein interaction properties in forming the protein complex, e.g., increased or decreased binding affinity, and the like. The nucleic acid variants encoding such protein variants may be introduced into host cells by the methods described above, preferably into host cells that normally do not express the interacting proteins.

### 7.2. Cell-Based Assays

The cell models of the present invention containing an aberrant form of a protein or protein complex identified in the tables are useful in screening assays for identifying compounds useful in treating diseases and disorders involving intracellular signaling such as inflammation and inflammatory disorders (e.g., asthma, rheumatoid arthritis, juvenile chronic arthritis, myositis, Chron's disease, gastritis, colitis, ulcerative colitis, inflammatory bowel disease, proctitis, pelvic inflammatory disease, systemic lupus erythematosus, rhinitis, conjunctivitis, scleritis, chronic inflammatory polyneuropathy, Tertiary Lyme disease, psoriasis, dermatitis, eczema, etc.). In addition, they may also be used in in vitro pre-clinical assays for testing compounds, such as those identified in the screening assays of the present invention.

For example, cells may be treated with compounds to be tested and assayed for the compound's activity. A variety of parameters relevant to particularly physiological disorders or diseases may be analyzed.

### 7.3. Transgenic Animals

In another aspect of the present invention, transgenic non-human animals are created expressing an aberrant form of one or more of the protein complexes of the present invention. Animals of any species may be used to generate the transgenic animal models, including but not limited to, mice, rats, hamsters, sheep, pigs, rabbits, guinea pigs, preferably non-human primates such as monkeys, chimpanzees, baboons, and the like.

In one embodiment, transgenic animals are made to over-express one or more protein complexes formed from a first protein, which is any one of the proteins described in the tables, or a derivative, fragment or homologue thereof (including the animal counterpart of the first protein, i.e., an orthologue) and a second protein, which is any of the proteins described in the tables that interacts with the first protein, or derivatives, fragments or homologues thereof (including orthologues). Over-expression may be directed in a tissue or cell type that normally expresses animal counterparts of such protein complexes. Consequently, the concentration of the protein complex(es) will be elevated to higher levels than normal. Alternatively, the one or more protein complexes are expressed in tissues or cells that do not normally express such proteins and hence do not normally contain the protein complexes of the present invention. In a specific embodiment, a first protein, which is any one of the proteins described in the tables which is a human protein and a second protein, which is any of the proteins described in the tables that interacts with the first protein and is a human protein, are expressed in the transgenic animals.

To achieve over-expression in transgenic animals, the transgenic animals are made such that they contain and express exogenous, orthologous genes encoding a first protein, which is any of the proteins identified in the tables or a homologue, derivative or mutant form thereof, and one or more second proteins, which are any of the proteins described in the tables that interact with the first protein, or homologues, derivatives or mutant forms thereof. Preferably, the exogenous genes are human genes. Such exogenous genes may be operably linked to a native or non-native promoter, preferably a non-native promoter. For example, an exogenous gene encoding one of the proteins described in the tables may be operably linked to a promoter that is not the native promoter of that protein. If the expression of the exogenous gene is desired to be limited to a particular tissue, an appropriate tissue-specific promoter may be used.

Over-expression may also be achieved by manipulating the native promoter to create mutations that lead to gene over-expression, or by a gene activation method such as that disclosed in U.S. Pat. No. 5,641,670 as described above.

In another embodiment, the transgenic animal expresses an abnormally low concentration of the complex comprising at least one of the interacting pairs of proteins described in the tables. In a specific embodiment, the transgenic animal is a “knockout” animal wherein the endogenous gene encoding the animal orthologue of a first protein, which is any of the proteins described in the tables, and/or an endogenous gene encoding an animal orthologue of a second protein, which is any of the proteins identified in the tables that interacts with the first protein, are knocked out. In a specific embodiment, the expression of the animal orthologues of both the first and second proteins are reduced or knocked out. The reduced expression may be achieved by knocking out the genes encoding one or both interacting protein members, typically by homologous recombination. Alternatively, mutations that can cause reduced expression (e.g., reduced transcription and/or translation efficiency, or decreased mRNA stability) may also be introduced into the endogenous genes by homologous recombination. Genes encoding ribozymes or antisense compounds specific to the mRNAs encoding the interacting protein members may also be introduced into the transgenic animal. In addition, genes encoding antibodies or fragments thereof specific to the interacting protein members may also be introduced into the transgenic animal.

In an alternate embodiment, transgenic animals are made in which the endogenous genes encoding the animal orthologues of any of the proteins described in the tables are replaced with orthologous human genes.

In yet another embodiment, the transgenic animal of this invention expresses specific mutant forms of any of the proteins described in the tables that exhibit aberrant interactions. For this purpose, variants of any of the proteins described in the tables exhibiting altered protein-protein interaction properties, and the nucleic acid variants encoding such variant proteins, may be obtained by random or site-specific mutagenesis in combination with a protein-protein interaction assay system, particularly the yeast two-hybrid system described in Section 5.3.1. For example, variants of PRAK and ERK3 exhibiting increased, decreased or abolished binding affinity to each other may be identified and isolated. The transgenic animal of the present invention may be made to express such protein variants by modifying the endogenous genes. Alternatively, the nucleic acid variants may be introduced exogenously into the transgenic animal genome to express the protein variants therein. In a specific embodiment, the exogenous nucleic acid variants are derived from orthologous human genes and the corresponding endogenous genes are knocked out.

Any techniques known in the art for making transgenic animals may be used for purposes of the present invention. For example, the transgenic animals of the present invention may be provided by methods described in, e.g., Jaenisch, Science, 240:1468-1474 (1988); Capecchi, et al., Science, 244:1288-1291 (1989); Hasty et al., Nature, 350:243 (1991); Shinkai et al., Cell, 68:855 (1992); Mombaerts et al., Cell, 68:869 (1992); Philpott et al., Science, 256:1448 (1992); Snouwaert et al., Science, 257:1083 (1992); Donehower et al., Nature, 356:215 (1992); Hogan et al., Manipulating the Mouse Embryo; A Laboratory Manual, 2^{nd} edition, Cold Spring Harbor Laboratory Press, 1994; and U.S. Pat. Nos. 4,873,191; 5,800,998; 5,891,628, all of which are incorporated herein by reference.

Generally, the founder lines may be established by introducing appropriate exogenous nucleic acids into, or modifying an endogenous gene in, germ lines, embryonic stem cells, embryos, or sperm which are then used in producing a transgenic animal. The gene introduction may be conducted by various methods including those described in Sections 2.2, 6.1 and 6.2. See also, Van der Putten et al., Proc. Natl. Acad. Sci. USA, 82:6148-6152 (1985); Thompson et al., Cell, 56:313-321 (1989); Lo, Mol. Cell. Biol., 3:1803-1814 (1983); Gordon, Trangenic Animals, Intl. Rev. Cytol. 115:171-229 (1989); and Lavitrano et al., Cell, 57:717-723 (1989). In a specific embodiment, the exogenous gene is incorporated into an appropriate vector, such as those described in Sections 2.2 and 6.2, and is transformed into embryonic stem (ES) cells. The transformed ES cells are then injected into a blastocyst. The blastocyst with the transformed ES cells is then implanted into a surrogate mother animal. In this manner, a chimeric founder line animal containing the exogenous nucleic acid (transgene) may be produced.

Preferably, site-specific recombination is employed to integrate the exogenous gene into a specific predetermined site in the animal genome, or to replace an endogenous gene or a portion thereof with the exogenous sequence. Various site-specific recombination systems may be used including those disclosed in Sauer, Curr. Opin. Biotechnol., 5:521-527 (1994); Capecchi, et al., Science, 244:1288-1291 (1989); and Gu et al., Science, 265:103-106 (1994). Specifically, the Cre/lox site-specific recombination system known in the art may be conveniently used which employs the bacteriophage P1 protein Cre recombinase and its recognition sequence loxP. See Rajewsky et al., J. Clin. Invest., 98:600-603 (1996); Sauer, Methods, 14:381-392 (1998); Gu et al., Cell, 73:1155-1164 (1993); Araki et al., Proc. Natl. Acad. Sci. USA, 92:160-164 (1995); Lakso et al., Proc. Natl. Acad. Sci. USA, 89:6232-6236 (1992); and Orban et al., Proc. Natl. Acad. Sci. USA, 89:6861-6865 (1992).

The transgenic animals of the present invention may be transgenic animals that carry a transgene in all cells or mosaic transgenic animals carrying a transgene only in certain cells, e.g., somatic cells. The transgenic animals may have a single copy or multiple copies of a particular transgene.

The founder transgenic animals thus produced may be bred to produce various offsprings. For example, they can be inbred, outbred, and crossbred to establish homozygous lines, heterozygous lines, and compound homozygous or heterozygous lines.

### 8. Pharmaceutical Compositions and Formulations

In another aspect of the present invention, pharmaceutical compositions are also provided containing one or more of the therapeutic agents provided in the present invention as described in Section 6. The compositions are prepared as a pharmaceutical formulation suitable for administration into a patient. Accordingly, the present invention also extends to pharmaceutical compositions, medicaments, drugs or other compositions containing one or more of the therapeutic agent in accordance with the present invention.

For example, such therapeutic agents include, but are not limited to, (1) small organic compounds selected based on the screening methods of the present invention capable of interfering with the interaction between a first protein which is any of the interacting proteins described in the tables and a second protein which is any of the proteins identified in the tables that interacts with the first protein, (2) antisense compounds specifically hybridizable to nucleic acids (gene or mRNA) encoding the first protein (3) antisense compounds specific to the gene or mRNA encoding the second protein, (4) ribozyme compounds specific to nucleic acids (gene or mRNA) encoding the first protein, (5) ribozyme compounds specific to the gene or mRNA encoding the second protein, (6) antibodies immunoreactive with the first protein or the second protein, (7) antibodies selectively immunoreactive with a protein complex of the present invention, (8) small organic compounds capable of binding a protein complex of the present invention, (9) small peptide compounds as described above (optionally linked to a transporter) capable of interacting with the first protein or the second protein, (10) nucleic acids encoding the antibodies or peptides, (11) siRNA compounds specific to the gene or mRNA encoding the first protein, (12) siRNA compounds specific to the gene or mRNA encoding the second protein, etc.

The compositions are prepared as a pharmaceutical formulation suitable for administration into a patient. Accordingly, the present invention also extends to pharmaceutical compositions, medicaments, drugs or other compositions containing one or more of the therapeutic agent in accordance with the present invention.

In the pharmaceutical composition, an active compound identified in accordance with the present invention can be in any pharmaceutically acceptable salt form. As used herein, the term “pharmaceutically acceptable salts” refers to the relatively non-toxic, organic or inorganic salts of the compounds of the present invention, including inorganic or organic acid addition salts of the compound. Examples of such salts include, but are not limited to, hydrochloride salts, sulfate salts, bisulfate salts, borate salts, nitrate salts, acetate salts, phosphate salts, hydrobromide salts, laurylsulfonate salts, glucoheptonate salts, oxalate salts, oleate salts, laurate salts, stearate salts, palmitate salts, valerate salts, benzoate salts, naththylate salts, mesylate salts, tosylate salts, citrate salts, lactate salts, maleate salts, succinate salts, tartrate salts, fumarate salts, and the like. See, e.g., Berge, et al., J. Pharm. Sci., 66:1-19 (1977).

For oral delivery, the active compounds can be incorporated into a formulation that includes pharmaceutically acceptable carriers such as binders (e.g., gelatin, cellulose, gum tragacanth), excipients (e.g., starch, lactose), lubricants (e.g., magnesium stearate, silicon dioxide), disintegrating agents (e.g., alginate, Primogel, and corn starch), and sweetening or flavoring agents (e.g., glucose, sucrose, saccharin, methyl salicylate, and peppermint). The formulation can be orally delivered in the form of enclosed gelatin capsules or compressed tablets. Capsules and tablets can be prepared in any conventional techniques. The capsules and tablets can also be coated with various coatings known in the art to modify the flavors, tastes, colors, and shapes of the capsules and tablets. In addition, liquid carriers such as fatty oil can also be included in capsules.

Suitable oral formulations can also be in the form of suspension, syrup, chewing gum, wafer, elixir, and the like. If desired, conventional agents for modifying flavors, tastes, colors, and shapes of the special forms can also be included. In addition, for convenient administration by enteral feeding tube in patients unable to swallow, the active compounds can be dissolved in an acceptable lipophilic vegetable oil vehicle such as olive oil, corn oil and safflower oil.

The active compounds can also be administered parenterally in the form of solution or suspension, or in lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacterial agents, surfactants, and antioxidants can all be included. For example, useful components include sodium chloride, acetate, citrate or phosphate buffers, glycerin, dextrose, fixed oils, methyl parabens, polyethylene glycol, propylene glycol, sodium bisulfate, benzyl alcohol, ascorbic acid, and the like. The parenteral formulations can be stored in any conventional containers such as vials and ampoules.

Routes of topical administration include nasal, bucal, mucosal, rectal, or vaginal applications. For topical administration, the active compounds can be formulated into lotions, creams, ointments, gels, powders, pastes, sprays, suspensions, drops and aerosols. Thus, one or more thickening agents, humectants, and stabilizing agents can be included in the formulations. Examples of such agents include, but are not limited to, polyethylene glycol, sorbitol, xanthan gum, petrolatum, beeswax, or mineral oil, lanolin, squalene, and the like. A special form of topical administration is delivery by a transdermal patch. Methods for preparing transdermal patches are disclosed, e.g., in Brown, et al., Annual Review of Medicine, 39:221-229 (1988), which is incorporated herein by reference.

Subcutaneous implantation for sustained release of the active compounds may also be a suitable route of administration. This entails surgical procedures for implanting an active compound in any suitable formulation into a subcutaneous space, e.g., beneath the anterior abdominal wall. See, e.g., Wilson et al., J. Clin. Psych. 45:242-247 (1984). Hydrogels can be used as a carrier for the sustained release of the active compounds. Hydrogels are generally known in the art. They are typically made by crosslinking high molecular weight biocompatible polymers into a network that swells in water to form a gel like material. Preferably, hydrogels is biodegradable or biosorbable. For purposes of this invention, hydrogels made of polyethylene glycols, collagen, or poly(glycolic-co-L-lactic acid) may be useful. See, e.g., Phillips et al., J. Pharmaceut. Sci. 73:1718-1720 (1984).

The active compounds can also be conjugated, to a water soluble non-immunogenic non-peptidic high molecular weight polymer to form a polymer conjugate. For example, an active compound is covalently linked to polyethylene glycol to form a conjugate. Typically, such a conjugate exhibits improved solubility, stability, and reduced toxicity and immunogenicity. Thus, when administered to a patient, the active compound in the conjugate can have a longer half-life in the body, and exhibit better efficacy. See generally, Burnham, Am. J. Hosp. Pharm., 15:210-218 (1994). PEGylated proteins are currently being used in protein replacement therapies and for other therapeutic uses. For example, PEGylated interferon (PEG-INTRON A®) is clinically used for treating Hepatitis B. PEGylated adenosine deaminase (ADAGEN®) is being used to treat severe combined immunodeficiency disease (SCIDS). PEGylated L-asparaginase (ONCAPSPAR®) is being used to treat acute lymphoblastic leukemia (ALL). It is preferred that the covalent linkage between the polymer and the active compound and/or the polymer itself is hydrolytically degradable under physiological conditions. Such conjugates known as “prodrugs” can readily release the active compound inside the body. Controlled release of an active compound can also be achieved by incorporating the active ingredient into microcapsules, nanocapsules, or hydrogels generally known in the art.

Liposomes can also be used as carriers for the active compounds of the present invention. Liposomes are micelles made of various lipids such as cholesterol, phospholipids, fatty acids, and derivatives thereof. Various modified lipids can also be used. Liposomes can reduce the toxicity of the active compounds, and increase their stability. Methods for preparing liposomal suspensions containing active ingredients therein are generally known in the art. See, e.g., U.S. Pat. No. 4,522,811; Prescott, Ed., Methods in Cell Biology, Volume XIV, Academic Press, New York, N.Y. (1976).

The active compounds can also be administered in combination with another active agent that synergistically treats or prevents the same symptoms or is effective for another disease or symptom in the patient treated so long as the other active agent does not interfere with or adversely affect the effects of the active compounds of this invention. Such other active agents include but are not limited to anti-inflammation agents, antiviral agents, antibiotics, antifungal agents, antithrombotic agents, cardiovascular drugs, cholesterol lowering agents, anti-cancer drugs, hypertension drugs, and the like.

Generally, the toxicity profile and therapeutic efficacy of the therapeutic agents can be determined by standard pharmaceutical procedures in cell models or animal models, e.g., those provided in Section 7. As is known in the art, the LD₅₀ represents the dose lethal to about 50% of a tested population. The ED₅₀ is a parameter indicating the dose therapeutically effective in about 50% of a tested population. Both LD₅₀ and ED₅₀ can be determined in cell models and animal models. In addition, the IC₅₀ may also be obtained in cell models and animal models, which stands for the circulating plasma concentration that is effective in achieving about 50% of the maximal inhibition of the symptoms of a disease or disorder. Such data may be used in designing a dosage range for clinical trials in humans. Typically, as will be apparent to skilled artisans, the dosage range for human use should be designed such that the range centers around the ED₅₀ and/or IC₅₀, but significantly below the LD₅₀ obtained from cell or animal models.

It will be apparent to skilled artisans that therapeutically effective amount for each active compound to be included in a pharmaceutical composition of the present invention can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like. The amount of administration can also be adjusted as the various factors change over time.

### EXAMPLES

1. Yeast Two-Hybrid System

The principles and methods of the yeast two-hybrid system have been described in detail in The Yeast Two-Hybrid System, Bartel and Fields, eds., pages 183-196, Oxford University Press, New York, N.Y., 1997. The following is thus a description of the particular procedure that we used to identify the interactions of the present invention.

The cDNA encoding the bait protein was generated by PCR from cDNA prepared from a desired tissue. The cDNA product was then introduced by recombination into the yeast expression vector PGBT.Q, which is a close derivative of pGBT.C (See Bartel et al., Nat Genet., 12:72-77 (1996)) in which the polylinker site has been modified to include M13 sequencing sites. The new construct was selected directly in the yeast strain PNY200 for its ability to drive tryptophane synthesis (genotype of this strain: MATα trp1-901 leu2-3, 112 ura3-52 his3-200 ade2 gal4Δ gal80). In these yeast cells, the bait was produced as a C-terminal fusion protein with the DNA binding domain of the transcription factor Gal4 (amino acids 1 to 147). Prey libraries were transformed into the yeast strain BK100 (genotype of this strain: MATα trp1-901 leu2-3,112 ura3-52 his3-200 gal4Δ gal80 LYS2::GAL-HIS3 GAL2-ADE2 met2::GAL7-lacZ), and selected for the ability to drive leucine synthesis. In these yeast cells, each cDNA was expressed as a fusion protein with the transcription activation domain of the transcription factor Gal4 (amino acids 768 to 881) and a 9 amino acid hemagglutinin epitope tag. PNY200 cells (MATOα mating type), expressing the bait, were then mated with BK100 cells (MATa mating type), expressing prey proteins from a prey library. The resulting diploid yeast cells expressing proteins interacting with the bait protein were selected for the ability to synthesize tryptophan, leucine, histidine, and adenine. DNA was prepared from each clone, transformed by electroporation into E. coli strain KC8 (Clontech KC8 electrocompetent cells, Catalog No. C2023-1), and the cells were selected on ampicillin-containing plates in the absence of either tryptophane (selection for the bait plasmid) or leucine (selection for the library plasmid). DNA for both plasmids was prepared and sequenced by the dideoxynucleotide chain termination method. The identity of the bait cDNA insert was confirmed and the cDNA insert from the prey library plasmid was identified using the BLAST program to search against public nucleotide and protein databases. Plasmids from the prey library were then individually transformed into yeast cells together with a plasmid driving the synthesis of lamin and 5 other test proteins, respectively, fused to the Gal4 DNA binding domain. Clones that gave a positive signal in the β-galactosidase assay were considered false-positives and discarded. Plasmids for the remaining clones were transformed into yeast cells together with the original bait plasmid. Clones that gave a positive signal in the β-galactosidase assay were considered true positives.

Bait sequences indicated in the tables were used in the yeast two-hybrid system described above. The isolated prey sequences are summarized in the tables. The GenBank Accession Nos. for the bait and prey proteins are also provided in the tables, upon which the bait and prey sequences are aligned.

2. Production of Antibodies Selectively Immunoreactive with Protein Complex

The PRAK-interacting region of ERK3 and the ERK3-interacting region of PRAK are indicated in Table 1. Both regions, or fragments thereof, are recombinantly-expressed in E. coli. and isolated and purified. Mixing the two purified interacting regions forms a protein complex. A protein complex is also formed by mixing recombinantly expressed intact complete PRAK and ERK3. The two protein complexes are used as antigens in immunizing a mouse. mRNA is isolated from the immunized mouse spleen cells, and first-strand cDNA is synthesized using the mRNA as a template. The V_{H} and V_{K} genes are amplified from the thus synthesized cDNAs by PCR using appropriate primers.

The amplified V_{H} and V_{K} genes are ligated together and subcloned into a phagemid vector for the construction of a phage display library. E. coli. cells are transformed with the ligation mixtures, and thus a phage display library is established. Alternatively, the ligated V_{H} and Vₖ genes are subcloned into a vector suitable for ribosome display in which the V_{H}-Vₖ sequence is under the control of a T7 promoter. See Schaffitzel et al., J Immun. Meth., 231:119-135 (1999).

The libraries are screened for their ability to bind PRAK-ERK3 complex and PRAK or ERK3, alone. Several rounds of screening are generally performed. Clones corresponding to scFv fragments that bind the PRAK-ERK3 complex, but not isolated PRAK or ERK3 are selected and purified. A single purified clone is used to prepare an antibody selectively immunoreactive with the complex comprising PRAK and ERK3. The antibody is then verified by an immunochemistry method such as RIA and ELISA.

In addition, the clones corresponding to scFv fragments that bind the complex comprising PRAK and ERK3, and also bind isolated PRAK and/or ERK3 may be selected. The scFv genes in the clones are diversified by mutagenesis methods such as oligonucleotide-directed mutagenesis, error-prone PCR (See Lin-Goerke et al., Biotechniques, 23:409 (1997)), dNTP analogues (See Zaccolo et al., J. Mol. Biol., 255:589 (1996)), and other methods. The diversified clones are further screened in phage display or ribosome display libraries. In this manner, scFv fragments selectively immunoreactive with the complex comprising PRAK and ERK3 may be obtained.

3. Yeast Screen to Identify Small Molecule Inhibitors of the Interaction Between PRAK and ERK3

Beta-galactosidase is used as a reporter enzyme to signal the interaction between yeast two-hybrid protein pairs expressed from plasmids in Saccharomyces cerevisiae. Yeast strain MY209 (ade2 his3 leu2 trp1 cyh2 ura3::GAL1p-lacZ gal4 gal80 lys2::GAL1p-HIS3) bearing one plasmid with the genotype of LEU2 CEN4 ARS1 ADH1p-SV40NLS-GAL4 (768-881)-ERK3-PGK1t AmpR ColE1_ori, and another plasmid having a genotype of TRP1 CEN4 ARS ADH1p-GAL4(1-147)—PRAK-ADH1t AmpR ColE1_ori is cultured in synthetic complete media lacking leucine and tryptophan (SC-Leu-Trp) overnight at 30° C. The PRAK and ERK3 nucleic acids in the plasmids can code for the full-length PRAK and ERK3 proteins, respectively, or fragments thereof. This culture is diluted to 0.01 OD₆₃₀ units/ml using SC-Leu-Trp media. The diluted MY209 culture is dispensed into 96-well microplates. Compounds from a library of small molecules are added to the microplates; the final concentration of test compounds is approximately 60 μM. The assay plates are incubated at 30° C. overnight.

The following day an aliquot of concentrated substrate/lysis buffer is added to each well and the plates incubated at 37° C. for 1-2 hours. At an appropriate time an aliquot of stop solution is added to each well to halt the beta-galactosidase reaction. For all microplates an absorbance reading is obtained to assay the generation of product from the enzyme substrate. The presence of putative inhibitors of the interaction between PRAK and ERK3 results in inhibition of the beta-galactosidase signal generated by MY209. Additional testing eliminates compounds that decreased expression of beta-galactosidase by affecting yeast cell growth and non-specific inhibitors that affected the beta-galactosidase signal generated by the interaction of an unrelated protein pair.

Once a hit, i.e., a compound which inhibits the interaction between the interacting proteins, is obtained, the compound is identified and subjected to further testing wherein the compounds are assayed at several concentrations to determine an IC₅₀ value, this being the concentration of the compound at which the signal seen in the two-hybrid assay described in this Example is 50% of the signal seen in the absence of the inhibitor.

4. Enzyme-Linked Immunosorbent Assay (ELISA)

pGEX5X-2 (Amersham Biosciences; Uppsala, Sweden) is used for the expression of a GST-ERK3 fusion protein. The pGEX5X-2-ERK3 construct is transfected into Escherichia coli strain DH5α (Invitrogen; Carlsbad, Calif.) and fusion protein is prepared by inducing log phase cells (O.D. 595=0.4) with 0.2 mM isopropyl-β-D-thiogalactopyranoside (IPTG). Cultures are harvested after approximately 4 hours of induction, and cells pelleted by centrifugation. Cell pellets are resuspended in lysis buffer (1% nonidet P-40 [NP-40], 150 mM NaCl, 10 mM Tris pH 7.4, 1 mM ABESF [4-(2-aminoethyl)benzenesulfonyl fluoride]), lysed by sonication and the lysate cleared of insoluble materials by centrifugation. Cleared lysate is incubated with Glutathione Sepharose beads (Amersham Biosciences; Uppsala, Sweden) followed by thorough washing with lysis buffer. The GST-ERK3 fusion protein is then eluted from the beads with 5 mM reduced glutathione. Eluted protein is dialyzed against phosphate buffer saline (PBS) to remove the reduced glutathione.

A stable Drosophila Schneider 2 (S2) myc-PRAK expression cell line is generated by transfecting S2 cells with pCoHygro (Invitrogen; Carlsbad, Calif.) and an expression vector that directs the expression of the myc-PRAK fusion protein. Briefly, S2 cells are washed and re-suspended in serum free Express Five media (Invitrogen; Carlsbad, Calif.). Plasmid/liposome complexes are then added (NovaFECTOR, Venn Nova; Pompano Beach, Fla.) and allowed to incubate with cells for 12 hours under standard growth conditions (room temperature, no CO₂ buffering). Following this incubation period fetal bovine serum is added to a final concentration of 20% and cells are allowed to recover for 24 hours. The media is replaced and cells are grown for an additional 24 hours. Transfected cells are then selected in 350 μg/ml hygromycin for three weeks. Expression of myc-PRAK is confirmed by Western blotting. This cell line is referred to as S2-myc-PRAK.

GST-ERK3 fusion protein is immobilized to wells of an ELISA plate as follows: Nunc Maxisorb 96 well ELISA plates (Nalge Nunc International; Rochester, N.Y.) are incubated with 100 μl of 10 μg/ml of GST-ERK3 in 50 mM carbonate buffer (pH 9.6) and stored overnight at 4° Celsius. This plate is referred to as the ELISA plate.

A compound dilution plate is generated in the following manner. In a 96 well polypropylene plate (Greiner, Germany) 50 μl of DMSO is pipetted into columns 2-12. In the same polypropylene plate pipette, 10 μl of each compound being tested for its ability to modulate protein-protein interactions is plated in the wells of column 1 followed by 90 μl of DMSO (final volume of 100 μl). Compounds selected from primary screens or from virtual screening, or designed based on the primary screen hits are then serially diluted by removing 50 μl from column 1 and transferring it to column 2 (50:50 dilution). Serial dilutions are continued until column 10. This plate is termed the compound dilution plate.

Next, 12 μl from each well of the compound dilution plate is transferred into its corresponding well in a new polypropylene plate. 108 μl of S2-myc-PRAK-containing lysate (1×10⁶ cell equivalents/ml) in phosphate buffered saline is added to all wells of columns 1-11. 108 μl of phosphate buffered saline without lysate is added into all wells of column 12. The plate is then mixed on a shaker for 15 minutes. This plate is referred to as the compound preincubation plate.

The ELISA plate is emptied of its contents and 400 μl of Superblock (Pierce Endogen; Rockford, Ill.) is added to all the wells and allowed to sit for 1 hour at room temperature. 100 μl from all columns of the compound preincubation plate are transferred into the corresponding wells of the ELISA binding plate. The plate is then covered and allowed to incubate for 1.5 hours room temperature.

The interaction of the myc-tagged PRAK with the immobilized GST-ERK3 is detected by washing the ELISA plate followed by an incubation with 100 μl/well of 1 μg/ml of mouse anti-myc IgG (clone 9E10; Roche Applied Science; Indianapolis, Ind.) in phosphate buffered saline. After 1 hour at room temperature, the plates are washed with phosphate buffered saline and incubated with 100 μl/well of 250 ng/ml of goat anti-mouse IgG conjugated to horseradish peroxidase in phosphate buffer saline. Plates are then washed again with phosphate buffered saline and incubated with the fluorescent substrate solution Quantiblu (Pierce Endogen; Rockford, Ill.). Horseradish peroxidase activity is then measured by reading the plates in a fluorescent plate reader (325 nm excitation, 420 nm emission).

5. Effects of Antisense Inhibitors on Protein Expression

The effects of antisense inhibitors on protein expression can be measured by a variety of methods known in the art. A preferred method is to measure mRNA levels using real-time quantitative polymerase chain reaction (PCR) methods. Real-time PCR can be performed using the ABI PRISM™ 7700 Sequence Detection System according to the manufacturer's instructions. The ABI PRISM™ 7700 Sequence Detection System is available from PE-APPLIED Biosystems, Foster City, Calif.

Other methods of measuring mRNA levels may also be used to determine the effects of anitisense inhibitors on proteins. For example competitive PCR and Northern blot analysis are well known in the art and may be performed to determine mRNA levels. Specifically, methods of RNA isolation and Northern blot analysis may be performed according to Ausubel, F. M. et al., Current Protocols in Molecular Biology, Volume 1, pp. 4.1.1-4.2.9 and 4.5.1-4.5.3, John Wiley & Sons, Inc., 1993.

The effects of antisense inhibitors on protein expression may also be determined by measuring protein levels of the proteins of interest. Various methods known in the art may be used, such as immunoprecipitation, Western blot analysis, ELISA, or fluorescence-activated cell sorting (FACS). Antibodies to the proteins of interest are often commercially available, and may be found by such sources as the MSRS catalogue of antibodies (Aerie Corporation, Birmingham, Mich.). Antibodies can also be prepared through conventional antibody generation methods, such as found in Ausubel, F. M. et al., Current Protocols in Molecular Biology, Volume 2, pp. 11.12.1-11.12.9 and 11.4.1-11.11.5 John Wiley & Sons, Inc., 1997. Furthermore, immunoprecipitation analysis can be performed according to Ausubel, F. M. et al., Current Protocols in Molecular Biology, Volume 2, pp. 10.16.1-10.16.11, John Wiley & Sons, Inc., 1997, and ELISA can be performed according to Ausubel, F. M. et al., Current Protocols in Molecular Biology, Volume 2, pp. 11.1.1-11.2.22, John Wiley & Sons, Inc., 1997 or as described in Example 4, above.

6. Cell-Based TNF-α Secretion Assay to Identify Anti-Inflammatory Compounds

Anti-inflammatory compounds can be identified in a cell-based assay by their ability to inhibit the secretion of the cytokine TNF-α from activated T cells. T cells play a central role in raising an inflammatory response upon stimulation by specific antigens. In this assay, Jurkat T leukemia cells are separated into control and test groups. Test compounds are added to test groups of Jurkat T leukemia cells. T cell receptor activation is achieved in both test and control groups by combined stimulation with anti CD3 and anti CD28 antibodies. TNF-α secretion by the cells is then measured by a commercially available ELISA kit and the amount of TNF-α secreted in test cells is compared to the amount of TNF-α secreted in control cells. Test cells showing decreased TNF-α secretion compared to control cells indicate that the test compound has anti-inflammatory effects.

7. Cell-Based IL-2 Secretion Assay to Identify Anti-Inflammatory Compounds

Anti-inflammatory compounds can be identified in a cell-based assay by their ability to inhibit the secretion of the cytokine IL-2 from activated T cells. Jurkat T leukemia cells are separated into control and test groups in which test compounds are added to test groups of Jurkat T leukemia cells. T cell receptor activation is achieved in both test and control groups by combined stimulation with anti CD3 and anti CD28 antibodies. After T cell receptor activation, IL-2 secretion by the cells is measured by a commercially available ELISA kit and the amount of IL-2 secreted in test cells is compared to the amount of IL-2 secreted in control cells. Test cells showing decreased IL-2 secretion compared to control cells indicate that the test compound has anti-inflammatory effects.

8. Animal-Based Assay to Identify Compounds with Anti-Inflammatory Effects

Anti-inflammatory compounds can be identified in the carrageenan-induced foot paw edema model (See Winter et al., Proc. Soc. Exp. Biol. Med. 111:544-547 (1962)). Male Sprague-Dawley rats are obtained and separated into control and test groups. Test groups are dosed orally with the test compound. One hour later, 0.1 mL of a solution containing 1% carrageenan and 0.9% sterile saline is injected to the right hind foot pad of both control and test rats. Paw volume is measured three hours after injection with a displacement plethysmometer. The degree of swelling after injection with carrageenan is a measure of the inflammatory response. Accordingly, test rats that show a lower paw volume increase indicate that the administered test compound has anti-inflammatory effects.

9. Assay to Identify Compounds with Anti-Inflammatory Effects in Arthritic Animals

Anti-inflammatory compounds can be identified by the rat adjuvant induced arthritis assay (Jaffee et al., Agents Actions 27:344-346 (1988)). In this assay, commercially available male Lewis rats (125-150 g) are obtained. Arthritis is induced in the rats by injecting the rats with 1 mg of Mycobacterium butyricum in 50 μL of mineral oil into the right the right hind foot pad. 14 days after injection the contralateral left foot volume is measured with a displacement plethsmometer. Rats with paw volumes 0.30 mL greater than normal paws are selected and randomly separated into control and test groups. For ten days test group rats are orally administered 1 mL of a suspension of 0.5% methyl cellulose and 0.025% Tween-20 containing a test compound. At the end of the 10 day treatment, the paw volume of control and test group rats are measured and compared. A reduction in paw volume of test group rats compared to control group rats indicates that the administered test compound has an anti-inflammatory effect.

Although the foregoing invention has been described in some detail by way of illustration and example for purposes of clarity of understanding, it will be obvious that certain changes and modifications may be practiced within the scope of the appended claims.

In various parts of this disclosure, certain publications or patents are discussed or cited. The mere discussion of, or reference to, such publications or patents is not intended as admission that they are prior art to the present invention.

1. A method for modulating, in a host cell, a protein-protein interaction between a first protein which is PRAK and a second protein which is ERK3, said method comprising:
administering to said cell a compound capable of modulating said protein-protein interaction.

2. The method of claim 1, wherein said compound is capable of interfering with the interaction between said first protein and said second protein.

3. The method of claim 1, wherein said compound is capable of binding at least one of said first protein and said second protein.

4. The method of claim 1, wherein said compound comprises a peptide having a contiguous amino acid sequence of ERK3 and is capable of binding PRAK.

5. The method of claim 1, wherein said compound comprises a peptide capable of binding PRAK and has an amino acid sequence that is at least 75% identical to a contiguous amino acid sequence of ERK3.

6. The method of claim 1, wherein said compound comprises a peptide having a contiguous amino acid sequence of PRAK and is capable of binding ERK3.

7. The method of claim 1, wherein said compound comprises a peptide capable of binding ERK3 and has an amino acid sequence that is at least 75% identical to a contiguous amino acid sequence of PRAK.

8. The method of claim 1, wherein said compound is an antibody immunoreactive with PRAK or ERK3.

9. The method of claim 1, wherein said compound is a nucleic acid encoding an antibody immunoreactive with PRAK or ERK3.

10. The method of claim 1, wherein said compound is selected from the group consisting of: (1) an antisense compound and/or ribozyme capable of specifically hybridizing to a nucleic acid having a sequence encoding ERK3, and (2) an antisense compound and/or ribozyme capable of specifically hybridizing to a PRAK nucleic acid.

11. The method of claim 1, wherein said compound is a peptide capable of interfering with the interaction between said first protein and said second protein, wherein said peptide is associated with a transporter capable of increasing cellular uptake of said peptide.

12. The method of claim 11, wherein said peptide is covalently linked to said transporter which is selected from the group consisting of penetratins, l-Tat₄₉₋₅₇, d-Tat₄₉₋₅₇, retro-inverso isomers of l- or d-Tat₄₉₋₅₇, L-arginine oligomers, D-arginine oligomers, L-lysine oligomers, D-lysine oligomers, L-histidine oligomers, D-histidine oligomers, L-ornithine oligomers, D-ornithine oligomers, short peptide sequences derived from fibroblast growth factor, Galparan, and HSV-1 structural protein VP22, and peptoid analogs thereof.

13. A method for treating inflammation and inflammatory disorders (e.g., asthma, rheumatoid arthritis, juvenile chronic arthritis, myositis, Chron's disease, gastritis, colitis, ulcerative colitis, inflammatory bowel disease, proctitis, pelvic inflammatory disease, systemic lupus erythematosus, rhinitis, conjunctivitis, scleritis, chronic inflammatory polyneuropathy, Tertiary Lyme disease, psoriasis, dermatitis, eczema, etc.), comprising:
identifying a patient in need of treatment of the disease; and
administering to a patient in need of such treatment a compound capable of interfering with the interaction between a first protein which is PRAK and a second protein which is ERK3.

14. The method of claim 13, wherein said compound is capable of binding at least one of said first protein and said second protein.

15. The method of claim 13, wherein said compound comprises a peptide having a contiguous amino acid sequence of ERK3 and is capable of binding PRAK.

16. The method of claim 15, wherein said peptide is associated with a transporter capable of increasing the cellular uptake of said peptide.

17. The method of claim 13, wherein said compound comprises a peptide having a contiguous amino acid sequence of PRAK and is capable of binding ERK3.

18. The method of claim 17, wherein said peptide is covalently linked to a transporter capable of increasing the cellular uptake of said peptide.

19. The method of claim 17, wherein said peptide is covalently linked to said transporter which is selected from the group consisting of penetratins, l-Tat₄₉₋₅₇, d-Tat₄₉₋₅₇, retro-inverso isomers of l- or d-Tat₄₉₋₅₇, L-arginine oligomers, D-arginine oligomers, L-lysine oligomers, D-lysine oligomers, L-histidine oligomers, D-histidine oligomers, L-ornithine oligomers, D-ornithine oligomers, short peptide sequences derived from fibroblast growth factor, Galparan, and HSV-1 structural protein VP22, and peptoid analogs thereof.

20. The method of claim 13, wherein said compound is selected from the group consisting of: (1) an antisense compound and/or ribozyme capable of specifically hybridizing to a nucleic acid having a sequence encoding ERK3, and (2) an antisense compound and/or ribozyme capable of specifically hybridizing to a PRAK nucleic acid.
